(19) United States
(12) Patent Application Publication
(10) Pub. No.: US 2022/0251077 A1
(43) Pub. Date: Aug. 11, 2022

(54) ORGANIC COMPOUND AND ORGANIC ELECTROLUMINESCENCE DEVICE USING THE SAME

(71) Applicants: FENG-WEN YEN, TAIPEI (TW); LI-CHIEH CHUANG, NANTOU (TW)

(72) Inventors: FENG-WEN YEN, TAIPEI (TW); LI-CHIEH CHUANG, NANTOU (TW)

(73) Assignee: LUMINESCENCE TECHNOLOGY CORP., Hsin-Chu (TW)

(21) Appl. No.: 17/248,775

(22) Filed: Feb. 8, 2021

(51) Int. Cl. C07D 419/04; C07D 419/14; C07D 421/04; H01L 51/00

(52) U.S. Cl. CPC C07D 419/04; C07D 419/14; C07D 421/04; H01L 51/0067; H01L 51/5072; H01L 51/0054; H01L 51/0072; H01L 51/0094; H01L 51/0056

(57) ABSTRACT

An organic compound is described. An organic electroluminescence device comprises the organic compound as a host or an electron transfer layer. The organic compound of the following formula may lower a driving voltage or increase a current efficiency or a half-life of the organic electroluminescence device.

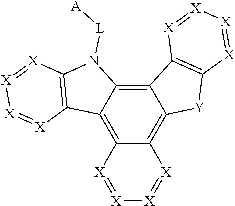

The same definition as described in the present invention.

### FIELD

The present invention relates generally to a compound, and, more specifically, to an organic electroluminescence (herein after referred to as organic EL) device using the compound.

### BACKGROUND

An organic electroluminescence (organic EL) devices, i.e., organic light-emitting diodes (OLEDs) that make use of organic compounds, are becoming increasingly desirable than before. One of the organic compounds has the following formula:

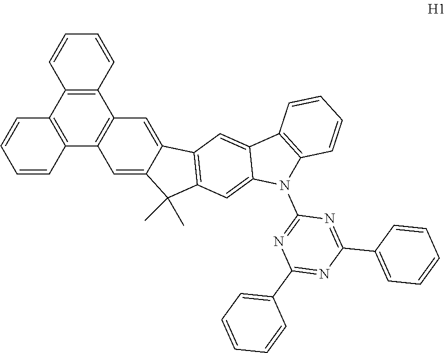 [SMILES: CC1(C)c2cc3c4ccccc4c4ccccc4c3cc2-c2cc3c4ccccc4n(-c4nc(-c5ccccc5)nc(-c5ccccc5)n4)c3cc21]

An organic EL device is a light-emitting diode (LED) in which the light emitting layer is a film made from organic compounds, which emits light in response to an electric current. The light emitting layer containing the organic compound is sandwiched between two electrodes. The organic EL device is applied to flat panel displays due to its high illumination, low weight, ultra-thin profile, self-illumination without back light, low power consumption, wide viewing angle, high contrast, simple fabrication methods and rapid response time.

However, there is still a need for improvement in the case of use of those organic materials in an organic EL device of some prior art displays, for example, in relation to the lifetime, current efficiency or driving voltage of the organic EL device.

### SUMMARY

According to the reasons described above, an object of the present invention is to resolve the problems of prior arts and to offer a novel compound.

Another object of the invention is to provide an organic EL device using the compound. The organic EL device of the present invention may operate under reduced voltage, or may exhibit higher current efficiency or longer lifetime.

The present invention discloses an organic compound of formula (1):

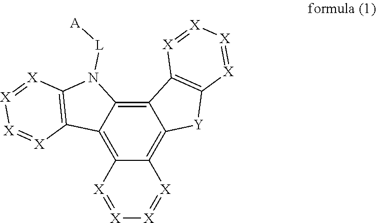

wherein Y is selected from the group consisting of O, S, Se, NR₁, CR₂R₃ and SiR₄R₅; X is CR₆ or N, and at least one X is N, and two adjacent X can form a five-membered ring, a six-membered ring or a combination thereof; L represents a single bond, a substituted or unsubstituted divalent arylene group having 6 to 30 ring carbon atoms, or a substituted or unsubstituted divalent heteroarylene group having 6 to 30 ring carbon atoms; A represents a substituted or unsubstituted aryl group having 6 to 30 ring carbon atoms, or a substituted or unsubstituted heteroaryl group having 5 to 30 ring carbon atoms; R₁ to R6 are independently selected from the group consisting of a hydrogen atom, a substituted or unsubstituted alkyl group having 1 to 20 carbon atoms, a substituted or unsubstituted aryl group having 6 to 30 carbon atoms, a substituted or unsubstituted aralkyl group having 6 to 30 carbon atoms and a substituted or unsubstituted heteroaryl group having 3 to 30 carbon atoms.

The present invention further discloses an organic EL device. The organic EL device may comprise an anode, a cathode and one or more organic layers formed between the anode and the cathode. At least one of the organic layers comprises the organic compound of formula (1).

### BRIEF DESCRIPTION OF THE DRAWINGS

FIG. 1 is a cross-sectional view of a first organic EL device according to a second embodiment of the present invention.

FIG. 2 is a cross-sectional view of an organic EL device without the host 340C of FIG. 1.

FIG. 3 is a cross-sectional view of a second organic EL device according to a third embodiment of the present invention.

FIG. 4 is a cross-sectional view of a third organic EL device according to a fourth embodiment of the present invention.

### DETAILED DESCRIPTION

Generally, an external voltage is applied across the organic EL device, electrons and holes are injected from the cathode and the anode, respectively. Electrons will be injected from a cathode into a LUMO (lowest unoccupied molecular orbital) and holes will be injected from an anode into a HOMO (highest occupied molecular orbital). Subsequently, the electrons recombine with holes in the light emitting layer to form excitons and then emit light. When luminescent molecules absorb energy to achieve an excited state, the exciton may either be in a singlet state or a triplet state, depending on how the spins of the electrons and holes have been combined.

The terms “halogen” and “halide” are used interchangeably and refer to fluorine, chlorine, bromine, and iodine.

The term “alkyl” or “alkyl group” refers to and includes both straight and branched chain alkyl radicals. Preferred alkyl groups are those containing from 1 to 20 carbon atoms, more preferably 1 to 15 carbon atoms. Suitable alkyl groups include methyl, ethyl, propyl, 1-methylethyl, butyl, 1-methylpropyl, 2-methylpropyl, pentyl, 1-methylbutyl, 2-methylbutyl, 3-methylbutyl, 1,1-dimethylpropyl, 1,2-dimethylpropyl, 2,2-dimethylpropyl, and the like. Additionally, the alkyl group is optionally substituted.

The term “aryl” or “aryl group” refers to and includes both single-ring aromatic hydrocarbonyl groups and polycyclic aromatic ring systems. The polycyclic rings may have two, three, four or more rings in which two carbons are common to two adjoining rings (the rings are “fused”) wherein at least one of the rings is an aromatic hydrocarbonyl group, e.g., the other rings can be cycloalkyls, cycloalkenyls, aryl, heterocycles, and/or heteroaryls. Preferred aryl groups are those containing 6 to 30 carbon atoms, preferably 6 to 20 carbon atoms, more preferably 6 to 12 carbon atoms. Especially preferred is an aryl group having 6 carbons, 10 carbons or 12 carbons. Suitable aryl groups include phenyl, biphenyl, triphenyl, triphenylene, tetraphenylene, naphthalene, anthracene, phenalene, phenanthrene, fluorene, pyrene, chrysene, perylene, and azulene, preferably phenyl, biphenyl, triphenyl, triphenylene, and naphthalene. Additionally, the aryl group is optionally substituted.

The terms “aralkyl”, “aralkyl group” or “arylalkyl” are used interchangeably and refer to an alkyl group that is substituted with an aryl group. Preferred aralkyl groups are those containing 6 to 30 carbon atoms. Additionally, the aralkyl group is optionally substituted.

The term “heteroaryl” or “heteroaryl group” refers to and includes both single-ring aromatic groups and polycyclic aromatic ring systems that include at least one heteroatom. The heteroatoms include, but are not limited to O, S, N, P, B, Si, and Se. In many instances, O, S, or N are the preferred heteroatoms. Hetero-single ring aromatic systems are preferably single rings with 5 or 6 ring atoms, and the ring can have from one to six heteroatoms. The hetero-polycyclic ring systems can have two or more rings in which two atoms are common to two adjoining rings (the rings are “fused”) wherein at least one of the rings is a heteroaryl, e.g., the other rings can be cycloalkyls, cycloalkenyls, aryl, heterocycles, and/or heteroaryls. The hetero-polycyclic aromatic ring systems can have from one to six heteroatoms per ring of the polycyclic aromatic ring system. Preferred heteroaryl groups are those containing 3 to 30 carbon atoms, preferably 3 to 20 carbon atoms, more preferably 3 to 12 carbon atoms. Suitable heteroaryl groups include pyrimidine, triazine, quinazoline, benzoquinazoline, phenylquinazoline, dibenzothiophene, dibenzofuran, dibenzoselenophene, furan, thiophene, benzofuran, benzothiophene, benzoselenophene, carbazole, indolocarbazole, pyridylindole, pyrrolodipyridine, pyrazole, imidazole, triazole, oxazole, thiazole, oxadiazole, oxatriazole, dioxazole, thiadiazole, pyridine, pyridazine, pyrazine, oxazine, oxathiazine, oxadiazine, indole, benzimidazole, indazole, indoxazine, benzoxazole, benzisoxazole, benzothiazole, quinoline, isoquinoline, cinnoline, quinoxaline, naphthyridine, phthalazine, pteridine, xanthene, acridine, phenazine, phenothiazine, phenoxazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group is optionally substituted.

The terms “R₁” to “R₁₈” may independently be H (hydrogen) or a substituent selected from the group consisting of deuterium, halogen, alkyl, cycloalkyl, heteroalkyl, heterocycloalkyl, aralkyl, alkoxy, aryloxy, amino, silyl, alkenyl, cycloalkenyl, heteroalkenyl, alkynyl, aryl, heteroaryl, and combinations thereof. R₁ to R₁₈ may preferably and independently be hydrogen or a substituent selected from the group consisting of hydrogen, alkyl, aryl, aralkyl, heteroaryl, and combinations thereof.

The “aza” designation in the fragments described herein, i.e. aza-dibenzofuran, aza-dibenzothiophene, etc. means that one or more of the C—H groups in the respective fragment can be replaced by a nitrogen atom, for example, and without any limitation, azatriphenylene encompasses both dibenzo[fh]quinoxaline and dibenzo[fh]quinoline. One of ordinary skill in the art can readily envision other nitrogen analogs of the aza-derivatives described above, and all such analogs are intended to be encompassed by the terms as set forth herein.

The terms alkyl, cycloalkyl, heteroalkyl, heterocycloalkyl, alkenyl, cycloalkenyl, heteroalkenyl, alkynyl, aralkyl, heterocyclic group, aryl, and heteroaryl, as used herein, are independently unsubstituted, or independently substituted, with one or more general substituents.

In many instances, the general substituents are selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof.

In some instances, the preferred general substituents are selected from the group consisting of deuterium, fluorine, alkyl, cycloalkyl, heteroalkyl, alkoxy, aryloxy, amino, silyl, alkenyl, cycloalkenyl, heteroalkenyl, aryl, heteroaryl, nitrile, isonitrile, sulfanyl, and combinations thereof.

In some instances, the preferred general substituents are selected from the group consisting of deuterium, fluorine, alkyl, cycloalkyl, alkoxy, aryloxy, amino, silyl, aryl, heteroaryl, sulfanyl, and combinations thereof.

In yet other instances, the more preferred general substituents are selected from the group consisting of deuterium, fluorine, alkyl, cycloalkyl, aryl, heteroaryl, and combinations thereof.

The terms “substituted” and “substitution” refer to a substituent other than H that is bonded to the relevant position, e.g., a carbon or nitrogen. For example, when R₁ represents mono-substitution, then one R₁ must be other than H (i.e., a substitution). Similarly, when R₁ represents di-substitution, then two of R₁ must be other than H. Similarly, when R¹ represents no substitution, R₁, for example, can be a hydrogen for available valencies of ring atoms, as in carbon atoms for benzene and the nitrogen atom in pyrrole, or simply represents nothing for ring atoms with fully filled valencies, e.g., the nitrogen atom in pyridine. The maximum number of substitutions possible in a ring structure will depend on the total number of available valencies in the ring atoms.

As used herein, “combinations thereof” indicates that one or more members of the applicable list are combined to form a known or chemically stable arrangement that one of ordinary skill in the art can envision from the applicable list. For example, two adjacent alkyls can form a five-membered ring, a six-membered ring or a combination thereof. Moreover, an alkyl and deuterium can be combined to form a partial or fully deuterated alkyl group; a halogen and alkyl can be combined to form a halogenated alkyl substituent; and a halogen, alkyl, and aryl can be combined to form a halogenated arylalkyl. In one instance, the term substitution includes a combination of two to four of the listed groups. In another instance, the term substitution includes a combination of two to three groups. In yet another instance, the term substitution includes a combination of two groups. Preferred combinations of substituent groups are those that contain up to fifty atoms that are not hydrogen or deuterium, or those which include up to forty atoms that are not hydrogen or deuterium, or those that include up to thirty atoms that are not hydrogen or deuterium. In many instances, a preferred combination of substituent groups will include up to twenty atoms that are not hydrogen or deuterium.

It is to be understood that when a molecular fragment is described as being a substituent or otherwise attached to another moiety, its name may be written as if it were a fragment (e.g., phenyl, phenylene, naphthyl, dibenzofuryl) or as if it were the whole molecule (e.g., benzene, naphthalene, dibenzofuran). As used herein, these different ways of designating a substituent or attached fragment are considered to be equivalent.

In a first embodiment of the present invention, an organic compound which can be used as the host material of the light emitting layer in the organic EL device is disclosed. The organic compound may be represented by the following formula (1):

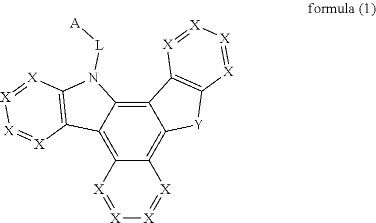

Y is selected from the group consisting of O, S, Se, NR₁, CR₂R₃ and SiR₄R₅; X is CR₆ or N, and at least one X is N, and two adjacent X can form a five-membered or six-membered ring; L represents a single bond, a substituted or unsubstituted divalent arylene group having 6 to 30 ring carbon atoms, or a substituted or unsubstituted divalent heteroarylene group having 6 to 30 ring carbon atoms; A represents a substituted or unsubstituted divalent arylene group having 6 to 30 ring carbon atoms, or a substituted or unsubstituted divalent heteroarylene group having 5 to 30 ring carbon atoms; R₁ to R₆ are independently selected from the group consisting of H, a substituted or unsubstituted alkyl group having 1 to 20 carbon atoms, a substituted or unsubstituted aryl group having 6 to 30 carbon atoms, a substituted or unsubstituted aralkyl group having 6 to 30 carbon atoms and a substituted or unsubstituted heteroaryl group having 3 to 30 carbon atoms.

Preferably, at least one of the following may be true:

only one X is N;

at least eleven X are not N; and

at least eleven X are CH.

The alkyl group, aralkyl group, aryl group, or heteroaryl group may be substituted by a halogen, an alkyl group, an aryl group, or a heteroaryl group.

A may be selected from the group consisting of pyrimidinyl, triazinyl, fluorenyl, quinazolinyl, benzoquinazolinyl, phenylquinazolinyl,

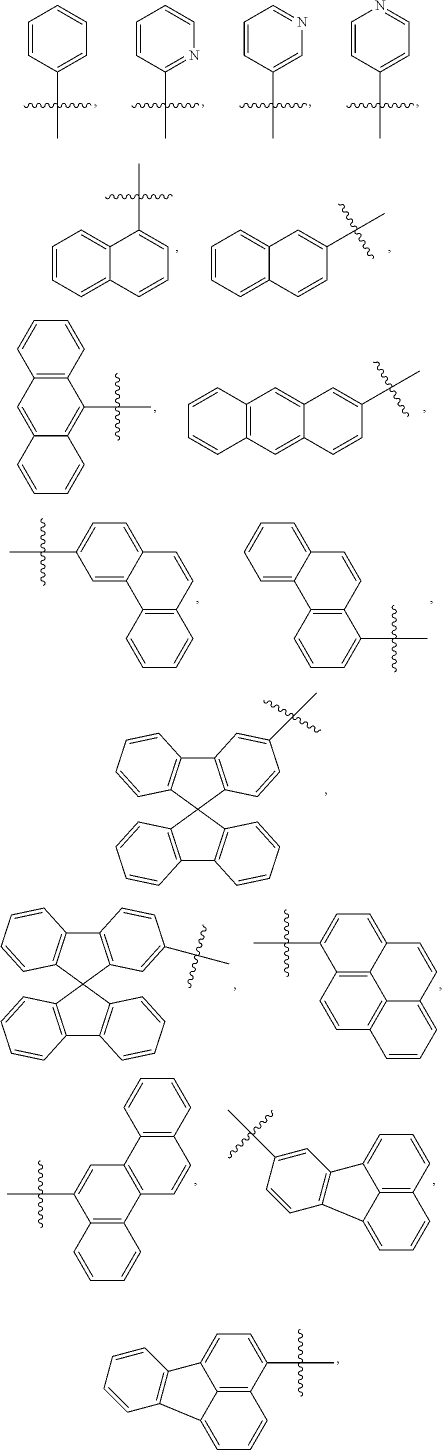

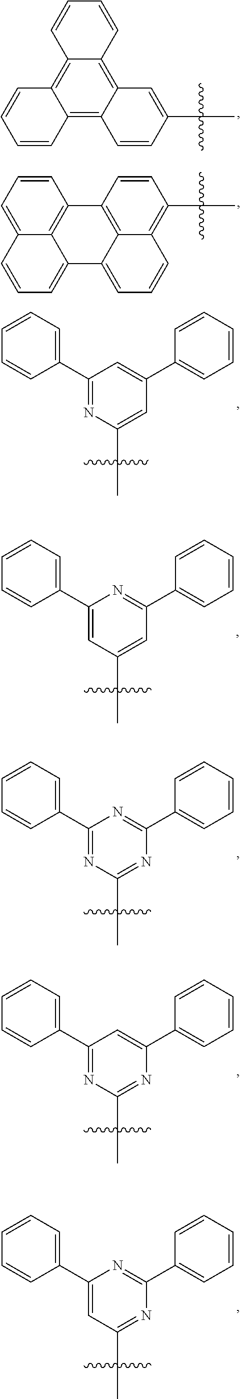

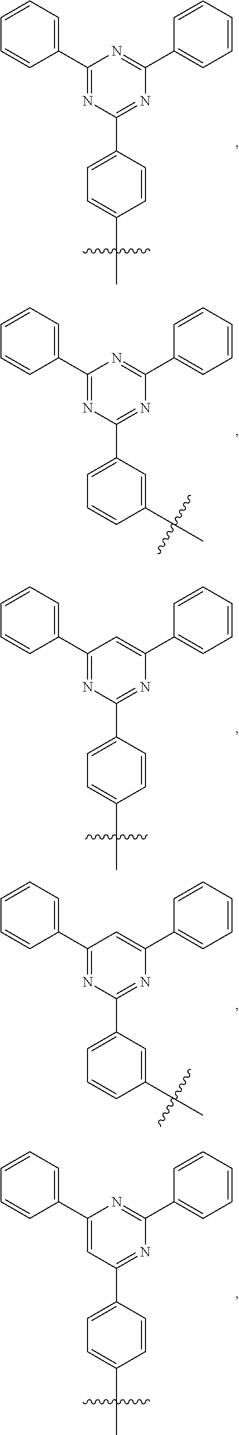 [SMILES: *c1ccc(-c2nc(-c3ccccc3)nc(-c3ccccc3)n2)cc1 ; *c1cccc(-c2nc(-c3ccccc3)nc(-c3ccccc3)n2)c1 ; *c1ccc(-c2nc(-c3ccccc3)cc(-c3ccccc3)n2)cc1 ; *c1cccc(-c2nc(-c3ccccc3)cc(-c3ccccc3)n2)c1 ; *c1ccc(-c2cc(-c3ccccc3)nc(-c3ccccc3)n2)cc1]

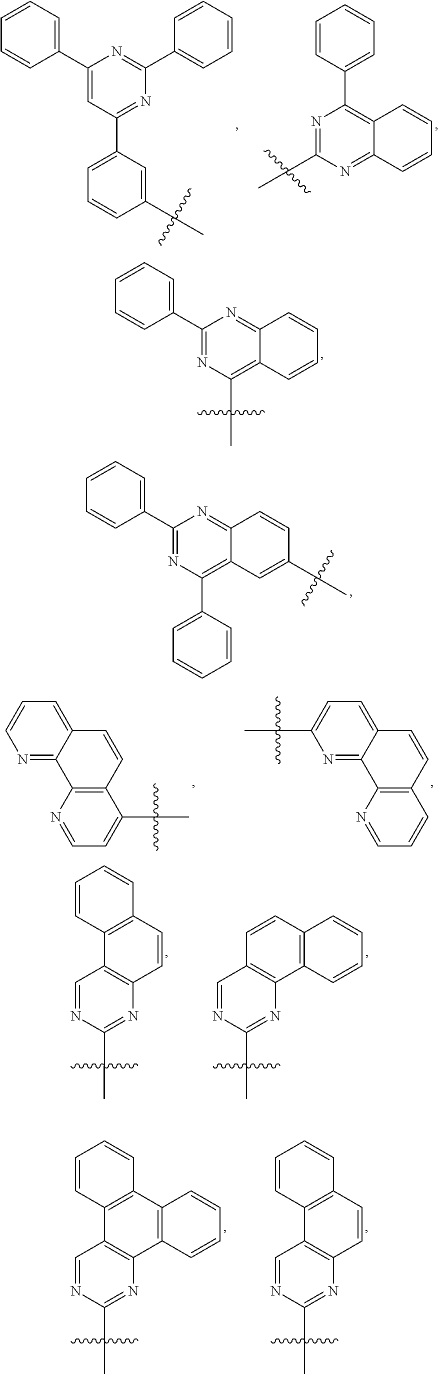

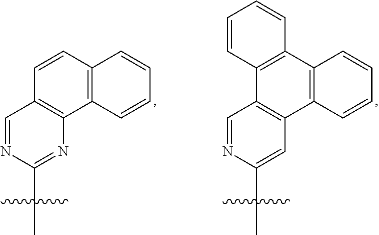 [SMILES: *c1ncc2ccc3ccccc3c2n1 ; *c1cc2c3ccccc3c3ccccc3c2cn1]

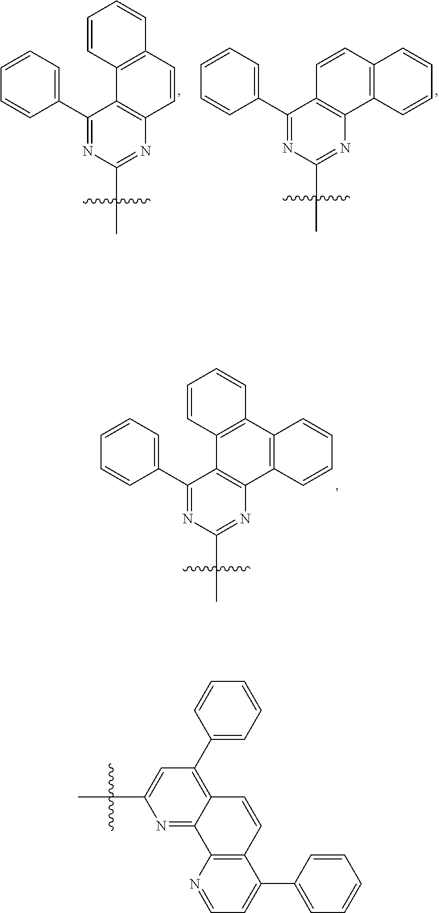

and combinations thereof.

The organic compound may be represented by the following formula (2):

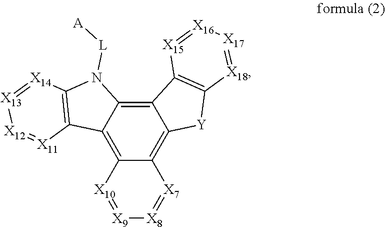

Y is selected from the group consisting of O, S, Se, NR₁, CR₂R₃ and SiR₄R₅;

L represents a single bond, a substituted or unsubstituted divalent arylene group having 6 to 30 ring carbon atoms, or a substituted or unsubstituted divalent heteroarylene group having 6 to 30 ring carbon atoms.

X₇ is N or CR₇;

wherein X₈ is N or CR₈;

wherein X₉ is N or CR₉;

wherein X₁₀ is N or CR₁₀;

wherein X₁₁ is N or CR₁₁;

wherein X₁₂ is N or CR₁₂;

wherein X₁₃ is N or CR₁₃;

wherein X₁₄ is N or CR₁₄;

wherein X₁₅ is N or CR₁₅;

wherein X₁₆ is N or CR₁₆;

wherein X₁₇ is N or CR₁₇;

wherein X₁₈ is N or CR₁₈; and

wherein at least one of X₇ to X₁₈ is N.

Y may be selected from the group consisting of O, S, Se, NR₁, CR₂R₃ and SiR₄R₅. R₁ to R₅ may be independently selected from the group consisting of methyl, ethyl, phenyl, naphthyl, hexylbenzenyl, pyrimidinyl, quinolinyl, and combinations thereof. R₇ to R₁₈ may be independently selected from the group consisting of H, an aryl group having 6 carbon atoms, an alkyl group having 1, 2 or 3 carbon atoms, and a heteroaryl group having 3, 4 or 5 carbon atoms.

Adjacent two of X₇ to X₁₈ may form a five-membered ring, a six-membered ring or a combination thereof.

In formula (2), A may represent an aryl group having 6 to 30 ring carbon atoms, or a heteroaryl group including one to two heteroatoms of N and having 5 to 30 ring carbon atoms.

At least one of the following may be true:

only one of X₇ to X₁₈ is N;

at least eleven of X₇ to X₁₈ are not N;

at least eleven of X₇ to X₁₈ are CH;

at least one of X₇ to X₉ may be N;

at least one of X₇ and X₈ may be N;

one of X₇ to X₉ may be N; and

one of X₇ and X₈ may be N.

Preferably, only one of X₇ to X₉ is N. Alternatively, only one of X₇ and X₈ is N.

More preferably, X₈ may be N.

In case of at least one of X₇ to X₉ is N, X₁₁ to X₁₄ may preferably be not N, the heteroaryl group represented by A may preferably include two heteroatoms of N. The two heteroatoms of N are more preferably located in a single aromatic ring. The organic compounds comprising such A may each serve as an emitting host material of an organic EL device. The organic EL device may be operated under reduced driving voltage of about 5.6 V to about 6.0 V. See compounds 261, 155, 135, 55, 45, 35, 126, 206, 257, 196, 173, 90, 235, 66, 187, 25 of Table 1.

R₁ to R₆ may be independently selected from the group consisting of H, an alkyl group having 1 to 6 carbon atoms,

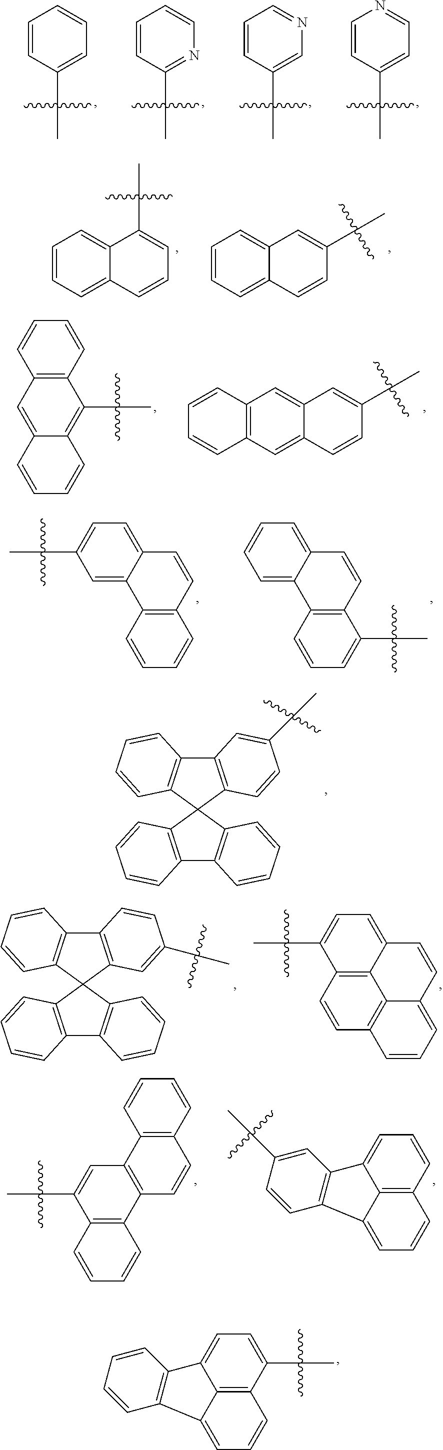

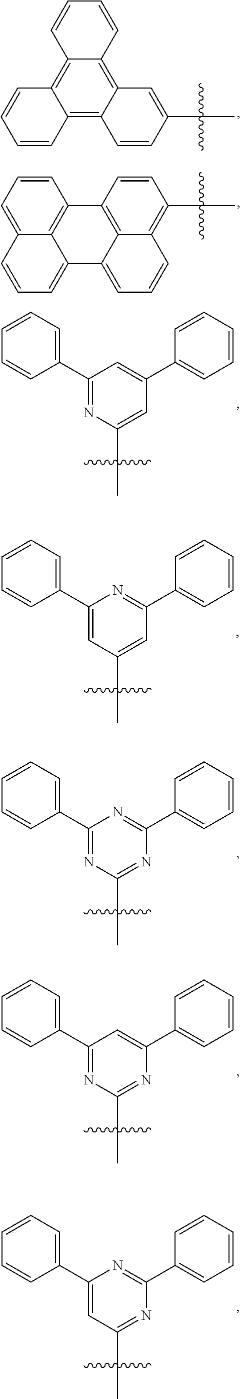

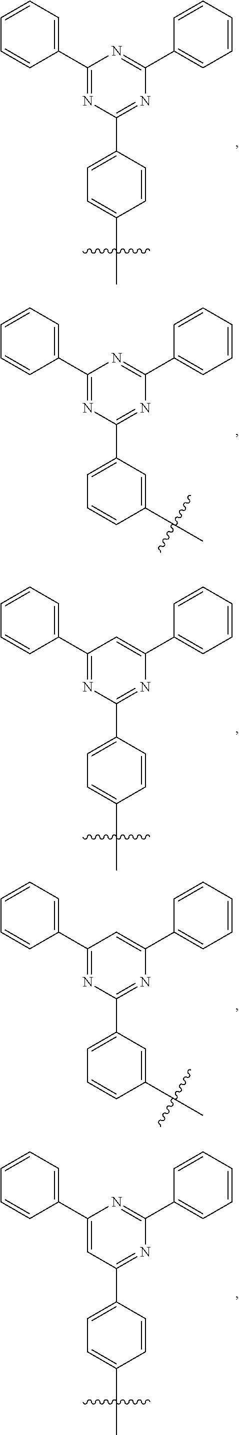 [SMILES: *c1ccc(-c2nc(-c3ccccc3)nc(-c3ccccc3)n2)cc1 ; *c1cccc(-c2nc(-c3ccccc3)nc(-c3ccccc3)n2)c1 ; *c1ccc(-c2nc(-c3ccccc3)cc(-c3ccccc3)n2)cc1 ; *c1cccc(-c2nc(-c3ccccc3)cc(-c3ccccc3)n2)c1 ; *c1ccc(-c2cc(-c3ccccc3)nc(-c3ccccc3)n2)cc1]

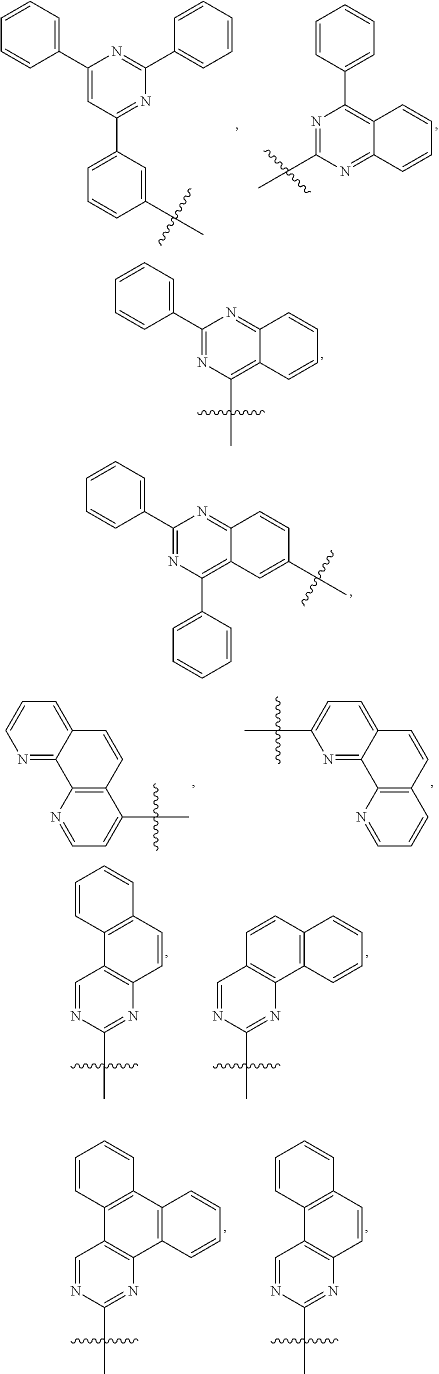

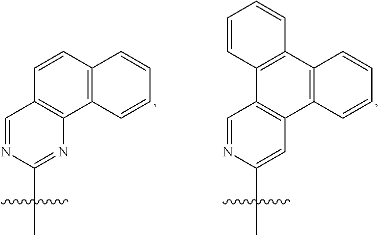 [SMILES: *c1ncc2ccc3ccccc3c2n1 ; *c1cc2c3ccccc3c3ccccc3c2cn1]

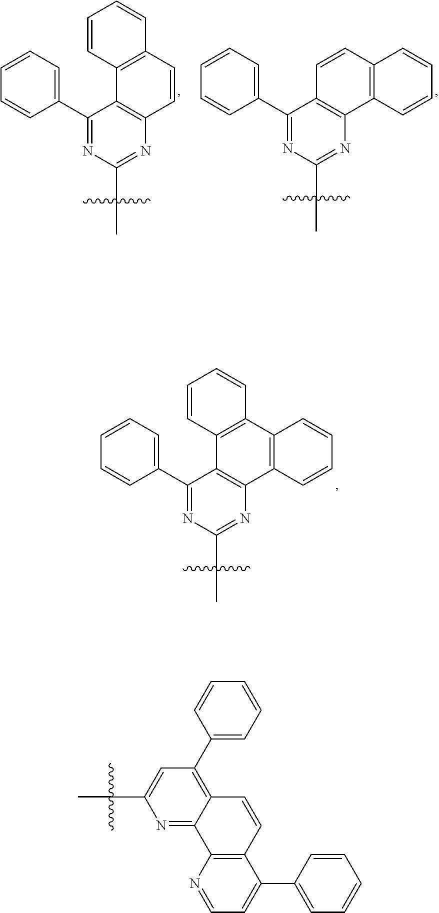

and combinations thereof.

R₇ to R₁₈ may be independently selected from the group consisting of H, an aryl group having 6 carbon atoms, an alkyl group having 1, 2 or 3 carbon atoms, and a heteroaryl group having 3, 4 or 5 carbon atoms. Adjacent two of X₇ to X₁₈ can form a five-membered ring, a six-membered ring or a combination thereof.

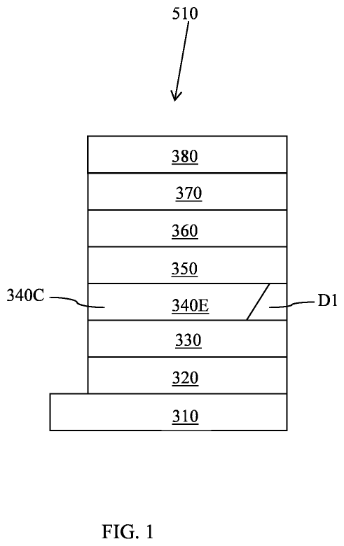

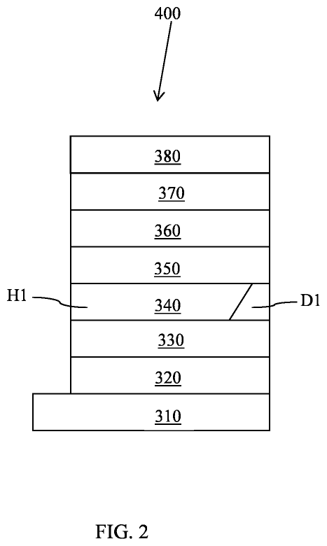

FIG. 2 is a cross-sectional view of an organic EL device without the organic compound of formula (1) (without 340C of FIG. 1). Referring to FIG. 2, the organic EL device 400 may have a driving voltage of about 6.3 V, a current efficiency of about 11 cd/A, or a half-life of about 202 hours.

Referring to FIG. 1, by comprising the organic compound of formula (1) as the host 340C, the first organic EL device 510 may have a driving voltage lower than that of the organic EL device 400 (FIG. 2). Moreover, by comprising the organic compound of formula (1) as the host 340C, the first organic EL device 510 of FIG. 1 may have a current efficiency higher than that of the organic EL device 400 (FIG. 2). Furthermore, by comprising the organic compound of formula (1) as the host 340C, the first organic EL device 510 of FIG. 1 may have a half-life longer than that of the organic EL device 400 (FIG. 2).

As the host 340C of the first organic EL device 510 of FIG. 1, the organic compound of formula (1) may lower the driving voltage to be about 5.6 V to about 6.2 V. Moreover, the organic compound of formula(1) may increase the current efficiency to be about 12 cd/A to about 24 cd/A. Furthermore, the organic compound of formula (1) may increase the half-life to be about 210 hours to about 296 hours.

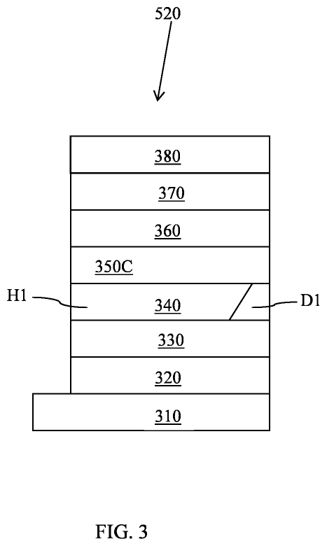

In a third embodiment of the present invention, a second organic EL device using the organic compound of formula (1) is disclosed. FIG. 3 is a cross-sectional view of the second organic EL device. Referring to FIG. 3, the second organic EL device 520 may comprise the organic compound of formula (1) as a hole blocking layer 350C.

FIG. 2 is a cross-sectional view of an organic EL device without the organic compound of formula (1) (without 350C of FIG. 3). Referring to FIG. 2, the organic EL device 400 may have a driving voltage of about 6.3 V, a current efficiency of about 11 cd/A, or a half-life of about 202 hours.

Referring to FIG. 3, by comprising the organic compound of formula (1) as the hole blocking layer 350C, the second organic EL device 520 may have a driving voltage lower than that of the organic EL device 400 (FIG. 2). Moreover, by comprising the organic compound of formula (1) as the hole blocking layer 350C, the second organic EL device 520 of FIG. 3 may have a current efficiency higher than that of the organic EL device 400 (FIG. 2). Furthermore, by comprising the organic compound of formula (1) as the hole blocking layer 350C, the second organic EL device 520 of FIG. 3 may have a half-life longer than that of the organic EL device 400 (FIG. 2).

Referring to FIG. 3, as the hole blocking layer 350C of the second organic EL device 520, the organic compound of formula (1) may lower the driving voltage to be about 6.0 V to about 6.3 V. Moreover, the organic compound of formula (1) may increase the current efficiency to be about 12 cd/A to about 14 cd/A. Furthermore, the organic compound of formula (1) may increase the half-life to be about 204 hours to about 215 hours.

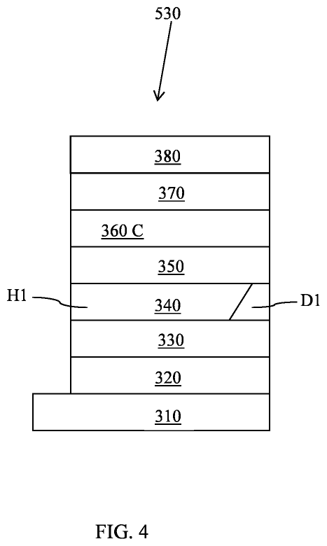

In a fourth embodiment of the present invention, a third organic EL device using the organic compound of formula (1) is disclosed. FIG. 4 is a cross-sectional view of the third organic EL device. Referring to FIG. 4, the second organic EL device 530 may comprise the organic compound of formula (1) as an electron transport layer 360C.

FIG. 2 is a cross-sectional view of an organic EL device without the organic compound of formula (1) (without 360C of FIG. 4). Referring to FIG. 2, the organic EL device 400 may have a driving voltage of about 6.3 V, a current efficiency of about 11 cd/A, or a half-life of about 202 hours.

Referring to FIG. 4, by comprising the organic compound of formula (1) as the electron transport layer 360C, the third organic EL device 530 may have a driving voltage lower than that of the organic EL device 400 (FIG. 2). Moreover, by comprising the organic compound of formula (1) as the electron transport layer 360C, the third organic EL device 530 of FIG. 4 may have a current efficiency higher than that of the organic EL device 400 (FIG. 2). Furthermore, by comprising the organic compound of formula (1) as the electron transport layer 360C, the second organic EL device 530 of FIG. 4 may have a half-life longer than that of the organic EL device 400 (FIG. 2).

Referring to FIG. 4, as the electron transport layer 360C of the third organic EL device 530, the organic compound of formula (1) may lower the driving voltage to be about 5.9 V to about 6.2 V. Moreover, the organic compound of formula (1) may increase the current efficiency to be about 13 cd/A to about 17 cd/A. Furthermore, the organic compound of formula (1) may increase the half-life to be about 213 hours to about 238 hours.

In the organic compound, the alkyl group, aralkyl group, aryl group, or heteroaryl group may be substituted by a halogen, an alkyl group, an aryl group, or a heteroaryl group.

In the organic compound of formula (1) or formula (2), A may be selected from phenyl, pyridinyl, triazinyl, naphthyl, fluorenyl, quinazolinyl, benzoquinazolinyl, phenylquinazolinyl,

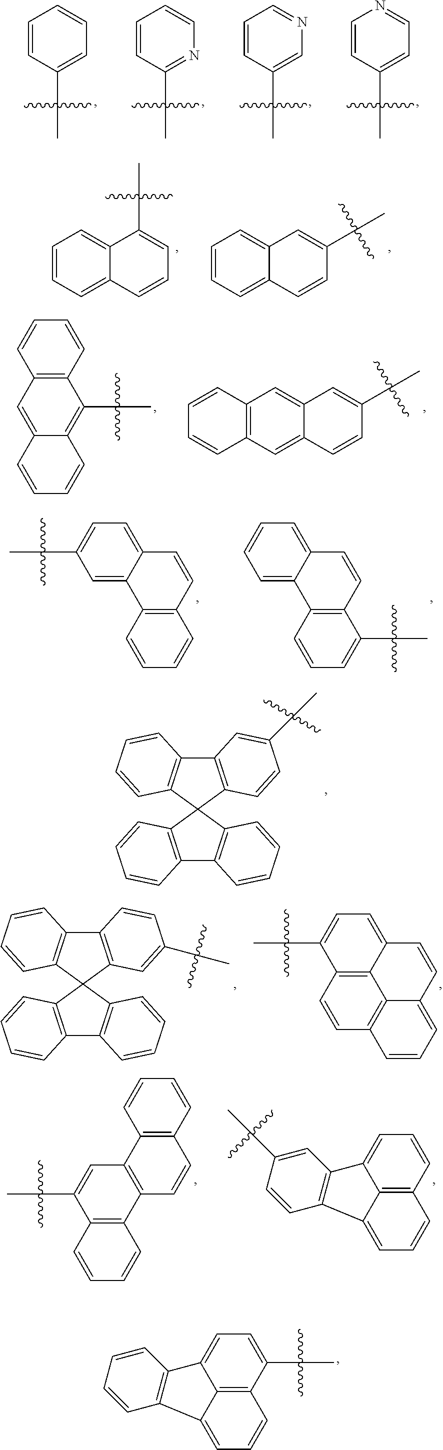

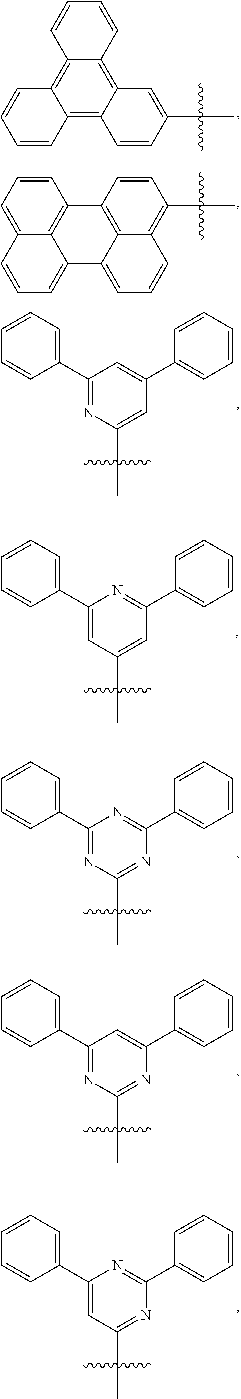

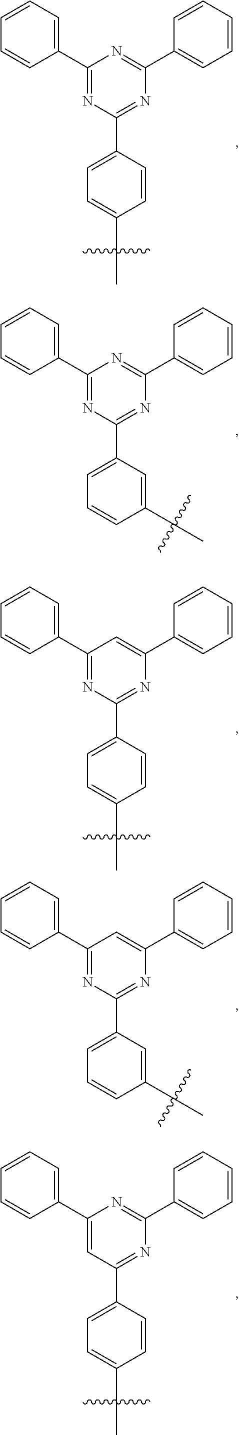 [SMILES: *c1ccc(-c2nc(-c3ccccc3)nc(-c3ccccc3)n2)cc1 ; *c1cccc(-c2nc(-c3ccccc3)nc(-c3ccccc3)n2)c1 ; *c1ccc(-c2nc(-c3ccccc3)cc(-c3ccccc3)n2)cc1 ; *c1cccc(-c2nc(-c3ccccc3)cc(-c3ccccc3)n2)c1 ; *c1ccc(-c2cc(-c3ccccc3)nc(-c3ccccc3)n2)cc1]

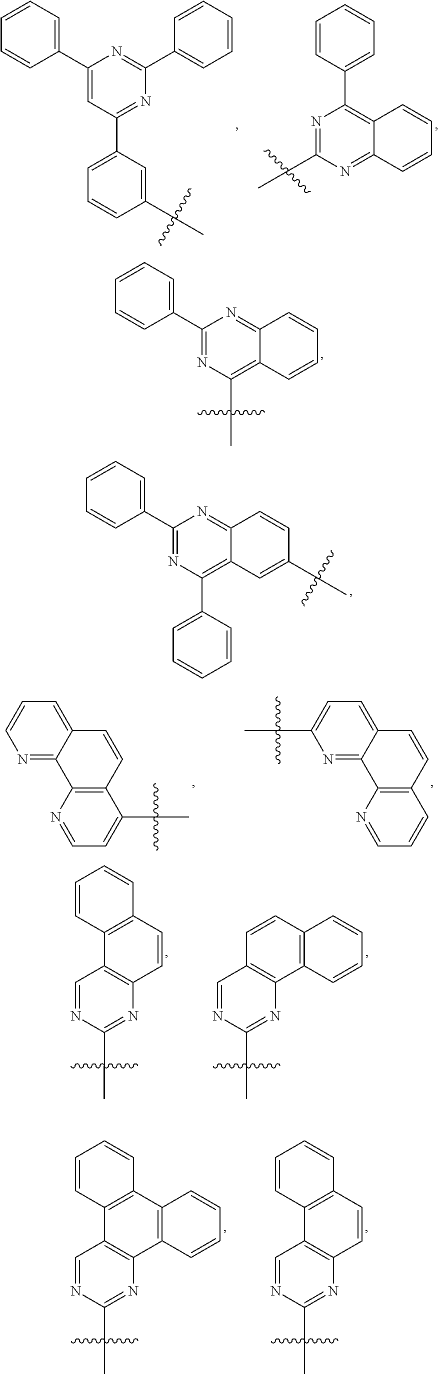

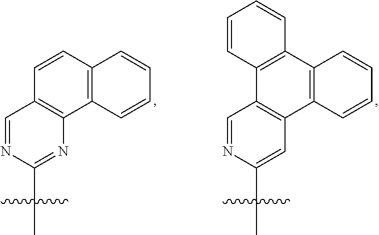 [SMILES: *c1ncc2ccc3ccccc3c2n1 ; *c1cc2c3ccccc3c3ccccc3c2cn1]

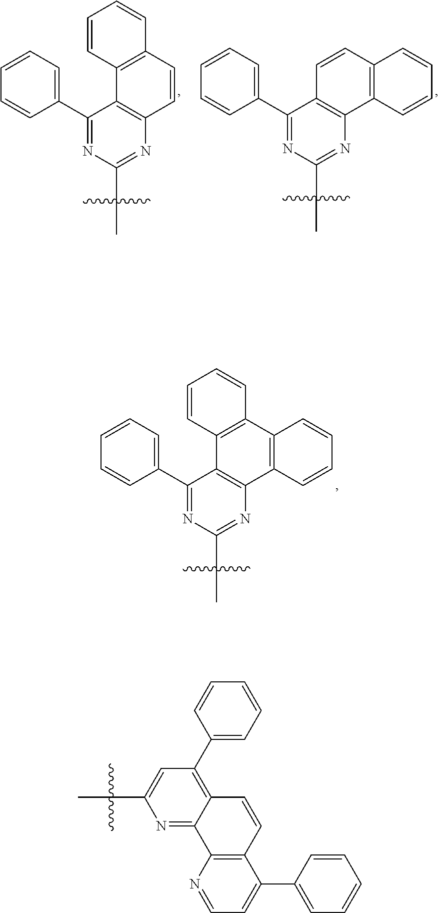

and combinations thereof. Preferably, A may be selected from

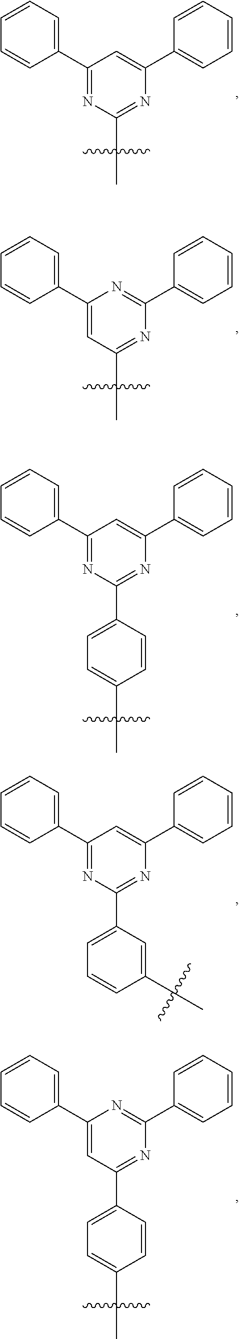 [SMILES: *c1nc(-c2ccccc2)cc(-c2ccccc2)n1 ; *c1cc(-c2ccccc2)nc(-c2ccccc2)n1 ; *c1ccc(-c2nc(-c3ccccc3)cc(-c3ccccc3)n2)cc1 ; *c1cccc(-c2nc(-c3ccccc3)cc(-c3ccccc3)n2)c1 ; *c1ccc(-c2cc(-c3ccccc3)nc(-c3ccccc3)n2)cc1]

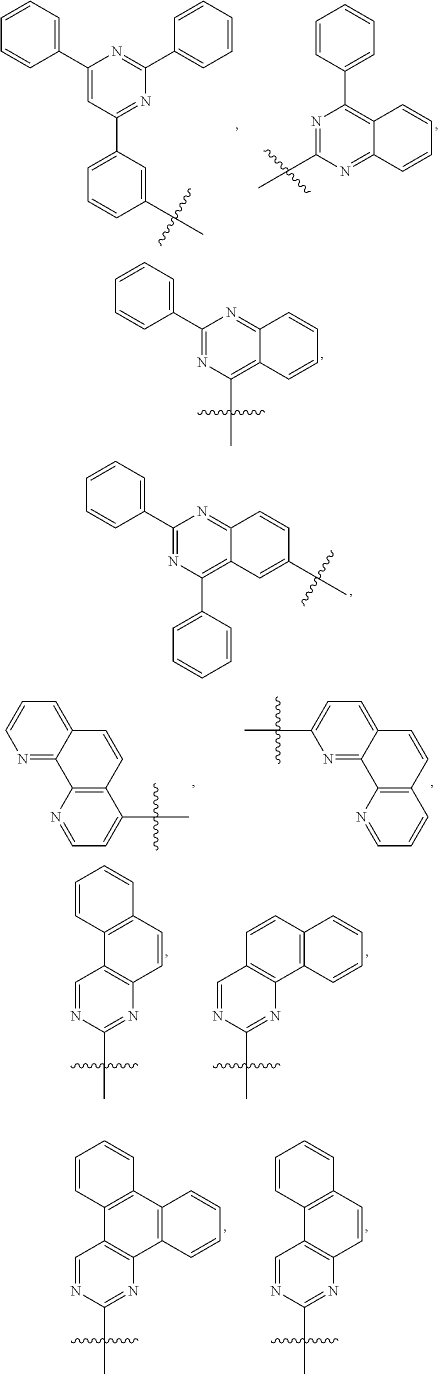

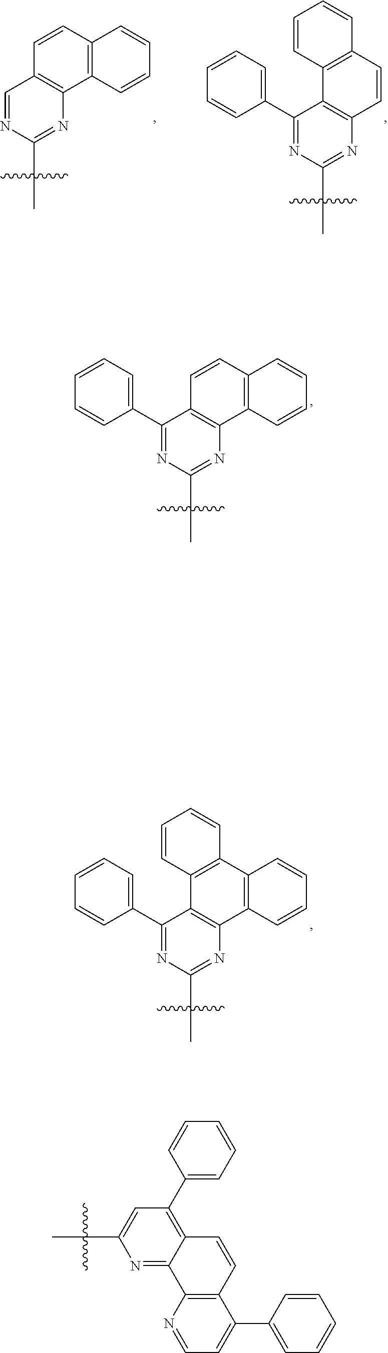 [SMILES: *c1ncc2ccc3ccccc3c2n1 ; *c1nc(-c2ccccc2)c2c(ccc3ccccc32)n1 ; *c1nc(-c2ccccc2)c2ccc3ccccc3c2n1 ; *c1nc(-c2ccccc2)c2c3ccccc3c3ccccc3c2n1 ; *c1cc(-c2ccccc2)c2ccc3c(-c4ccccc4)ccnc3c2n1]

and combinations thereof.

Preferably, the organic compound may be selected from the group consisting of the following compounds:

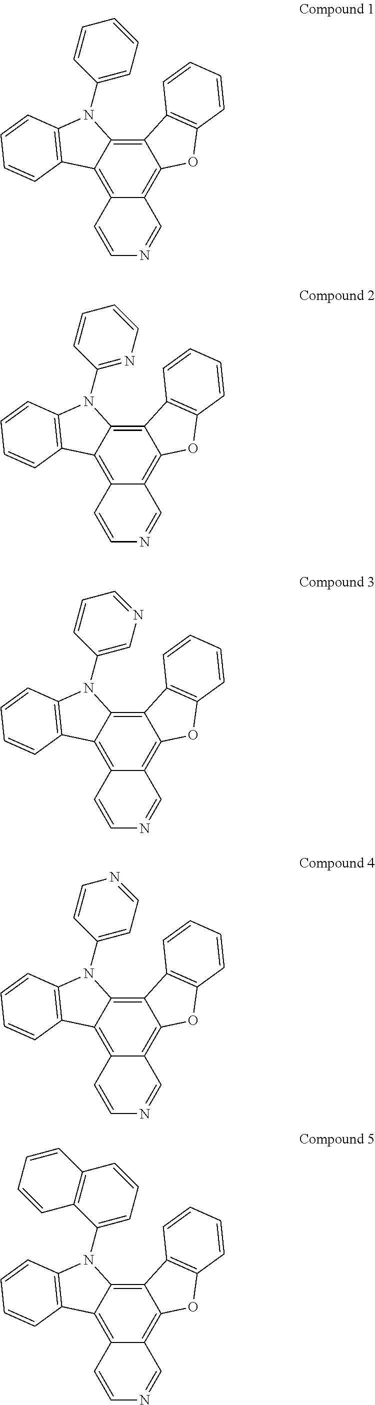 [SMILES: c1ccc(-n2c3ccccc3c3c4ccncc4c4oc5ccccc5c4c32)cc1 ; c1ccc(-n2c3ccccc3c3c4ccncc4c4oc5ccccc5c4c32)nc1 ; c1cncc(-n2c3ccccc3c3c4ccncc4c4oc5ccccc5c4c32)c1 ; c1ccc2c(c1)oc1c3cnccc3c3c4ccccc4n(-c4ccncc4)c3c21 ; c1ccc2c(-n3c4ccccc4c4c5ccncc5c5oc6ccccc6c5c43)cccc2c1]

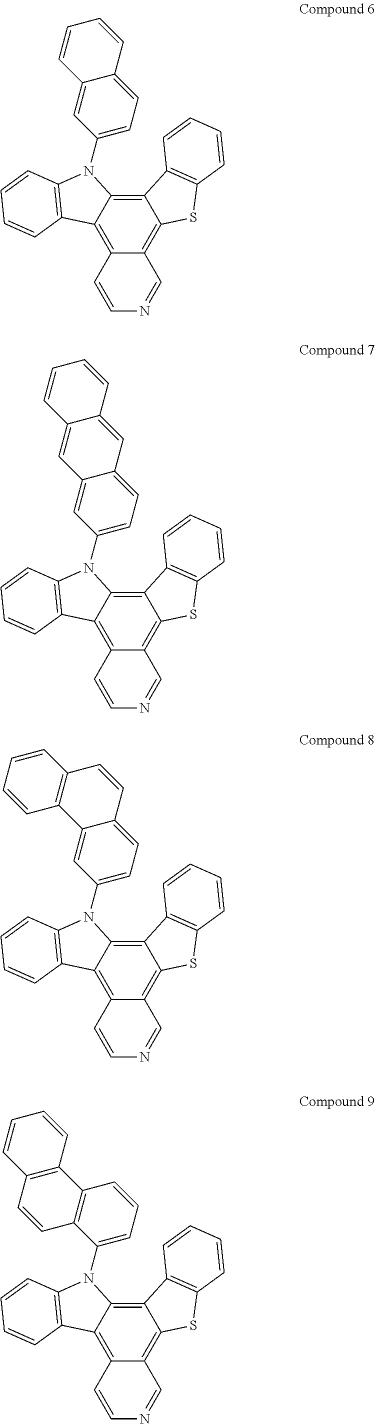 [SMILES: c1ccc2cc(-n3c4ccccc4c4c5ccncc5c5sc6ccccc6c5c43)ccc2c1 ; c1ccc2cc3cc(-n4c5ccccc5c5c6ccncc6c6sc7ccccc7c6c54)ccc3cc2c1 ; c1ccc2c(c1)ccc1ccc(-n3c4ccccc4c4c5ccncc5c5sc6ccccc6c5c43)cc12 ; c1ccc2c(c1)ccc1c(-n3c4ccccc4c4c5ccncc5c5sc6ccccc6c5c43)cccc12]

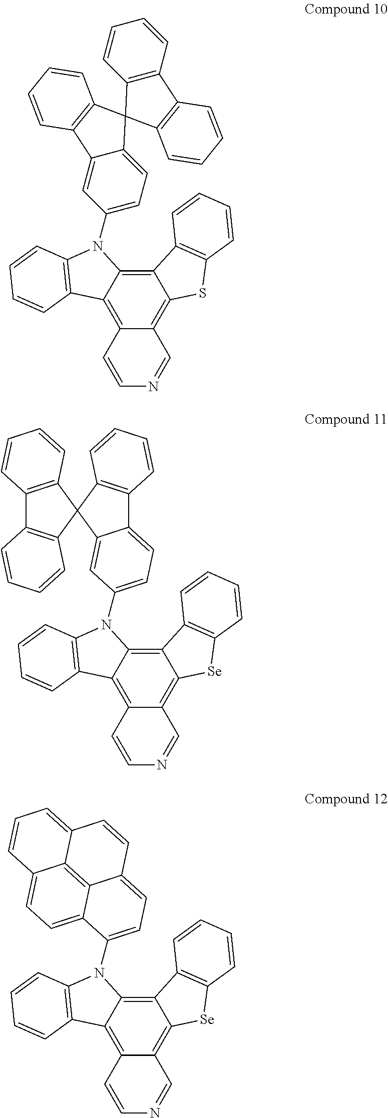

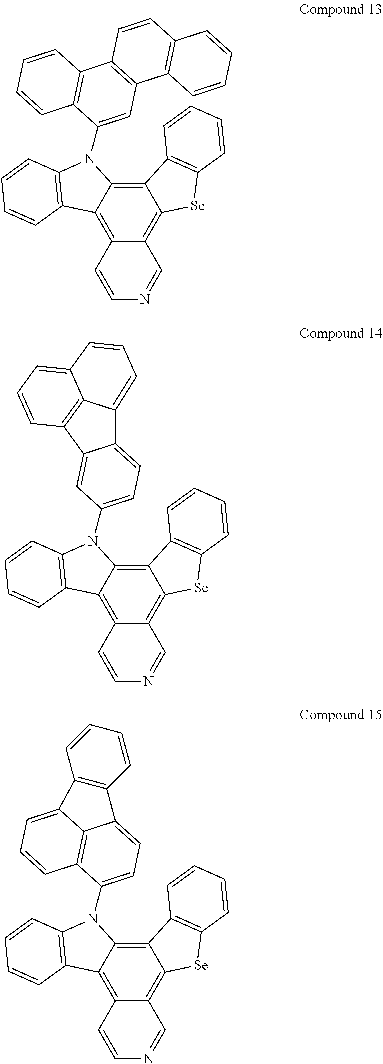 [SMILES: c1ccc2c(c1)ccc1c3ccccc3c(-n3c4ccccc4c4c5ccncc5c5[se]c6ccccc6c5c43)cc21 ; c1cc2c3c(cccc3c1)-c1cc(-n3c4ccccc4c4c5ccncc5c5[se]c6ccccc6c5c43)ccc1-2 ; c1ccc2c(c1)-c1cccc3c(-n4c5ccccc5c5c6ccncc6c6[se]c7ccccc7c6c54)ccc-2c13]

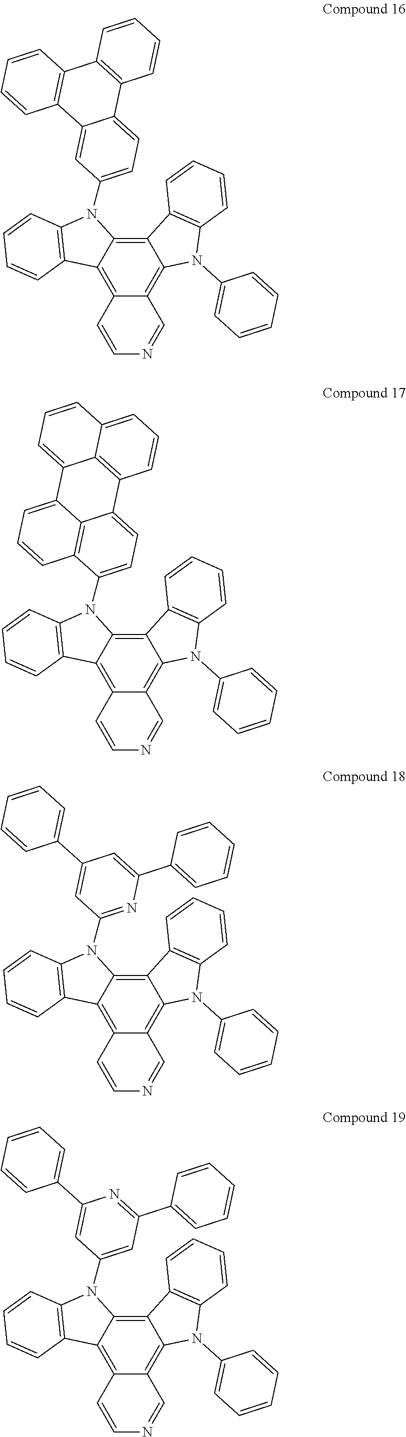 [SMILES: c1ccc(-n2c3ccccc3c3c2c2cnccc2c2c4ccccc4n(-c4ccc5c6ccccc6c6ccccc6c5c4)c23)cc1 ; c1ccc(-n2c3ccccc3c3c2c2cnccc2c2c4ccccc4n(-c4ccc5c6cccc7cccc(c8cccc4c85)c76)c23)cc1 ; c1ccc(-c2cc(-c3ccccc3)nc(-n3c4ccccc4c4c5ccncc5c5c(c6ccccc6n5-c5ccccc5)c43)c2)cc1 ; c1ccc(-c2cc(-n3c4ccccc4c4c5ccncc5c5c(c6ccccc6n5-c5ccccc5)c43)cc(-c3ccccc3)n2)cc1]

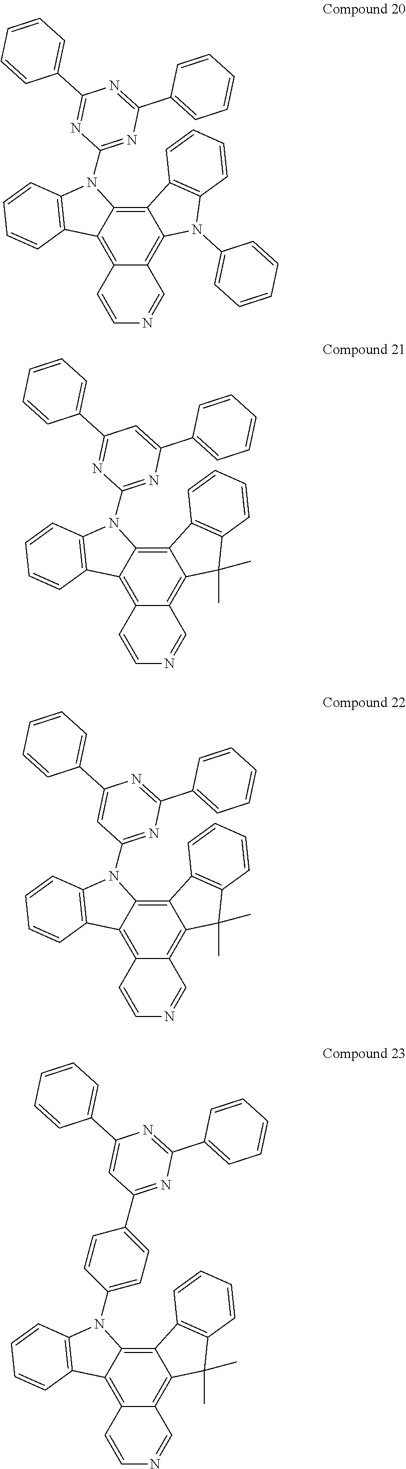 [SMILES: c1ccc(-c2nc(-c3ccccc3)nc(-n3c4ccccc4c4c5ccncc5c5c(c6ccccc6n5-c5ccccc5)c43)n2)cc1 ; CC1(C)c2ccccc2-c2c1c1cnccc1c1c3ccccc3n(-c3nc(-c4ccccc4)cc(-c4ccccc4)n3)c21 ; CC1(C)c2ccccc2-c2c1c1cnccc1c1c3ccccc3n(-c3cc(-c4ccccc4)nc(-c4ccccc4)n3)c21 ; CC1(C)c2ccccc2-c2c1c1cnccc1c1c3ccccc3n(-c3ccc(-c4cc(-c5ccccc5)nc(-c5ccccc5)n4)cc3)c21]

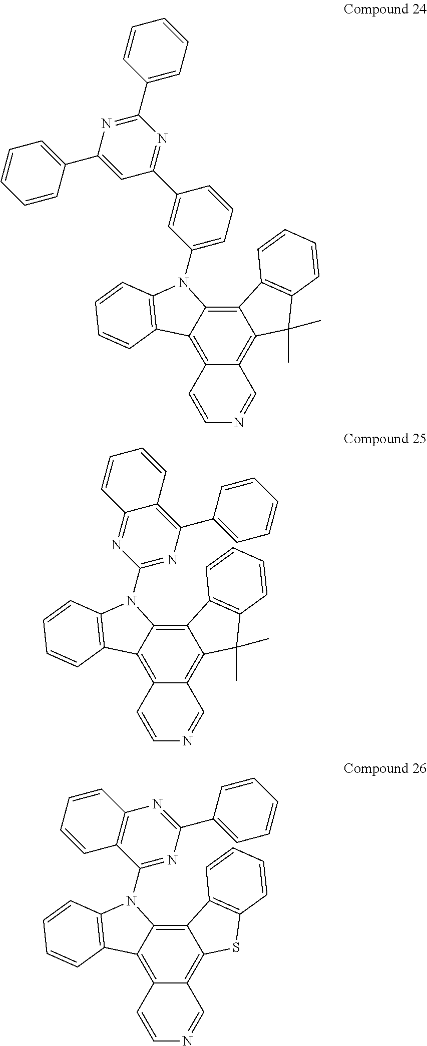 [SMILES: CC1(C)c2ccccc2-c2c1c1cnccc1c1c3ccccc3n(-c3cccc(-c4cc(-c5ccccc5)nc(-c5ccccc5)n4)c3)c21 ; CC1(C)c2ccccc2-c2c1c1cnccc1c1c3ccccc3n(-c3nc(-c4ccccc4)c4ccccc4n3)c21 ; c1ccc(-c2nc(-n3c4ccccc4c4c5ccncc5c5sc6ccccc6c5c43)c3ccccc3n2)cc1]

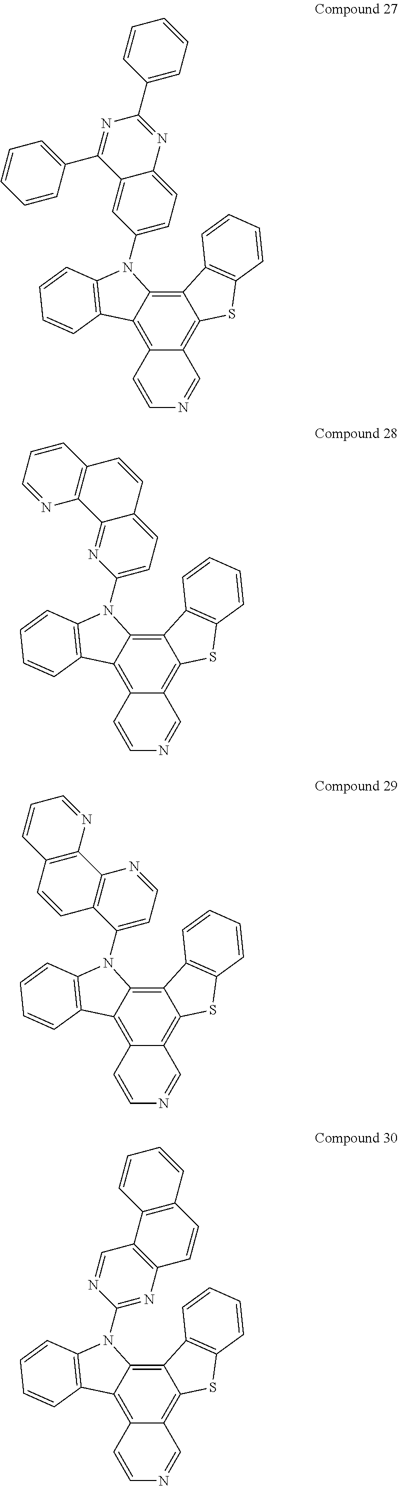

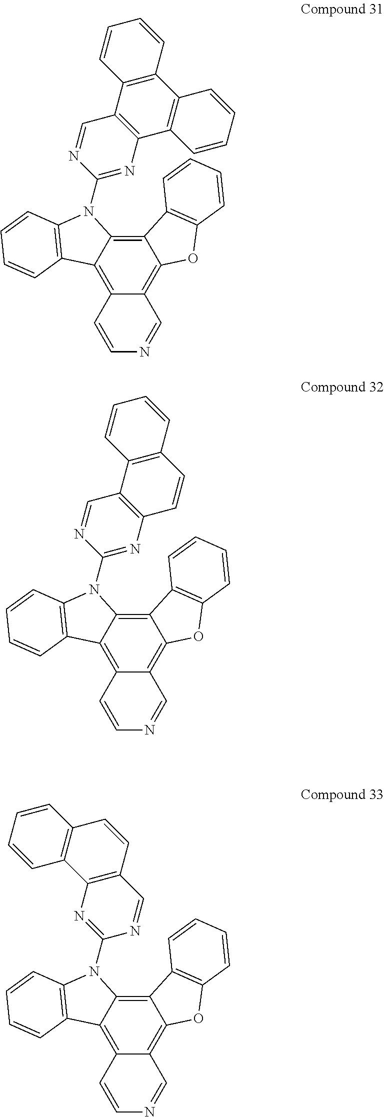

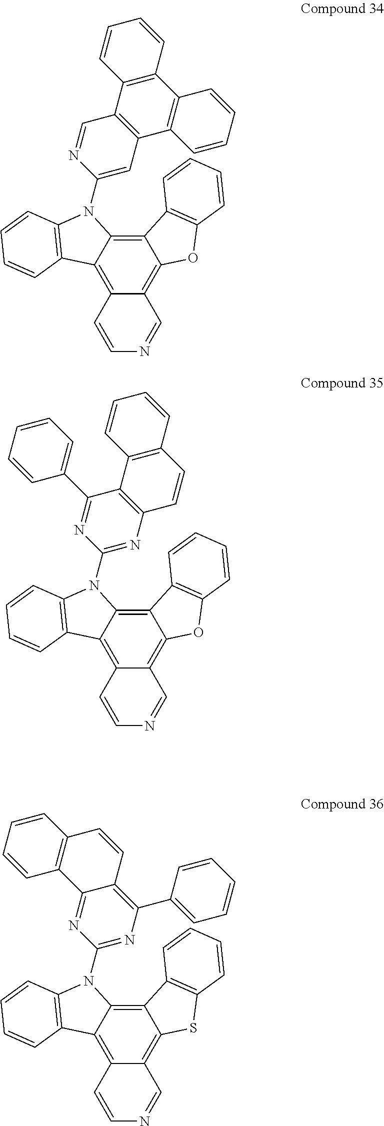

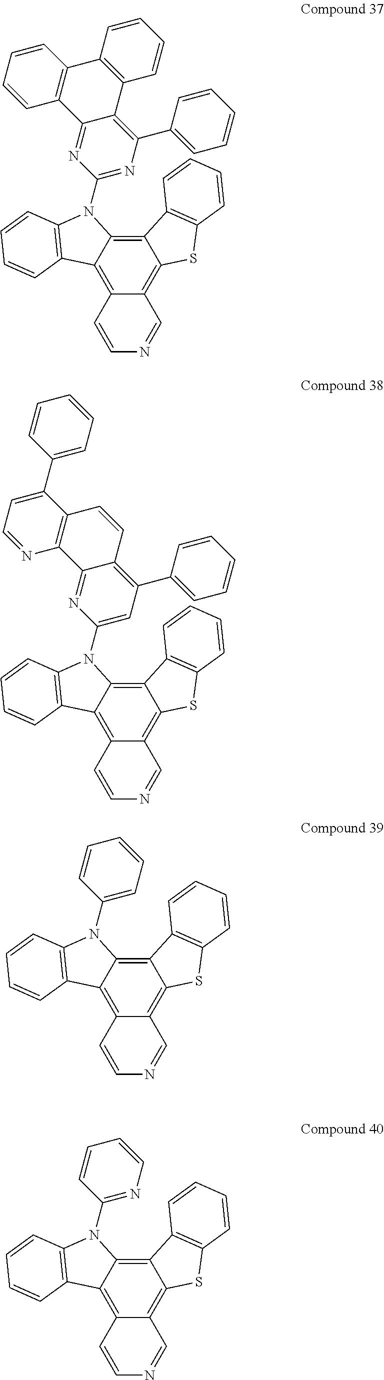

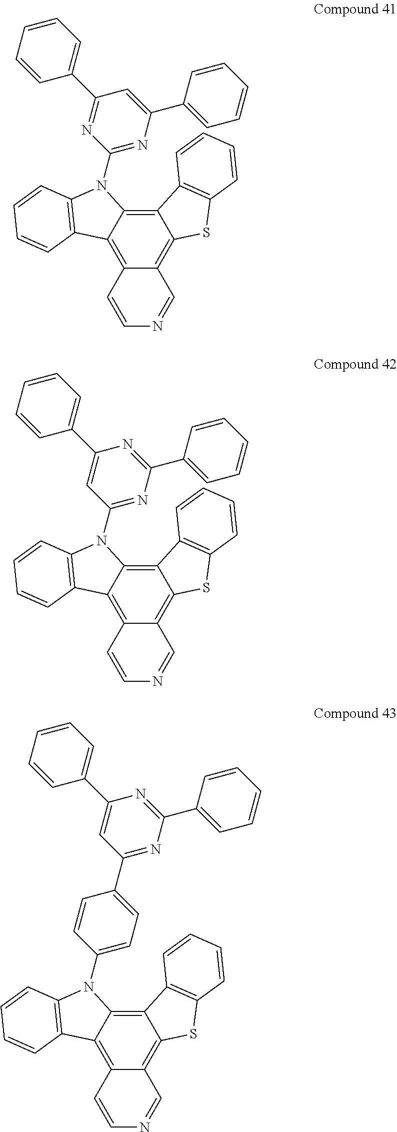 [SMILES: c1ccc(-c2cc(-c3ccccc3)nc(-n3c4ccccc4c4c5ccncc5c5sc6ccccc6c5c43)n2)cc1 ; c1ccc(-c2cc(-n3c4ccccc4c4c5ccncc5c5sc6ccccc6c5c43)nc(-c3ccccc3)n2)cc1 ; c1ccc(-c2cc(-c3ccc(-n4c5ccccc5c5c6ccncc6c6sc7ccccc7c6c54)cc3)nc(-c3ccccc3)n2)cc1]

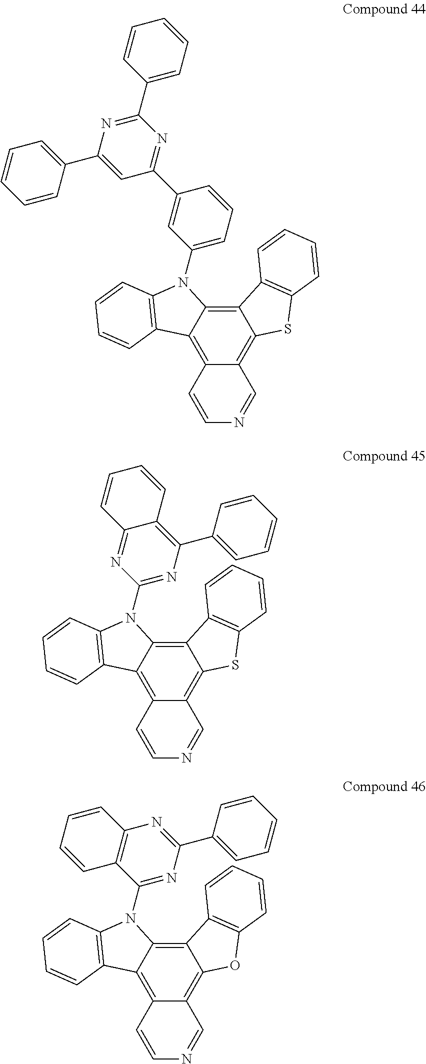 [SMILES: c1ccc(-c2cc(-c3cccc(-n4c5ccccc5c5c6ccncc6c6sc7ccccc7c6c54)c3)nc(-c3ccccc3)n2)cc1 ; c1ccc(-c2nc(-n3c4ccccc4c4c5ccncc5c5sc6ccccc6c5c43)nc3ccccc23)cc1 ; c1ccc(-c2nc(-n3c4ccccc4c4c5ccncc5c5oc6ccccc6c5c43)c3ccccc3n2)cc1]

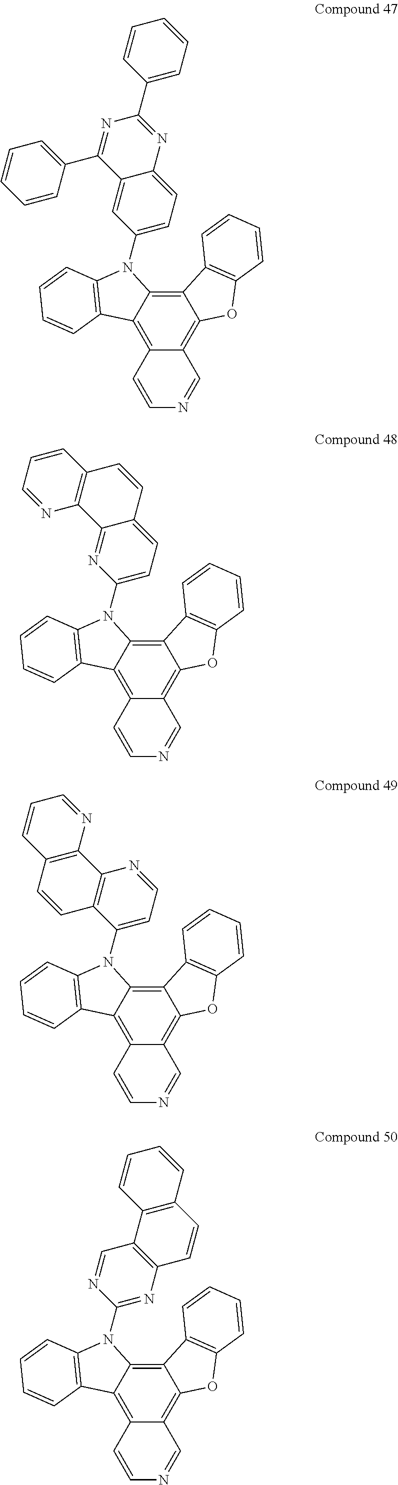

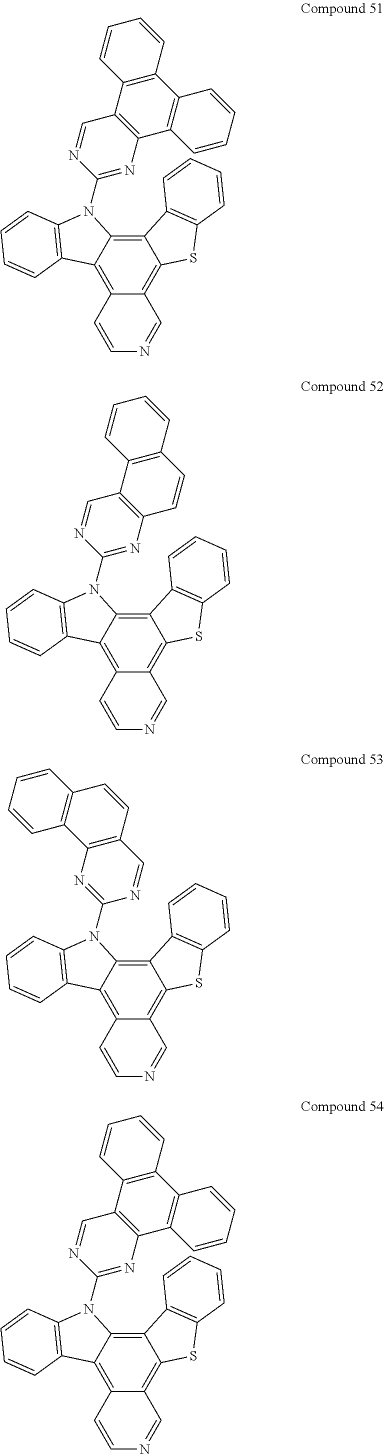

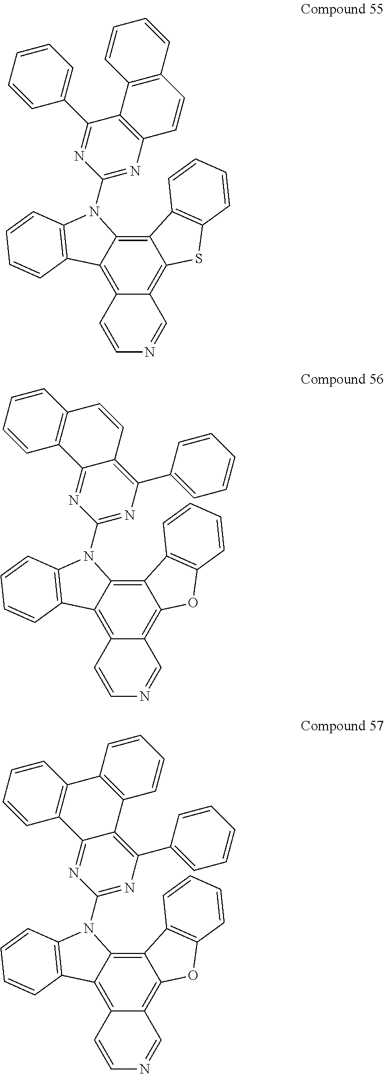

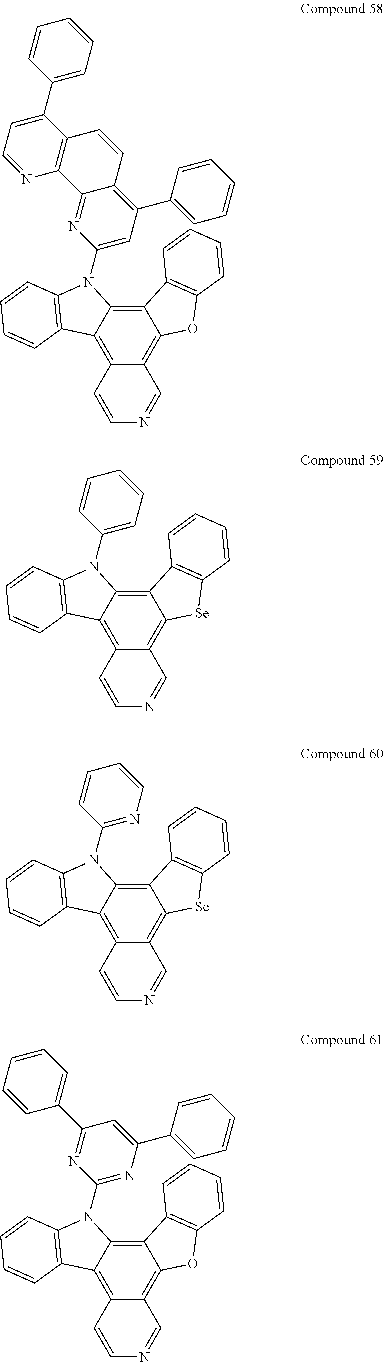 [SMILES: c1ccc(-c2ccnc3c2ccc2c(-c4ccccc4)cc(-n4c5ccccc5c5c6ccncc6c6oc7ccccc7c6c54)nc23)cc1 ; c1ccc(-n2c3ccccc3c3c4ccncc4c4[se]c5ccccc5c4c32)cc1 ; c1ccc(-n2c3ccccc3c3c4ccncc4c4[se]c5ccccc5c4c32)nc1 ; c1ccc(-c2cc(-c3ccccc3)nc(-n3c4ccccc4c4c5ccncc5c5oc6ccccc6c5c43)n2)cc1]

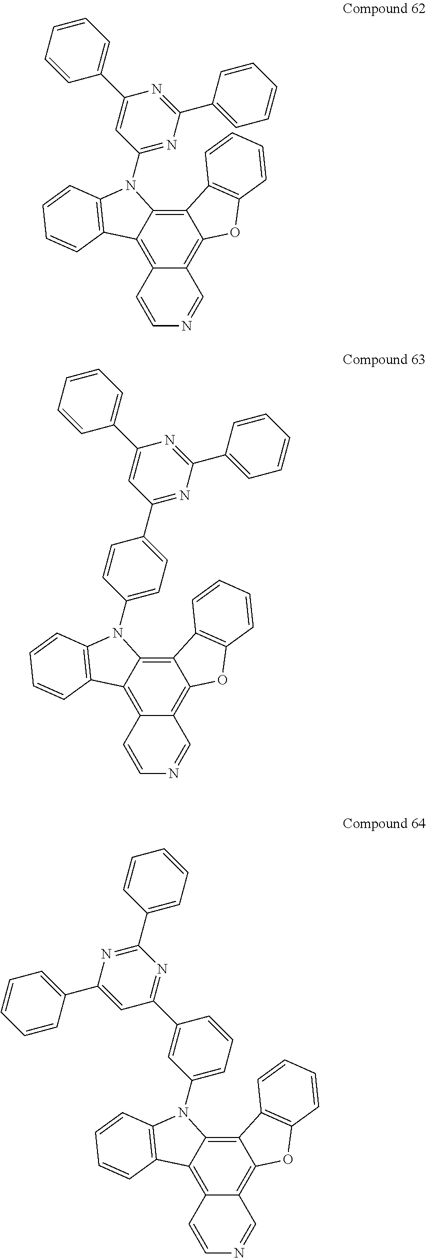 [SMILES: c1ccc(-c2cc(-n3c4ccccc4c4c5ccncc5c5oc6ccccc6c5c43)nc(-c3ccccc3)n2)cc1 ; c1ccc(-c2cc(-c3ccc(-n4c5ccccc5c5c6ccncc6c6oc7ccccc7c6c54)cc3)nc(-c3ccccc3)n2)cc1 ; c1ccc(-c2cc(-c3cccc(-n4c5ccccc5c5c6ccncc6c6oc7ccccc7c6c54)c3)nc(-c3ccccc3)n2)cc1]

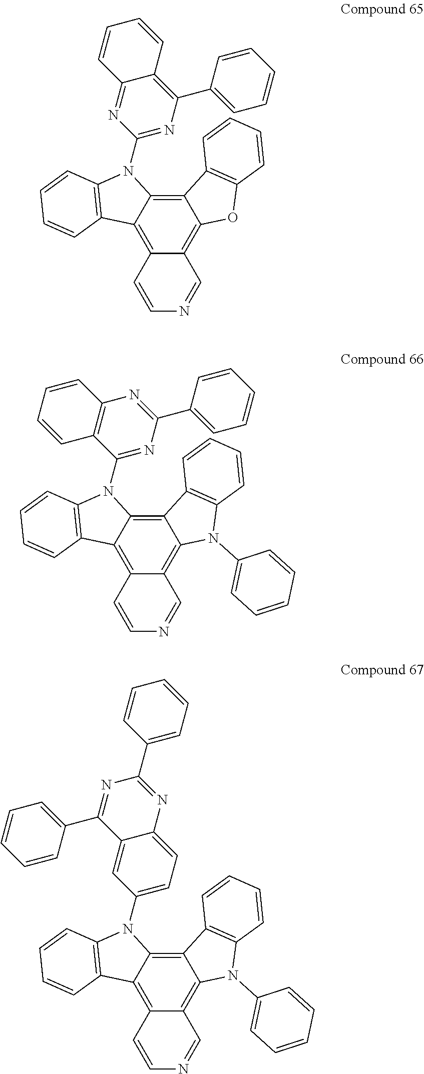 [SMILES: c1ccc(-c2nc(-n3c4ccccc4c4c5ccncc5c5oc6ccccc6c5c43)nc3ccccc23)cc1 ; c1ccc(-c2nc(-n3c4ccccc4c4c5ccncc5c5c(c6ccccc6n5-c5ccccc5)c43)c3ccccc3n2)cc1 ; c1ccc(-c2nc(-c3ccccc3)c3cc(-n4c5ccccc5c5c6ccncc6c6c(c7ccccc7n6-c6ccccc6)c54)ccc3n2)cc1]

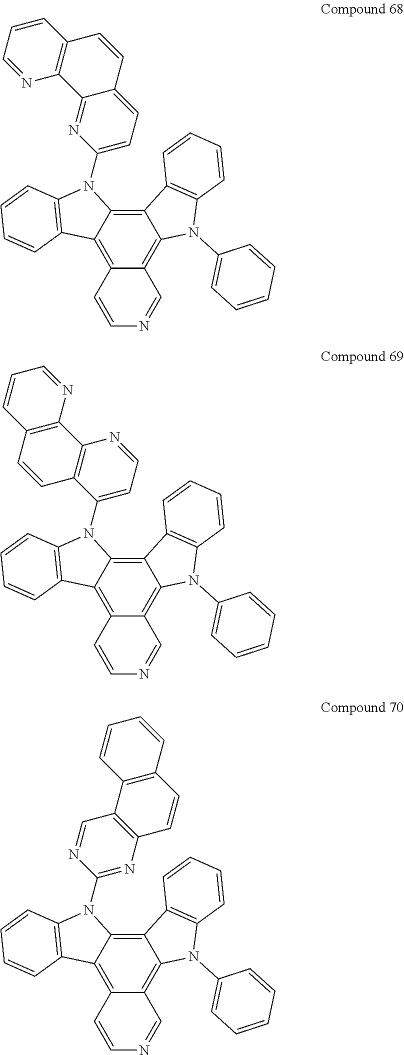

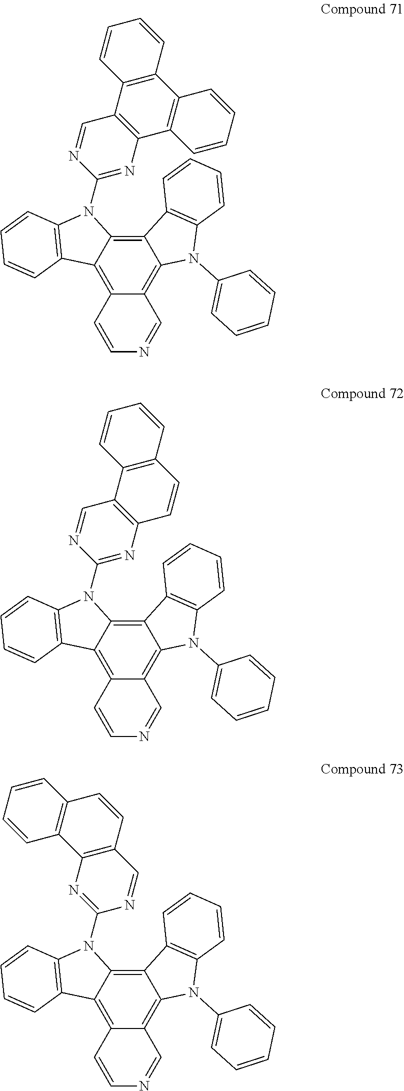 [SMILES: c1ccc(-n2c3ccccc3c3c2c2cnccc2c2c4ccccc4n(-c4ncc5c6ccccc6c6ccccc6c5n4)c23)cc1 ; c1ccc(-n2c3ccccc3c3c2c2cnccc2c2c4ccccc4n(-c4ncc5c(ccc6ccccc65)n4)c23)cc1 ; c1ccc(-n2c3ccccc3c3c2c2cnccc2c2c4ccccc4n(-c4ncc5ccc6ccccc6c5n4)c23)cc1]

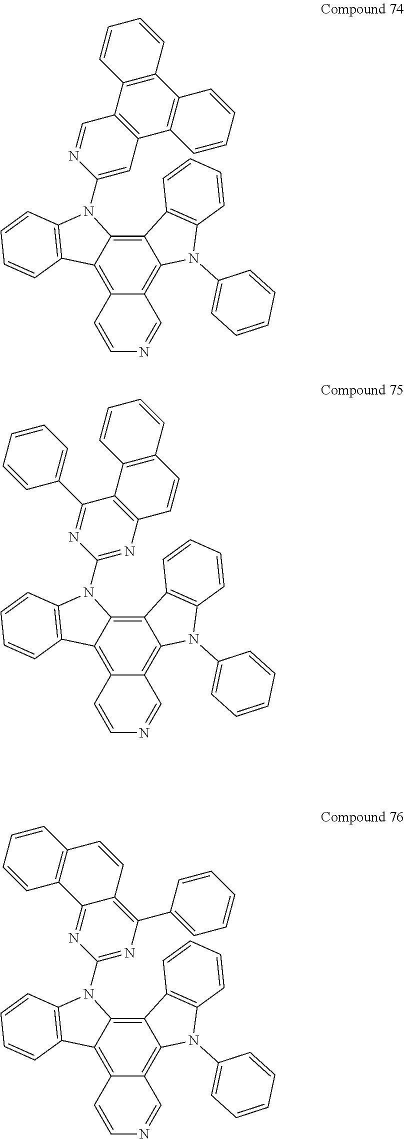 [SMILES: c1ccc(-n2c3ccccc3c3c2c2cnccc2c2c4ccccc4n(-c4cc5c6ccccc6c6ccccc6c5cn4)c23)cc1 ; c1ccc(-c2nc(-n3c4ccccc4c4c5ccncc5c5c(c6ccccc6n5-c5ccccc5)c43)nc3ccc4ccccc4c23)cc1 ; c1ccc(-c2nc(-n3c4ccccc4c4c5ccncc5c5c(c6ccccc6n5-c5ccccc5)c43)nc3c2ccc2ccccc23)cc1]

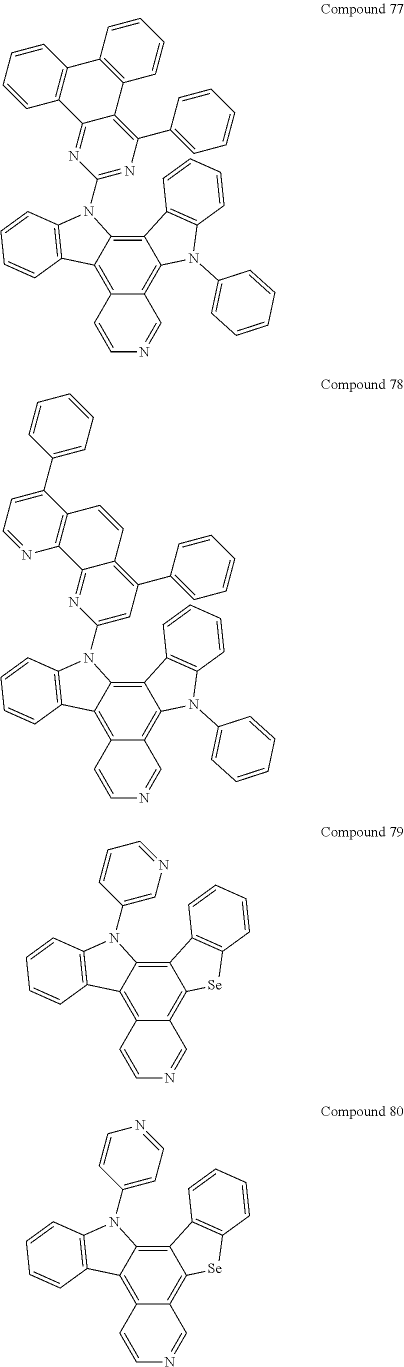

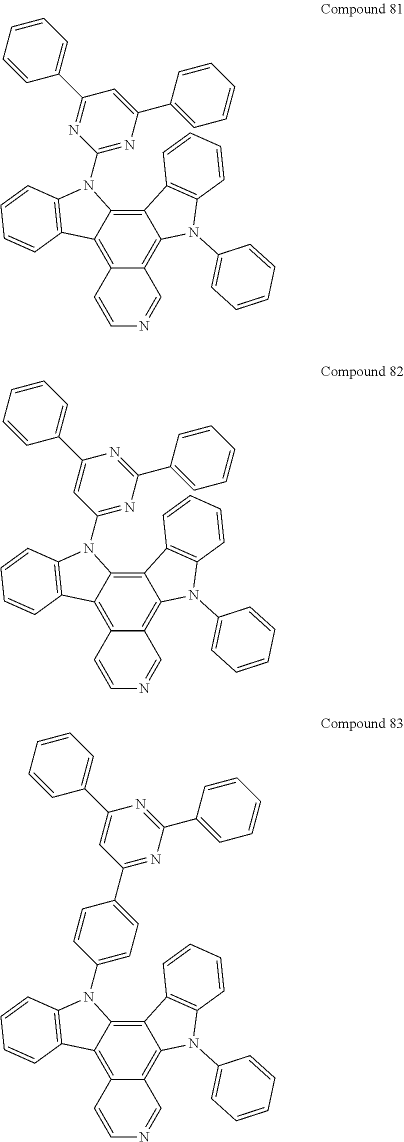 [SMILES: c1ccc(-c2cc(-c3ccccc3)nc(-n3c4ccccc4c4c5ccncc5c5c(c6ccccc6n5-c5ccccc5)c43)n2)cc1 ; c1ccc(-c2cc(-n3c4ccccc4c4c5ccncc5c5c(c6ccccc6n5-c5ccccc5)c43)nc(-c3ccccc3)n2)cc1 ; c1ccc(-c2cc(-c3ccc(-n4c5ccccc5c5c6ccncc6c6c(c7ccccc7n6-c6ccccc6)c54)cc3)nc(-c3ccccc3)n2)cc1]

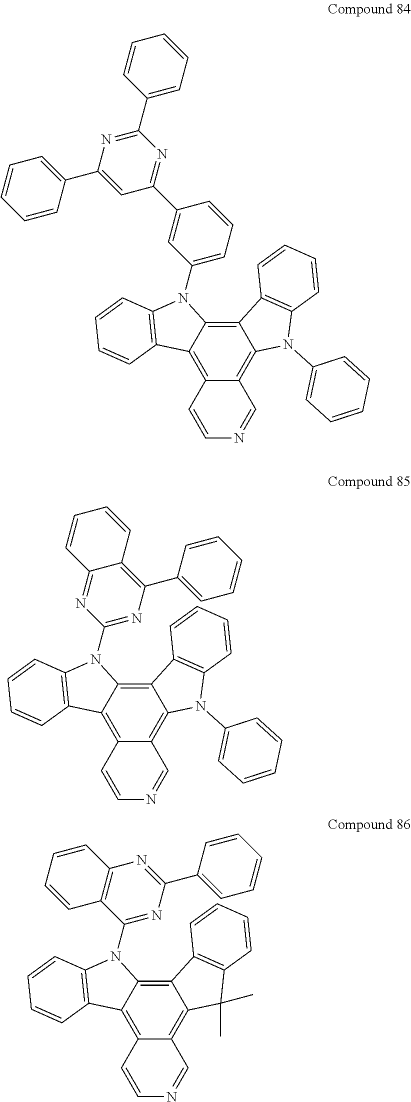 [SMILES: c1ccc(-c2cc(-c3cccc(-n4c5ccccc5c5c6ccncc6c6c(c7ccccc7n6-c6ccccc6)c54)c3)nc(-c3ccccc3)n2)cc1 ; c1ccc(-c2nc(-n3c4ccccc4c4c5ccncc5c5c(c6ccccc6n5-c5ccccc5)c43)nc3ccccc23)cc1 ; CC1(C)c2ccccc2-c2c1c1cnccc1c1c3ccccc3n(-c3nc(-c4ccccc4)nc4ccccc34)c21]

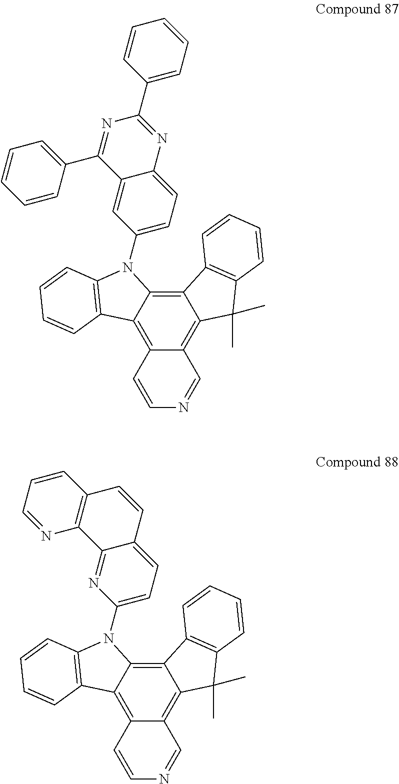 [SMILES: CC1(C)c2ccccc2-c2c1c1cnccc1c1c3ccccc3n(-c3ccc4nc(-c5ccccc5)nc(-c5ccccc5)c4c3)c21 ; CC1(C)c2ccccc2-c2c1c1cnccc1c1c3ccccc3n(-c3ccc4ccc5cccnc5c4n3)c21]

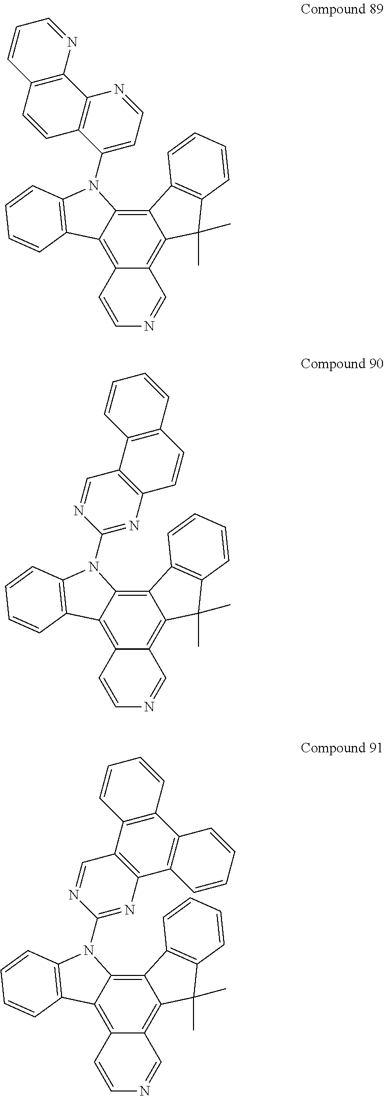 [SMILES: CC1(C)c2ccccc2-c2c1c1cnccc1c1c3ccccc3n(-c3ccnc4c3ccc3cccnc34)c21 ; CC1(C)c2ccccc2-c2c1c1cnccc1c1c3ccccc3n(-c3ncc4c(ccc5ccccc54)n3)c21 ; CC1(C)c2ccccc2-c2c1c1cnccc1c1c3ccccc3n(-c3ncc4c5ccccc5c5ccccc5c4n3)c21]

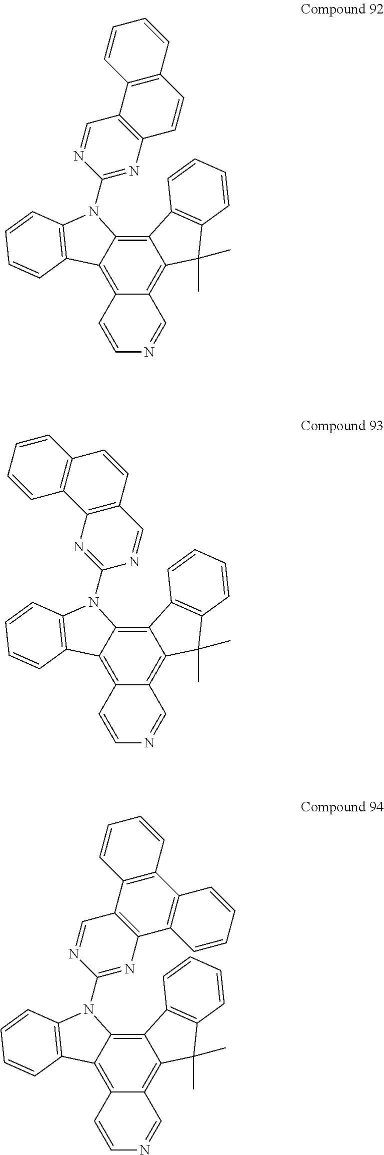 [SMILES: CC1(C)c2ccccc2-c2c1c1cnccc1c1c3ccccc3n(-c3ncc4c(ccc5ccccc54)n3)c21 ; CC1(C)c2ccccc2-c2c1c1cnccc1c1c3ccccc3n(-c3ncc4ccc5ccccc5c4n3)c21 ; CC1(C)c2ccccc2-c2c1c1cnccc1c1c3ccccc3n(-c3ncc4c5ccccc5c5ccccc5c4n3)c21]

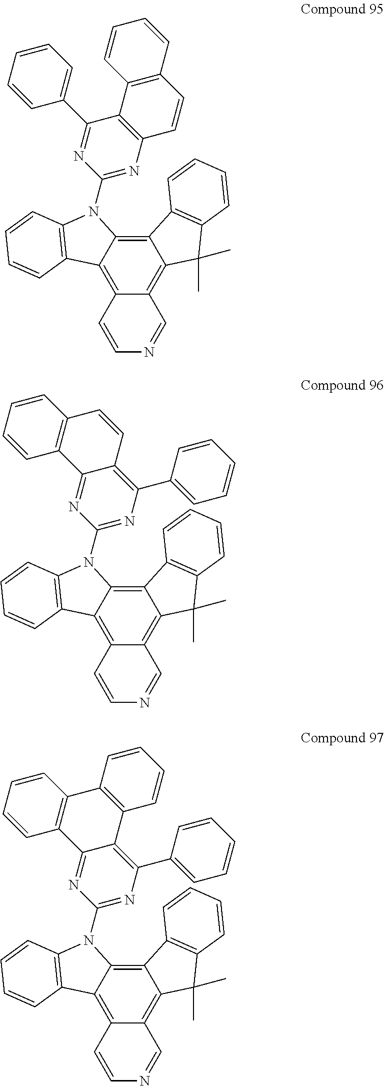

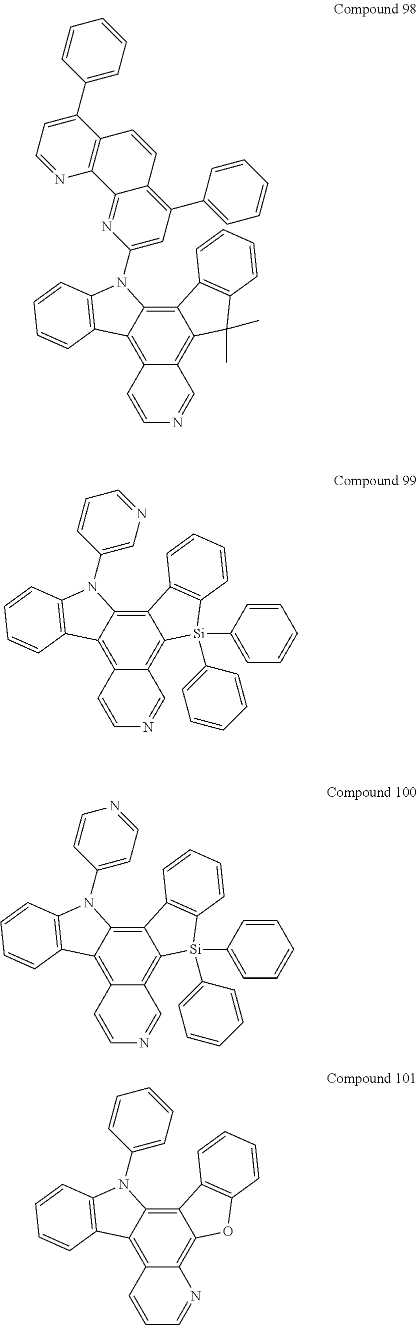

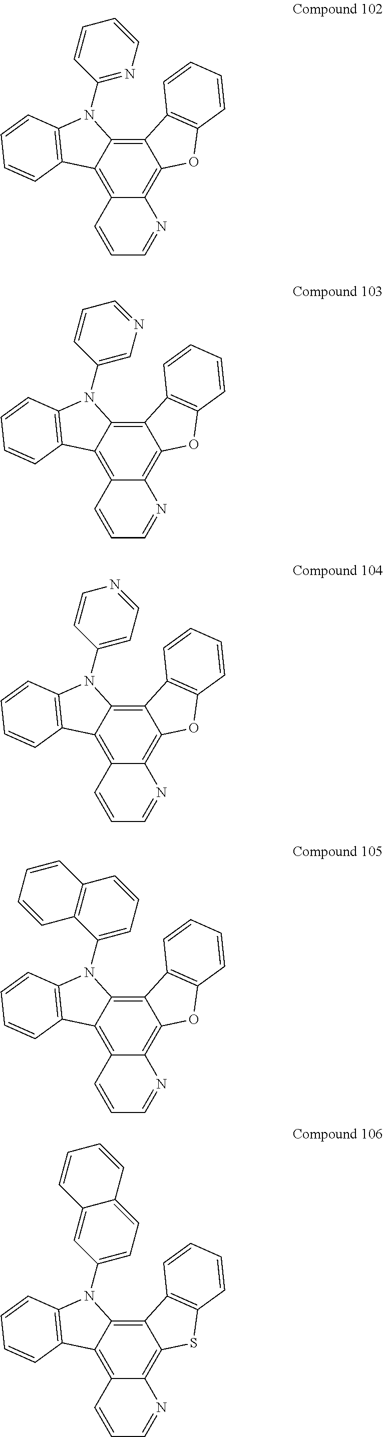

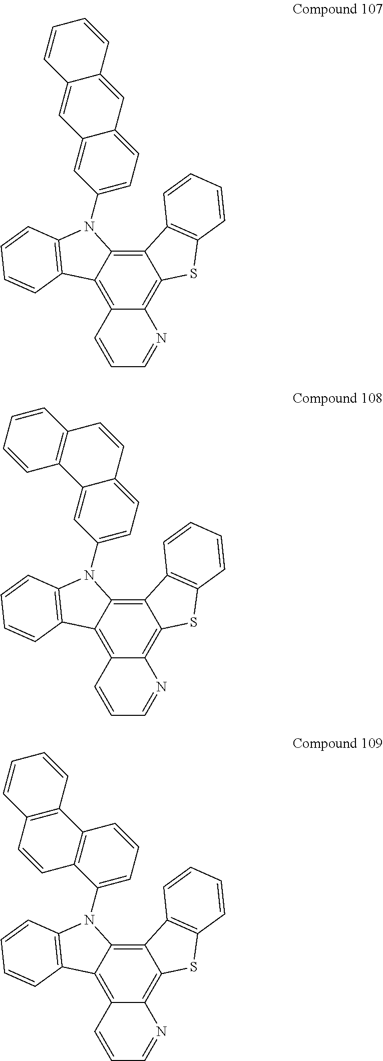 [SMILES: c1ccc2cc3cc(-n4c5ccccc5c5c6cccnc6c6sc7ccccc7c6c54)ccc3cc2c1 ; c1ccc2c(c1)ccc1ccc(-n3c4ccccc4c4c5cccnc5c5sc6ccccc6c5c43)cc12 ; c1ccc2c(c1)ccc1c(-n3c4ccccc4c4c5cccnc5c5sc6ccccc6c5c43)cccc12]

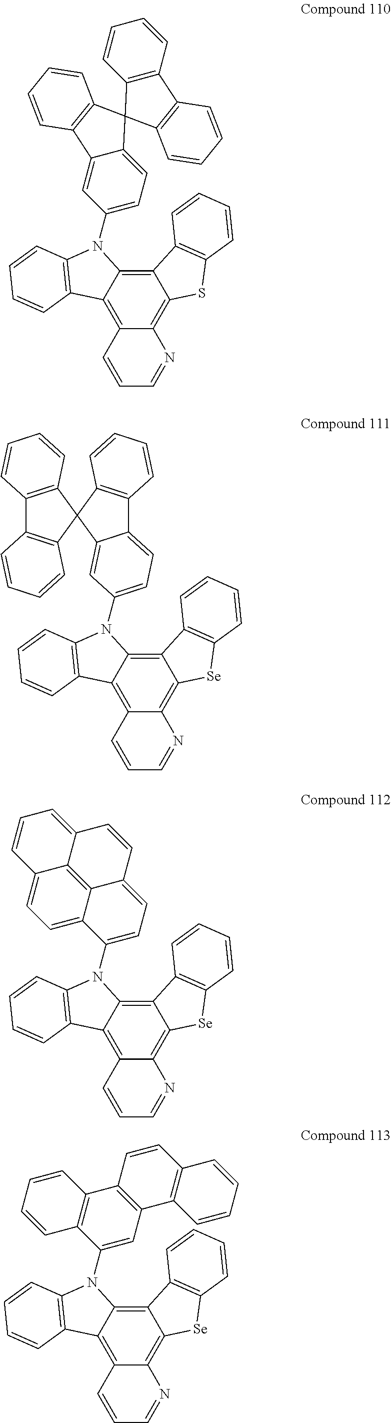

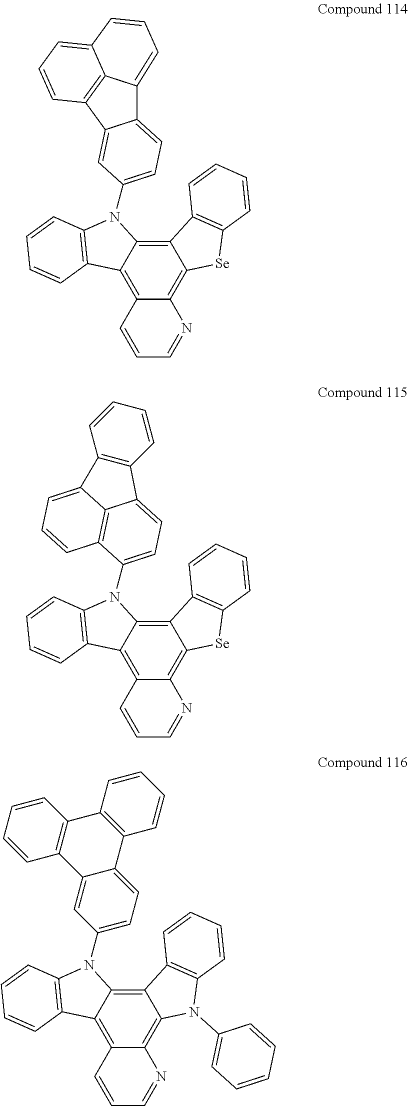

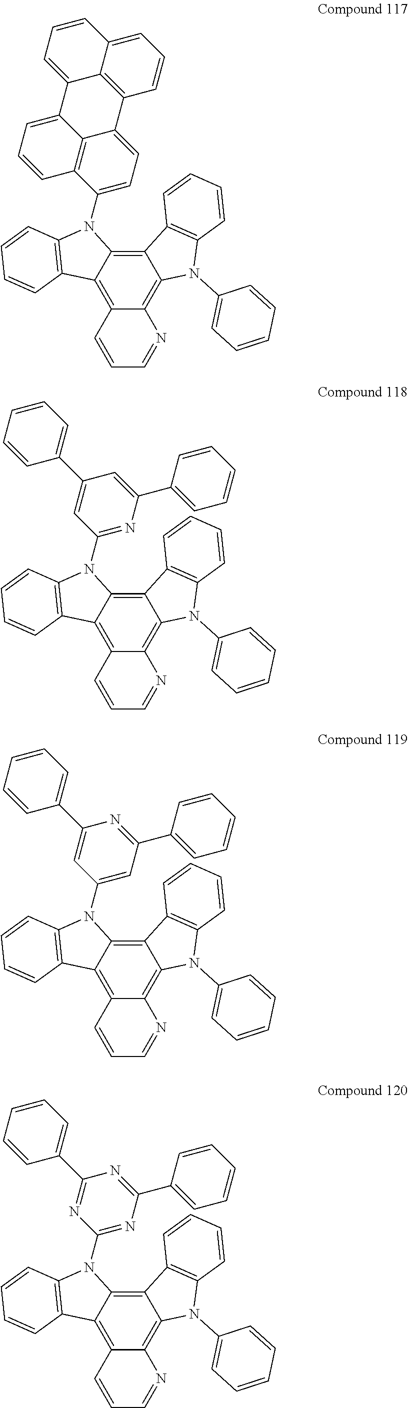 [SMILES: c1ccc(-n2c3ccccc3c3c2c2ncccc2c2c4ccccc4n(-c4ccc5c6cccc7cccc(c8cccc4c85)c76)c23)cc1 ; c1ccc(-c2cc(-c3ccccc3)nc(-n3c4ccccc4c4c5cccnc5c5c(c6ccccc6n5-c5ccccc5)c43)c2)cc1 ; c1ccc(-c2cc(-n3c4ccccc4c4c5cccnc5c5c(c6ccccc6n5-c5ccccc5)c43)cc(-c3ccccc3)n2)cc1 ; c1ccc(-c2nc(-c3ccccc3)nc(-n3c4ccccc4c4c5cccnc5c5c(c6ccccc6n5-c5ccccc5)c43)n2)cc1]

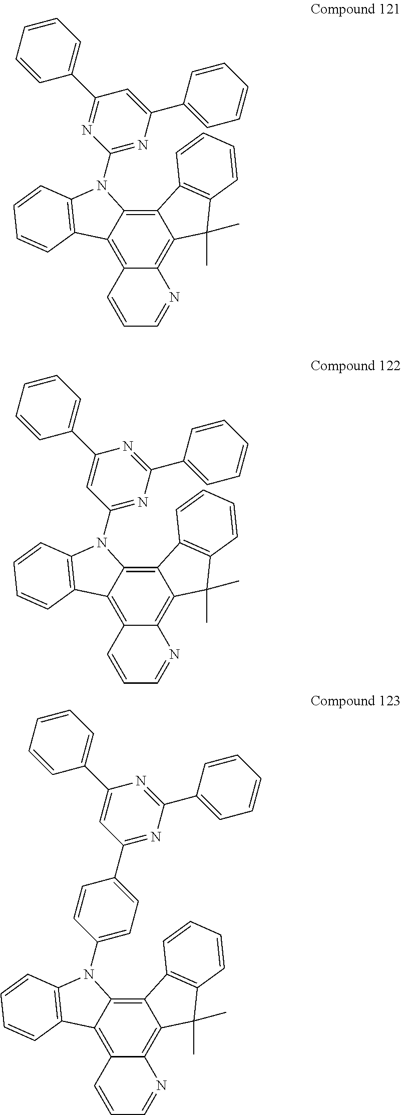 [SMILES: CC1(C)c2ccccc2-c2c1c1ncccc1c1c3ccccc3n(-c3nc(-c4ccccc4)cc(-c4ccccc4)n3)c21 ; CC1(C)c2ccccc2-c2c1c1ncccc1c1c3ccccc3n(-c3cc(-c4ccccc4)nc(-c4ccccc4)n3)c21 ; CC1(C)c2ccccc2-c2c1c1ncccc1c1c3ccccc3n(-c3ccc(-c4cc(-c5ccccc5)nc(-c5ccccc5)n4)cc3)c21]

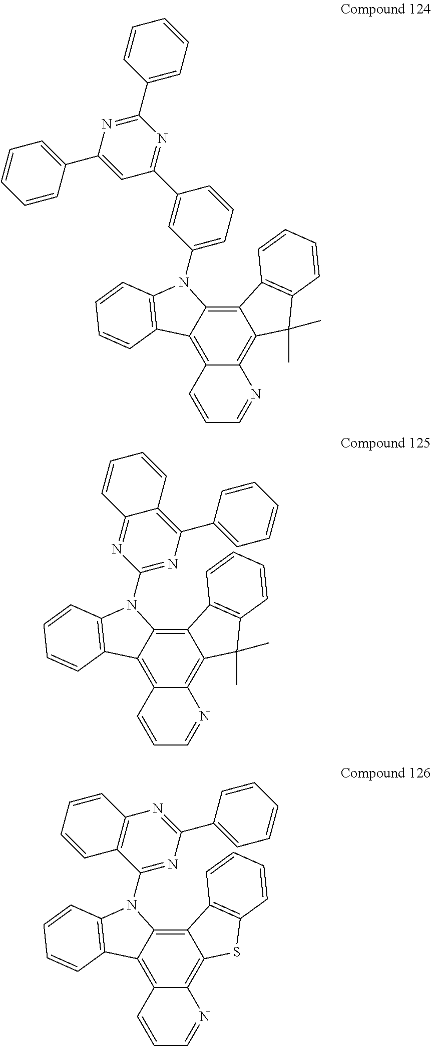 [SMILES: CC1(C)c2ccccc2-c2c1c1ncccc1c1c3ccccc3n(-c3cccc(-c4cc(-c5ccccc5)nc(-c5ccccc5)n4)c3)c21 ; CC1(C)c2ccccc2-c2c1c1ncccc1c1c3ccccc3n(-c3nc(-c4ccccc4)c4ccccc4n3)c21 ; c1ccc(-c2nc(-n3c4ccccc4c4c5cccnc5c5sc6ccccc6c5c43)c3ccccc3n2)cc1]

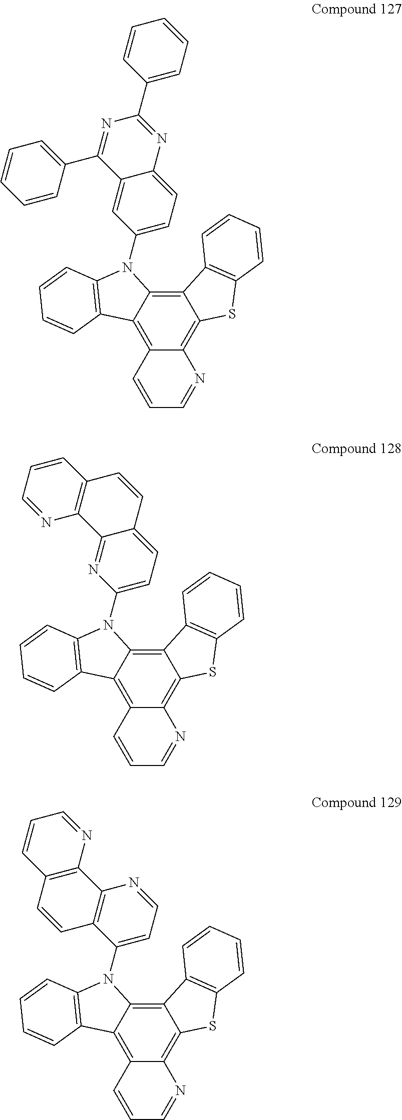 [SMILES: c1ccc(-c2nc(-c3ccccc3)c3cc(-n4c5ccccc5c5c6cccnc6c6sc7ccccc7c6c54)ccc3n2)cc1 ; c1cnc2c(c1)ccc1ccc(-n3c4ccccc4c4c5cccnc5c5sc6ccccc6c5c43)nc12 ; c1cnc2c(c1)ccc1c(-n3c4ccccc4c4c5cccnc5c5sc6ccccc6c5c43)ccnc12]

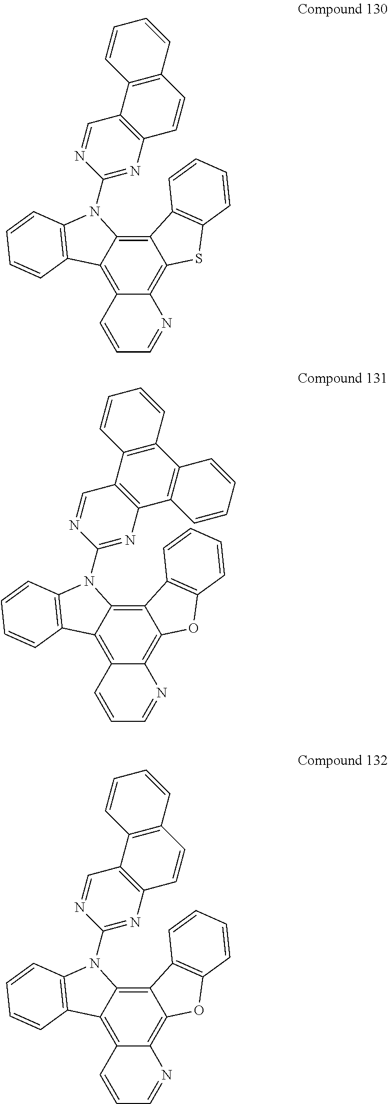

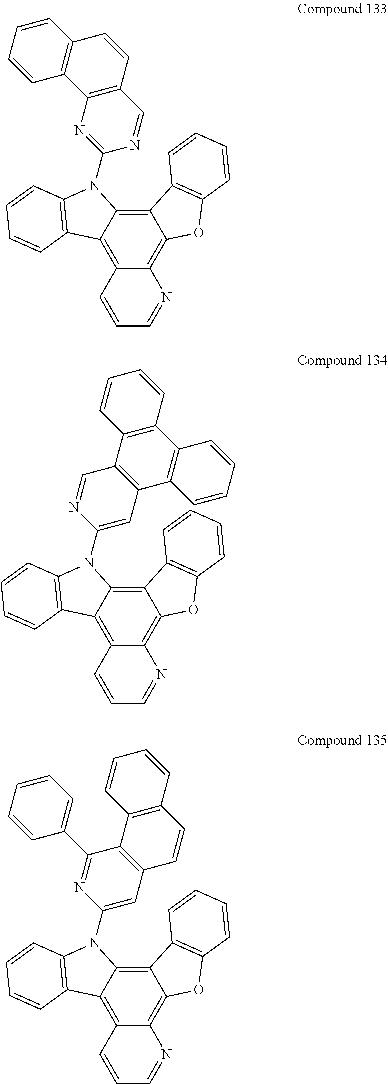

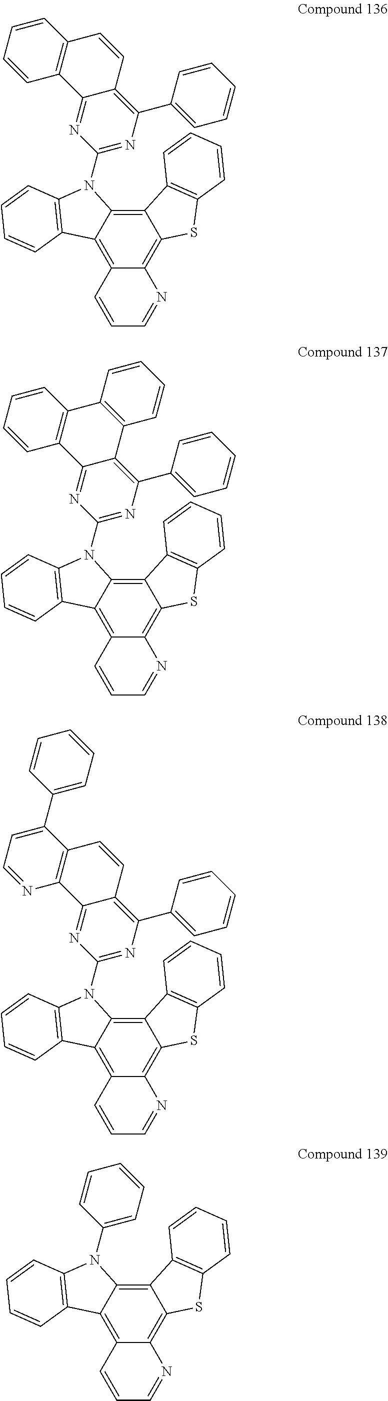

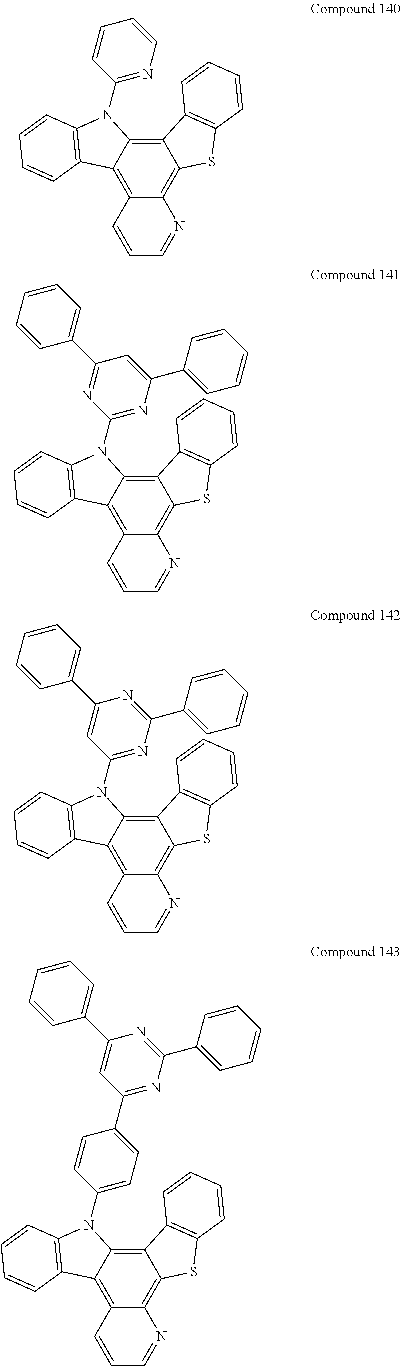 [SMILES: c1ccc(-n2c3ccccc3c3c4cccnc4c4sc5ccccc5c4c32)nc1 ; c1ccc(-c2cc(-c3ccccc3)nc(-n3c4ccccc4c4c5cccnc5c5sc6ccccc6c5c43)n2)cc1 ; c1ccc(-c2cc(-n3c4ccccc4c4c5cccnc5c5sc6ccccc6c5c43)nc(-c3ccccc3)n2)cc1 ; c1ccc(-c2cc(-c3ccc(-n4c5ccccc5c5c6cccnc6c6sc7ccccc7c6c54)cc3)nc(-c3ccccc3)n2)cc1]

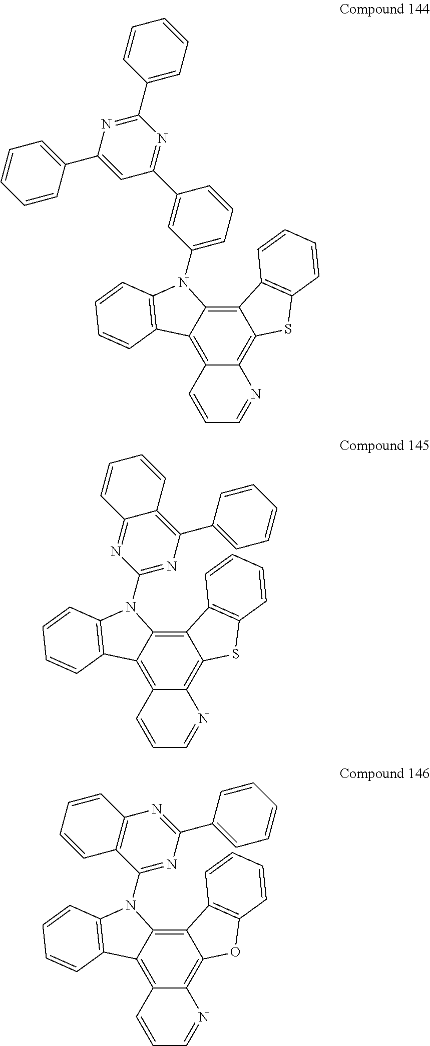 [SMILES: c1ccc(-c2cc(-c3cccc(-n4c5ccccc5c5c6cccnc6c6sc7ccccc7c6c54)c3)nc(-c3ccccc3)n2)cc1 ; c1ccc(-c2nc(-n3c4ccccc4c4c5cccnc5c5sc6ccccc6c5c43)nc3ccccc23)cc1 ; c1ccc(-c2nc(-n3c4ccccc4c4c5cccnc5c5oc6ccccc6c5c43)c3ccccc3n2)cc1]

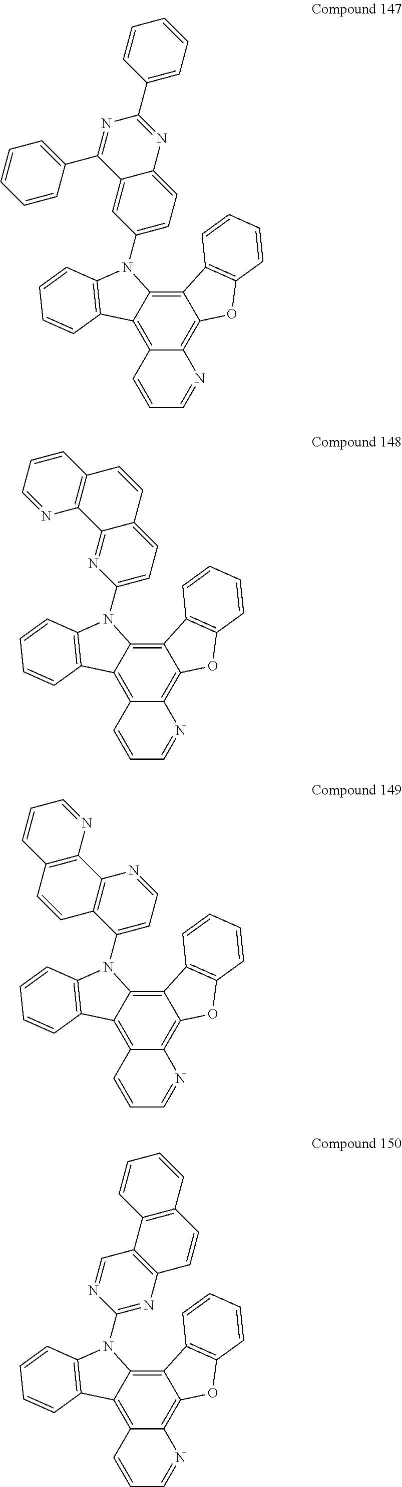

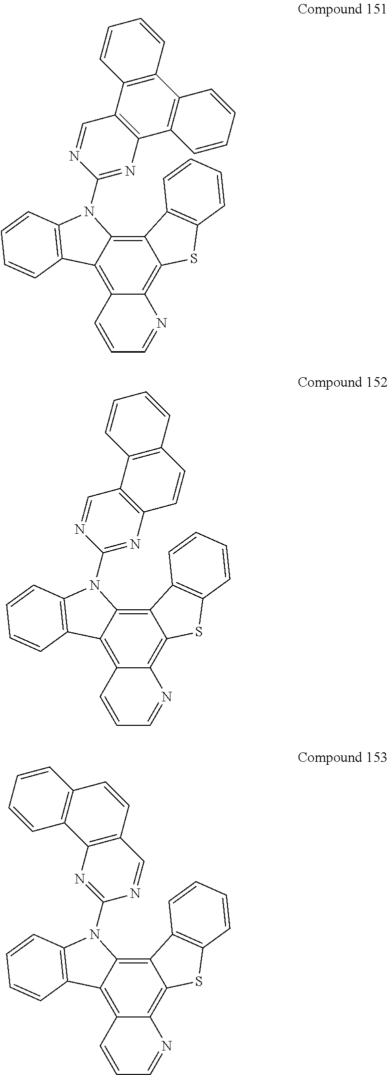

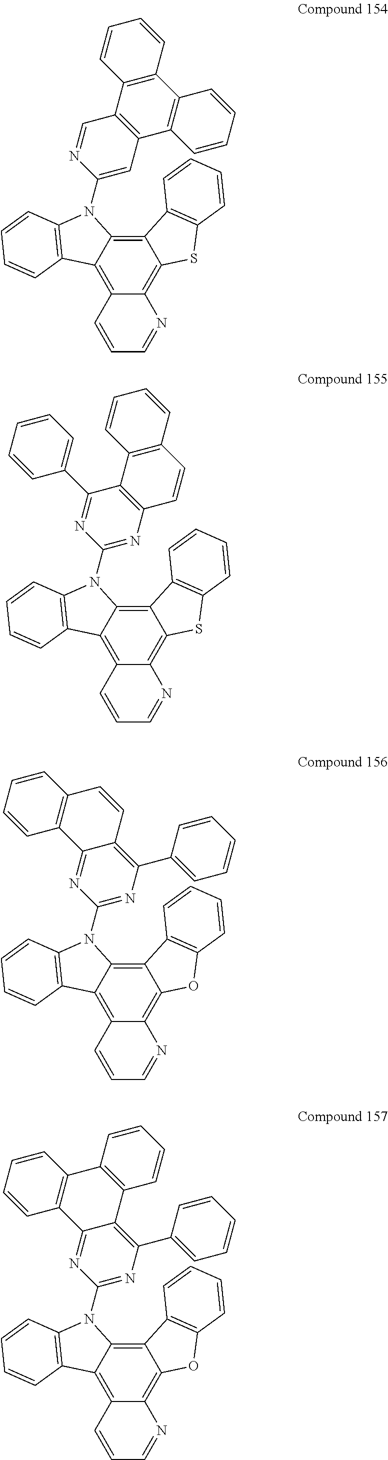

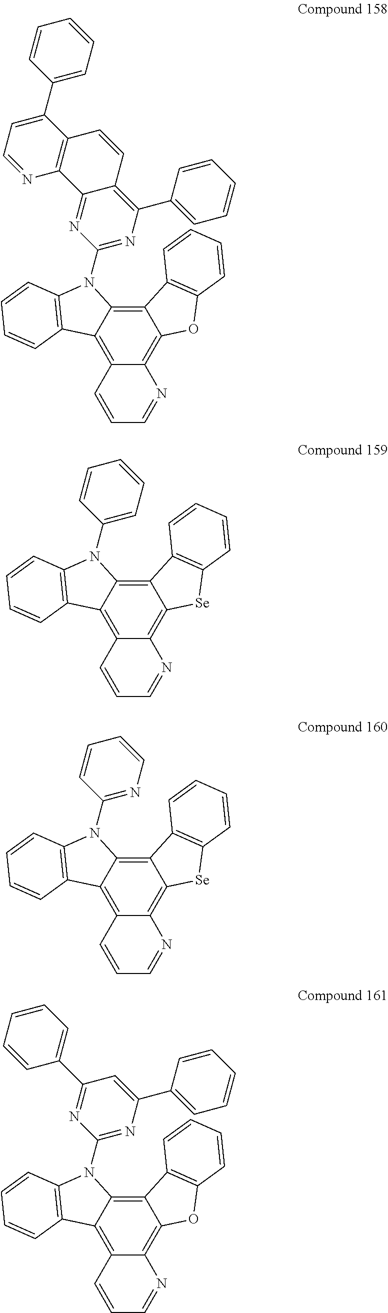

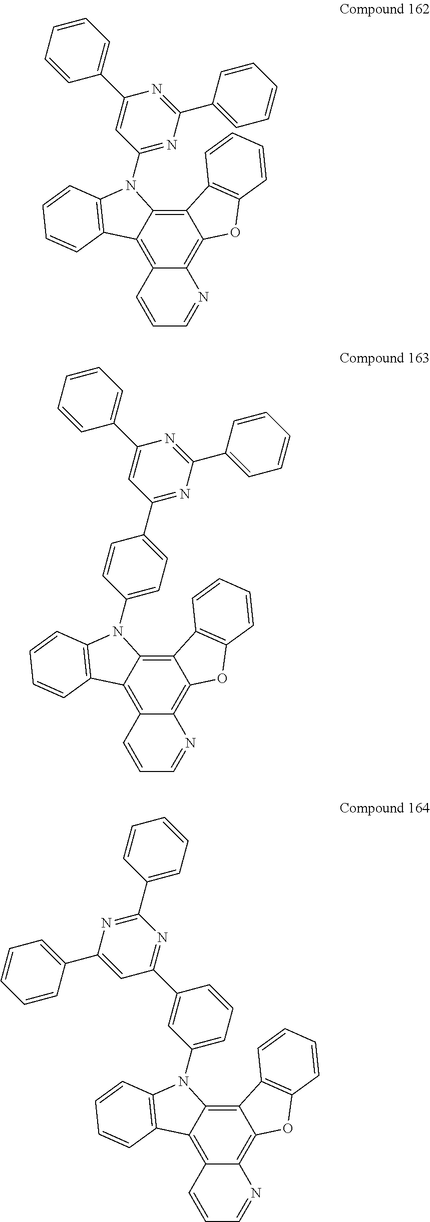 [SMILES: c1ccc(-c2cc(-n3c4ccccc4c4c5cccnc5c5oc6ccccc6c5c43)nc(-c3ccccc3)n2)cc1 ; c1ccc(-c2cc(-c3ccc(-n4c5ccccc5c5c6cccnc6c6oc7ccccc7c6c54)cc3)nc(-c3ccccc3)n2)cc1 ; c1ccc(-c2cc(-c3cccc(-n4c5ccccc5c5c6cccnc6c6oc7ccccc7c6c54)c3)nc(-c3ccccc3)n2)cc1]

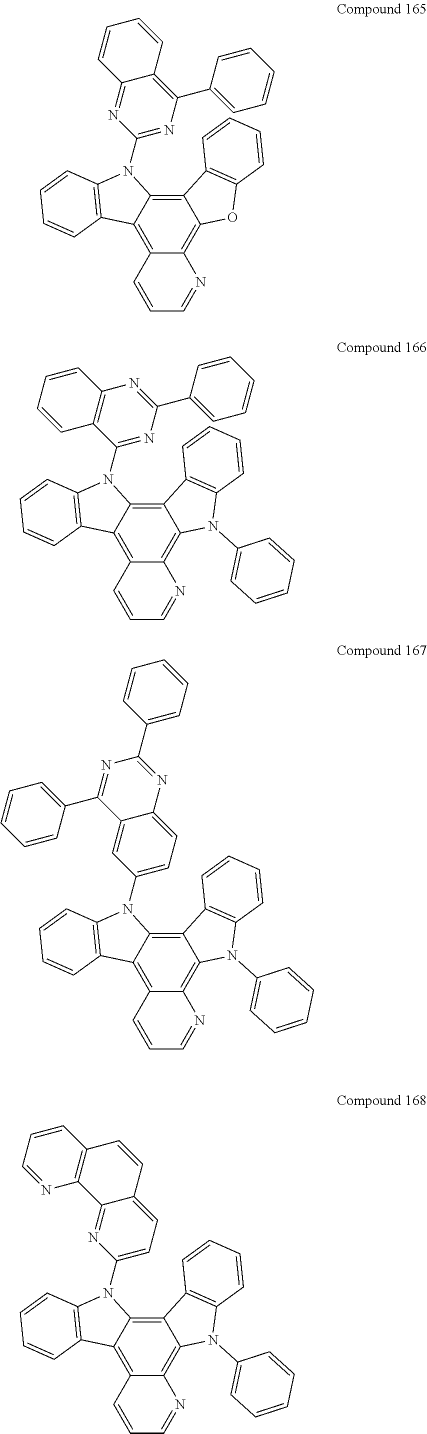

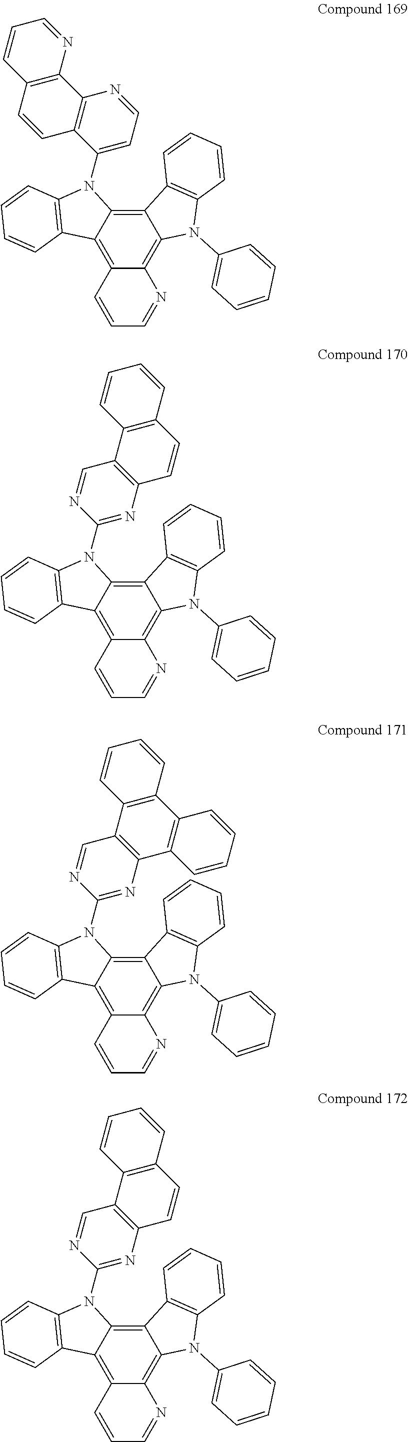

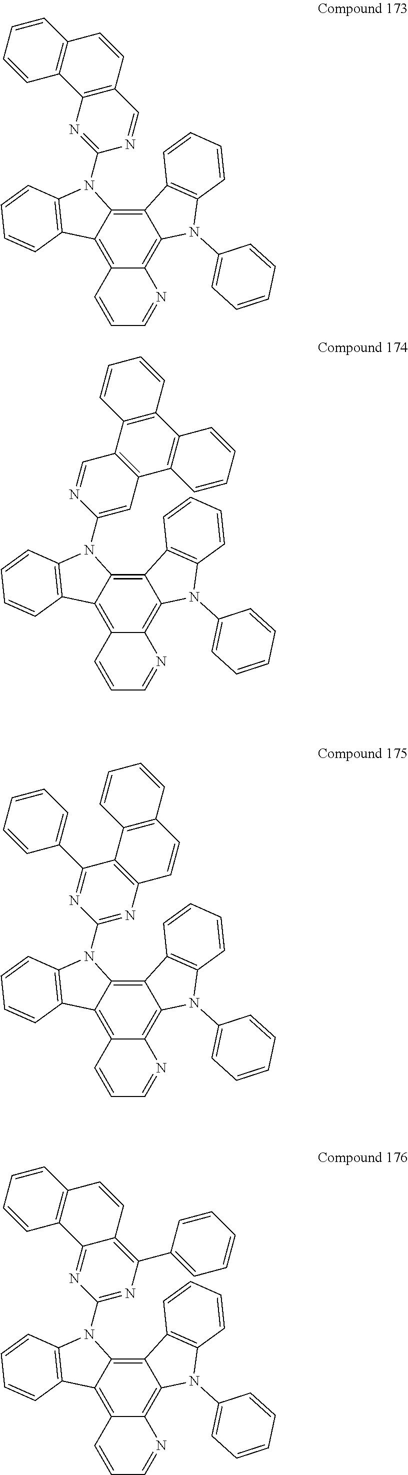

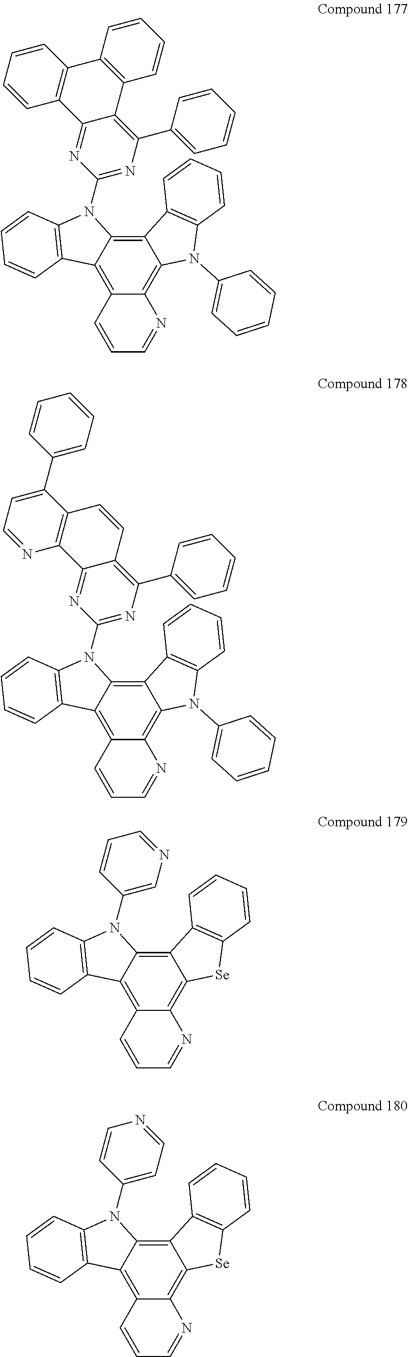

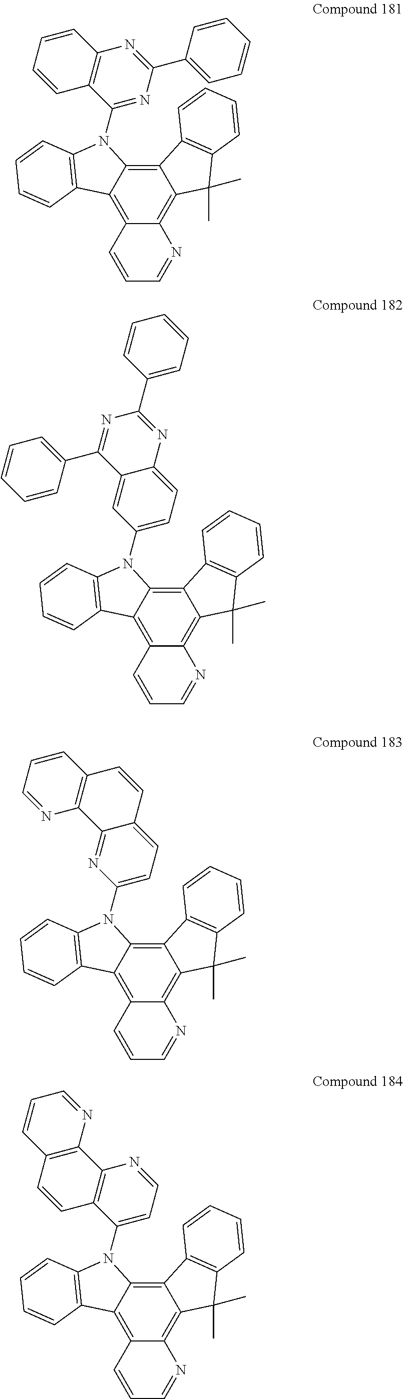

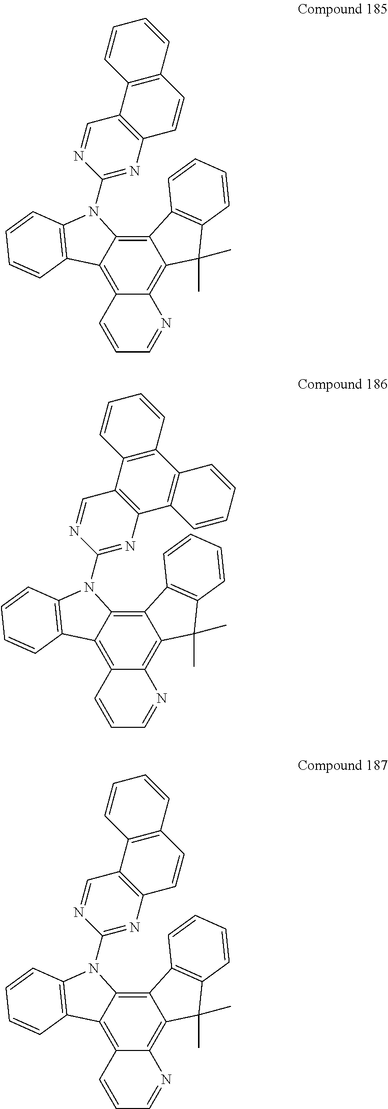

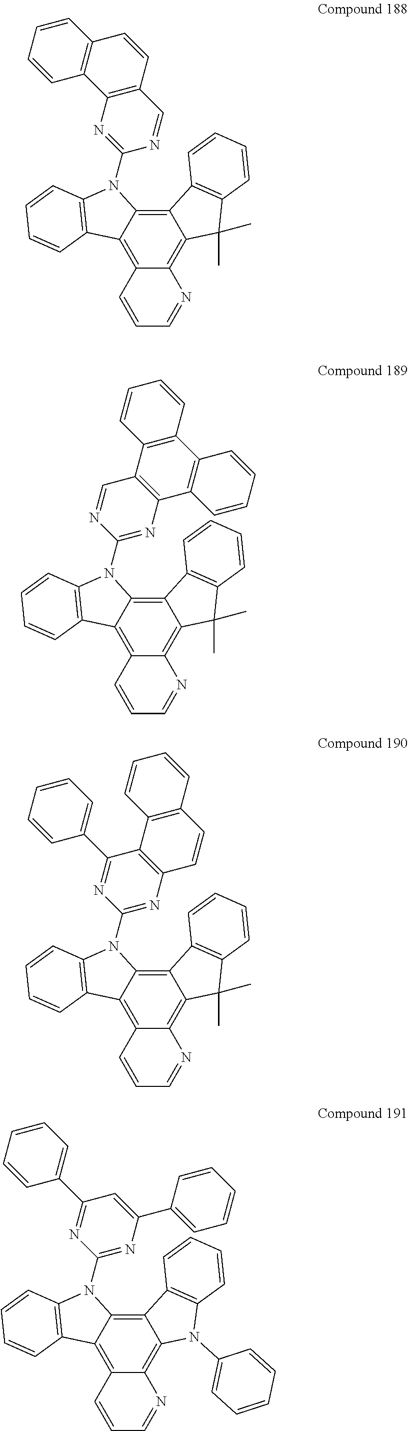

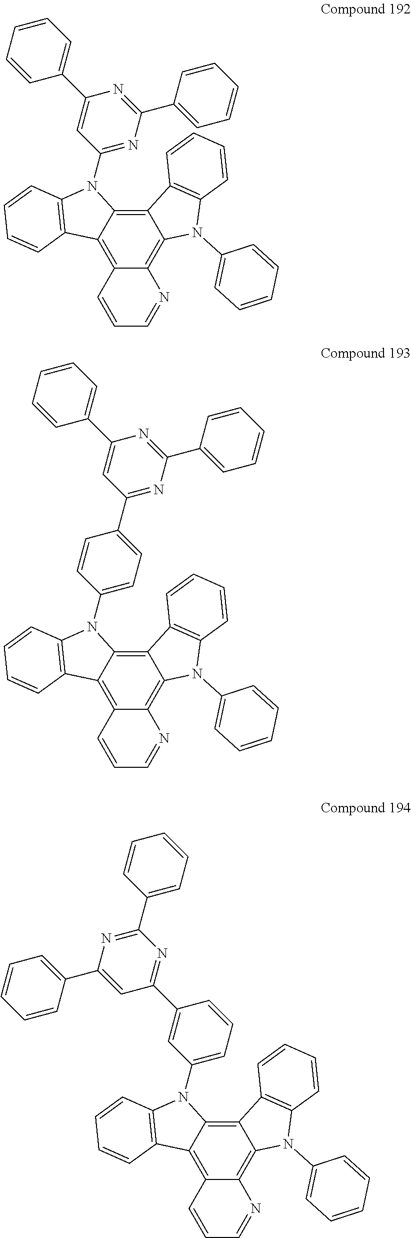 [SMILES: c1ccc(-c2cc(-n3c4ccccc4c4c5cccnc5c5c(c6ccccc6n5-c5ccccc5)c43)nc(-c3ccccc3)n2)cc1 ; c1ccc(-c2cc(-c3ccc(-n4c5ccccc5c5c6cccnc6c6c(c7ccccc7n6-c6ccccc6)c54)cc3)nc(-c3ccccc3)n2)cc1 ; c1ccc(-c2cc(-c3cccc(-n4c5ccccc5c5c6cccnc6c6c(c7ccccc7n6-c6ccccc6)c54)c3)nc(-c3ccccc3)n2)cc1]

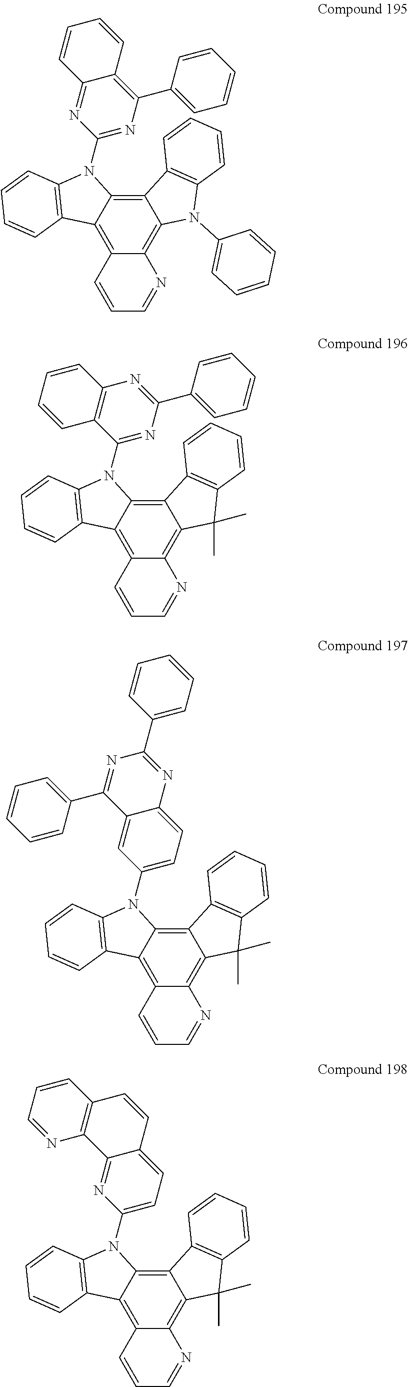

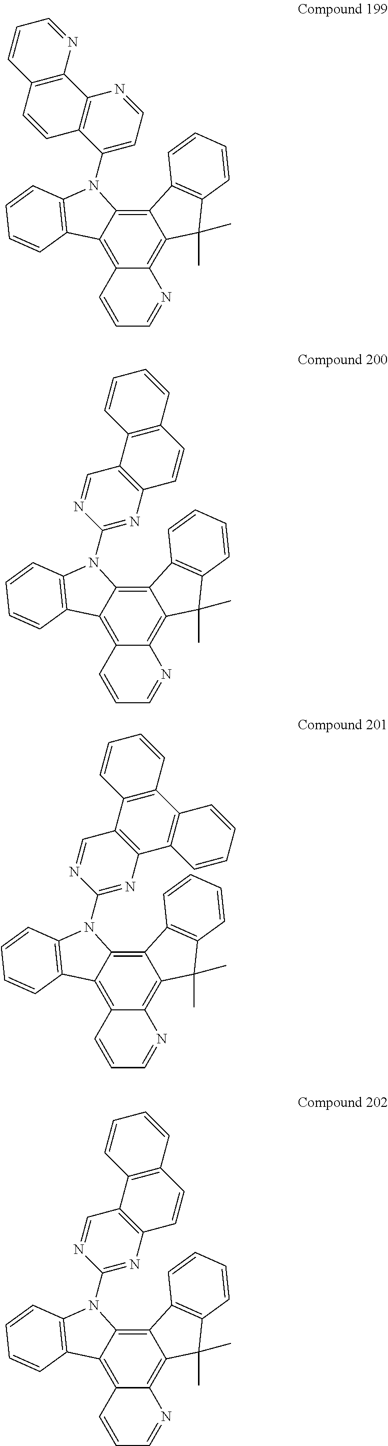

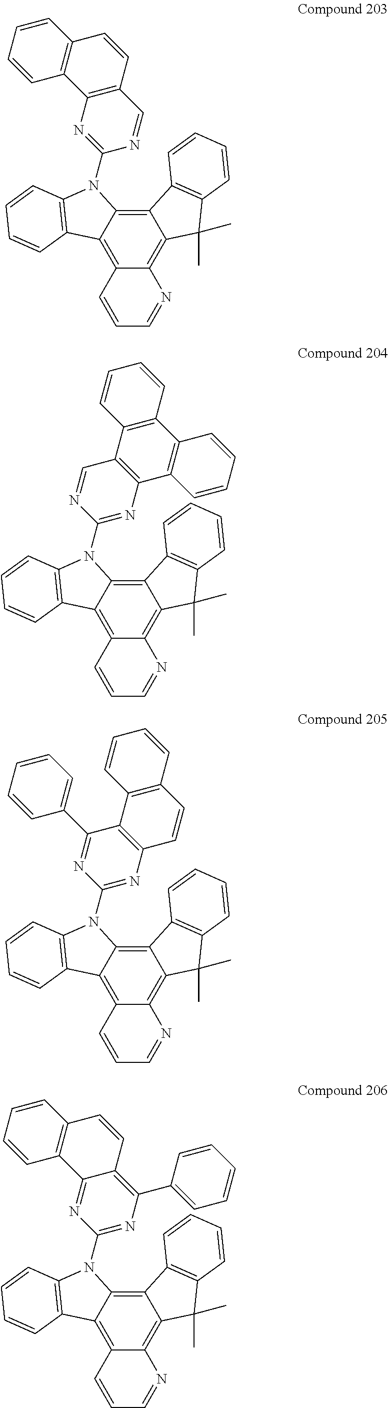

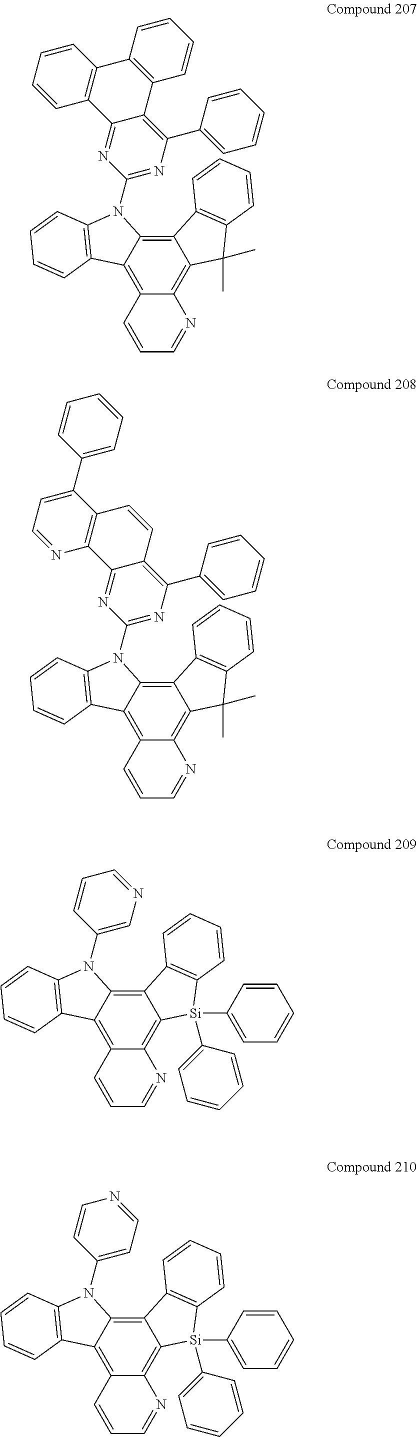 [SMILES: CC1(C)c2ccccc2-c2c1c1ncccc1c1c3ccccc3n(-c3nc(-c4ccccc4)c4c5ccccc5c5ccccc5c4n3)c21 ; CC1(C)c2ccccc2-c2c1c1ncccc1c1c3ccccc3n(-c3nc(-c4ccccc4)c4ccc5c(-c6ccccc6)ccnc5c4n3)c21 ; c1ccc([Si]2(c3ccccc3)c3ccccc3-c3c2c2ncccc2c2c4ccccc4n(-c4cccnc4)c32)cc1 ; c1ccc([Si]2(c3ccccc3)c3ccccc3-c3c2c2ncccc2c2c4ccccc4n(-c4ccncc4)c32)cc1]

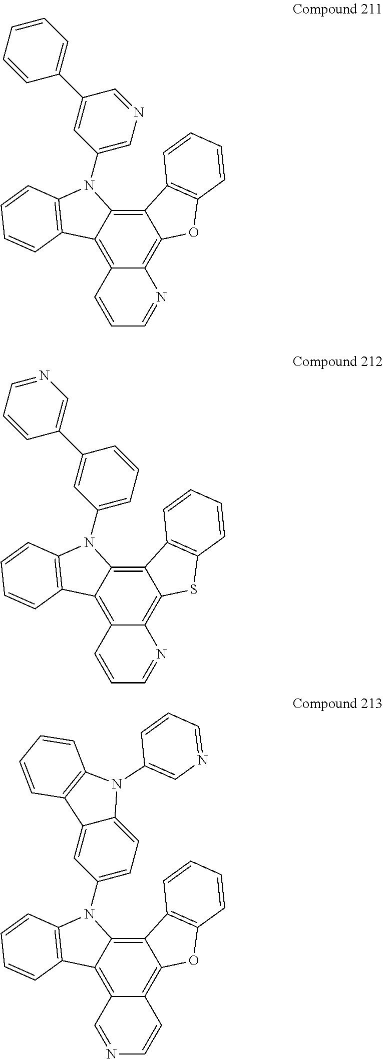

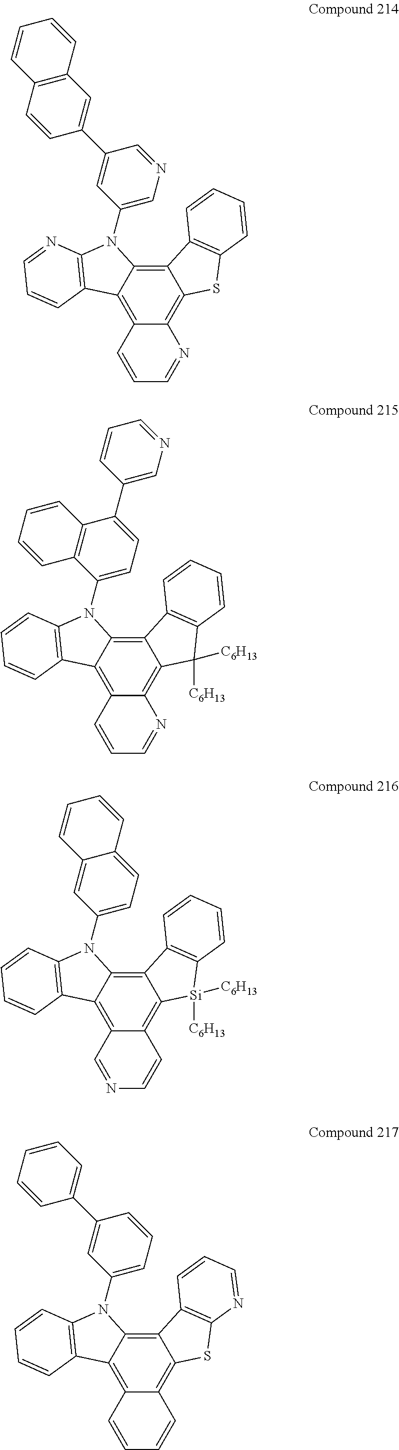

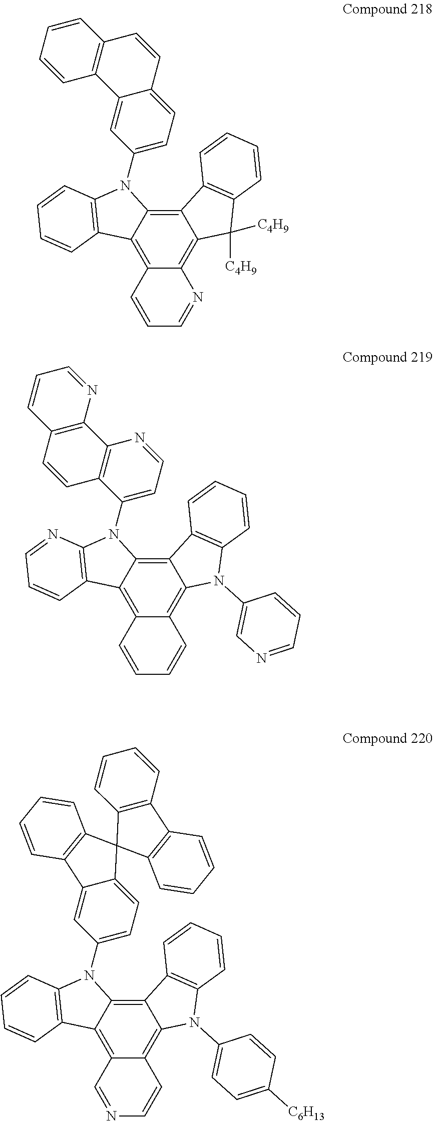

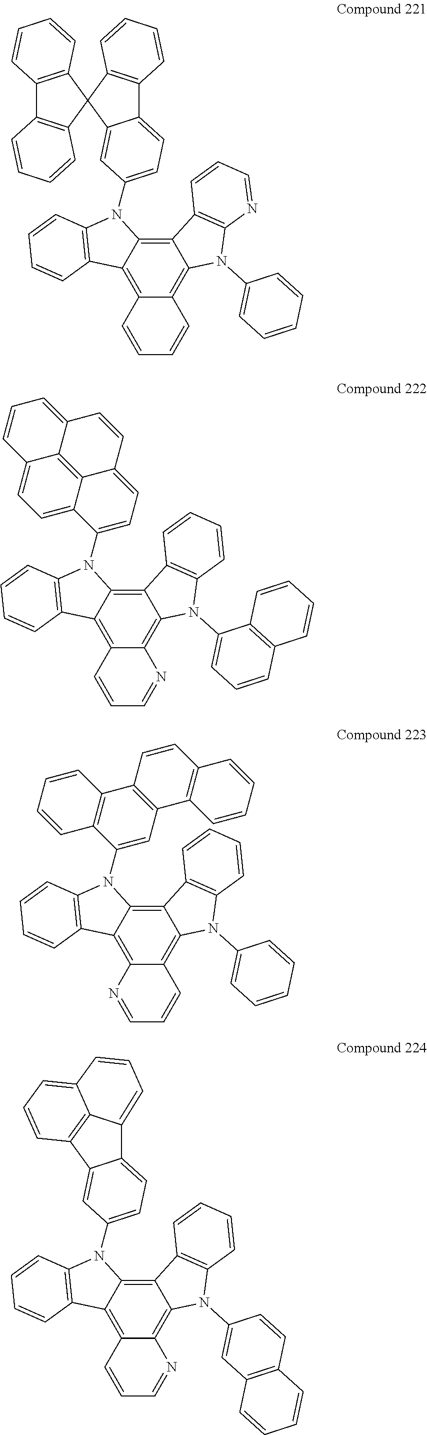

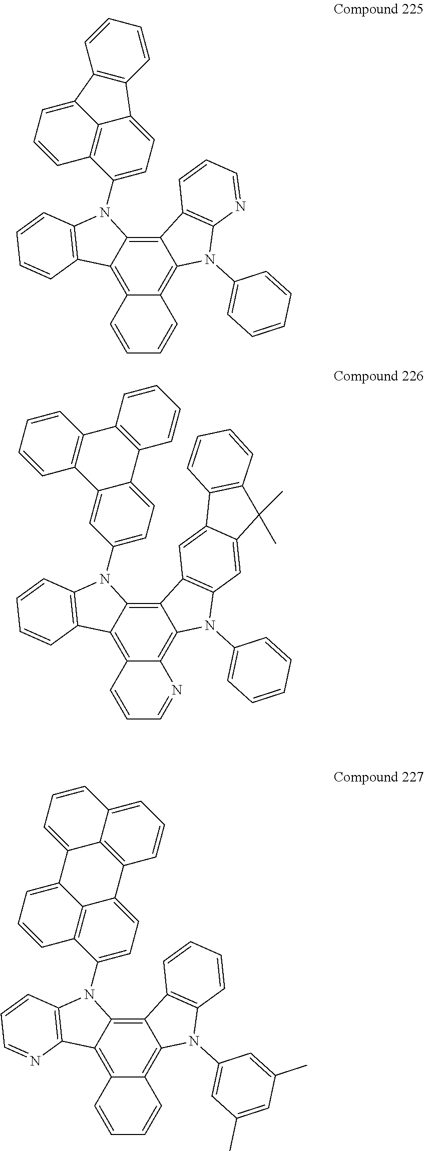

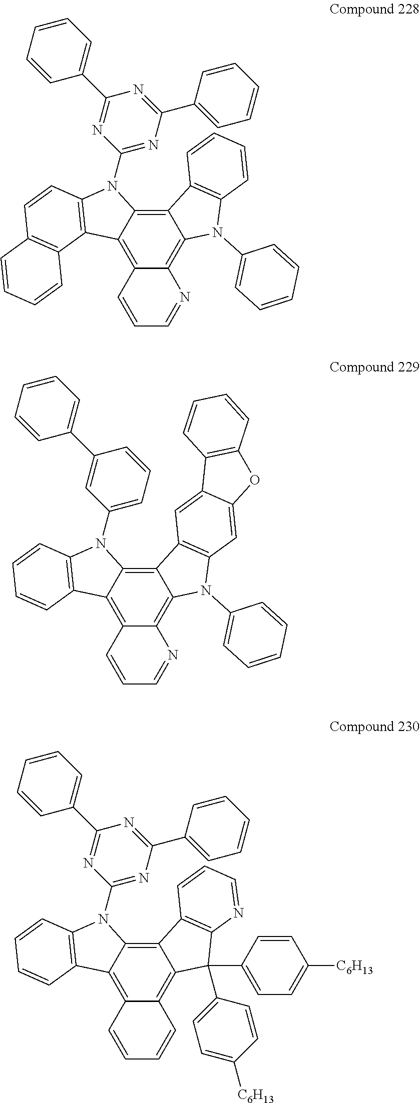

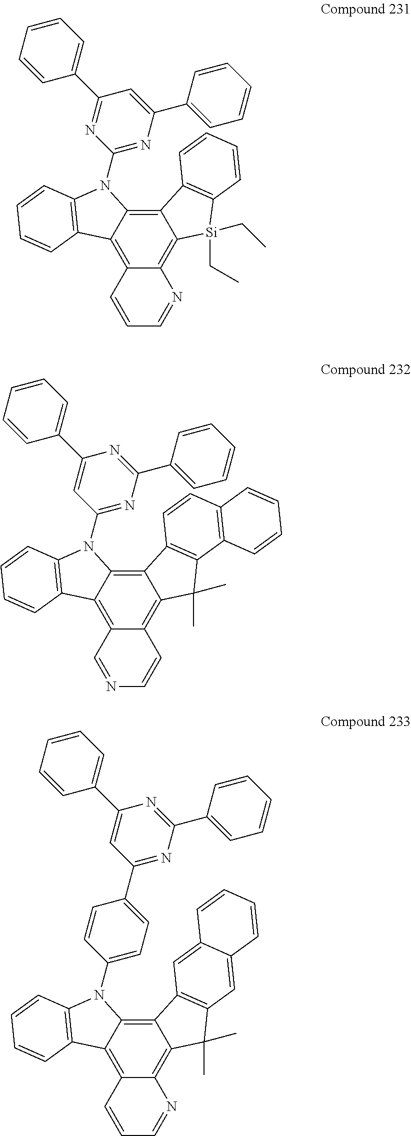 [SMILES: CC[Si]1(CC)c2ccccc2-c2c1c1ncccc1c1c3ccccc3n(-c3nc(-c4ccccc4)cc(-c4ccccc4)n3)c21 ; CC1(C)c2c(ccc3ccccc23)-c2c1c1ccncc1c1c3ccccc3n(-c3cc(-c4ccccc4)nc(-c4ccccc4)n3)c21 ; CC1(C)c2cc3ccccc3cc2-c2c1c1ncccc1c1c3ccccc3n(-c3ccc(-c4cc(-c5ccccc5)nc(-c5ccccc5)n4)cc3)c21]

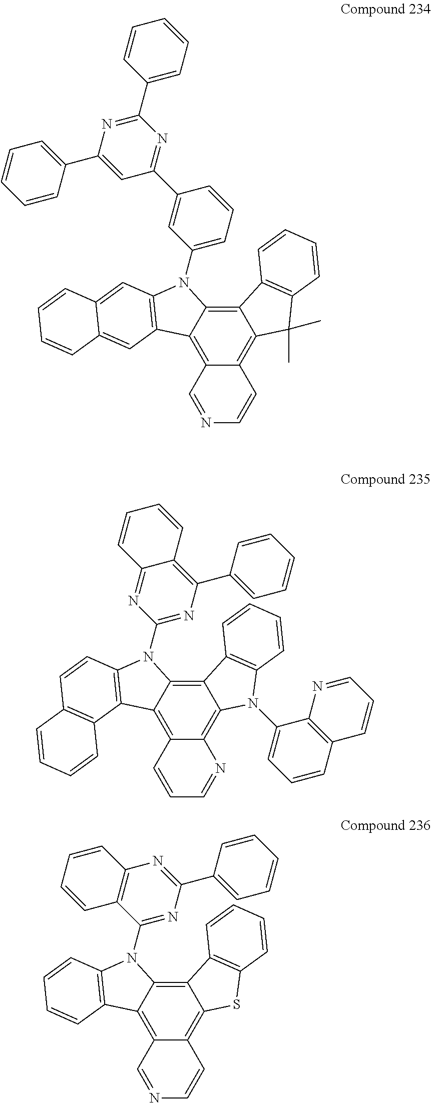 [SMILES: CC1(C)c2ccccc2-c2c1c1ccncc1c1c3cc4ccccc4cc3n(-c3cccc(-c4cc(-c5ccccc5)nc(-c5ccccc5)n4)c3)c21 ; c1ccc(-c2nc(-n3c4ccc5ccccc5c4c4c5cccnc5c5c(c6ccccc6n5-c5cccc6cccnc56)c43)nc3ccccc23)cc1 ; c1ccc(-c2nc(-n3c4ccccc4c4c5cnccc5c5sc6ccccc6c5c43)c3ccccc3n2)cc1]

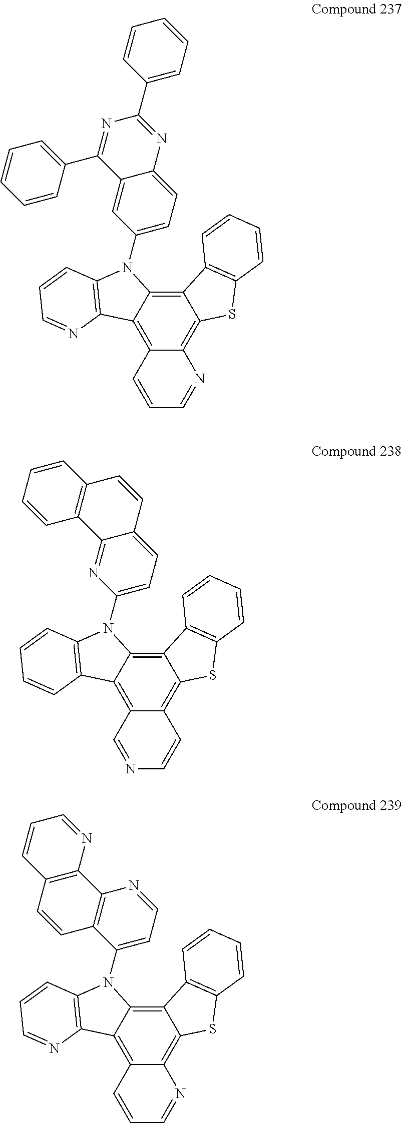 [SMILES: c1ccc(-c2nc(-c3ccccc3)c3cc(-n4c5cccnc5c5c6cccnc6c6sc7ccccc7c6c54)ccc3n2)cc1 ; c1ccc2c(c1)ccc1ccc(-n3c4ccccc4c4c5cnccc5c5sc6ccccc6c5c43)nc12 ; c1cnc2c(c1)ccc1c(-n3c4cccnc4c4c5cccnc5c5sc6ccccc6c5c43)ccnc12]

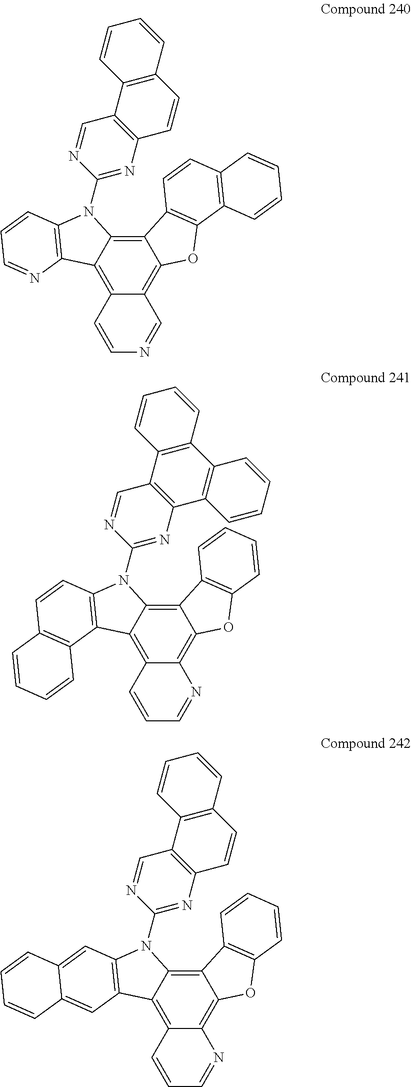

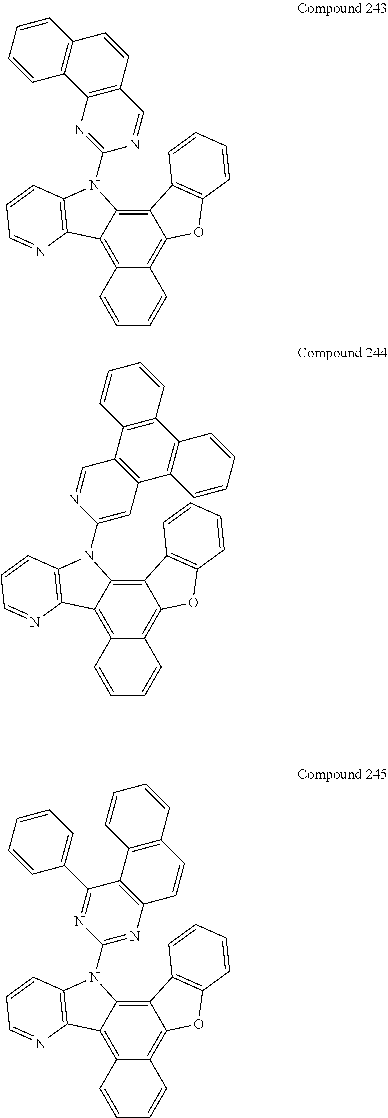

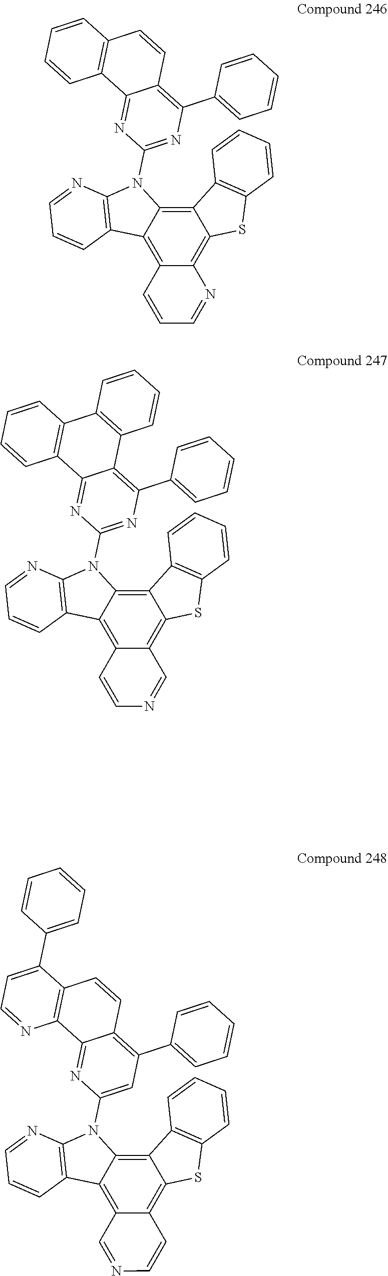 [SMILES: c1ccc(-c2nc(-n3c4ncccc4c4c5cccnc5c5sc6ccccc6c5c43)nc3c2ccc2ccccc23)cc1 ; c1ccc(-c2nc(-n3c4ncccc4c4c5ccncc5c5sc6ccccc6c5c43)nc3c4ccccc4c4ccccc4c23)cc1 ; c1ccc(-c2ccnc3c2ccc2c(-c4ccccc4)cc(-n4c5ncccc5c5c6cnccc6c6sc7ccccc7c6c54)nc23)cc1]

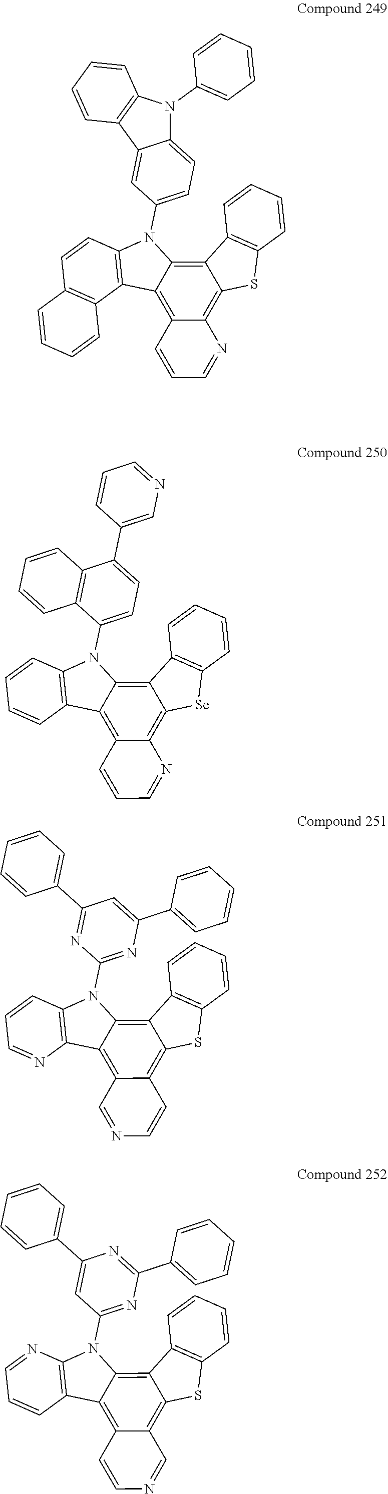 [SMILES: c1ccc(-n2c3ccccc3c3cc(-n4c5ccc6ccccc6c5c5c6cccnc6c6sc7ccccc7c6c54)ccc32)cc1 ; c1cncc(-c2ccc(-n3c4ccccc4c4c5cccnc5c5[se]c6ccccc6c5c43)c3ccccc23)c1 ; c1ccc(-c2cc(-c3ccccc3)nc(-n3c4cccnc4c4c5cnccc5c5sc6ccccc6c5c43)n2)cc1 ; c1ccc(-c2cc(-n3c4ncccc4c4c5ccncc5c5sc6ccccc6c5c43)nc(-c3ccccc3)n2)cc1]

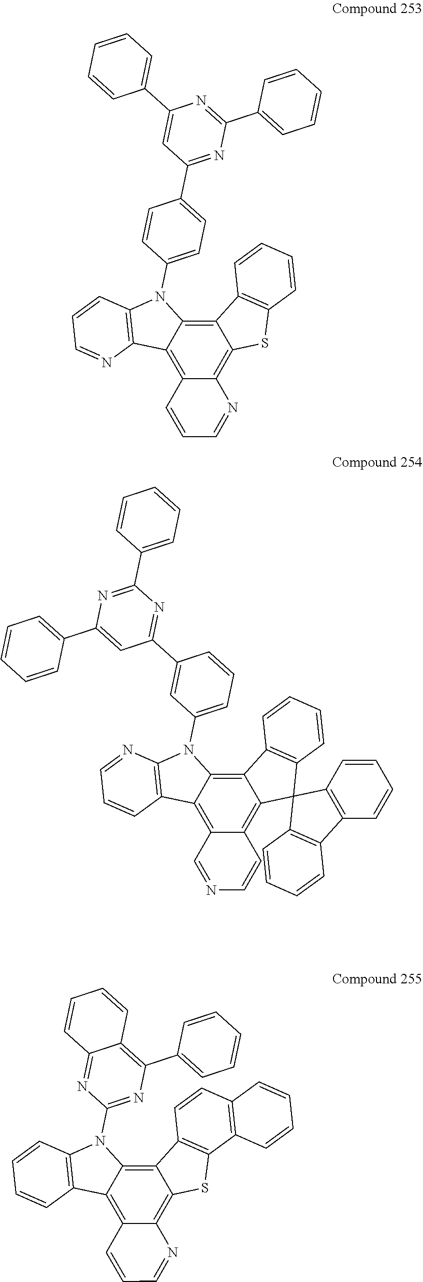 [SMILES: c1ccc(-c2cc(-c3ccc(-n4c5cccnc5c5c6cccnc6c6sc7ccccc7c6c54)cc3)nc(-c3ccccc3)n2)cc1 ; c1ccc(-c2cc(-c3cccc(-n4c5ncccc5c5c6cnccc6c6c(c54)-c4ccccc4C64c5ccccc5-c5ccccc54)c3)nc(-c3ccccc3)n2)cc1 ; c1ccc(-c2nc(-n3c4ccccc4c4c5cccnc5c5sc6c7ccccc7ccc6c5c43)nc3ccccc23)cc1]

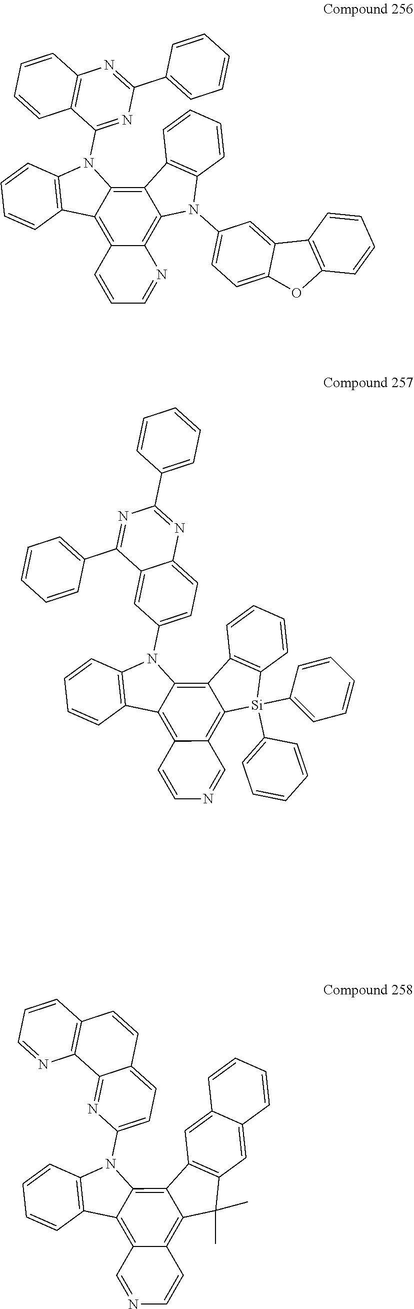 [SMILES: c1ccc(-c2nc(-n3c4ccccc4c4c5cccnc5c5c(c6ccccc6n5-c5ccc6oc7ccccc7c6c5)c43)c3ccccc3n2)cc1 ; c1ccc(-c2nc(-c3ccccc3)c3cc(-n4c5ccccc5c5c6ccncc6c6c(c54)-c4ccccc4[Si]6(c4ccccc4)c4ccccc4)ccc3n2)cc1 ; CC1(C)c2cc3ccccc3cc2-c2c1c1ccncc1c1c3ccccc3n(-c3ccc4ccc5cccnc5c4n3)c21]

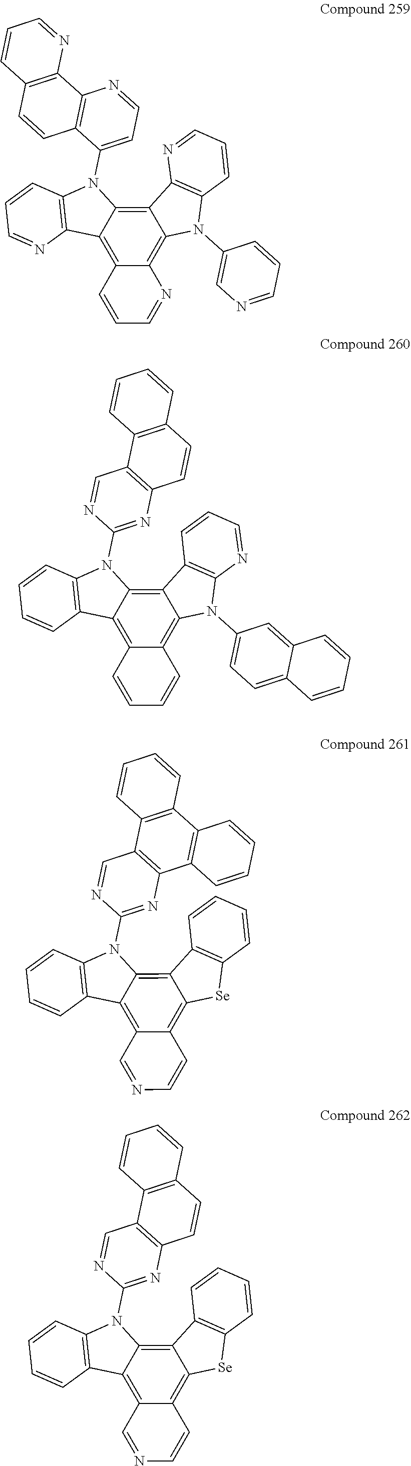

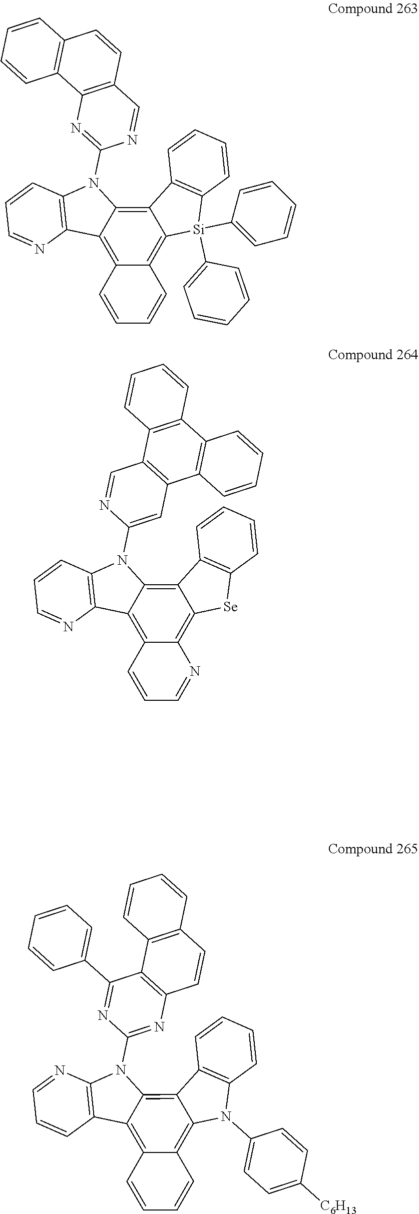

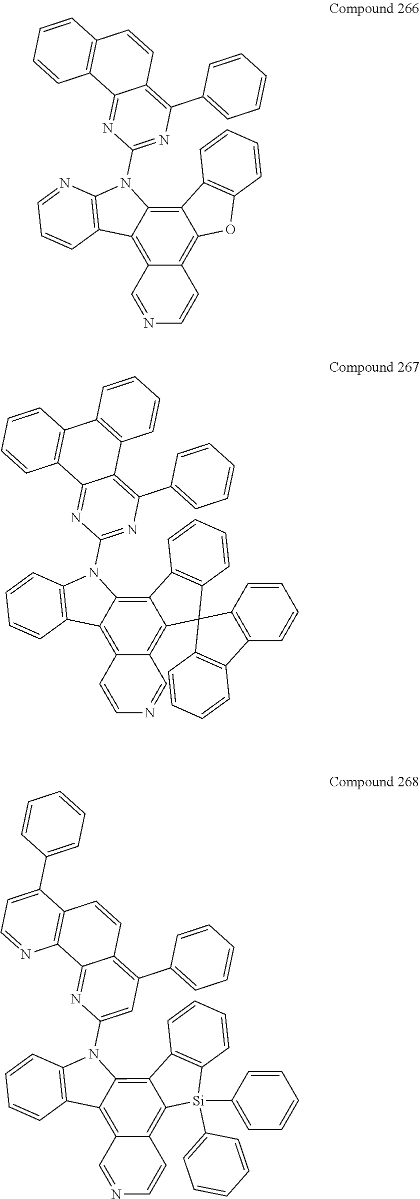 [SMILES: c1ccc(-c2nc(-n3c4ncccc4c4c5cnccc5c5oc6ccccc6c5c43)nc3c2ccc2ccccc23)cc1 ; c1ccc(-c2nc(-n3c4ccccc4c4c5ccncc5c5c(c43)-c3ccccc3C53c4ccccc4-c4ccccc43)nc3c4ccccc4c4ccccc4c23)cc1 ; c1ccc(-c2ccnc3c2ccc2c(-c4ccccc4)cc(-n4c5ccccc5c5c6cnccc6c6c(c54)-c4ccccc4[Si]6(c4ccccc4)c4ccccc4)nc23)cc1]

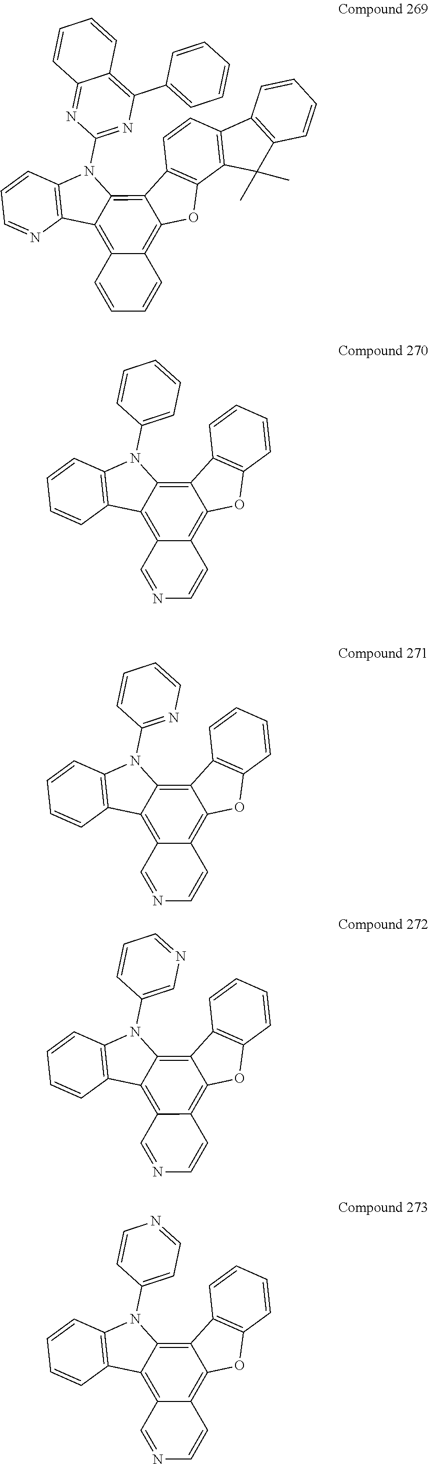

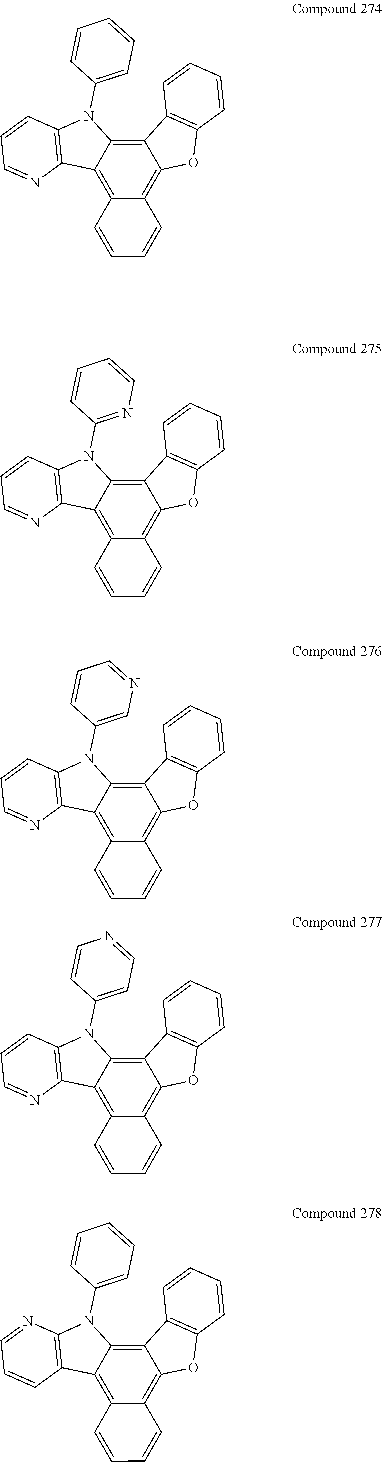

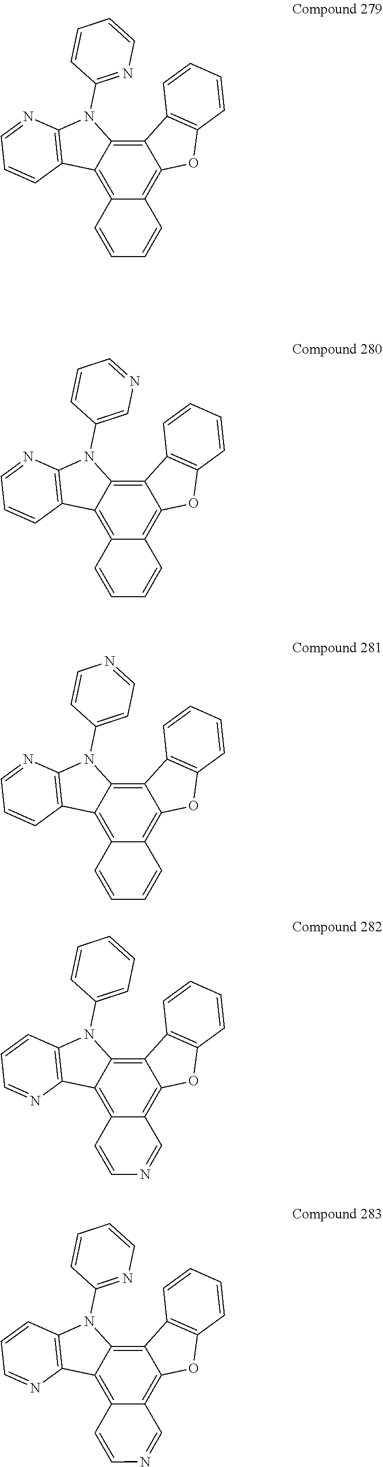

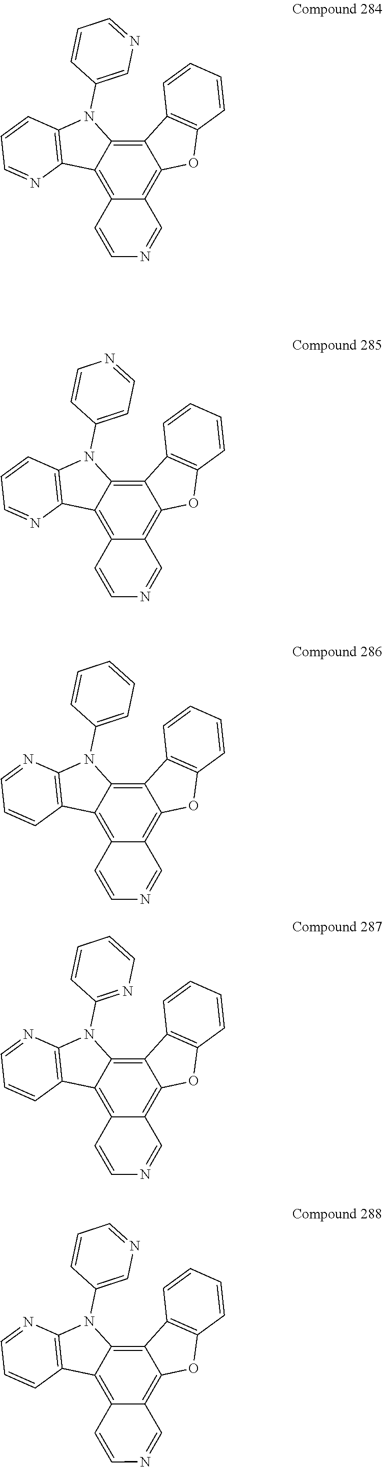

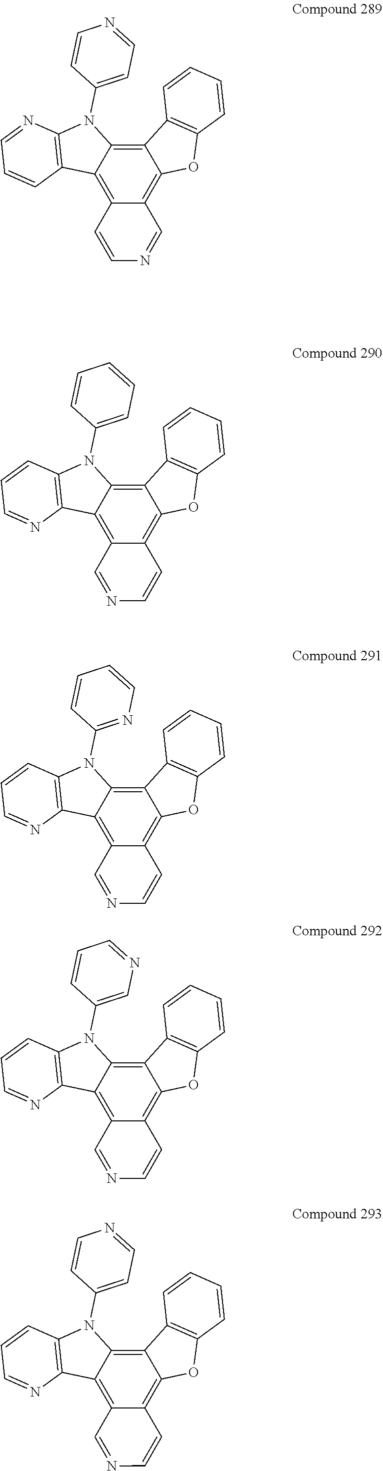

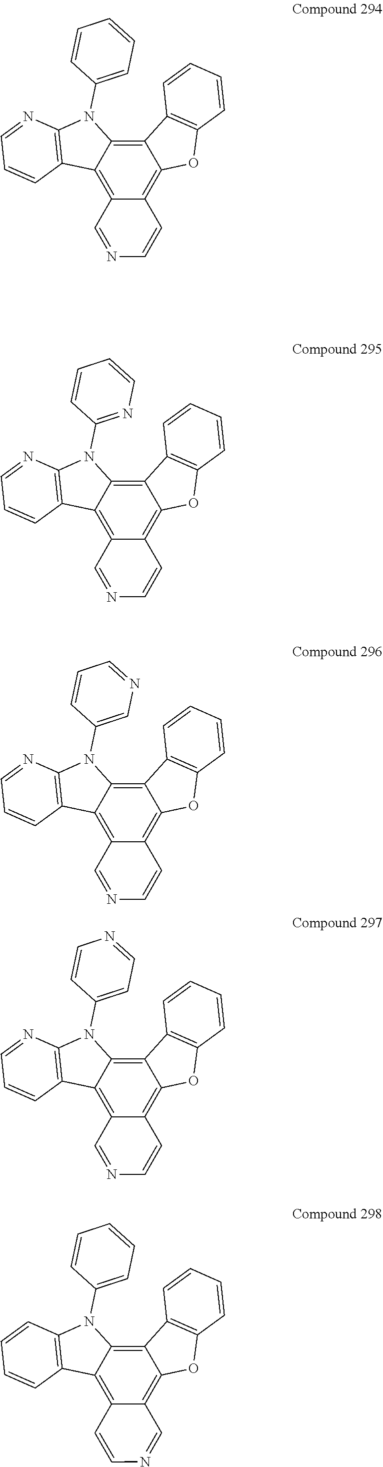

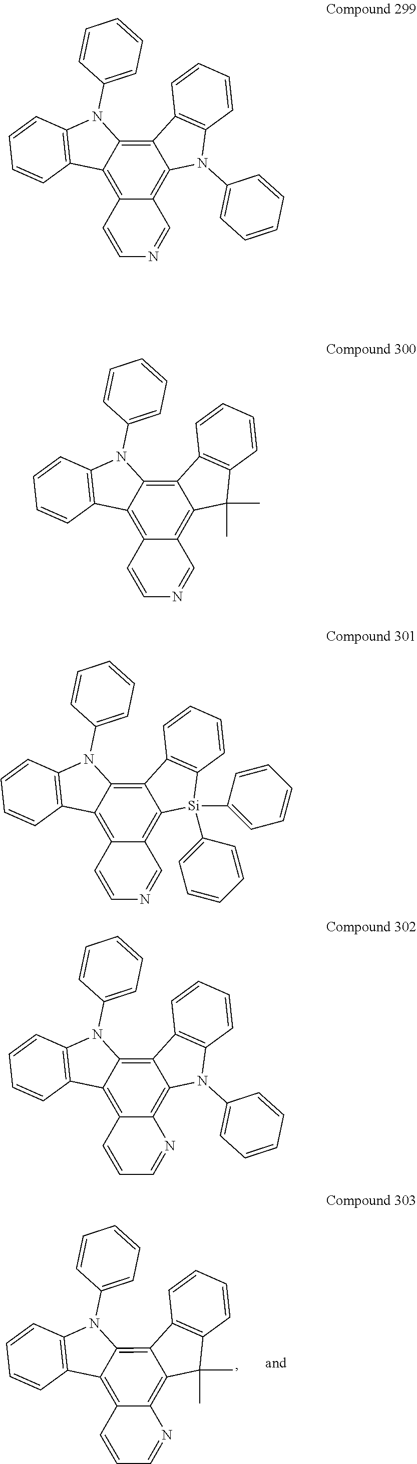

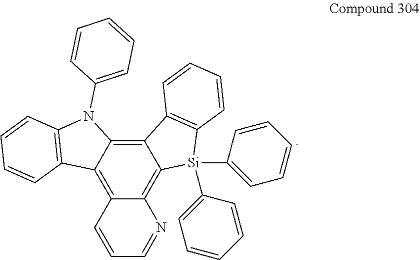 [SMILES: c1ccc(-n2c3ccccc3c3c4cccnc4c4c(c32)-c2ccccc2[Si]4(c2ccccc2)c2ccccc2)cc1]

An organic electroluminescence device comprising an anode, a cathode and one or more organic layers formed between the anode and the cathode, wherein at least one of the organic layers comprises the organic compound of formula (1).

The organic layers may comprise an emissive layer having a host, and wherein the organic compound is comprised as the host.

The organic layers may comprise an electron transfer layer, and wherein the organic compound of formula (1) is comprised as the electron transfer layer.

The organic compound of formula (1) may be a hole blocking material.

The organic electroluminescence device may be a lighting panel.

The organic electroluminescence device may be a backlight panel.

Referring to FIG. 1, the first organic EL device 510 may comprise an anode 310, a cathode 380 and one or more organic layers 320, 330, 340E, 350, 360, 370 formed between the anode 310 and the cathode 380. From the bottom to the top, the one or more organic layers may comprise a hole injection layer 320, a hole transport layer 330, an emissive layer 340E, a hole blocking layer 350, an electron transport layer 360 and an electron injection layer 370.

The emissive layer 340E may comprise a 15% dopant D1 and the organic compound of formula (1) 340C doped with the dopant D1. The dopant D1 may be a red guest material for tuning the wavelength at which the emissive layer 340E emits light, so that the color of emitted light may be green. The organic compound of formula (1) may be a host 340C of the emissive layer 340E.

FIG. 2 is a cross-sectional view of an organic EL device without the organic compound of formula (1). Referring to FIG. 2, the organic EL device 400 may comprise an anode 310, a cathode 380 and one or more organic layers 320, 330, 340, 350, 360, 370 formed between the anode 310 and the cathode 380. From the bottom to the top, the one or more organic layers may comprise a hole injection layer 320, a hole transport layer 330, an emissive layer 340, a hole blocking layer 350, an electron transport layer 360 and an electron injection layer 370. The emissive layer 340 may comprise a 15% dopant D1 and an organic compound H1 doped with the dopant D1. The dopant D1 may be a red guest material. The organic compound H1 is a host of the emissive layer 340.

To those organic EL devices of FIG. 1 and FIG. 2, EL spectra and CIE coordination are measured by using a PR650 spectra scan spectrometer. Furthermore, the current/voltage, luminescence/voltage, and yield/voltage characteristics are taken with a Keithley 2400 programmable voltage-current source. The above-mentioned apparatuses are operated at room temperature (about 25° C.) and under atmospheric pressure.

The I-V-B (at 1000 nits) test reports of those organic EL devices of FIG. 1 and FIG. 2 may be summarized in Table 1 below. The half-life is defined as the time that the initial luminance of 1000 cd/m² has dropped to half.

TABLE 1

| Emitting | | | | | Half- |
|---|---|---|---|---|---|
| Host | Emitting | Driving | Current | | life |
| Material | Guest | Voltage | Efficiency | | time |
| (for EML 40) | Material | (V) | (cd/A) | CIE(x) | (hours) |
| H1 | D1 | 6.3 | 11 | 0.64 | 202 |
| Compound 6 | D1 | 6.2 | 12 | 0.65 | 210 |
| Compound 10 | D1 | 6.2 | 13 | 0.65 | 216 |
| Compound 25 | D1 | 6.0 | 15 | 0.65 | 230 |
| Compound 35 | D1 | 5.7 | 20 | 0.66 | 256 |
| Compound 45 | D1 | 5.8 | 21 | 0.66 | 266 |
| Compound 55 | D1 | 5.7 | 22 | 0.66 | 278 |
| Compound 66 | D1 | 6.0 | 16 | 0.65 | 236 |
| Compound 90 | D1 | 5.9 | 17 | 0.65 | 239 |
| Compound 126 | D1 | 5.8 | 19 | 0.65 | 250 |
| Compound 135 | D1 | 5.6 | 22 | 0.66 | 281 |
| Compound 155 | D1 | 5.7 | 23 | 0.66 | 293 |
| Compound 173 | D1 | 5.9 | 18 | 0.66 | 230 |
| Compound 187 | D1 | 6.0 | 16 | 0.65 | 234 |
| Compound 196 | D1 | 5.9 | 18 | 0.65 | 239 |
| Compound 206 | D1 | 6.0 | 19 | 0.65 | 242 |
| Compound 220 | D1 | 6.1 | 14 | 0.65 | 220 |
| Compound 235 | D1 | 5.9 | 17 | 0.65 | 236 |
| Compound 249 | D1 | 6.1 | 16 | 0.65 | 233 |
| Compound 257 | D1 | 5.9 | 18 | 0.65 | 241 |
| Compound 261 | D1 | 5.6 | 24 | 0.66 | 296 |

According to Table 1, in the first organic EL device 510, the organic compound of formula (1) comprised as a host 340 of FIG. 1 exhibits performance better than a prior art organic EL material (H1). The organic EL device of the present invention may be operated under reduced voltage,

A method of producing the first organic EL device 510 of FIG. 1 and the organic EL device 400 of FIG. 2 is described. ITO-coated glasses with 9-12 ohm/square in resistance and 120-160 nm in thickness are provided (hereinafter ITO substrate) and cleaned in a number of cleaning steps in an ultrasonic bath (e.g., detergent, deionized water).

Before vapor deposition of the organic layers, cleaned ITO substrates may be further treated by UV and ozone. All pre-treatment processes for ITO substrate are under clean room (class 100), so that an anode 310 may be formed.

One or more organic layers 320, 330, 340 (FIG. 2), 340E (FIG. 1), 350, 360, 370 are applied onto the anode 310 in order by vapor deposition in a high-vacuum unit (10⁻⁷ Torr), such as resistively heated quartz boats. The thickness of the respective layer and the vapor deposition rate (0.1˜0.3 nm/sec) are precisely monitored or set with the aid of a quartz-crystal monitor. It is also possible, as described above, each of the organic layers may comprise more than one organic compound. For example, an emissive layer 340E or 340 may be formed of a dopant and a host doped with the dopant. An emissive layer 340E or 340 may also be formed of a co-host and a host co-deposited with the co-host. This may be successfully achieved by co-vaporization from two or more sources. Accordingly, the compounds for the organic layers of the present invention are thermally stable.

Referring to FIG. 1 and FIG. 2, onto the anode 310, Dipyrazino [2,3-f:2,3-] quinoxaline-2,3,6,7,10,11-hexacarbonitrile (HAT-CN) may be applied to form a hole injection layer (HIL) 320 having a thickness of about 20 nm in the organic EL device 510 or 400.

N,N-Bis(naphthalene-1-yl)-N,N-bis(phenyl)-benzidine (NPB) may be applied to form a hole transporting layer(HTL) 330 having a thickness of about 110 nm.

Referring to FIG. 1 and FIG. 2, in the organic EL device 510 (FIG. 1) or 400 (FIG. 2), an emissive layer (EML) 340E or 340 may be formed to have a thickness of about 30 nm.

Referring to FIG. 2, in the organic EL device 400, 12-(4,6-diphenyl-1,3,5-triazin-2-yl) -10,10-dimethyl-10,12-dihydrophenanthro[9′,10′:5,6]indeno[2,1-b]carbazole (i.e., H1 of paragraph [0002]) may be applied to form a host H1 of an emissive layer 340 of FIG. 2. The emissive layer 340 may further comprise bis(1-phenylisoquinoline)(acetylacetonate)-iridium(III) as a dopant D1, also a red guest of the emissive layer 340.

On the emissive layer 340 having a thickness of about 30 nm, a compound HB1 may be a hole blocking material (HBM) to form a hole blocking layer (HBL) 350 having a thickness of about 10 nm. 2-(naphthalen-1-yl)-9-(4-(1-(4-(10-(naphthalene-2-yl)anthracen-9-yl)-phenyl)-1H-benzo[d]imidazol-2-yl)phenyl)-1,10-phenanthroline(ET1) may be applied as an electron transporting material to co-deposit with 8-hydroxyquinolato-lithium(LiQ) at a ratio of 1:1, thereby forming an electron transporting layer 360 of the organic EL device 510 or 400. The electron transporting layer (ETL) 360 may have a thickness of about 35 nm. Table 2 shows the layer thickness and materials of the organic EL device 510 (FIG. 1) or 400 (FIG. 2).

TABLE 2

| Ref. No. in | | | Thickness |
|---|---|---|---|
| FIG.1 or FIG. 2 | Layer | Material | (nm) |
| 380 | Cathode | Al | 160 |
| 370 | EIL | LiQ | 1 |
| 360 | ETL | LiQ:ET1 (50%) | 35 |
| 350 | HBL | HB1 | 10 |
| 340E (FIG. 1) | EML | 340C or H1:D1 (5%) | 30 |
| or | | | |
| 340 (FIG. 2) | | | |
| 330 | HTL | NPB | 110 |
| 320 | HIL | HAT-CN | 20 |
| 310 | Anode | ITO substrate | 120~160 |

The organic compounds HAT-CN, NPB, D1, H1, HB1 and ET1 for producing the organic EL device 400 or 510 in this invention may have the formulas as follows:

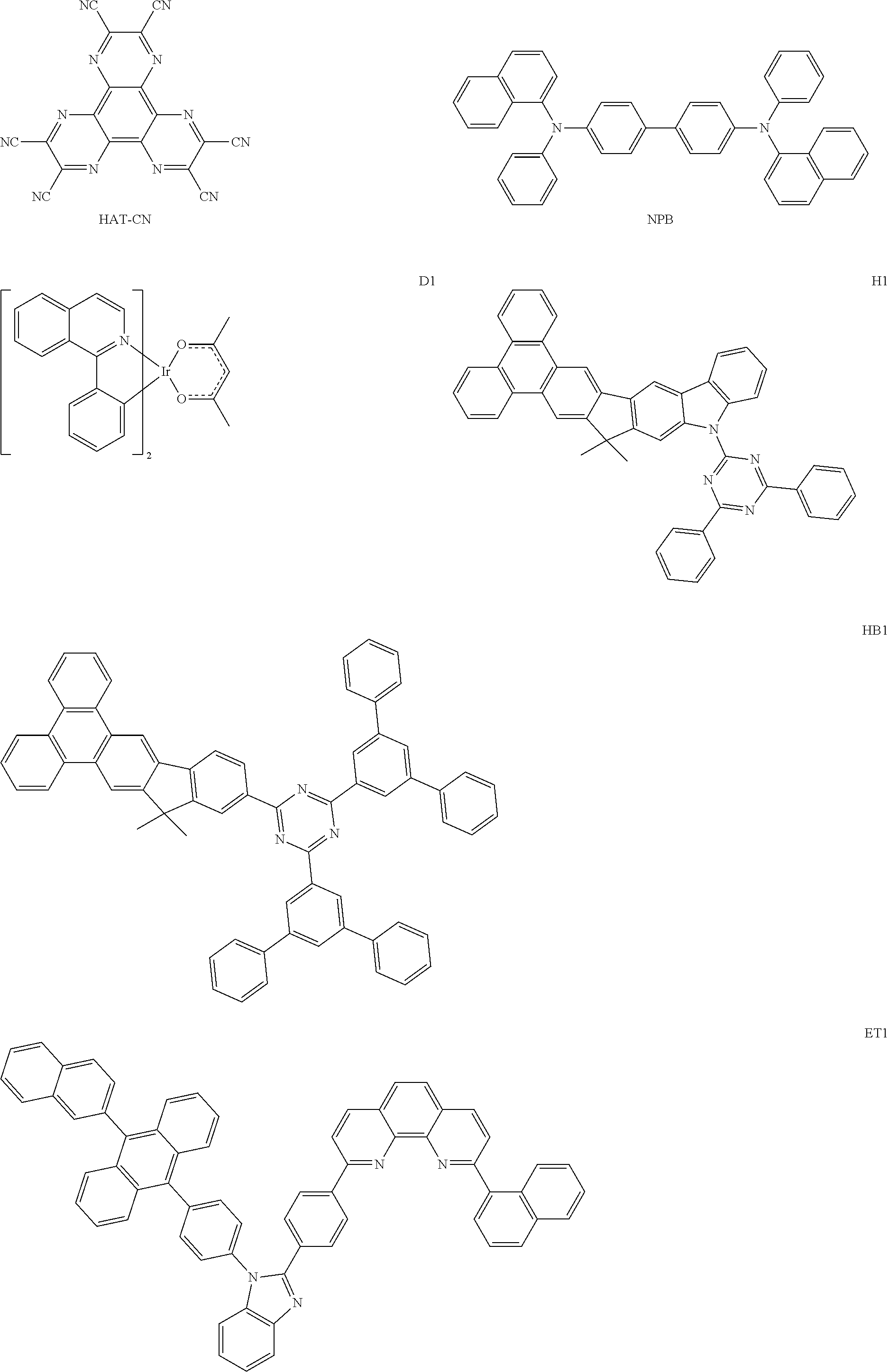

Referring to FIG. 1 and FIG. 2, the organic EL device 510 or 400 may further comprise a low work function metal, such as Al, Mg, Ca, Li or K, as a cathode 380 by thermal evaporation. The cathode 380 having a thickness of about 160 nm may help electrons injecting the electron transporting layer 360 from cathode 380. Between the cathode 380 (e.g., Al in Table 2) and the electron transporting layer 360, a thin electron injecting layer (EIL) 370 of LiQ is introduced. The electron injecting layer (EIL) 370 has a thickness of about 1 nm is to reduce the electron injection barrier and to improve the performance of the organic EL device 510 or 400. The material of the electron injecting layer 370 may alternatively be metal halide or metal oxide with low work function, such as LiF, MgO, or Li₂O.

In a third embodiment of the present invention, a second organic EL device using the organic compound of formula (1) is disclosed. The method of producing the second organic EL device 520 of FIG. 3 is substantially the same as the method of producing the organic EL device 400 of FIG. 2. The difference is that the hole blocking layer (HBL) 350C of FIG. 3 is made by using the organic compound of formula (1), rather than HB1.

Table 3 shows the layer thickness and materials of the organic EL device 520 (FIG. 3) or 400 (FIG. 2).

TABLE 3

| Ref. No. in | | | Thickness |
|---|---|---|---|
| FIG.2 or FIG. 3 | Layer | Material | (nm) |
| 380 | Cathode | Al | 160 |
| 370 | EIL | LiQ | 1 |
| 360 | ETL | LiQ:ET1 (50%) | 35 |
| 350C(FIG. 3) | HBL | 350C or HB1 | 10 |
| or | | | |
| 350(FIG. 2) | | | |
| 340 | EML | H1:D1 (5%) | 30 |
| 330 | HTL | NPB | 110 |
| 320 | HIL | HAT-CN | 20 |
| 310 | Anode | ITO substrate | 120~160 |

To those organic EL devices of FIG. 3 and FIG. 2, EL spectra and CIE coordination are measured by using a PR650 spectra scan spectrometer. Furthermore, the current/voltage, luminescence/voltage, and yield/voltage characteristics are taken with a Keithley 2400 programmable voltage-current source. The above-mentioned apparatuses are operated at room temperature (about 25° C.) and under atmospheric pressure.

In a fourth embodiment of the present invention, a third organic EL device using the organic compound of formula (1) is disclosed. The method of producing the third organic EL device 530 of FIG. 4 is substantially the same as the method of producing the organic EL device 400 of FIG. 2. The difference is that the electron transfer layer (ETL) 360C of FIG. 4 is made by using the organic compound of formula (1), rather than ET1.

Table 4 shows the layer thickness and materials of the organic EL device 530 (FIG. 4) or 400 (FIG. 2).

TABLE 4

| Ref. No. in | | | Thickness |
|---|---|---|---|
| FIG.2 or FIG. 4 | Layer | Material | (nm) |
| 380 | Cathode | Al | 160 |
| 370 | EIL | LiQ | 1 |
| 360C(FIG. 4) | ETL | LiQ:ET1(50%)or 360C | 35 |
| or | | | |
| 360(FIG. 2) | | | |
| 350 | HBL | HB1 | 10 |
| 340 | EML | H1:D1 (5%) | 30 |
| 330 | HTL | NPB | 110 |
| 320 | HIL | HAT-CN | 20 |
| 310 | Anode | ITO substrate | 120~160 |

To those organic EL devices of FIG. 4 and FIG. 2, EL spectra and CIE coordination are measured by using a PR650 spectra scan spectrometer. Furthermore, the current/voltage, luminescence/voltage, and yield/voltage characteristics are taken with a Keithley 2400 programmable voltage-current source. The above-mentioned apparatuses are operated at room temperature (about 2° C.) and under atmospheric pressure.

The I-V-B(at 1000 nits) test reports of those organic EL devices of FIG. 3, FIG. 4 and FIG. 2 may be summarized in Table 5 below. The half-life of the phosphorescent green-emitting organic EL device 520, 530 or 400 is defined as the time that the initial luminance of 1000 cd/m² has dropped to half.

According to Table 5, in the second organic EL device 520, the organic compound of formula (1) comprised as a hole blocking layer 350C of FIG. 3 exhibits performance better than a prior art hole blocking material (HB1 as a HBL 350 of FIG. 2).

TABLE 5
(The Comp. is short for Compound)

| | | | Current | | |
|---|---|---|---|---|---|
| Material | Material | Driving | Efficiency | | Half-life |
| of | of | Voltage | (Yield; | | time |
| HBL | ETL | (V) | cd/A) | CIE(y) | (hours) |
| HB1 | ET1 | 6.3 | 11 | 0.64 | 202 |
| HB1 | Comp.20 | 6.1 | 15 | 0.65 | 227 |
| HB1 | Comp.24 | 5.9 | 16 | 0.65 | 234 |
| HB1 | Comp.28 | 5.9 | 17 | 0.65 | 238 |
| HB1 | Comp.38 | 6.1 | 14 | 0.64 | 213 |
| HB1 | Comp.42 | 6.0 | 16 | 0.65 | 231 |
| HB1 | Comp.82 | 6.2 | 14 | 0.65 | 220 |
| HB1 | Comp.144 | 6.0 | 15 | 0.65 | 226 |
| HB1 | Comp.198 | 6.1 | 13 | 0.64 | 219 |
| HB1 | Comp.259 | 6.1 | 15 | 0.65 | 224 |
| Comp.16 | ET1 | 6.0 | 14 | 0.65 | 215 |
| Comp.54 | ET1 | 6.3 | 12 | 0.65 | 205 |
| Comp.99 | ET1 | 6.1 | 13 | 0.65 | 212 |
| Comp.119 | ET1 | 6.2 | 12 | 0.64 | 204 |
| Comp.160 | ET1 | 6.2 | 12 | 0.65 | 208 |
| Comp.210 | ET1 | 6.1 | 13 | 0.65 | 213 |
| Comp.230 | ET1 | 6.3 | 12 | 0.64 | 207 |

According to Table 5, in the third organic EL device 530, the organic compound of formula (1) comprised as an electron transfer layer 360C of FIG. 4 exhibits performance better than a prior art electron transfer material (ET1 as a ETL 360 of FIG. 2).

Referring to FIG. 1 or FIG. 3, the organic EL device 510 or 520 of the present invention may alternatively be a lighting panel or a backlight panel.

Referring to FIG. 1 or FIG. 4, the organic EL device 510 or 530 of the present invention may alternatively be a lighting panel or a backlight panel.

Detailed preparation of the organic compounds of the present invention will be clarified by exemplary embodiments below, but the present invention is not limited thereto. EXAMPLES 1 to 34 show the preparation of the organic compounds of the present invention.

### EXAMPLE 1

### Synthesis of 5-(4,4,5,5-tetramethyl-1,3,2-dioxaborolan-2-yl)benzo-[4,5]thieno[3,2-h]isoquinoline

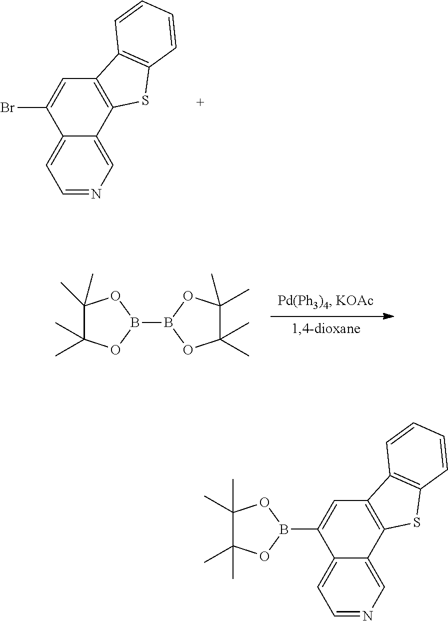

A mixture of 10 g (31.8 mmol) of 5-bromobenzo[4,5]thieno[3,2-h]-isoquinoline, 9.7 g (38.2 mmol) of bis(pinacolato)diboron, 0.74 g (0.6 mmol) of Pd(Ph₃)₄, 6.24 g (63.6 mmol) of potassium acetate, and 150 ml of 1,4-dioxane was degassed and placed under nitrogen, and then heated to reflux for 12 hrs. After the reaction finished, the mixture was allowed to cool to room temperature. Subsequently, the solvent was removed under reduced pressure, and the crude product was purified by column chromatography, yielding 8.8 g of 5-(4,4,5,5-tetramethyl-1,3,2-dioxaborolan-2-yl)benzo-[4,5]thieno[3,2-h]isoquinoline as white solid (76.5%). ¹H NMR (CDCl₃, 400 MHz): chemical shift (ppm) 8.88 (s, 1H), 8.41 (d, 1H), 8.19 (d, 1H), 7.98 (d, 1H), 7.51-7.47 (m, 3H), 7.41 (d, 1H), 1.26 (s, 12H).

### Synthesis of 5-(2-nitrophenyl)benzo[4,5]thieno[3,2-h]isoquinoline

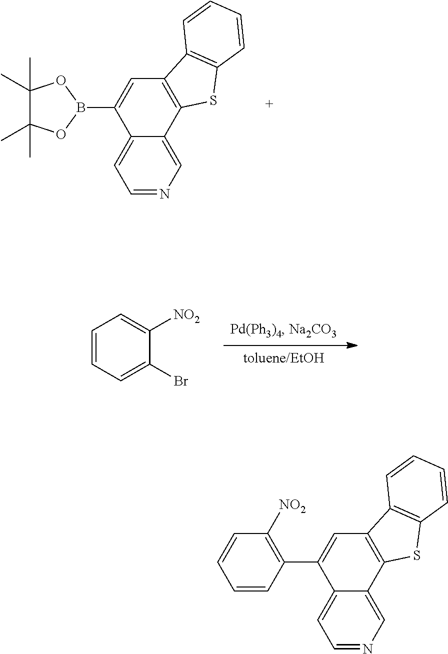 [SMILES: CC1(C)OB(c2cc3c4ccccc4sc3c3cnccc23)OC1(C)C ; O=[N+]([O-])c1ccccc1Br ; O=[N+]([O-])c1ccccc1-c1cc2c3ccccc3sc2c2cnccc12]

A mixture of 8.8 g (24.4 mmol) of 5-(4,4,5,5-tetramethyl-1,3,2-dioxaborolan-2-yl)benzo[4,5]thieno[3,2-h]isoquinoline, 5.4 g (26.8 mmol) of 1-bromo-2-nitrobenzene, 0.56 g (0.5 mmol) of Pd(Ph₃)₄, 24.4 ml of 2 M Na₂CO₃, 30 ml of EtOH and 90 ml of toluene was degassed and placed under nitrogen, and then heated to reflux for 12 hrs. After the reaction finished, the mixture was allowed to cool to room temperature. Subsequently, the solvent was removed under reduced pressure, and the crude product was purified by column chromatography, yielding 6 g of 5-(2-nitrophenyl)benzo[4,5]thieno-[3,2-h]isoquinoline as yellow solid (69.1%). ¹H NMR (CDCl₃, 400 MHz): chemical shift (ppm) 8.89 (s, 1H), 8.43 (m, 2H), 8.03-7.99 (m, 3H), 7.88 (m,1H), 7.79 (s, 1H), 7.66 (m, 1H), 7.53-7.48 (m, 3H).

### Synthesis of 14H-benzo[4,5]thieno[3,2-a]pyrido[3,4-c]carbazole

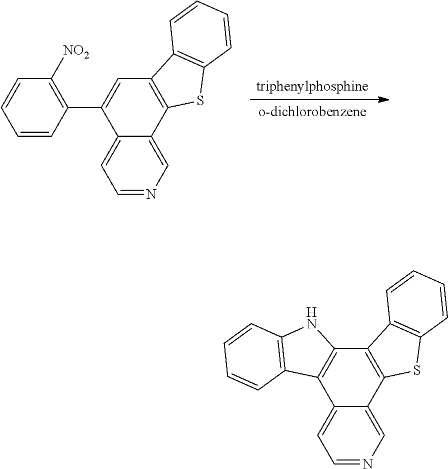 [SMILES: O=[N+]([O-])c1ccccc1-c1cc2c3ccccc3sc2c2cnccc12 ; c1ccc2c(c1)[nH]c1c2c2ccncc2c2sc3ccccc3c12]

A mixture of 6 g (16.8 mmol) of 5-(2-nitrophenyl)benzo[4,5]thieno-[3,2-h]isoquinoline, 17.7 g (67.3 mmol) of triphenylphosphine, and 60 ml of o-dichlorobenzene was degassed and placed under nitrogen, and then heated to reflux for 12 hrs. After the reaction finished, the mixture was allowed to cool to room temperature. Subsequently, the solvent was removed under reduced pressure, and the crude product was purified by column chromatography, yielding 3.4 g of 14H-benzo[4,5]thieno[3,2-a]pyrido[3,4-c]carbazole as white solid (62.3%). ¹H NMR (CDCl₃, 400 MHz): chemical shift (ppm) 10.1 (s, 1H), 8.91 (s, 1H), 8.45 (m, 2H), 8.13 (d, 1H), 7.98 (d,1H), 7.64 (m, 1H), 7.53-7.48 (m, 4H), 7.26 (m, 1H).

### Synthesis of 14-(4-phenylquinazolin-2-yl)-14H-benzo[4,5]thieno[3,2-a]pyrido[3,4-c]-carbazole (Compound 45)

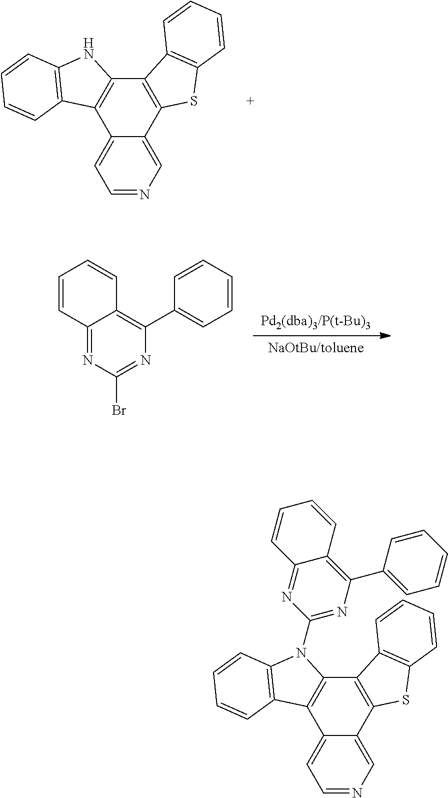 [SMILES: c1ccc2c(c1)[nH]c1c2c2ccncc2c2sc3ccccc3c12 ; Brc1nc(-c2ccccc2)c2ccccc2n1 ; c1ccc(-c2nc(-n3c4ccccc4c4c5ccncc5c5sc6ccccc6c5c43)nc3ccccc23)cc1]

A mixture of 3.4 g (10.5 mmol) of 14H-benzo[4,5]thieno[3,2-a]pyrido-[3,4-c]carbazole, 3.6 g (12.6 mmol) of 2-bromo-4-phenylquinazoline, 0.2 g (0.2 mmol) of Pd₂(dba)₃, 0.21 g (1 mmol) of P(t-Bu)₃, 2 g (21 mmol) of NaOtBu, 40 ml of toluene was degassed and placed under nitrogen, and then heated to reflux for 12 hrs. After the reaction finished, the mixture was allowed to cool to room temperature. Subsequently, the solvent was removed under reduced pressure, and the crude product was purified by column chromatography, yielding 3.8 g of 14-(4-phenylquinazolin-2-yl)-14H-benzo[4,5]thieno[3,2-a]-pyrido[3,4-c]carbazole as yellow solid (68.6%). ¹H NMR (CDCl₃, 400 MHz): chemical shift (ppm) 8.92 (s, 1H), 8.53 (d, 1H), 8.45 (m, 2H), 8.18 (d, 1H), 8.03-7.95 (m, 4H), 7.81 (m, 3H), 7.53-7.48 (m, 5H), 7.40 (m, 1H), 7.34 (m, 1H), 7.24 (m, 1H).

### EXAMPLE 2

### Synthesis of 5-(4,4,5,5-tetramethyl-1,3,2-dioxaborolan-2-yl)benzofuro-[3,2-h]quinoline

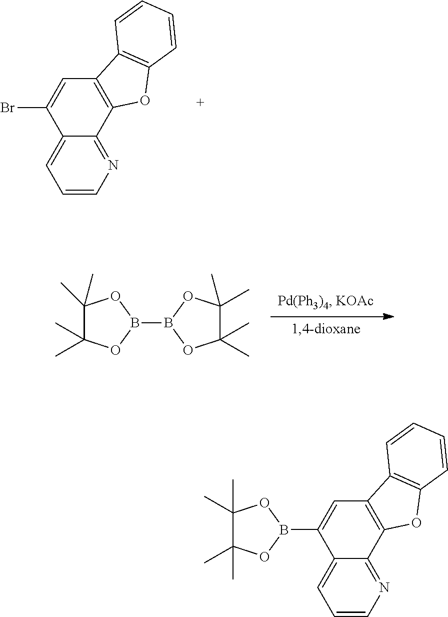

A mixture of 10 g (33.5 mmol) of 5-bromobenzofuro[3,2-h]quinoline, 10.2 g (40.2 mmol) of bis(pinacolato)diboron, 0.78 g (0.67 mmol) of Pd(Ph₃)₄, 6.58 g (67.1 mmol) of potassium acetate, and 150 ml of 1,4-dioxane was degassed and placed under nitrogen, and then heated to reflux for 12 hrs. After the reaction finished, the mixture was allowed to cool to room temperature. Subsequently, the solvent was removed under reduced pressure, and the crude product was purified by column chromatography, yielding 8.2 g of 5-(4,4,5,5-tetramethyl-1,3,2-dioxaborolan-2-yl)benzofuro-[3,2-h]quinoline as white solid (70.8%). ¹H NMR (CDCl₃, 400 MHz): chemical shift (ppm) 8.85 (d, 1H), 8.39 (d, 1H), 7.89 (d, 1H), 7.64-7.59 (d, 3H), 7.37-7.33 (m, 2H), 1.26 (s, 12H).

### Synthesis of 5-(2-nitrophenyl)benzofuro[3,2-h]quinoline

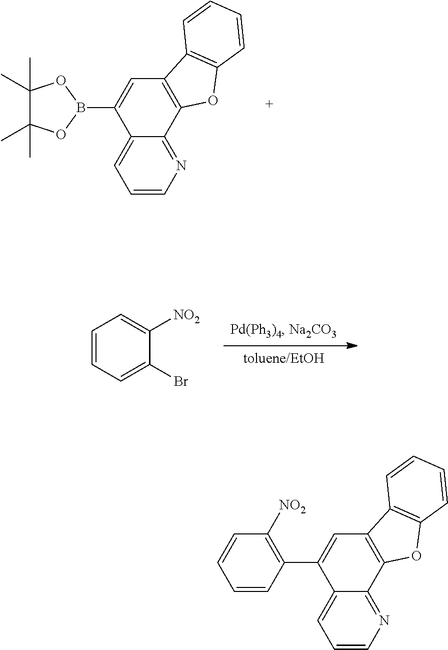 [SMILES: CC1(C)OB(c2cc3c4ccccc4oc3c3ncccc23)OC1(C)C ; O=[N+]([O-])c1ccccc1Br ; O=[N+]([O-])c1ccccc1-c1cc2c3ccccc3oc2c2ncccc12]

A mixture of 8.2 g (23.8 mmol) of 5-(4,4,5,5-tetramethyl-1,3,2-dioxaborolan-2-yl)benzofuro [3,2-h]quinoline, 5.3 g (26.1 mmol) of 1-bromo-2-nitrobenzene, 0.55 g (0.48 mmol) of Pd(Ph₃)₄, 23.8 ml of 2M Na₂CO₃, 30 ml of EtOH and 90 ml of toluene was degassed and placed under nitrogen, and then heated to reflux for 12 hrs. After the reaction finished, the mixture was allowed to cool to room temperature. Subsequently, the solvent was removed under reduced pressure, and the crude product was purified by column chromatography, yielding 5.8 g of 5-(2-nitrophenyl)benzofuro[3,2-h]quinoline as yellow solid (71.8%). ¹H NMR (CDCl₃, 400 MHz): chemical shift (ppm) 8.84 (d, 1H), 8.39 (d, 1H), 8.05-8.01 (m, 2H), 7.91-7.88 (m, 2H), 7-68-7.64 (m, 3H), 7.57 (m, 1H), 7.37-7.33 (m, 2H).

### Synthesis of 14H-benzofuro[3,2-a]pyrido[2,3-c]carbazole

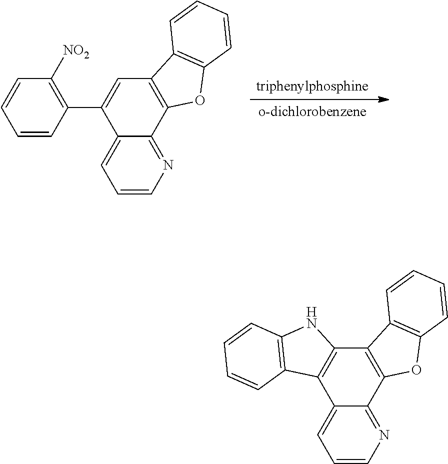 [SMILES: O=[N+]([O-])c1ccccc1-c1cc2c3ccccc3oc2c2ncccc12 ; c1ccc2c(c1)[nH]c1c2c2cccnc2c2oc3ccccc3c12]

A mixture of 5.8 g (17 mmol) of 5-(2-nitrophenyl)benzofuro[3,2-h]-quinoline, 17.9 g (68.1 mmol) of triphenylphosphine, and 60 ml of o-dichlorobenzene was degassed and placed under nitrogen, and then heated to reflux for 12 hrs. After the reaction finished, the mixture was allowed to cool to room temperature. Subsequently, the solvent was removed under reduced pressure, and the crude product was purified by column chromatography, yielding 3.3 g of 14H-benzofuro[3,2-a]pyrido[2,3-c]carbazole as white solid (62.8%). ¹H NMR (CDCl₃, 400 MHz): chemical shift (ppm) 10.1 (s, 1H), 8.84 (d, 1H), 8.39 (d, 1H), 8.14 (d, 1H), 7.91 (d, 1H), 7.65-7.59 (m, 3H), 7.48(m, 1H), 7.36-7.30 (m, 3H).

### Synthesis of 14-(4,6-diphenylpyrimidin-2-yl)-14H-benzofuro[3,2-a]pyrido[2,3-c]carbazole (Compound 161)

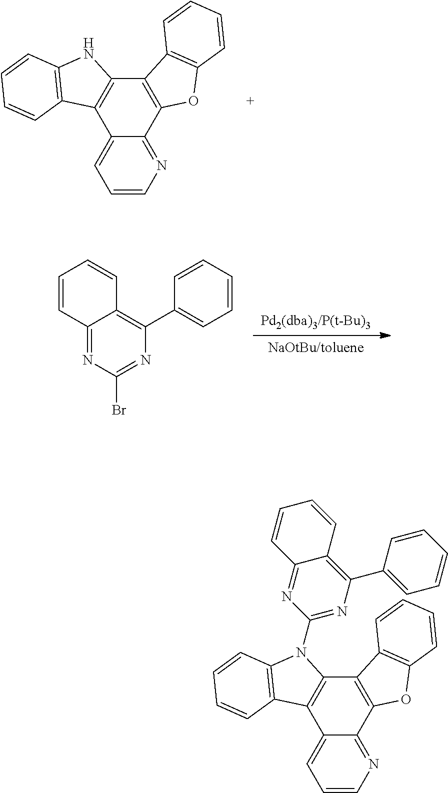 [SMILES: c1ccc2c(c1)[nH]c1c2c2cccnc2c2oc3ccccc3c12 ; Brc1nc(-c2ccccc2)c2ccccc2n1 ; c1ccc(-c2nc(-n3c4ccccc4c4c5cccnc5c5oc6ccccc6c5c43)nc3ccccc23)cc1]

A mixture of 3.3 g (10.7 mmol) of 14H-benzofuro[3,2-a]pyrido[2,3-c]-carbazole, 3.66 g (11.8 mmol) of 2-bromo-4,6-diphenylpyrimidine, 0.2 g (0.2 mmol) of Pd₂(dba)₃, 0.22 g (1 mmol) of P(t-Bu)₃, 2.1 g (21.4 mmol) of NaOtBu, 40 ml of toluene was degassed and placed under nitrogen, and then heated to reflux for 12 hrs. After the reaction finished, the mixture was allowed to cool to room temperature. Subsequently, the solvent was removed under reduced pressure, and the crude product was purified by column chromatography, yielding 3.9 g of 14-(4,6-diphenylpyrimidin-2-yl)-14H-benzofuro[3,2-a]pyrido[2,3-c]carbazole as white solid (67.7%). ¹H NMR (CDCl₃, 400 MHz): chemical shift (ppm) 8.84 (d, 1H), 8.62 (s, 1H), 8.54 (d, 1H), 8.39 (d, 1H), 7.93-7.90 (m, 2H), 7.78 (d, 4H), 7.67 (d, 1H), 7.56-7.43 (m, 8H), 7.32-7.27 (m, 3H).

We have used the same synthesis methods to get a series of intermediates and the following compounds are synthesized analogously.

| Ex. | Intermediate III | Intermediate IV | Product | Yield |
|---|---|---|---|---|
| 3 | 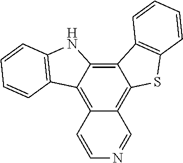 [SMILES: c1ccc2c(c1)[nH]c1c2c2ccncc2c2sc3ccccc3c12] | 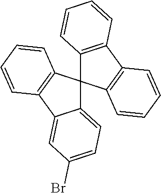 [SMILES: Brc1ccc2c(c1)-c1ccccc1C21c2ccccc2-c2ccccc21] | 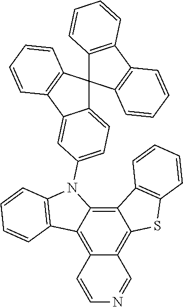 [SMILES: c1ccc2c(c1)-c1ccccc1C21c2ccccc2-c2cc(-n3c4ccccc4c4c5ccncc5c5sc6ccccc6c5c43)ccc21] | 63% |
| | | | Compound 10 | |
| 4 | 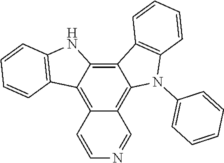 [SMILES: c1ccc(-n2c3ccccc3c3c4[nH]c5ccccc5c4c4ccncc4c32)cc1] | 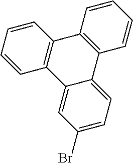 [SMILES: Brc1ccc2c3ccccc3c3ccccc3c2c1] | 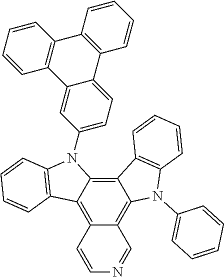 [SMILES: c1ccc(-n2c3ccccc3c3c2c2cnccc2c2c4ccccc4n(-c4ccc5c6ccccc6c6ccccc6c5c4)c23)cc1] | 67% |
| | | | Compound 16 | |
| 5 | 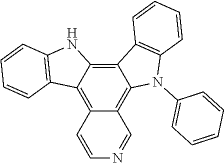 [SMILES: c1ccc(-n2c3ccccc3c3c4[nH]c5ccccc5c4c4ccncc4c32)cc1] | 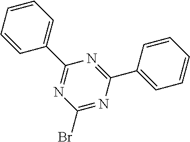 [SMILES: Brc1nc(-c2ccccc2)nc(-c2ccccc2)n1] | 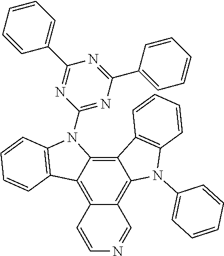 [SMILES: c1ccc(-c2nc(-c3ccccc3)nc(-n3c4ccccc4c4c5ccncc5c5c(c6ccccc6n5-c5ccccc5)c43)n2)cc1] | 59% |
| | | | Compound 20 | |
| 6 | 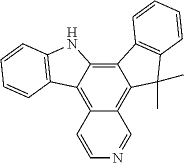 [SMILES: CC1(C)c2ccccc2-c2c1c1cnccc1c1c2[nH]c2ccccc21] | 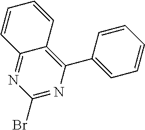 [SMILES: Brc1nc(-c2ccccc2)c2ccccc2n1] | 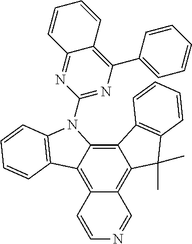 [SMILES: CC1(C)c2ccccc2-c2c1c1cnccc1c1c3ccccc3n(-c3nc(-c4ccccc4)c4ccccc4n3)c21] | 57% |
| | | | Compound 25 | |
| 7 | 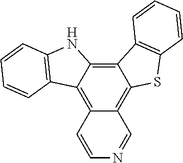 [SMILES: c1ccc2c(c1)[nH]c1c2c2ccncc2c2sc3ccccc3c12] | 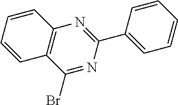 [SMILES: Brc1nc(-c2ccccc2)nc2ccccc12] | 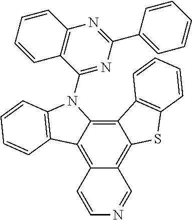 [SMILES: c1ccc(-c2nc(-n3c4ccccc4c4c5ccncc5c5sc6ccccc6c5c43)c3ccccc3n2)cc1] | 58% |
| | | | Compound 26 | |
| 8 | 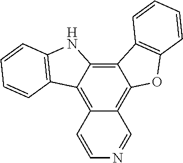 [SMILES: c1ccc2c(c1)[nH]c1c2c2ccncc2c2oc3ccccc3c12] | 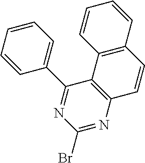 [SMILES: Brc1nc(-c2ccccc2)c2c(ccc3ccccc32)n1] | 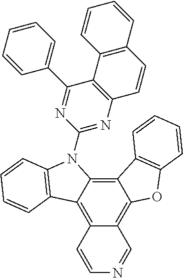 [SMILES: c1ccc(-c2nc(-n3c4ccccc4c4c5ccncc5c5oc6ccccc6c5c43)nc3ccc4ccccc4c23)cc1] | 61% |
| | | | Compound 35 | |
| 9 | 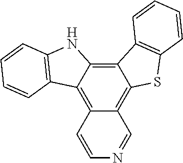 [SMILES: c1ccc2c(c1)[nH]c1c2c2ccncc2c2sc3ccccc3c12] | 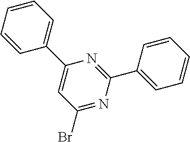 [SMILES: Brc1cc(-c2ccccc2)nc(-c2ccccc2)n1] | 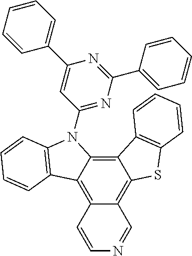 [SMILES: c1ccc(-c2cc(-n3c4ccccc4c4c5ccncc5c5sc6ccccc6c5c43)nc(-c3ccccc3)n2)cc1] | 62% |
| | | | Compound 42 | |
| 10 | 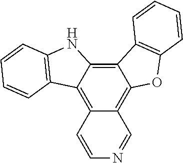 [SMILES: c1ccc2c(c1)[nH]c1c2c2ccncc2c2oc3ccccc3c12] | 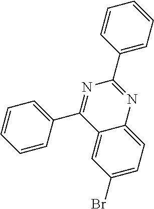 [SMILES: Brc1ccc2nc(-c3ccccc3)nc(-c3ccccc3)c2c1] | 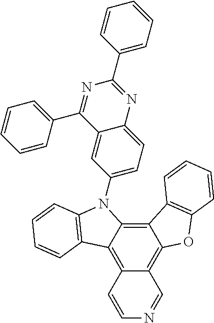 [SMILES: c1ccc(-c2nc(-c3ccccc3)c3cc(-n4c5ccccc5c5c6ccncc6c6oc7ccccc7c6c54)ccc3n2)cc1] | 58% |
| | | | Compound 47 | |
| 11 | 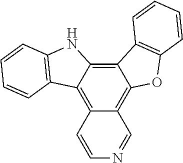 [SMILES: c1ccc2c(c1)[nH]c1c2c2ccncc2c2oc3ccccc3c12] | 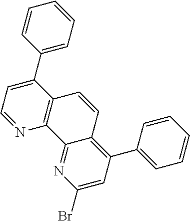 [SMILES: Brc1cc(-c2ccccc2)c2ccc3c(-c4ccccc4)ccnc3c2n1] | 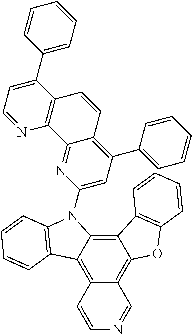 [SMILES: c1ccc(-c2ccnc3c2ccc2c(-c4ccccc4)cc(-n4c5ccccc5c5c6ccncc6c6oc7ccccc7c6c54)nc23)cc1] | 56% |
| | | | Compound 58 | |
| 12 | 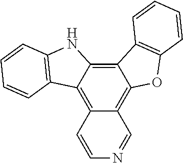 [SMILES: c1ccc2c(c1)[nH]c1c2c2ccncc2c2oc3ccccc3c12] | 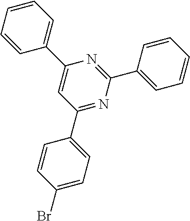 [SMILES: Brc1ccc(-c2cc(-c3ccccc3)nc(-c3ccccc3)n2)cc1] | 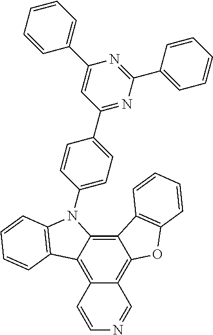 [SMILES: c1ccc(-c2cc(-c3ccc(-n4c5ccccc5c5c6ccncc6c6oc7ccccc7c6c54)cc3)nc(-c3ccccc3)n2)cc1] | 65% |
| | | | Compound 63 | |
| 13 | 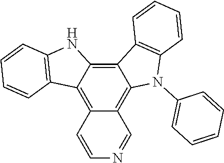 [SMILES: c1ccc(-n2c3ccccc3c3c4[nH]c5ccccc5c4c4ccncc4c32)cc1] | 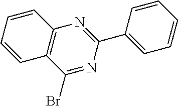 [SMILES: Brc1nc(-c2ccccc2)nc2ccccc12] | 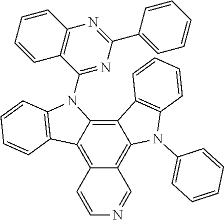 [SMILES: c1ccc(-c2nc(-n3c4ccccc4c4c5ccncc5c5c(c6ccccc6n5-c5ccccc5)c43)c3ccccc3n2)cc1] | 63% |
| | | | Compound 66 | |
| 14 | 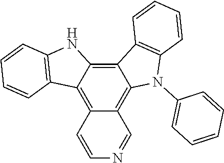 [SMILES: c1ccc(-n2c3ccccc3c3c4[nH]c5ccccc5c4c4ccncc4c32)cc1] | 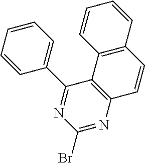 [SMILES: Brc1nc(-c2ccccc2)c2c(ccc3ccccc32)n1] | 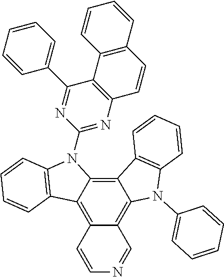 [SMILES: c1ccc(-c2nc(-n3c4ccccc4c4c5ccncc5c5c(c6ccccc6n5-c5ccccc5)c43)nc3ccc4ccccc4c23)cc1] | 67% |
| | | | Compound 75 | |
| 15 | 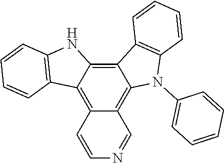 [SMILES: c1ccc(-n2c3ccccc3c3c4[nH]c5ccccc5c4c4ccncc4c32)cc1] | 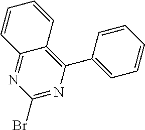 [SMILES: Brc1nc(-c2ccccc2)c2ccccc2n1] | 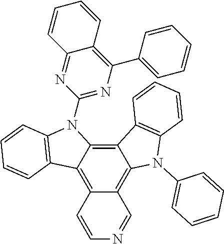 [SMILES: c1ccc(-c2nc(-n3c4ccccc4c4c5ccncc5c5c(c6ccccc6n5-c5ccccc5)c43)nc3ccccc23)cc1] | 61% |
| | | | Compound 85 | |
| 16 | 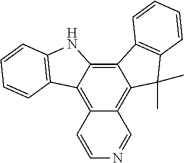 [SMILES: CC1(C)c2ccccc2-c2c1c1cnccc1c1c2[nH]c2ccccc21] | 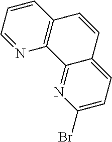 [SMILES: Brc1ccc2ccc3cccnc3c2n1] | 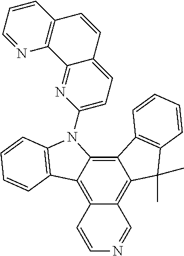 [SMILES: CC1(C)c2ccccc2-c2c1c1cnccc1c1c3ccccc3n(-c3ccc4ccc5cccnc5c4n3)c21] | 59% |
| | | | Compound 88 | |
| 17 | 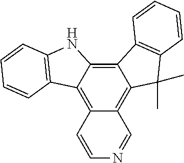 [SMILES: CC1(C)c2ccccc2-c2c1c1cnccc1c1c2[nH]c2ccccc21] | 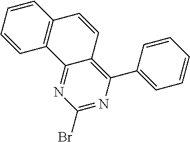 [SMILES: Brc1nc(-c2ccccc2)c2ccc3ccccc3c2n1] | 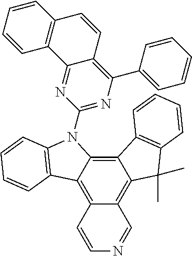 [SMILES: CC1(C)c2ccccc2-c2c1c1cnccc1c1c3ccccc3n(-c3nc(-c4ccccc4)c4ccc5ccccc5c4n3)c21] | 60% |
| | | | Compound 96 | |
| 18 | 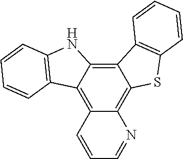 [SMILES: c1ccc2c(c1)[nH]c1c2c2cccnc2c2sc3ccccc3c12] | 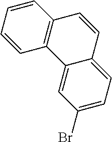 [SMILES: Brc1ccc2ccc3ccccc3c2c1] | 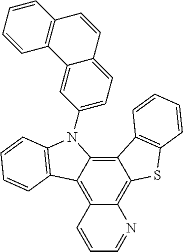 [SMILES: c1ccc2c(c1)ccc1ccc(-n3c4ccccc4c4c5cccnc5c5sc6ccccc6c5c43)cc12] | 62% |
| | | | Compound 108 | |
| 19 | 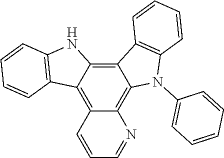 [SMILES: c1ccc(-n2c3ccccc3c3c4[nH]c5ccccc5c4c4cccnc4c32)cc1] | 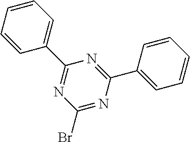 [SMILES: Brc1nc(-c2ccccc2)nc(-c2ccccc2)n1] | 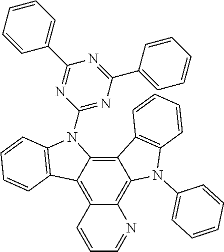 [SMILES: c1ccc(-c2nc(-c3ccccc3)nc(-n3c4ccccc4c4c5cccnc5c5c(c6ccccc6n5-c5ccccc5)c43)n2)cc1] | 61% |
| | | | Compound 120 | |
| 20 | 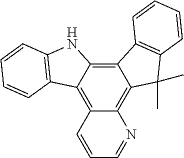 [SMILES: CC1(C)c2ccccc2-c2c1c1ncccc1c1c2[nH]c2ccccc21] | 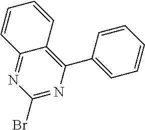 [SMILES: Brc1nc(-c2ccccc2)c2ccccc2n1] | 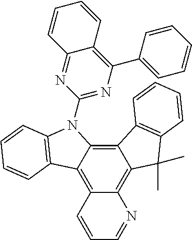 [SMILES: CC1(C)c2ccccc2-c2c1c1ncccc1c1c3ccccc3n(-c3nc(-c4ccccc4)c4ccccc4n3)c21] | 64% |
| | | | Compound 125 | |
| 21 | 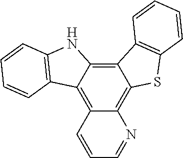 [SMILES: c1ccc2c(c1)[nH]c1c2c2cccnc2c2sc3ccccc3c12] | 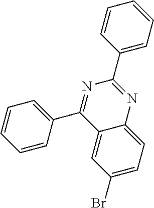 [SMILES: Brc1ccc2nc(-c3ccccc3)nc(-c3ccccc3)c2c1] | 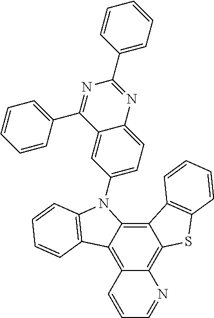 [SMILES: c1ccc(-c2nc(-c3ccccc3)c3cc(-n4c5ccccc5c5c6cccnc6c6sc7ccccc7c6c54)ccc3n2)cc1] | 68% |
| | | | Compound 127 | |
| 22 | 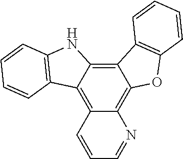 [SMILES: c1ccc2c(c1)[nH]c1c2c2cccnc2c2oc3ccccc3c12] | 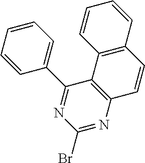 [SMILES: Brc1nc(-c2ccccc2)c2c(ccc3ccccc32)n1] | 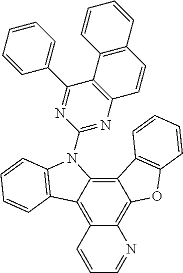 [SMILES: c1ccc(-c2nc(-n3c4ccccc4c4c5cccnc5c5oc6ccccc6c5c43)nc3ccc4ccccc4c23)cc1] | 61% |
| | | | Compound 135 | |
| 23 | 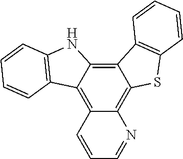 [SMILES: c1ccc2c(c1)[nH]c1c2c2cccnc2c2sc3ccccc3c12] | 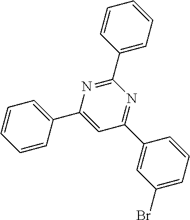 [SMILES: Brc1cccc(-c2cc(-c3ccccc3)nc(-c3ccccc3)n2)c1] | 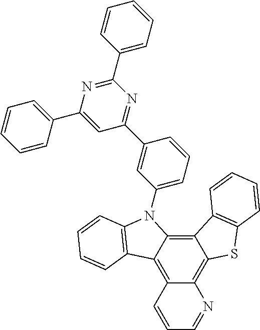 [SMILES: c1ccc(-c2cc(-c3cccc(-n4c5ccccc5c5c6cccnc6c6sc7ccccc7c6c54)c3)nc(-c3ccccc3)n2)cc1] | 57% |
| | | | Compound 144 | |
| 24 | 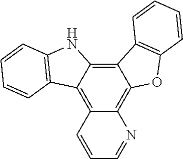 [SMILES: c1ccc2c(c1)[nH]c1c2c2cccnc2c2oc3ccccc3c12] | 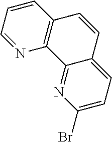 [SMILES: Brc1ccc2ccc3cccnc3c2n1] | 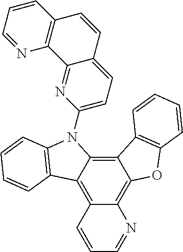 [SMILES: c1cnc2c(c1)ccc1ccc(-n3c4ccccc4c4c5cccnc5c5oc6ccccc6c5c43)nc12] | 62% |
| | | | Compound 148 | |
| 25 | 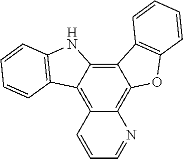 [SMILES: c1ccc2c(c1)[nH]c1c2c2cccnc2c2oc3ccccc3c12] | 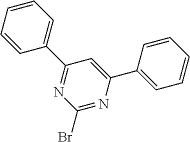 [SMILES: Brc1nc(-c2ccccc2)cc(-c2ccccc2)n1] | 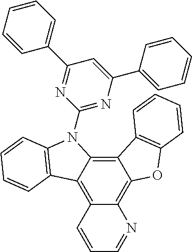 [SMILES: c1ccc(-c2cc(-c3ccccc3)nc(-n3c4ccccc4c4c5cccnc5c5oc6ccccc6c5c43)n2)cc1] | 59% |
| | | | Compound 161 | |
| 26 | 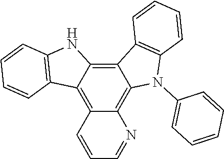 [SMILES: c1ccc(-n2c3ccccc3c3c4[nH]c5ccccc5c4c4cccnc4c32)cc1] | 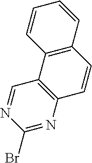 [SMILES: Brc1ncc2c(ccc3ccccc32)n1] | 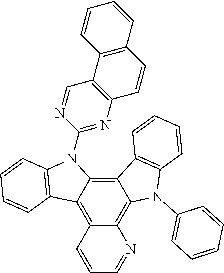 [SMILES: c1ccc(-n2c3ccccc3c3c2c2ncccc2c2c4ccccc4n(-c4ncc5c(ccc6ccccc65)n4)c23)cc1] | 63% |
| | | | Compound 170 | |
| 27 | 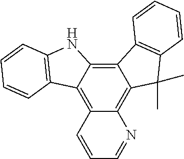 [SMILES: CC1(C)c2ccccc2-c2c1c1ncccc1c1c2[nH]c2ccccc21] | 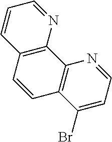 [SMILES: Brc1ccnc2c1ccc1cccnc12] | 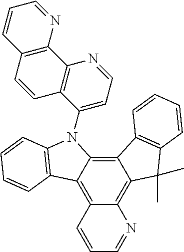 [SMILES: CC1(C)c2ccccc2-c2c1c1ncccc1c1c3ccccc3n(-c3ccnc4c3ccc3cccnc34)c21] | 64% |
| | | | Compound 184 | |
| 28 | 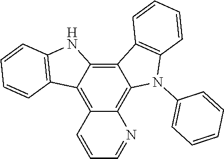 [SMILES: c1ccc(-n2c3ccccc3c3c4[nH]c5ccccc5c4c4cccnc4c32)cc1] | 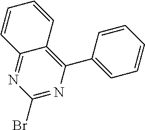 [SMILES: Brc1nc(-c2ccccc2)c2ccccc2n1] | 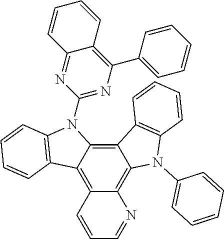 [SMILES: c1ccc(-c2nc(-n3c4ccccc4c4c5cccnc5c5c(c6ccccc6n5-c5ccccc5)c43)nc3ccccc23)cc1] | 66% |
| | | | Compound 195 | |
| 29 | 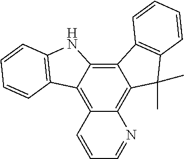 [SMILES: CC1(C)c2ccccc2-c2c1c1ncccc1c1c2[nH]c2ccccc21] | 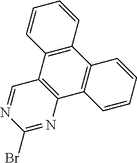 [SMILES: Brc1ncc2c3ccccc3c3ccccc3c2n1] | 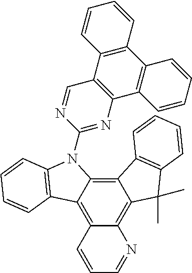 [SMILES: CC1(C)c2ccccc2-c2c1c1ncccc1c1c3ccccc3n(-c3ncc4c5ccccc5c5ccccc5c4n3)c21] | 56% |
| | | | Compound 201 | |
| 30 | 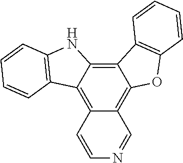 [SMILES: c1ccc2c(c1)[nH]c1c2c2ccncc2c2oc3ccccc3c12] | 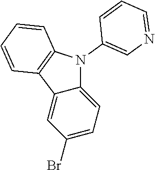 [SMILES: Brc1ccc2c(c1)c1ccccc1n2-c1cccnc1] | 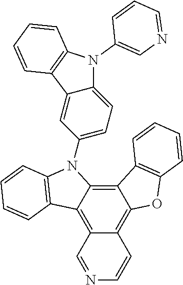 [SMILES: c1cncc(-n2c3ccccc3c3cc(-n4c5ccccc5c5c6cnccc6c6oc7ccccc7c6c54)ccc32)c1] | 68% |
| | | | Compound 213 | |
| 31 | 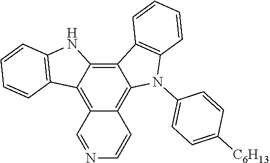 | 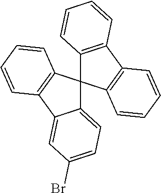 [SMILES: Brc1ccc2c(c1)-c1ccccc1C21c2ccccc2-c2ccccc21] | 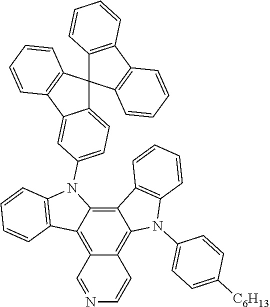 | 61% |
| | | | Compound 220 | |
| 32 | 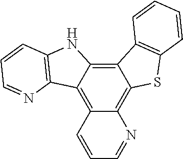 [SMILES: c1cnc2c(c1)[nH]c1c2c2cccnc2c2sc3ccccc3c12] | 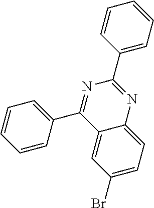 [SMILES: Brc1ccc2nc(-c3ccccc3)nc(-c3ccccc3)c2c1] | 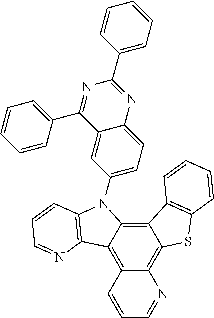 [SMILES: c1ccc(-c2nc(-c3ccccc3)c3cc(-n4c5cccnc5c5c6cccnc6c6sc7ccccc7c6c54)ccc3n2)cc1] | 60% |
| | | | Compound 237 | |
| 33 | 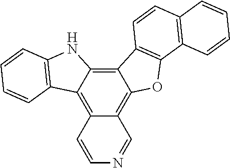 [SMILES: c1ccc2c(c1)ccc1c2oc2c3cnccc3c3c4ccccc4[nH]c3c12] | 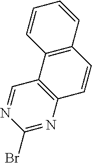 [SMILES: Brc1ncc2c(ccc3ccccc32)n1] | 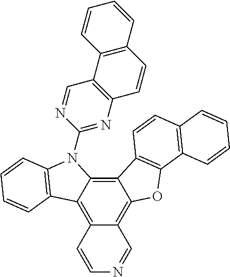 [SMILES: c1ccc2c(c1)ccc1nc(-n3c4ccccc4c4c5ccncc5c5oc6c7ccccc7ccc6c5c43)ncc12] | 63% |
| | | | Compound 240 | |
| 34 | 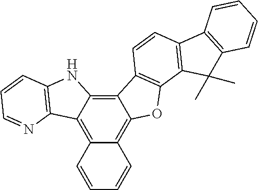 [SMILES: CC1(C)c2ccccc2-c2ccc3c(oc4c5ccccc5c5c6ncccc6[nH]c5c34)c21] | 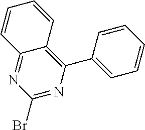 [SMILES: Brc1nc(-c2ccccc2)c2ccccc2n1] | 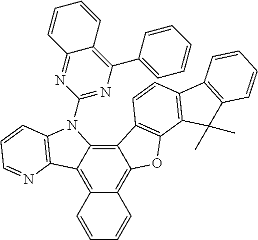 [SMILES: CC1(C)c2ccccc2-c2ccc3c(oc4c5ccccc5c5c6ncccc6n(-c6nc(-c7ccccc7)c7ccccc7n6)c5c34)c21] | 61% |
| | | | Compound 269 | |

It is understood that the various embodiments described herein are by way of example only, and are not intended to limit the scope of the invention. For example, many of the materials and structures described herein may be substituted with other materials and structures without deviating from the spirit of the invention. The present invention as claimed may therefore include variations from the particular examples and preferred embodiments described herein, as will be apparent to one of skill in the art. It is understood that various theories as to why the invention works are not intended to be limiting solely by the appended claims.

what is claimed is:

1. An organic compound represented by the following formula (1): 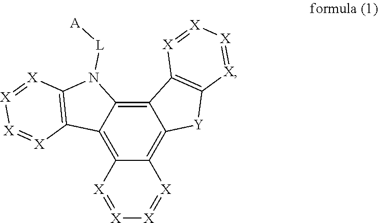
wherein Y is selected from the group consisting of O, S, Se, NR₁, CR₂R₃ and SiR₄R₆; X is CR₆ or N, and at least one X is N, and two adjacent X can form a five-membered ring, a six-membered ring or a combination thereof; L represents a single bond, a substituted or unsubstituted divalent arylene group having 6 to 30 ring carbon atoms, or a substituted or unsubstituted divalent heteroarylene group having 6 to 30 ring carbon atoms; A represents a aryl group having 6 to 30 ring carbon atoms, or a heteroaryl group having 5 to 30 ring carbon atoms; R₁ to R₆ are independently selected from the group consisting of H, a substituted or unsubstituted alkyl group having 1 to 20 carbon atoms, a substituted or unsubstituted aryl group having 6 to 30 carbon atoms, a substituted or unsubstituted aralkyl group having 6 to 30 carbon atoms and a substituted or unsubstituted heteroaryl group having 3 to 30 carbon atoms.

2. The organic compound according to claim 1, wherein at least one of the following is true:
only one X is N;
at least eleven X are not N; and
at least eleven X are CH.

3. The organic compound according to claim 1, wherein the alkyl group, aralkyl group, aryl group, or heteroaryl group is substituted by a halogen, an alkyl group, an aryl group, or a heteroaryl group.

4. The organic compound according to claim 1, wherein A is selected from the group consisting of pyrimidinyl, triazinyl, fluorenyl, quinazolinyl, benzoquinazolinyl, phenylquinazolinyl, 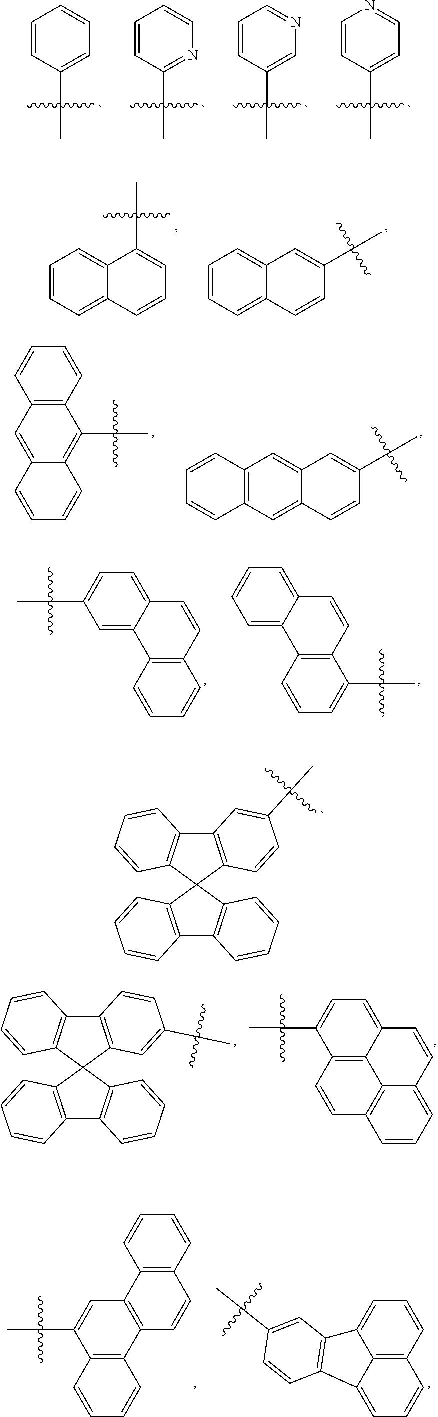 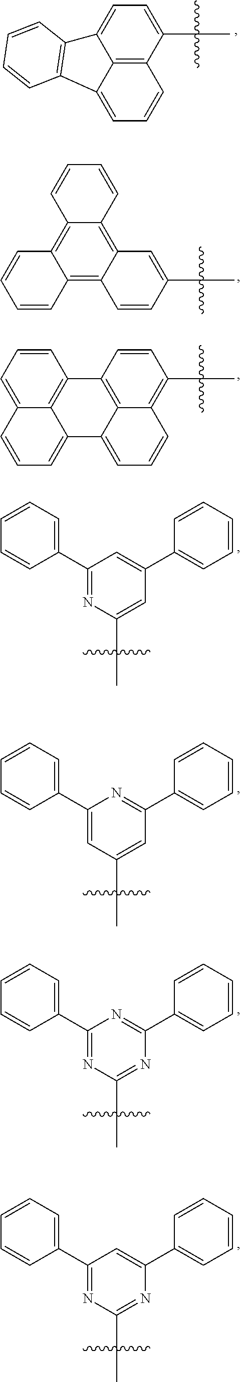 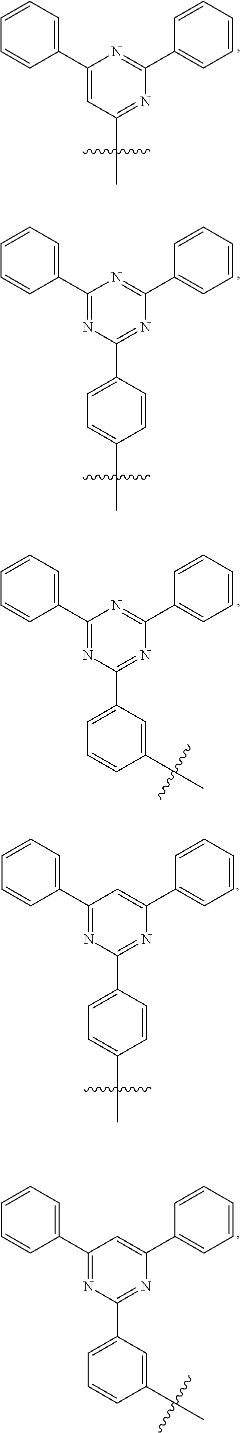 [SMILES: *c1cc(-c2ccccc2)nc(-c2ccccc2)n1 ; *c1ccc(-c2nc(-c3ccccc3)nc(-c3ccccc3)n2)cc1 ; *c1cccc(-c2nc(-c3ccccc3)nc(-c3ccccc3)n2)c1 ; *c1ccc(-c2nc(-c3ccccc3)cc(-c3ccccc3)n2)cc1 ; *c1cccc(-c2nc(-c3ccccc3)cc(-c3ccccc3)n2)c1] 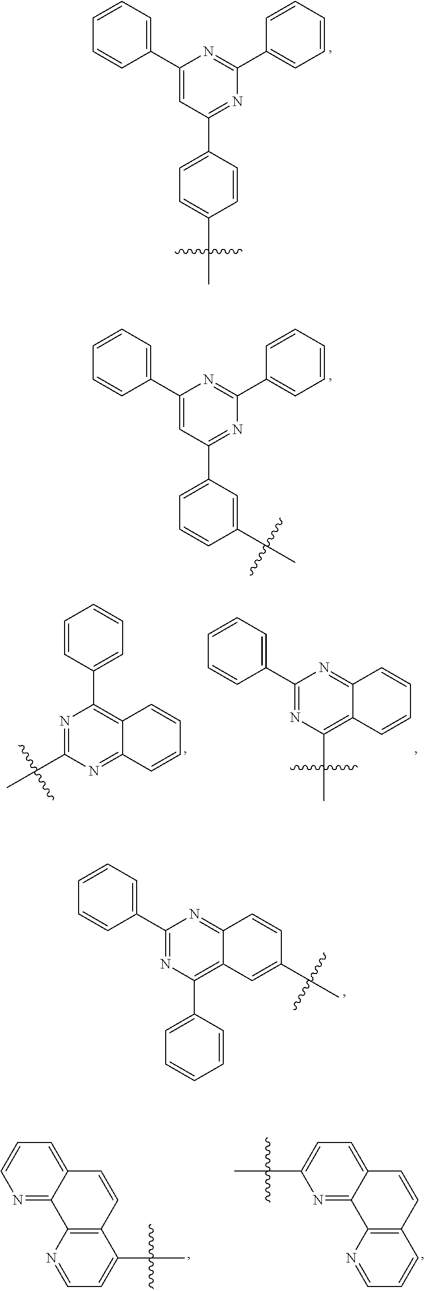 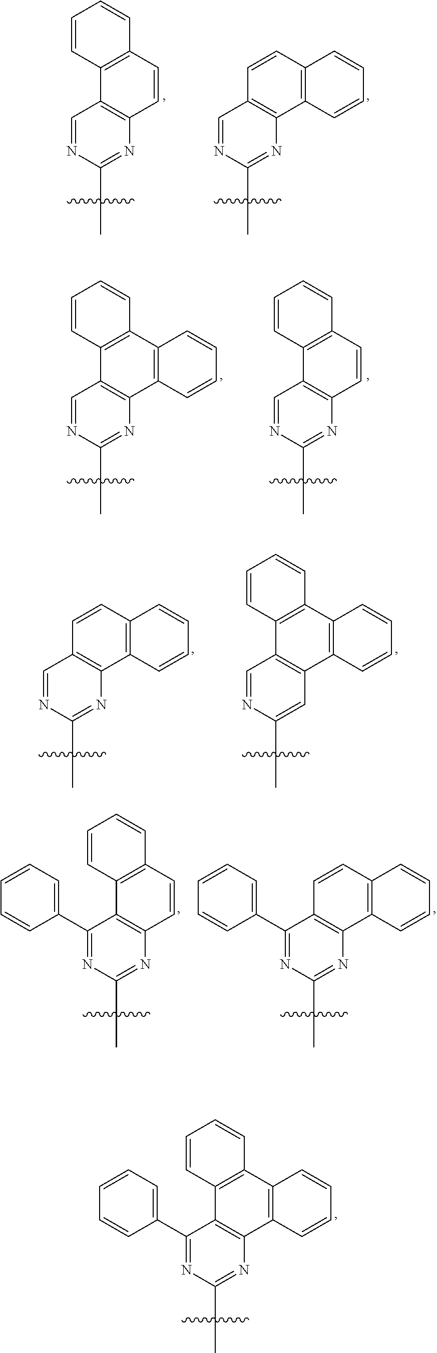 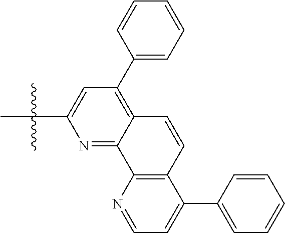 [SMILES: *c1cc(-c2ccccc2)c2ccc3c(-c4ccccc4)ccnc3c2n1]
and combinations thereof.

5. The organic compound according to claim 1, wherein the organic compound is selected from the group consisting of the following compounds:

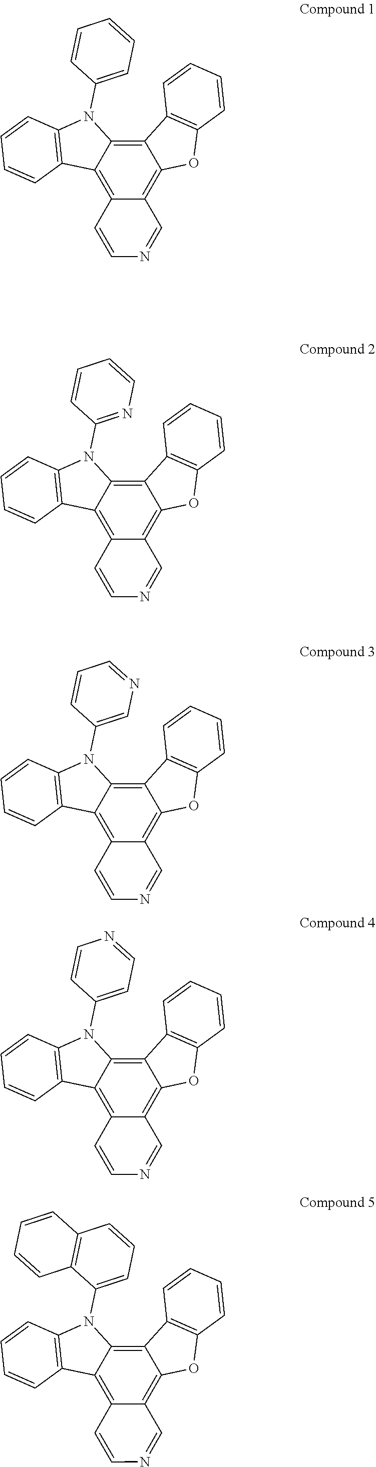

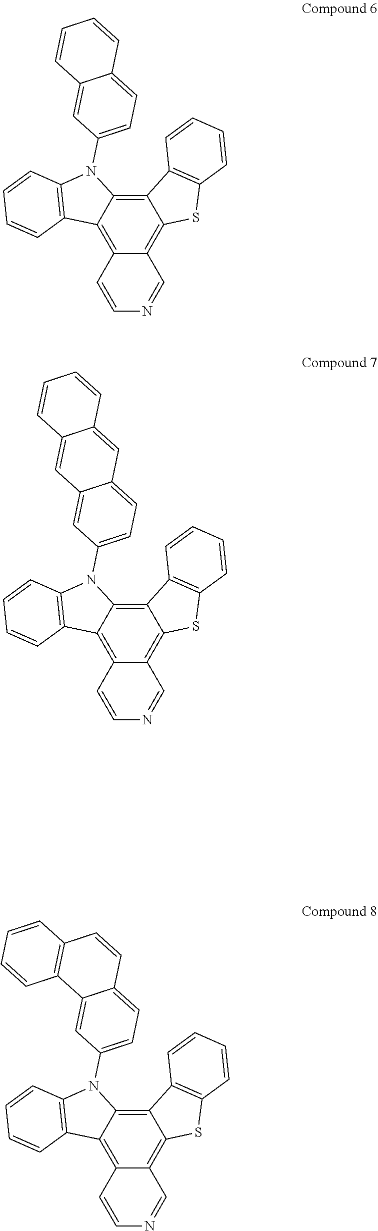 [SMILES: c1ccc2cc(-n3c4ccccc4c4c5ccncc5c5sc6ccccc6c5c43)ccc2c1 ; c1ccc2cc3cc(-n4c5ccccc5c5c6ccncc6c6sc7ccccc7c6c54)ccc3cc2c1 ; c1ccc2c(c1)ccc1ccc(-n3c4ccccc4c4c5ccncc5c5sc6ccccc6c5c43)cc12]

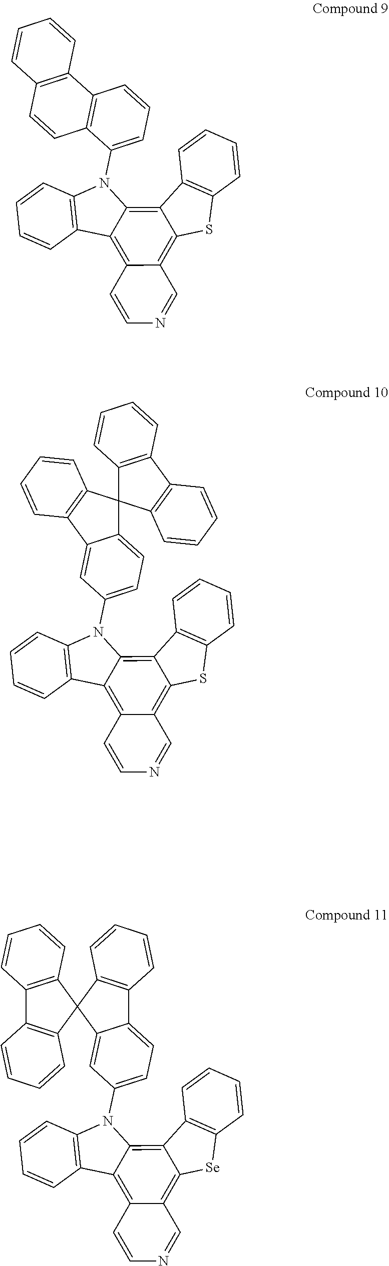 [SMILES: c1ccc2c(c1)ccc1c(-n3c4ccccc4c4c5ccncc5c5sc6ccccc6c5c43)cccc12 ; c1ccc2c(c1)-c1ccccc1C21c2ccccc2-c2cc(-n3c4ccccc4c4c5ccncc5c5sc6ccccc6c5c43)ccc21 ; c1ccc2c(c1)-c1ccccc1C21c2ccccc2-c2ccc(-n3c4ccccc4c4c5ccncc5c5[se]c6ccccc6c5c43)cc21]

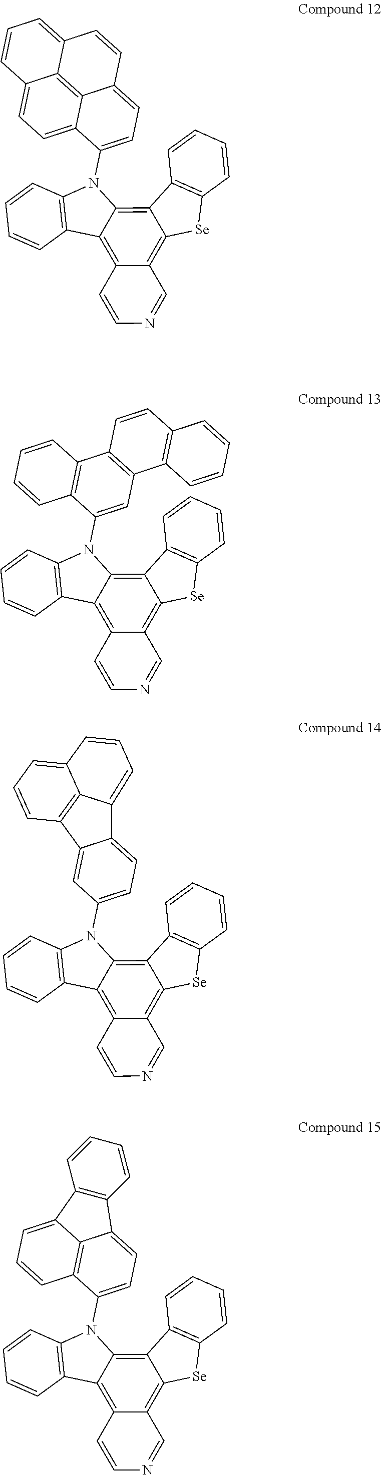 [SMILES: c1cc2ccc3ccc(-n4c5ccccc5c5c6ccncc6c6[se]c7ccccc7c6c54)c4ccc(c1)c2c34 ; c1ccc2c(c1)ccc1c3ccccc3c(-n3c4ccccc4c4c5ccncc5c5[se]c6ccccc6c5c43)cc21 ; c1cc2c3c(cccc3c1)-c1cc(-n3c4ccccc4c4c5ccncc5c5[se]c6ccccc6c5c43)ccc1-2 ; c1ccc2c(c1)-c1cccc3c(-n4c5ccccc5c5c6ccncc6c6[se]c7ccccc7c6c54)ccc-2c13]

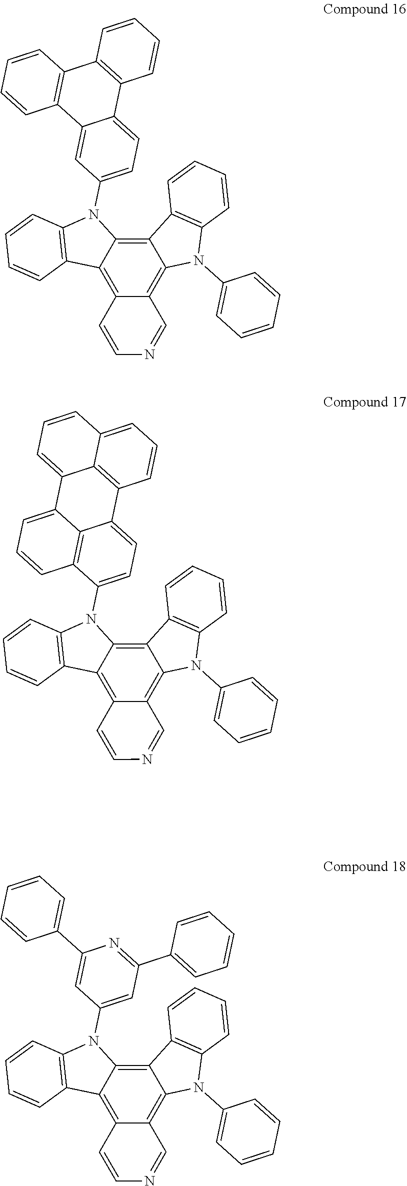 [SMILES: c1ccc(-n2c3ccccc3c3c2c2cnccc2c2c4ccccc4n(-c4ccc5c6ccccc6c6ccccc6c5c4)c23)cc1 ; c1ccc(-n2c3ccccc3c3c2c2cnccc2c2c4ccccc4n(-c4ccc5c6cccc7cccc(c8cccc4c85)c76)c23)cc1 ; c1ccc(-c2cc(-n3c4ccccc4c4c5ccncc5c5c(c6ccccc6n5-c5ccccc5)c43)cc(-c3ccccc3)n2)cc1]

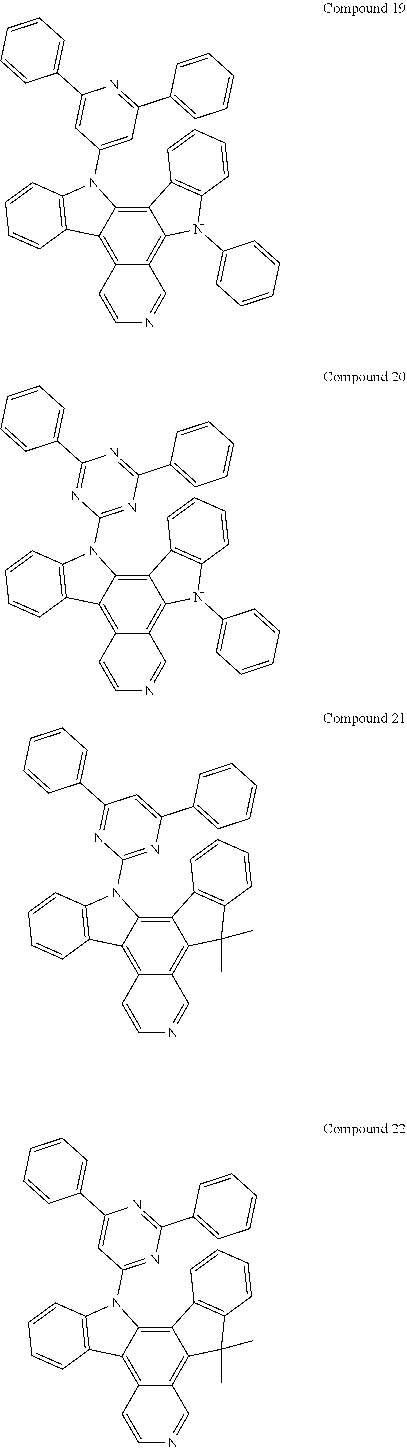 [SMILES: c1ccc(-c2cc(-n3c4ccccc4c4c5ccncc5c5c(c6ccccc6n5-c5ccccc5)c43)cc(-c3ccccc3)n2)cc1 ; c1ccc(-c2nc(-c3ccccc3)nc(-n3c4ccccc4c4c5ccncc5c5c(c6ccccc6n5-c5ccccc5)c43)n2)cc1 ; CC1(C)c2ccccc2-c2c1c1cnccc1c1c3ccccc3n(-c3nc(-c4ccccc4)cc(-c4ccccc4)n3)c21 ; CC1(C)c2ccccc2-c2c1c1cnccc1c1c3ccccc3n(-c3cc(-c4ccccc4)nc(-c4ccccc4)n3)c21]

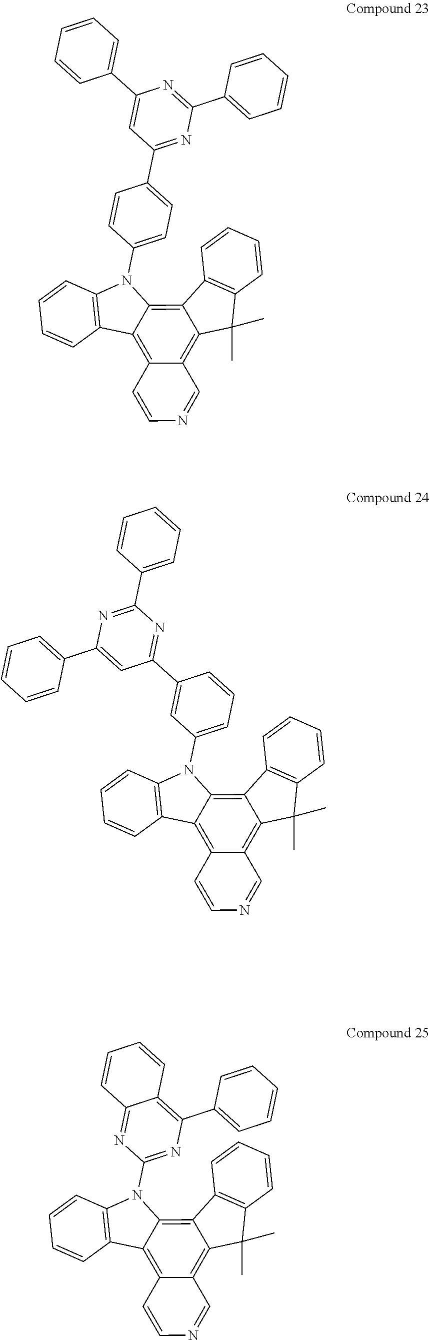 [SMILES: CC1(C)c2ccccc2-c2c1c1cnccc1c1c3ccccc3n(-c3ccc(-c4cc(-c5ccccc5)nc(-c5ccccc5)n4)cc3)c21 ; CC1(C)c2ccccc2-c2c1c1cnccc1c1c3ccccc3n(-c3cccc(-c4cc(-c5ccccc5)nc(-c5ccccc5)n4)c3)c21 ; CC1(C)c2ccccc2-c2c1c1cnccc1c1c3ccccc3n(-c3nc(-c4ccccc4)c4ccccc4n3)c21]

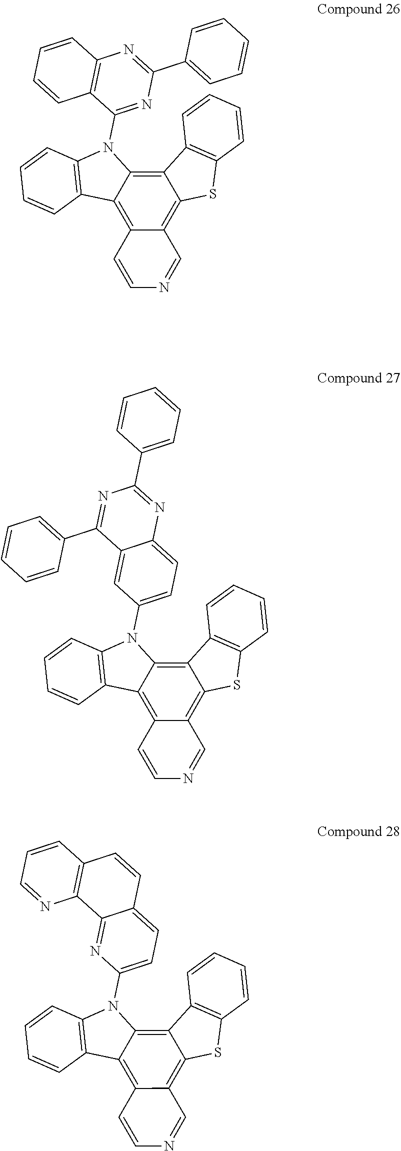 [SMILES: c1ccc(-c2nc(-n3c4ccccc4c4c5ccncc5c5sc6ccccc6c5c43)c3ccccc3n2)cc1 ; c1ccc(-c2nc(-c3ccccc3)c3cc(-n4c5ccccc5c5c6ccncc6c6sc7ccccc7c6c54)ccc3n2)cc1 ; c1cnc2c(c1)ccc1ccc(-n3c4ccccc4c4c5ccncc5c5sc6ccccc6c5c43)nc12]

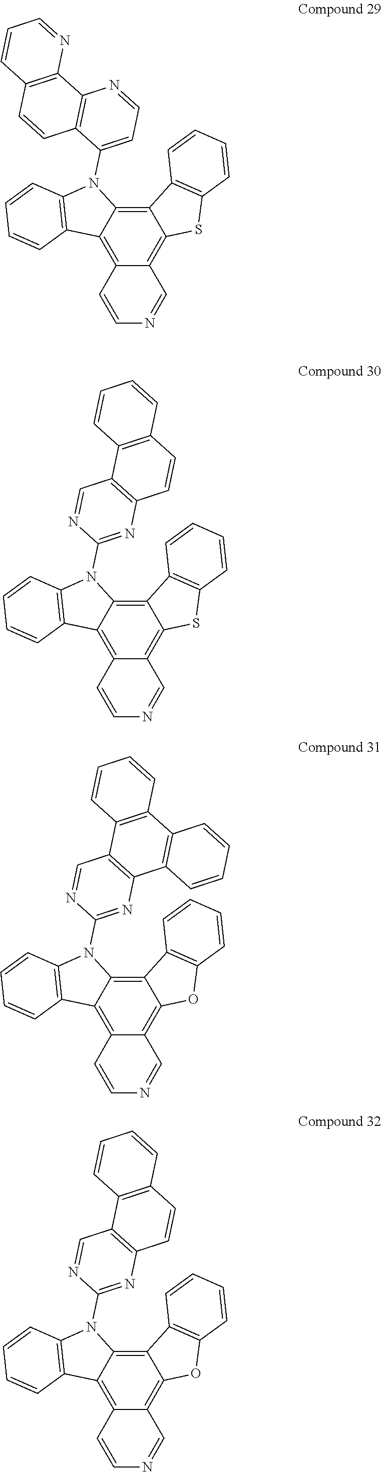

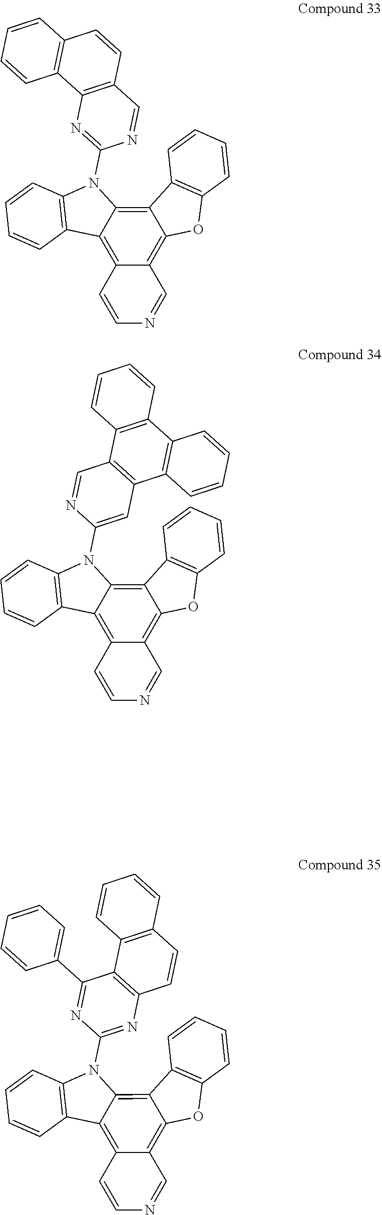

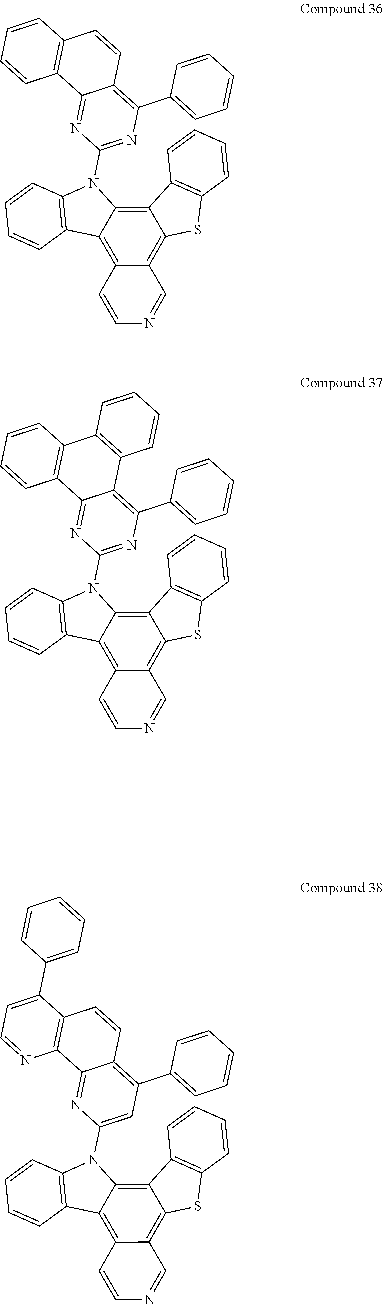 [SMILES: c1ccc(-c2nc(-n3c4ccccc4c4c5ccncc5c5sc6ccccc6c5c43)nc3c2ccc2ccccc23)cc1 ; c1ccc(-c2nc(-n3c4ccccc4c4c5ccncc5c5sc6ccccc6c5c43)nc3c4ccccc4c4ccccc4c23)cc1 ; c1ccc(-c2ccnc3c2ccc2c(-c4ccccc4)cc(-n4c5ccccc5c5c6ccncc6c6sc7ccccc7c6c54)nc23)cc1]

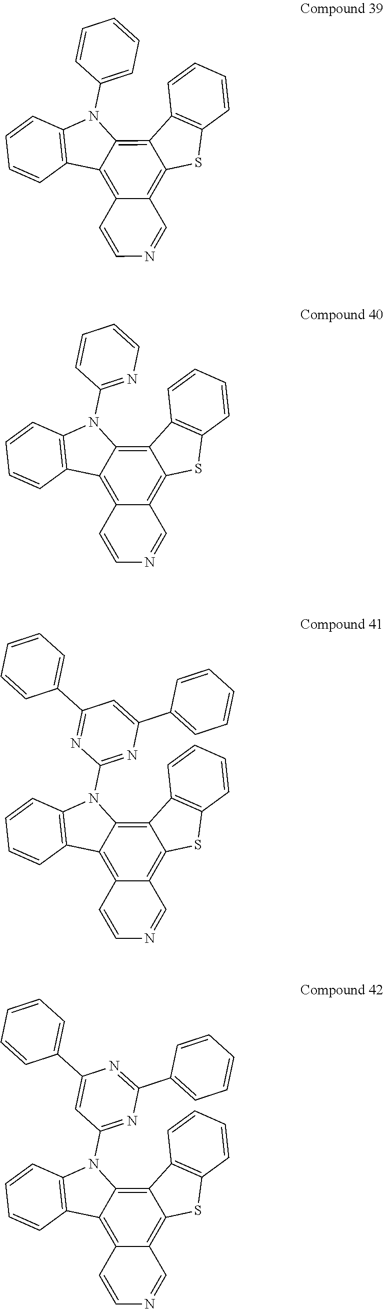 [SMILES: c1ccc(-n2c3ccccc3c3c4ccncc4c4sc5ccccc5c4c32)cc1 ; c1ccc(-n2c3ccccc3c3c4ccncc4c4sc5ccccc5c4c32)nc1 ; c1ccc(-c2cc(-c3ccccc3)nc(-n3c4ccccc4c4c5ccncc5c5sc6ccccc6c5c43)n2)cc1 ; c1ccc(-c2cc(-n3c4ccccc4c4c5ccncc5c5sc6ccccc6c5c43)nc(-c3ccccc3)n2)cc1]

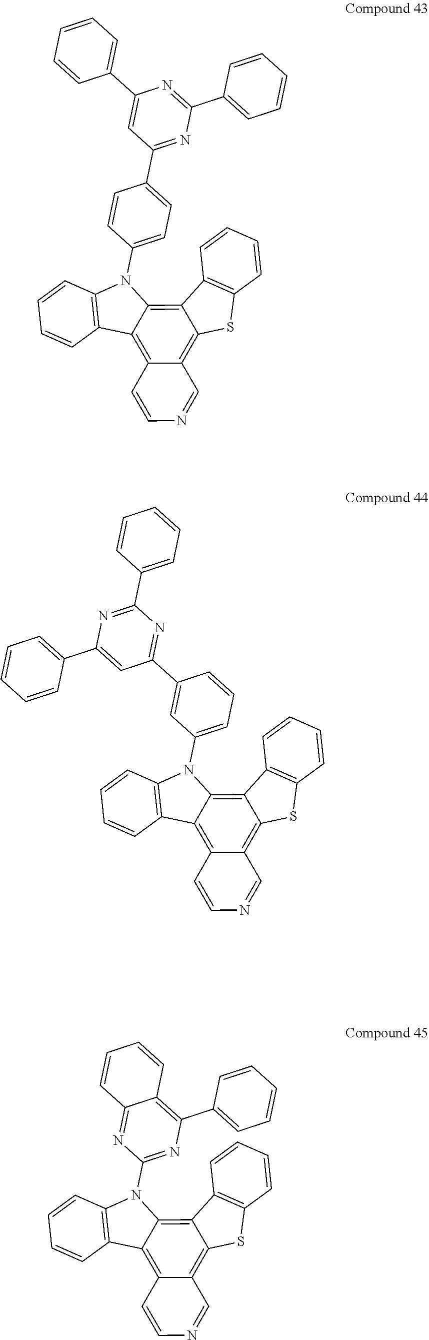 [SMILES: c1ccc(-c2cc(-c3ccc(-n4c5ccccc5c5c6ccncc6c6sc7ccccc7c6c54)cc3)nc(-c3ccccc3)n2)cc1 ; c1ccc(-c2cc(-c3cccc(-n4c5ccccc5c5c6ccncc6c6sc7ccccc7c6c54)c3)nc(-c3ccccc3)n2)cc1 ; c1ccc(-c2nc(-n3c4ccccc4c4c5ccncc5c5sc6ccccc6c5c43)nc3ccccc23)cc1]

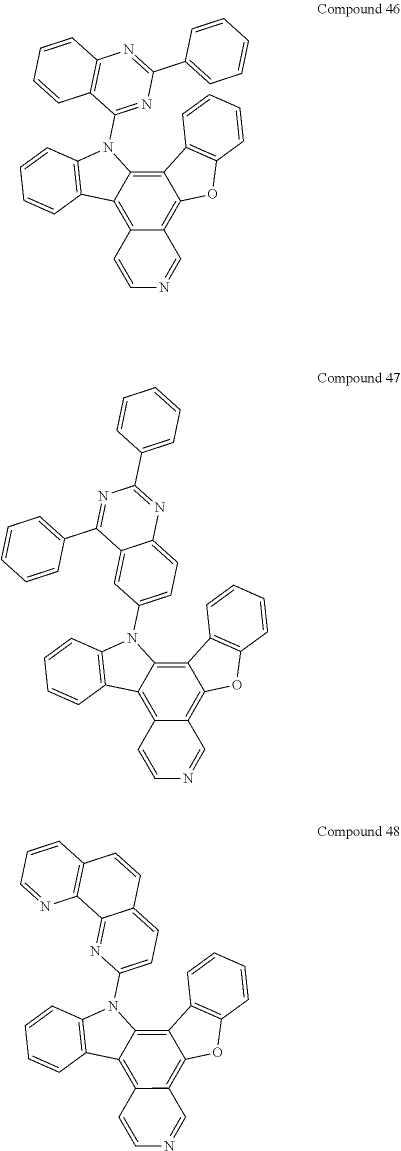 [SMILES: c1ccc(-c2nc(-n3c4ccccc4c4c5ccncc5c5oc6ccccc6c5c43)c3ccccc3n2)cc1 ; c1ccc(-c2nc(-c3ccccc3)c3cc(-n4c5ccccc5c5c6ccncc6c6oc7ccccc7c6c54)ccc3n2)cc1 ; c1cnc2c(c1)ccc1ccc(-n3c4ccccc4c4c5ccncc5c5oc6ccccc6c5c43)nc12]

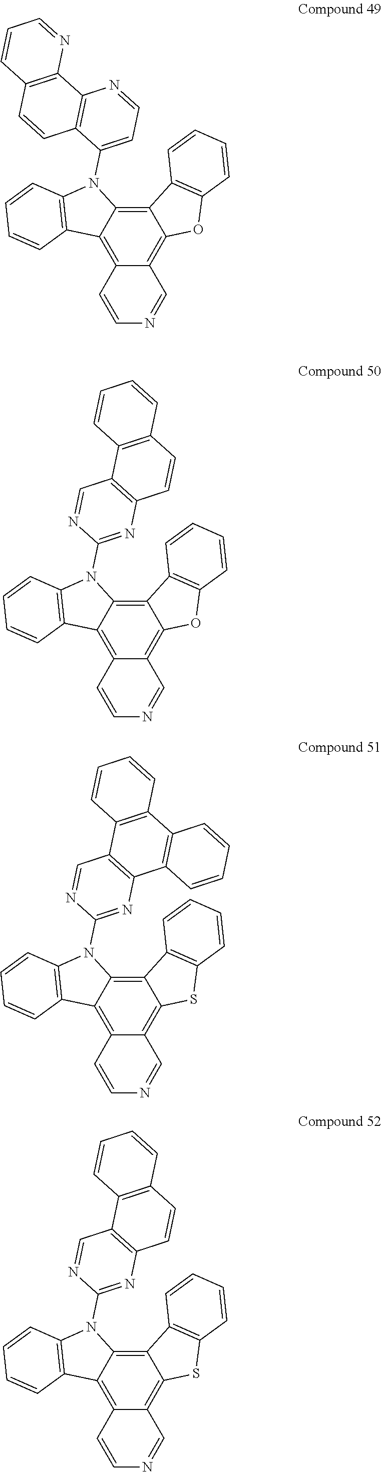

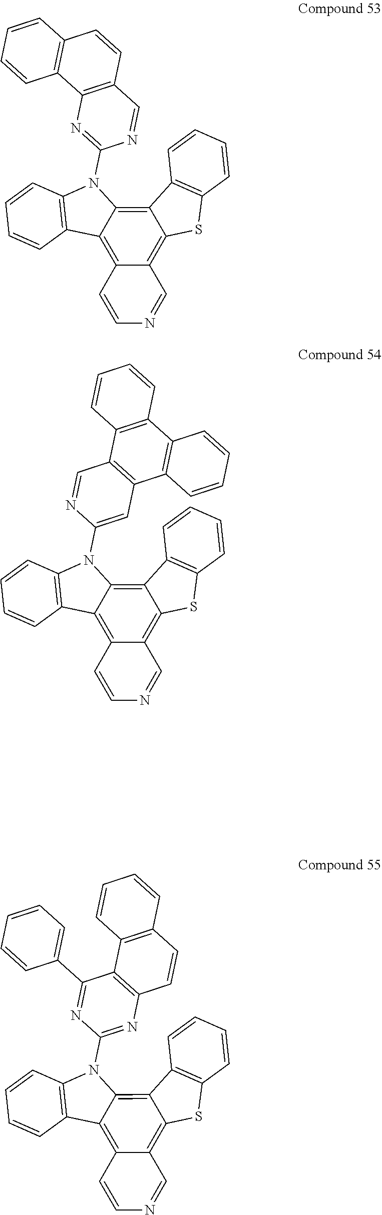

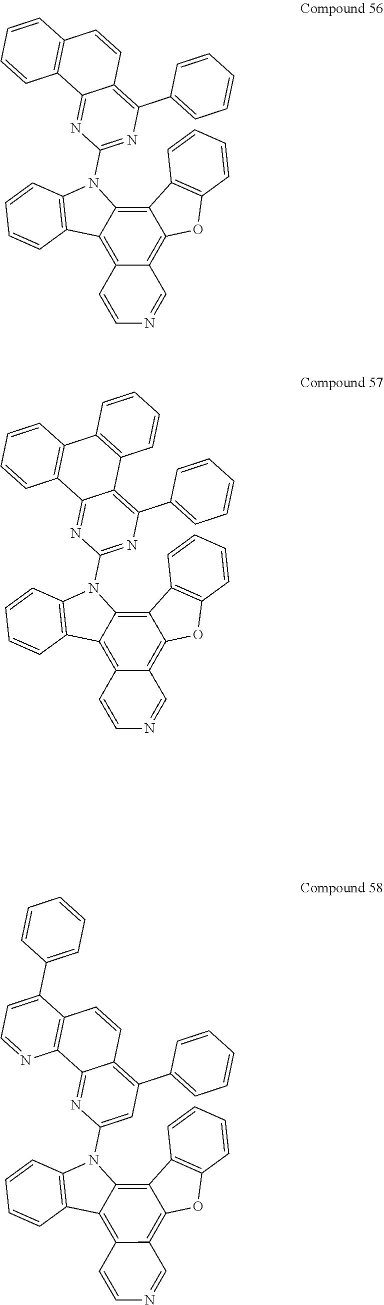 [SMILES: c1ccc(-c2nc(-n3c4ccccc4c4c5ccncc5c5oc6ccccc6c5c43)nc3c2ccc2ccccc23)cc1 ; c1ccc(-c2nc(-n3c4ccccc4c4c5ccncc5c5oc6ccccc6c5c43)nc3c4ccccc4c4ccccc4c23)cc1 ; c1ccc(-c2ccnc3c2ccc2c(-c4ccccc4)cc(-n4c5ccccc5c5c6ccncc6c6oc7ccccc7c6c54)nc23)cc1]

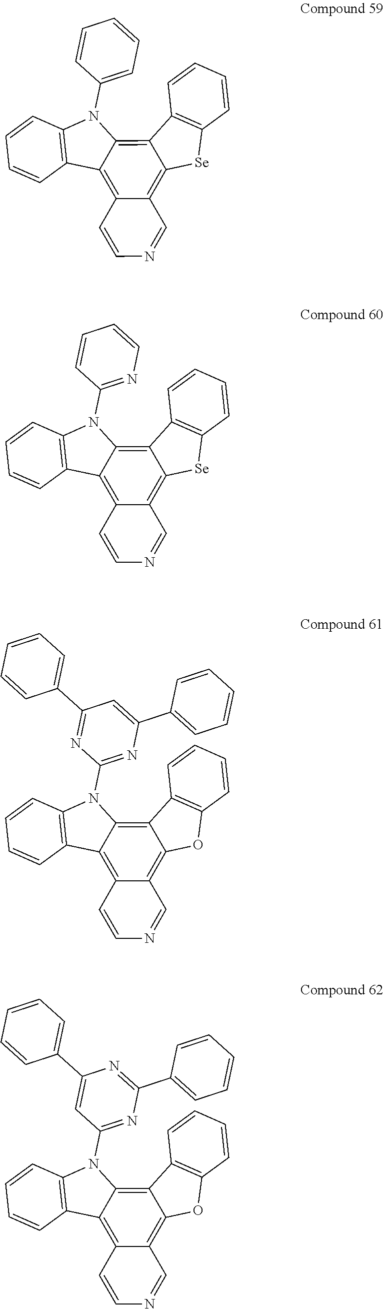 [SMILES: c1ccc(-n2c3ccccc3c3c4ccncc4c4[se]c5ccccc5c4c32)cc1 ; c1ccc(-n2c3ccccc3c3c4ccncc4c4[se]c5ccccc5c4c32)nc1 ; c1ccc(-c2cc(-c3ccccc3)nc(-n3c4ccccc4c4c5ccncc5c5oc6ccccc6c5c43)n2)cc1 ; c1ccc(-c2cc(-n3c4ccccc4c4c5ccncc5c5oc6ccccc6c5c43)nc(-c3ccccc3)n2)cc1]

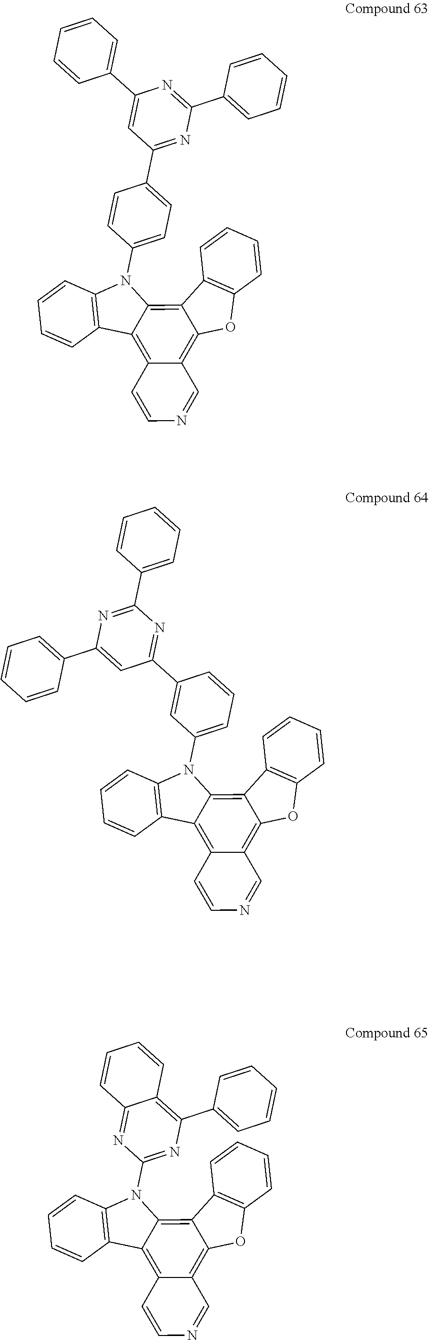 [SMILES: c1ccc(-c2cc(-c3ccc(-n4c5ccccc5c5c6ccncc6c6oc7ccccc7c6c54)cc3)nc(-c3ccccc3)n2)cc1 ; c1ccc(-c2cc(-c3cccc(-n4c5ccccc5c5c6ccncc6c6oc7ccccc7c6c54)c3)nc(-c3ccccc3)n2)cc1 ; c1ccc(-c2nc(-n3c4ccccc4c4c5ccncc5c5oc6ccccc6c5c43)nc3ccccc23)cc1]

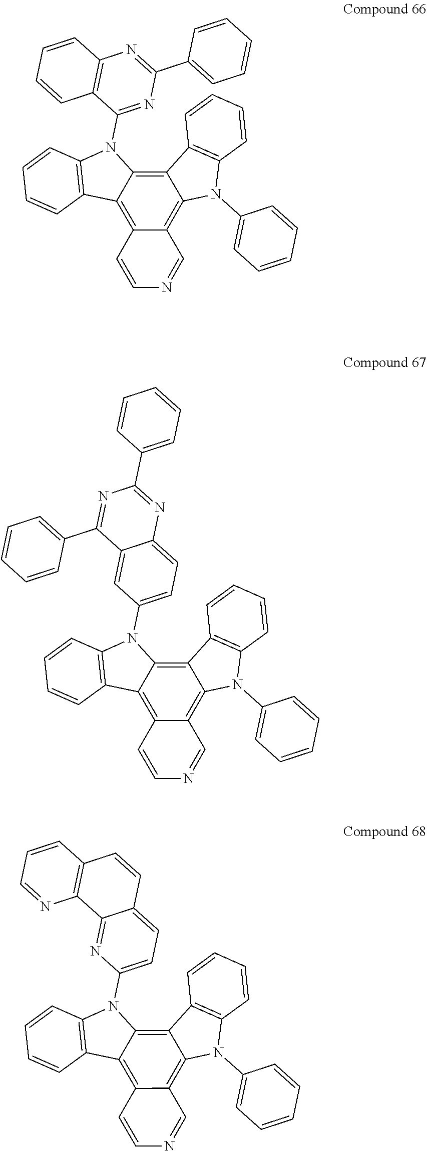 [SMILES: c1ccc(-c2nc(-n3c4ccccc4c4c5ccncc5c5c(c6ccccc6n5-c5ccccc5)c43)c3ccccc3n2)cc1 ; c1ccc(-c2nc(-c3ccccc3)c3cc(-n4c5ccccc5c5c6ccncc6c6c(c7ccccc7n6-c6ccccc6)c54)ccc3n2)cc1 ; c1ccc(-n2c3ccccc3c3c2c2cnccc2c2c4ccccc4n(-c4ccc5ccc6cccnc6c5n4)c23)cc1]

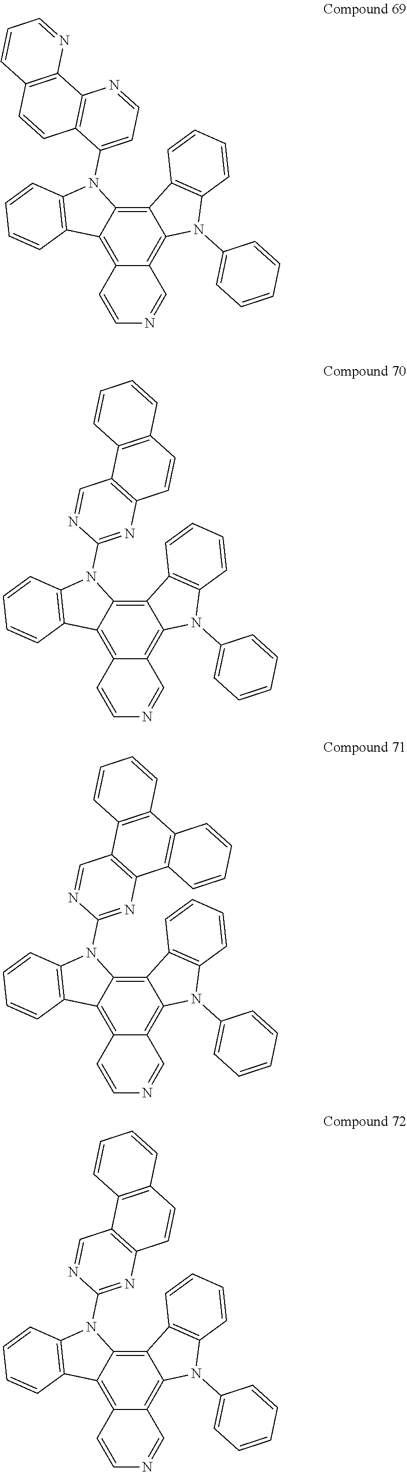

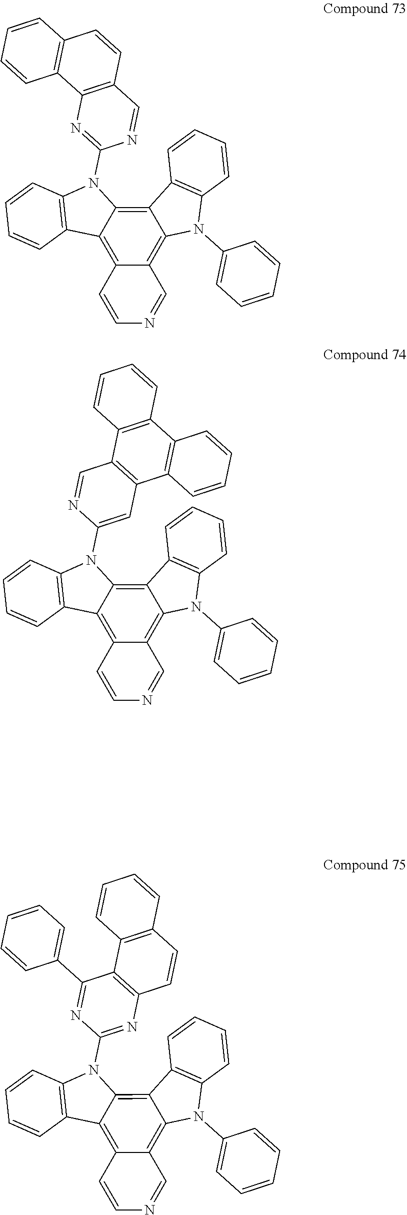

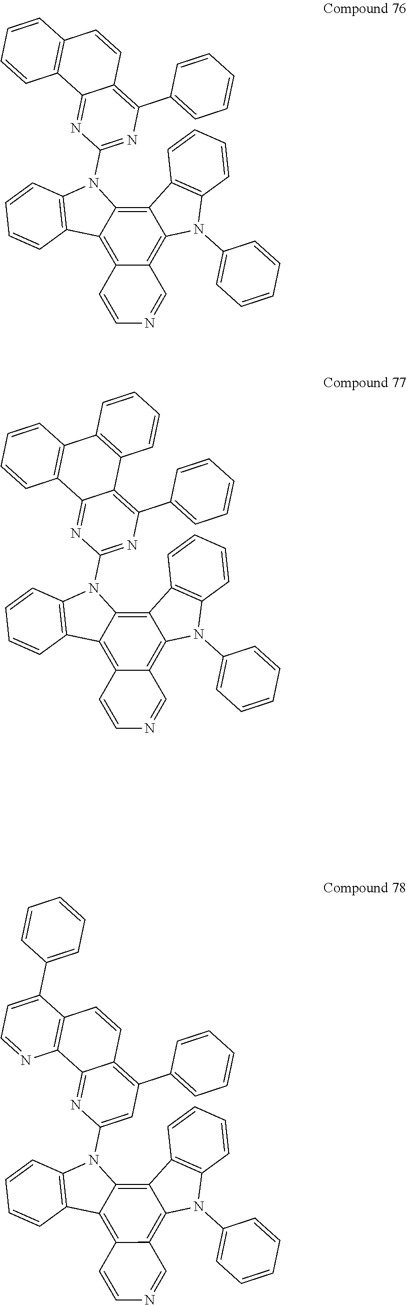 [SMILES: c1ccc(-c2nc(-n3c4ccccc4c4c5ccncc5c5c(c6ccccc6n5-c5ccccc5)c43)nc3c2ccc2ccccc23)cc1 ; c1ccc(-c2nc(-n3c4ccccc4c4c5ccncc5c5c(c6ccccc6n5-c5ccccc5)c43)nc3c4ccccc4c4ccccc4c23)cc1 ; c1ccc(-c2ccnc3c2ccc2c(-c4ccccc4)cc(-n4c5ccccc5c5c6ccncc6c6c(c7ccccc7n6-c6ccccc6)c54)nc23)cc1]

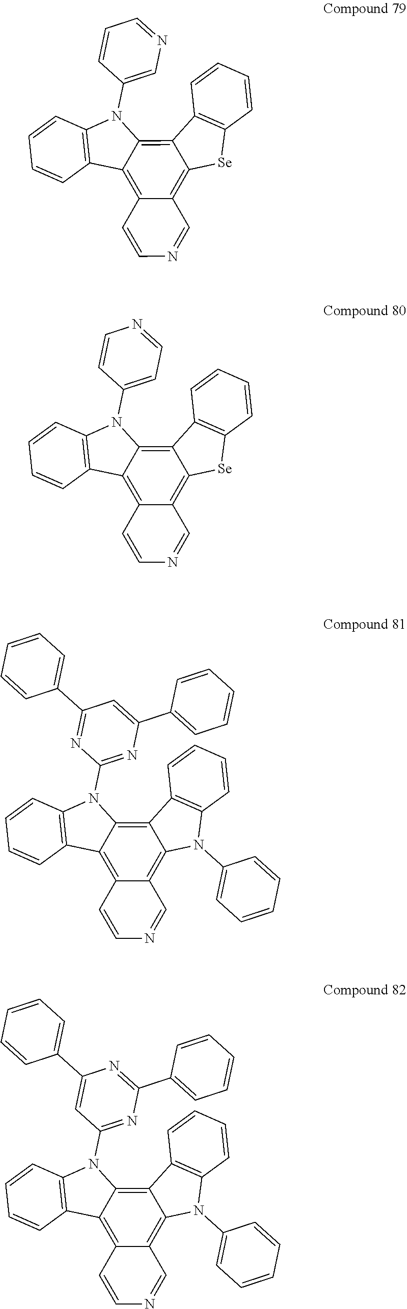 [SMILES: c1cncc(-n2c3ccccc3c3c4ccncc4c4[se]c5ccccc5c4c32)c1 ; c1ccc2c(c1)[se]c1c3cnccc3c3c4ccccc4n(-c4ccncc4)c3c21 ; c1ccc(-c2cc(-c3ccccc3)nc(-n3c4ccccc4c4c5ccncc5c5c(c6ccccc6n5-c5ccccc5)c43)n2)cc1 ; c1ccc(-c2cc(-n3c4ccccc4c4c5ccncc5c5c(c6ccccc6n5-c5ccccc5)c43)nc(-c3ccccc3)n2)cc1]

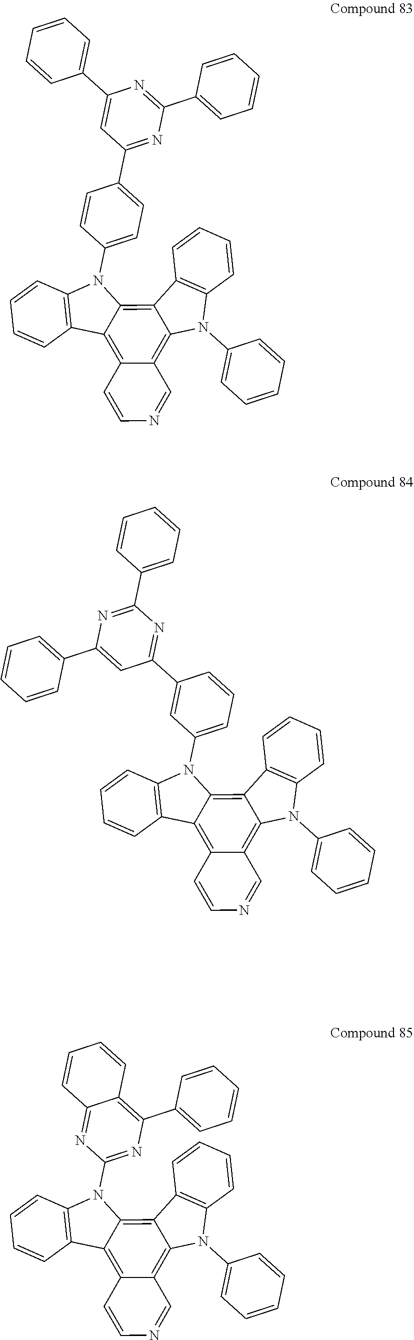 [SMILES: c1ccc(-c2cc(-c3ccc(-n4c5ccccc5c5c6ccncc6c6c(c7ccccc7n6-c6ccccc6)c54)cc3)nc(-c3ccccc3)n2)cc1 ; c1ccc(-c2cc(-c3cccc(-n4c5ccccc5c5c6ccncc6c6c(c7ccccc7n6-c6ccccc6)c54)c3)nc(-c3ccccc3)n2)cc1 ; c1ccc(-c2nc(-n3c4ccccc4c4c5ccncc5c5c(c6ccccc6n5-c5ccccc5)c43)nc3ccccc23)cc1]

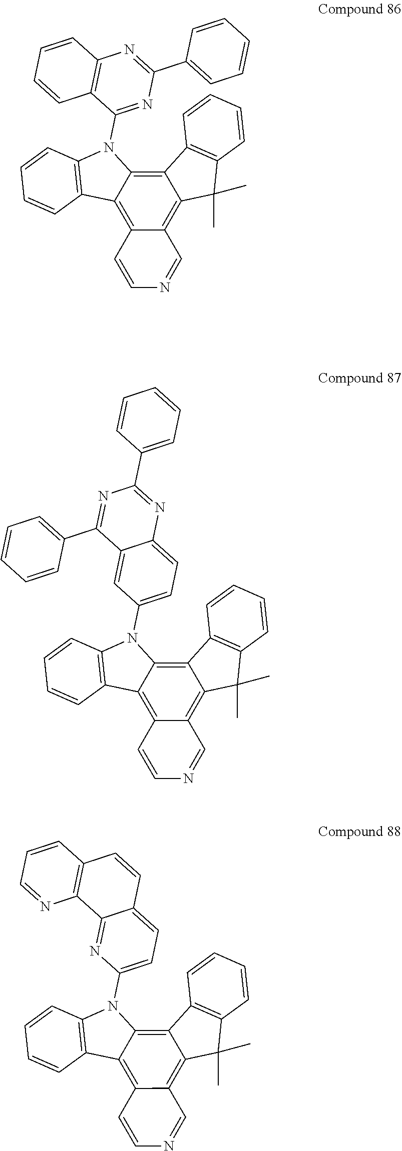 [SMILES: CC1(C)c2ccccc2-c2c1c1cnccc1c1c3ccccc3n(-c3nc(-c4ccccc4)nc4ccccc34)c21 ; CC1(C)c2ccccc2-c2c1c1cnccc1c1c3ccccc3n(-c3ccc4nc(-c5ccccc5)nc(-c5ccccc5)c4c3)c21 ; CC1(C)c2ccccc2-c2c1c1cnccc1c1c3ccccc3n(-c3ccc4ccc5cccnc5c4n3)c21]

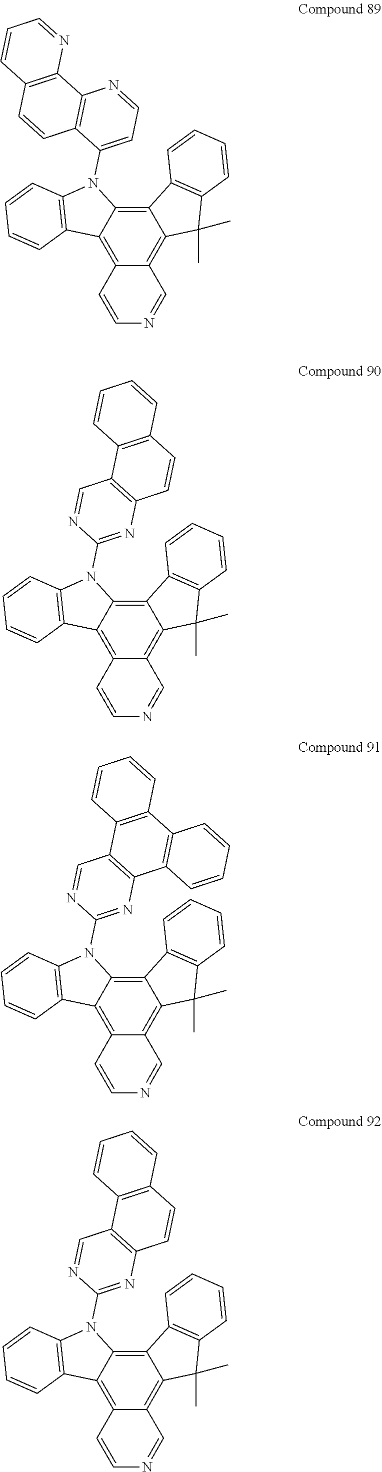

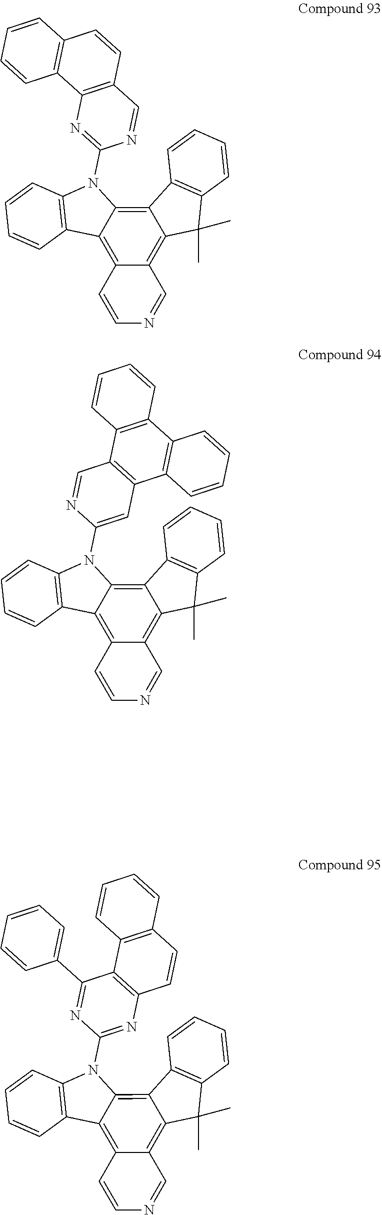

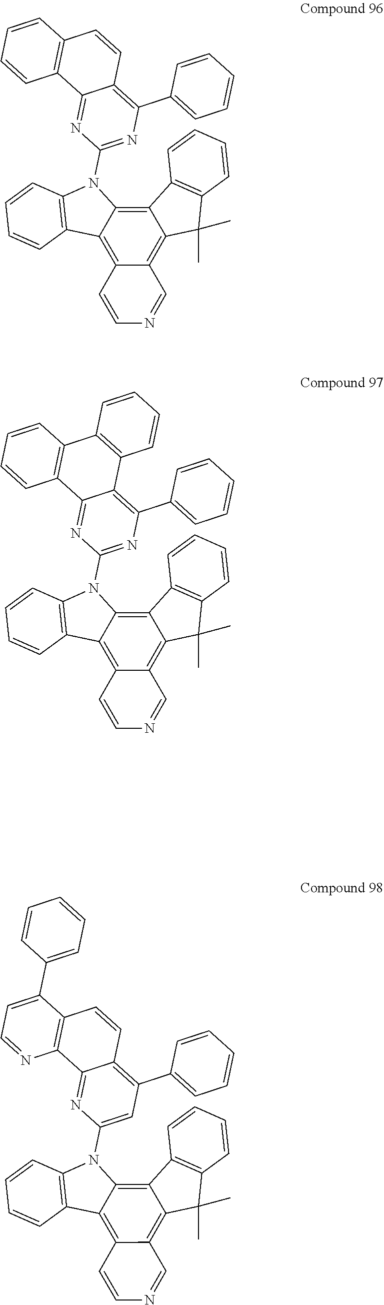 [SMILES: CC1(C)c2ccccc2-c2c1c1cnccc1c1c3ccccc3n(-c3nc(-c4ccccc4)c4ccc5ccccc5c4n3)c21 ; CC1(C)c2ccccc2-c2c1c1cnccc1c1c3ccccc3n(-c3nc(-c4ccccc4)c4c5ccccc5c5ccccc5c4n3)c21 ; CC1(C)c2ccccc2-c2c1c1cnccc1c1c3ccccc3n(-c3cc(-c4ccccc4)c4ccc5c(-c6ccccc6)ccnc5c4n3)c21]

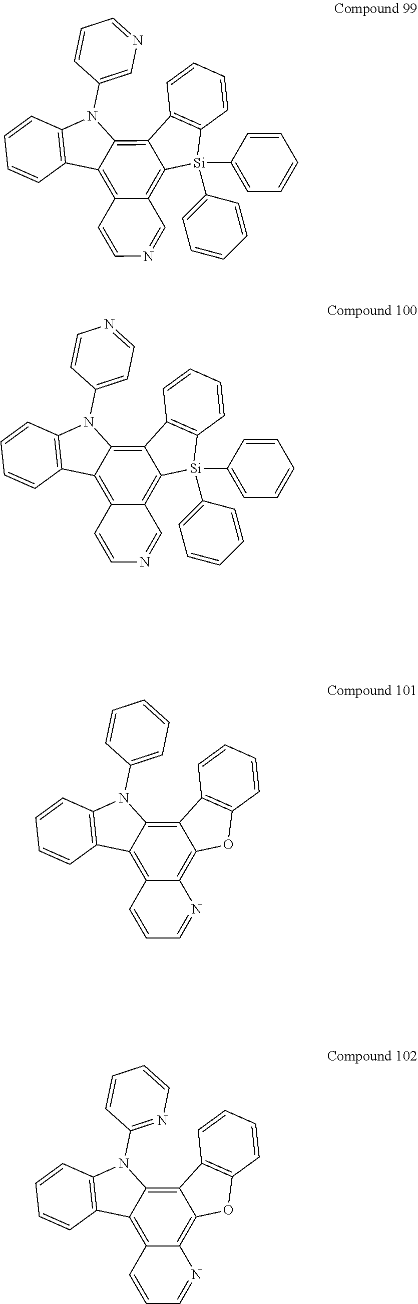 [SMILES: c1ccc([Si]2(c3ccccc3)c3ccccc3-c3c2c2cnccc2c2c4ccccc4n(-c4cccnc4)c32)cc1 ; c1ccc([Si]2(c3ccccc3)c3ccccc3-c3c2c2cnccc2c2c4ccccc4n(-c4ccncc4)c32)cc1 ; c1ccc(-n2c3ccccc3c3c4cccnc4c4oc5ccccc5c4c32)cc1 ; c1ccc(-n2c3ccccc3c3c4cccnc4c4oc5ccccc5c4c32)nc1]

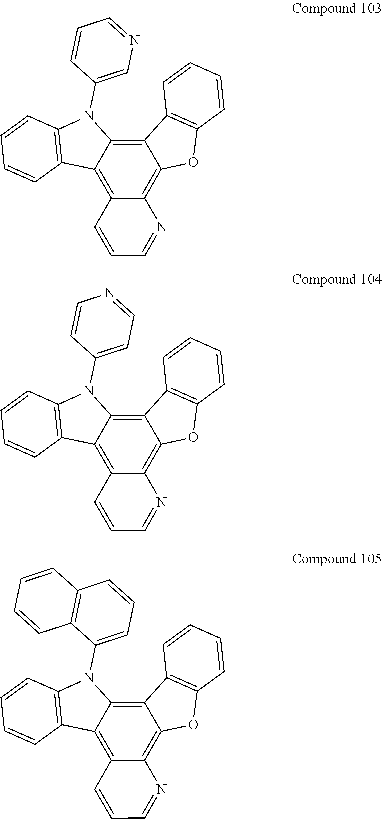

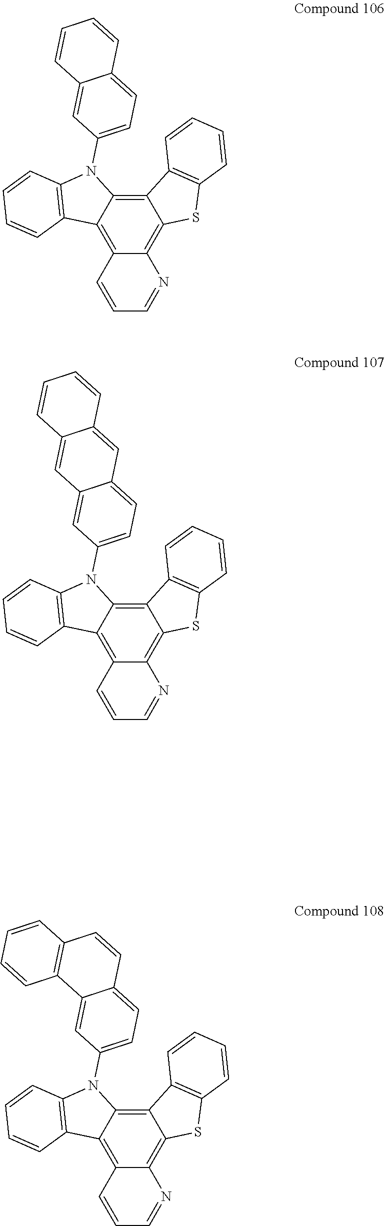 [SMILES: c1ccc2cc(-n3c4ccccc4c4c5cccnc5c5sc6ccccc6c5c43)ccc2c1 ; c1ccc2cc3cc(-n4c5ccccc5c5c6cccnc6c6sc7ccccc7c6c54)ccc3cc2c1 ; c1ccc2c(c1)ccc1ccc(-n3c4ccccc4c4c5cccnc5c5sc6ccccc6c5c43)cc12]

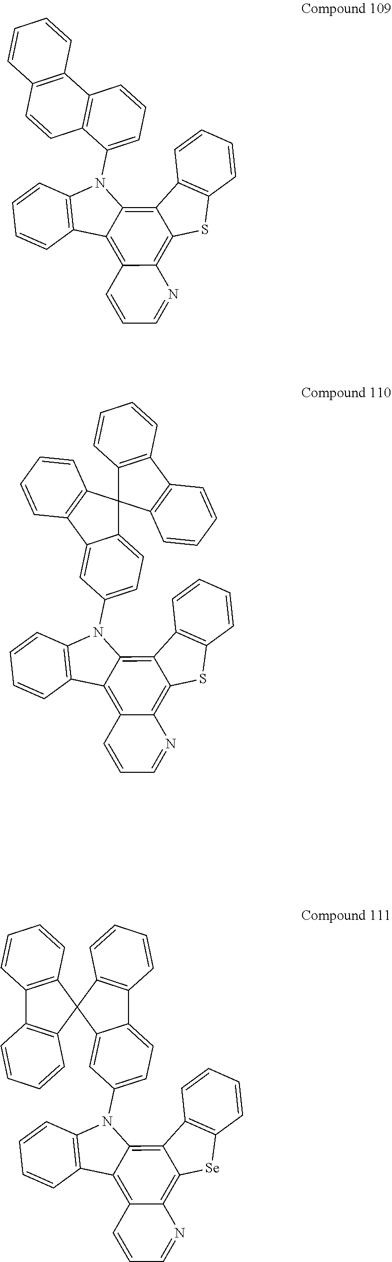 [SMILES: c1ccc2c(c1)ccc1c(-n3c4ccccc4c4c5cccnc5c5sc6ccccc6c5c43)cccc12 ; c1ccc2c(c1)-c1ccccc1C21c2ccccc2-c2cc(-n3c4ccccc4c4c5cccnc5c5sc6ccccc6c5c43)ccc21 ; c1ccc2c(c1)-c1ccccc1C21c2ccccc2-c2ccc(-n3c4ccccc4c4c5cccnc5c5[se]c6ccccc6c5c43)cc21]

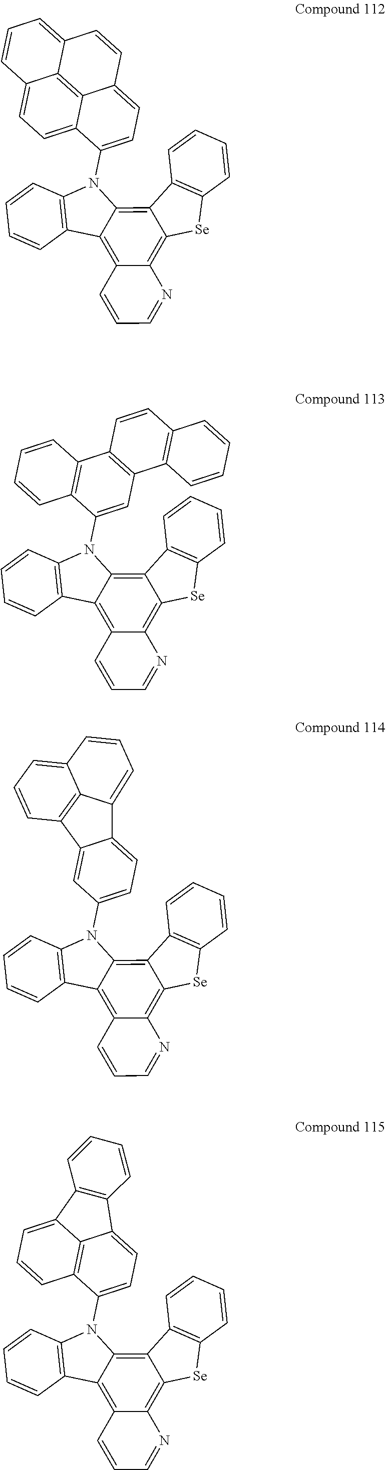 [SMILES: c1cc2ccc3ccc(-n4c5ccccc5c5c6cccnc6c6[se]c7ccccc7c6c54)c4ccc(c1)c2c34 ; c1ccc2c(c1)ccc1c3ccccc3c(-n3c4ccccc4c4c5cccnc5c5[se]c6ccccc6c5c43)cc21 ; c1cc2c3c(cccc3c1)-c1cc(-n3c4ccccc4c4c5cccnc5c5[se]c6ccccc6c5c43)ccc1-2 ; c1ccc2c(c1)-c1cccc3c(-n4c5ccccc5c5c6cccnc6c6[se]c7ccccc7c6c54)ccc-2c13]

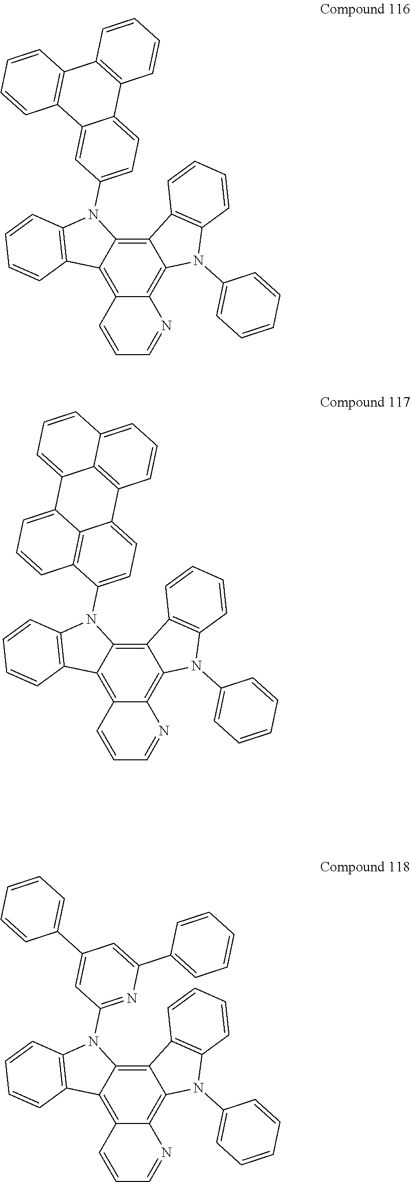 [SMILES: c1ccc(-n2c3ccccc3c3c2c2ncccc2c2c4ccccc4n(-c4ccc5c6ccccc6c6ccccc6c5c4)c23)cc1 ; c1ccc(-n2c3ccccc3c3c2c2ncccc2c2c4ccccc4n(-c4ccc5c6cccc7cccc(c8cccc4c85)c76)c23)cc1 ; c1ccc(-c2cc(-c3ccccc3)nc(-n3c4ccccc4c4c5cccnc5c5c(c6ccccc6n5-c5ccccc5)c43)c2)cc1]

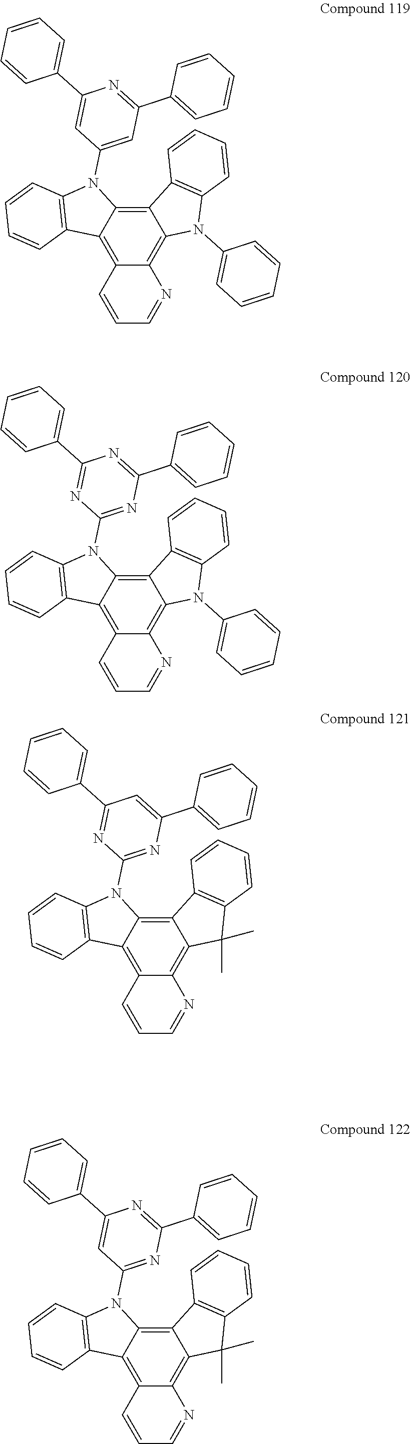 [SMILES: c1ccc(-c2cc(-n3c4ccccc4c4c5cccnc5c5c(c6ccccc6n5-c5ccccc5)c43)cc(-c3ccccc3)n2)cc1 ; c1ccc(-c2nc(-c3ccccc3)nc(-n3c4ccccc4c4c5cccnc5c5c(c6ccccc6n5-c5ccccc5)c43)n2)cc1 ; CC1(C)c2ccccc2-c2c1c1ncccc1c1c3ccccc3n(-c3nc(-c4ccccc4)cc(-c4ccccc4)n3)c21 ; CC1(C)c2ccccc2-c2c1c1ncccc1c1c3ccccc3n(-c3cc(-c4ccccc4)nc(-c4ccccc4)n3)c21]

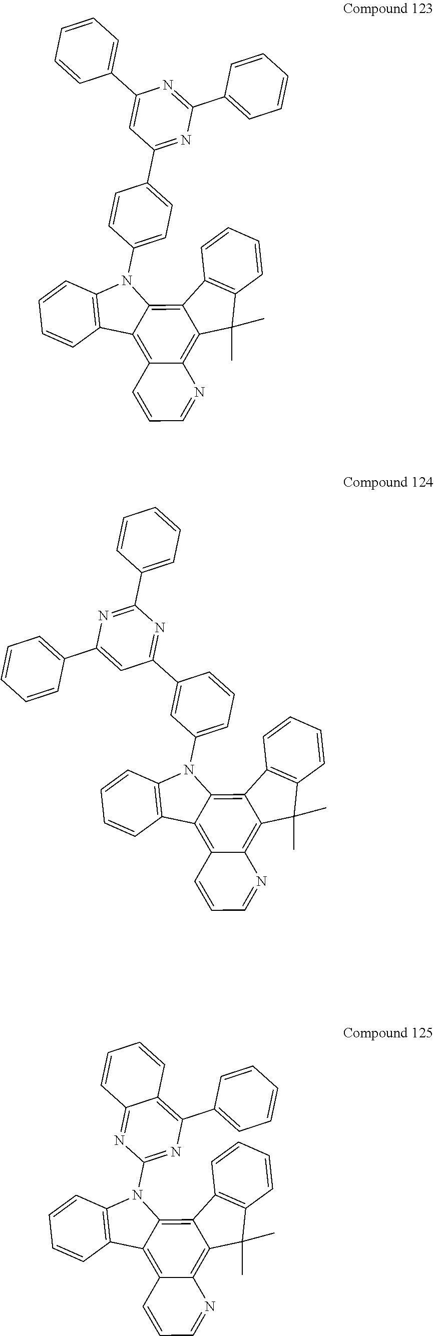 [SMILES: CC1(C)c2ccccc2-c2c1c1ncccc1c1c3ccccc3n(-c3ccc(-c4cc(-c5ccccc5)nc(-c5ccccc5)n4)cc3)c21 ; CC1(C)c2ccccc2-c2c1c1ncccc1c1c3ccccc3n(-c3cccc(-c4cc(-c5ccccc5)nc(-c5ccccc5)n4)c3)c21 ; CC1(C)c2ccccc2-c2c1c1ncccc1c1c3ccccc3n(-c3nc(-c4ccccc4)c4ccccc4n3)c21]

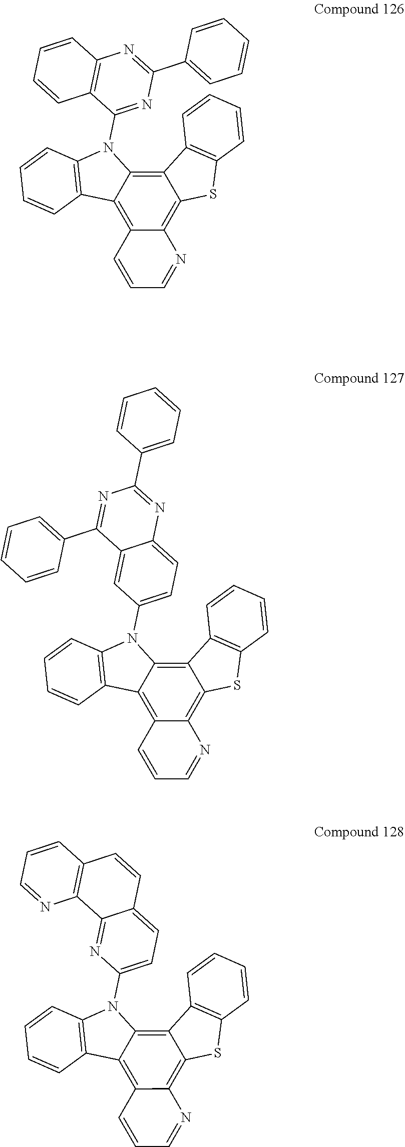 [SMILES: c1ccc(-c2nc(-n3c4ccccc4c4c5cccnc5c5sc6ccccc6c5c43)c3ccccc3n2)cc1 ; c1ccc(-c2nc(-c3ccccc3)c3cc(-n4c5ccccc5c5c6cccnc6c6sc7ccccc7c6c54)ccc3n2)cc1 ; c1cnc2c(c1)ccc1ccc(-n3c4ccccc4c4c5cccnc5c5sc6ccccc6c5c43)nc12]

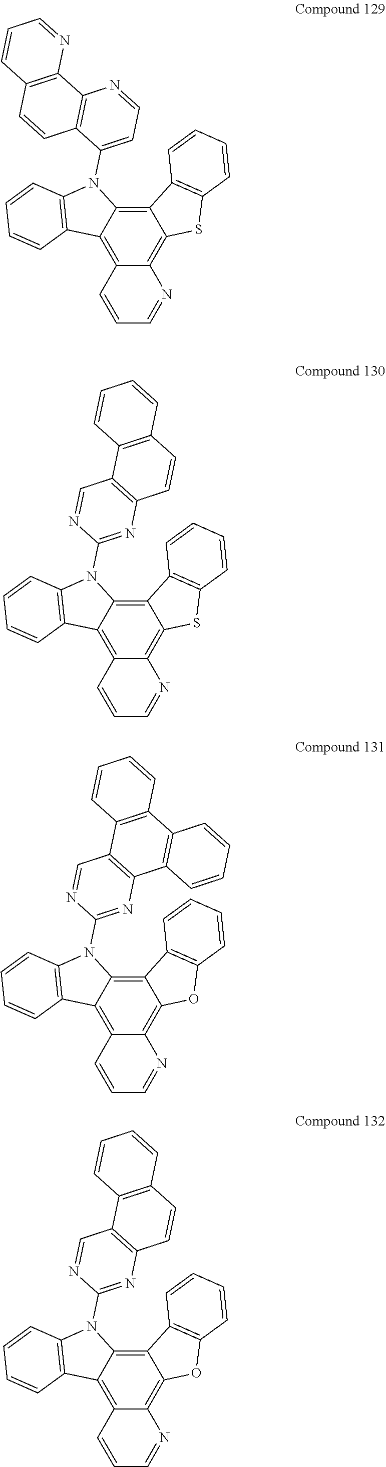

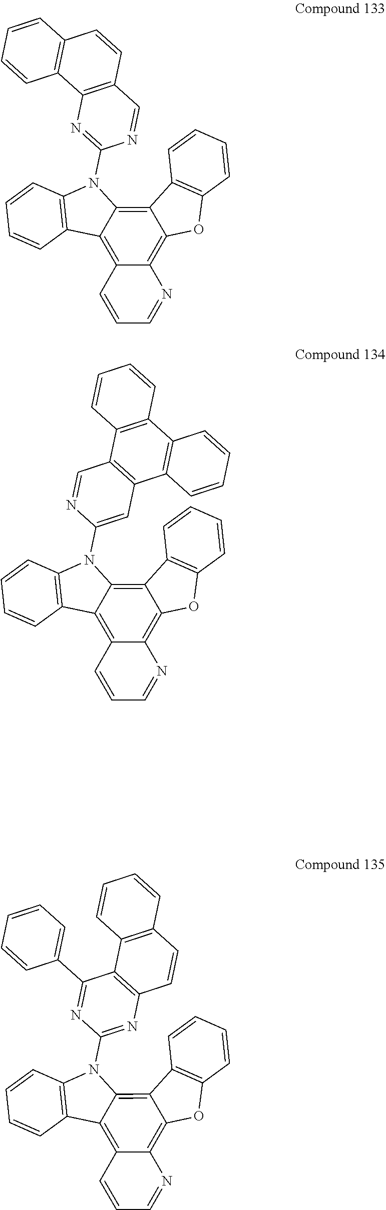

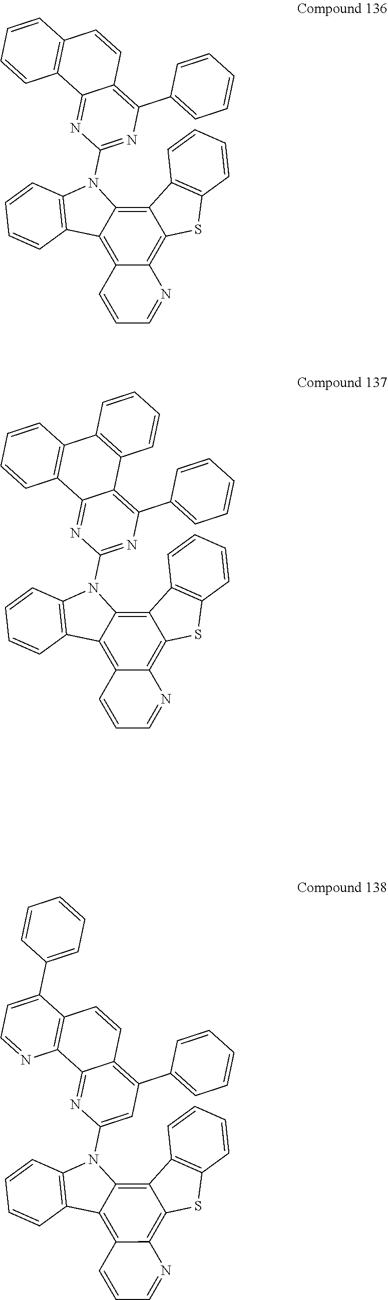 [SMILES: c1ccc(-c2nc(-n3c4ccccc4c4c5cccnc5c5sc6ccccc6c5c43)nc3c2ccc2ccccc23)cc1 ; c1ccc(-c2nc(-n3c4ccccc4c4c5cccnc5c5sc6ccccc6c5c43)nc3c4ccccc4c4ccccc4c23)cc1 ; c1ccc(-c2ccnc3c2ccc2c(-c4ccccc4)cc(-n4c5ccccc5c5c6cccnc6c6sc7ccccc7c6c54)nc23)cc1]

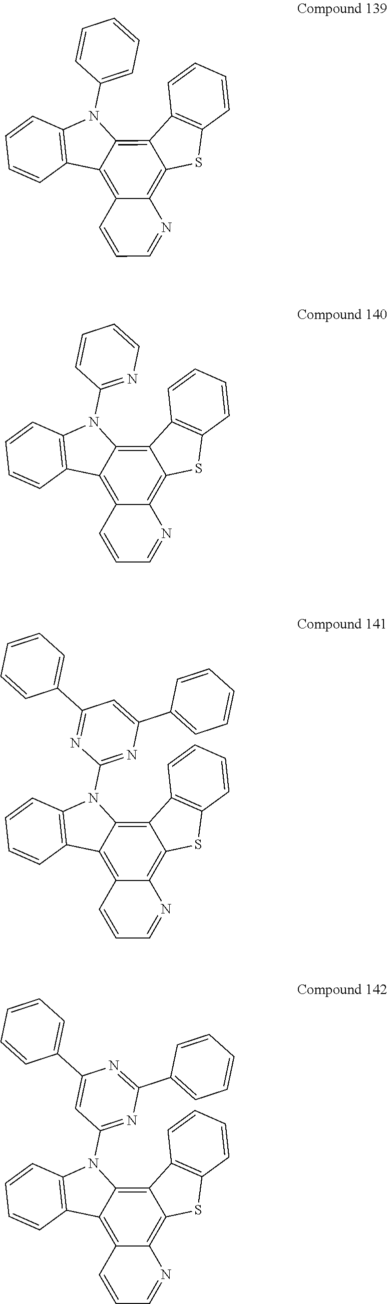 [SMILES: c1ccc(-n2c3ccccc3c3c4cccnc4c4sc5ccccc5c4c32)cc1 ; c1ccc(-n2c3ccccc3c3c4cccnc4c4sc5ccccc5c4c32)nc1 ; c1ccc(-c2cc(-c3ccccc3)nc(-n3c4ccccc4c4c5cccnc5c5sc6ccccc6c5c43)n2)cc1 ; c1ccc(-c2cc(-n3c4ccccc4c4c5cccnc5c5sc6ccccc6c5c43)nc(-c3ccccc3)n2)cc1]

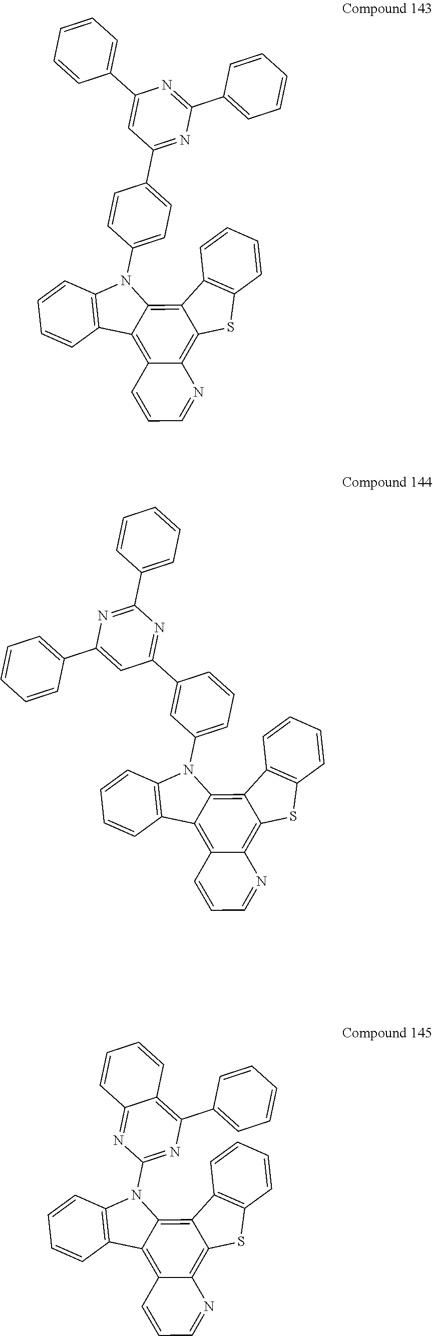 [SMILES: c1ccc(-c2cc(-c3ccc(-n4c5ccccc5c5c6cccnc6c6sc7ccccc7c6c54)cc3)nc(-c3ccccc3)n2)cc1 ; c1ccc(-c2cc(-c3cccc(-n4c5ccccc5c5c6cccnc6c6sc7ccccc7c6c54)c3)nc(-c3ccccc3)n2)cc1 ; c1ccc(-c2nc(-n3c4ccccc4c4c5cccnc5c5sc6ccccc6c5c43)nc3ccccc23)cc1]

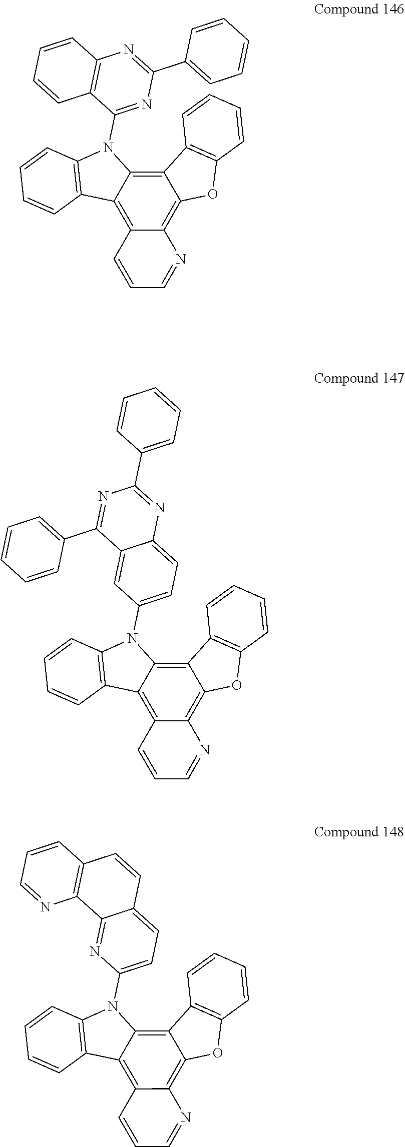 [SMILES: c1ccc(-c2nc(-n3c4ccccc4c4c5cccnc5c5oc6ccccc6c5c43)c3ccccc3n2)cc1 ; c1ccc(-c2nc(-c3ccccc3)c3cc(-n4c5ccccc5c5c6cccnc6c6oc7ccccc7c6c54)ccc3n2)cc1 ; c1cnc2c(c1)ccc1ccc(-n3c4ccccc4c4c5cccnc5c5oc6ccccc6c5c43)nc12]

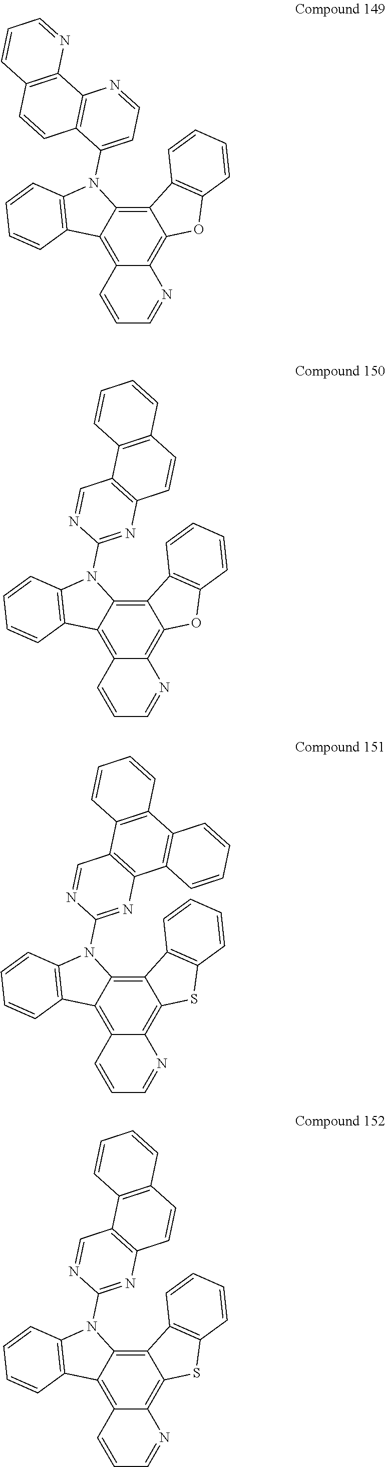

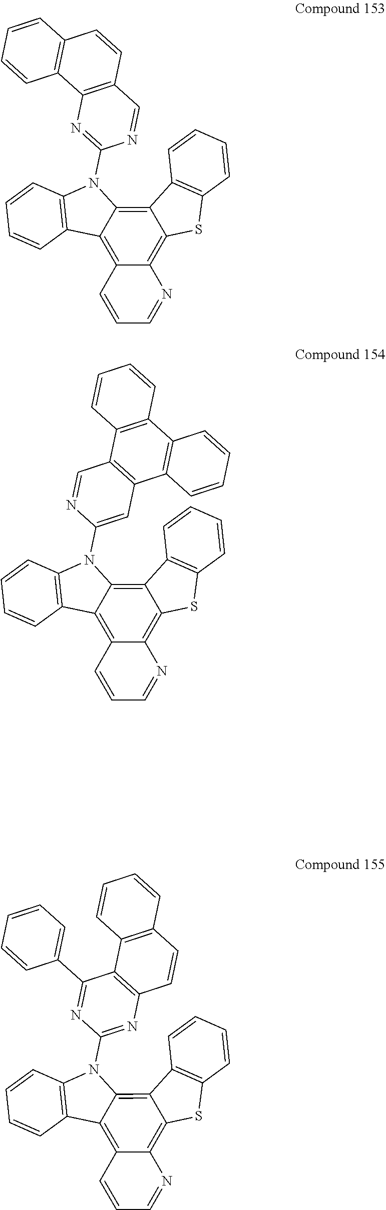

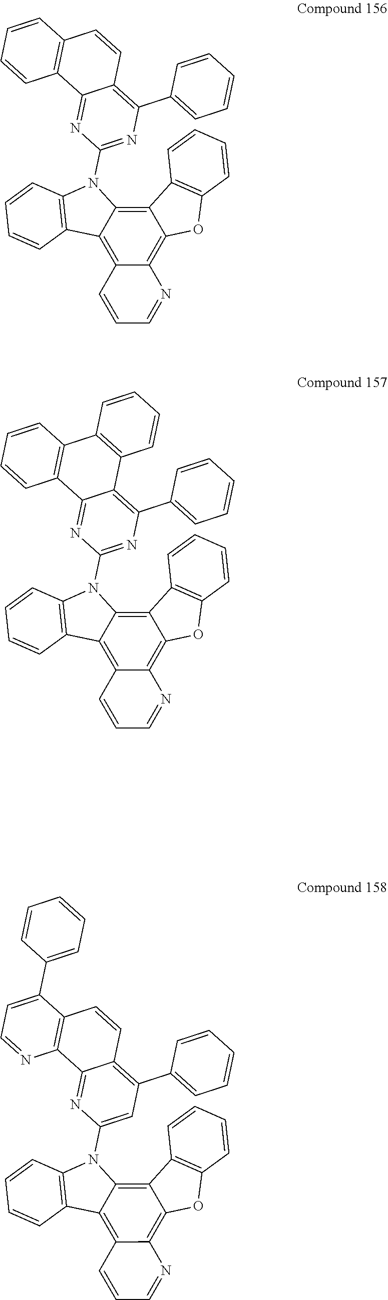 [SMILES: c1ccc(-c2nc(-n3c4ccccc4c4c5cccnc5c5oc6ccccc6c5c43)nc3c2ccc2ccccc23)cc1 ; c1ccc(-c2nc(-n3c4ccccc4c4c5cccnc5c5oc6ccccc6c5c43)nc3c4ccccc4c4ccccc4c23)cc1 ; c1ccc(-c2ccnc3c2ccc2c(-c4ccccc4)cc(-n4c5ccccc5c5c6cccnc6c6oc7ccccc7c6c54)nc23)cc1]

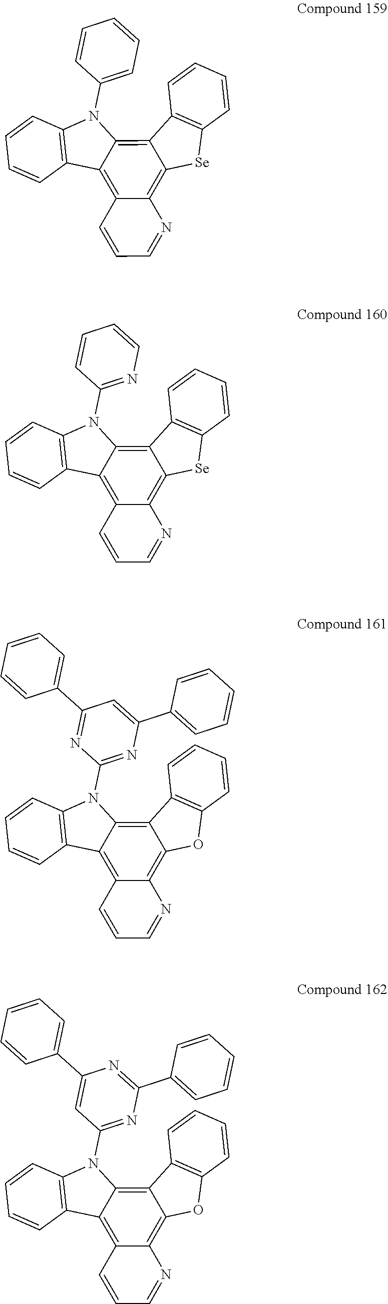 [SMILES: c1ccc(-n2c3ccccc3c3c4cccnc4c4[se]c5ccccc5c4c32)cc1 ; c1ccc(-n2c3ccccc3c3c4cccnc4c4[se]c5ccccc5c4c32)nc1 ; c1ccc(-c2cc(-c3ccccc3)nc(-n3c4ccccc4c4c5cccnc5c5oc6ccccc6c5c43)n2)cc1 ; c1ccc(-c2cc(-n3c4ccccc4c4c5cccnc5c5oc6ccccc6c5c43)nc(-c3ccccc3)n2)cc1]

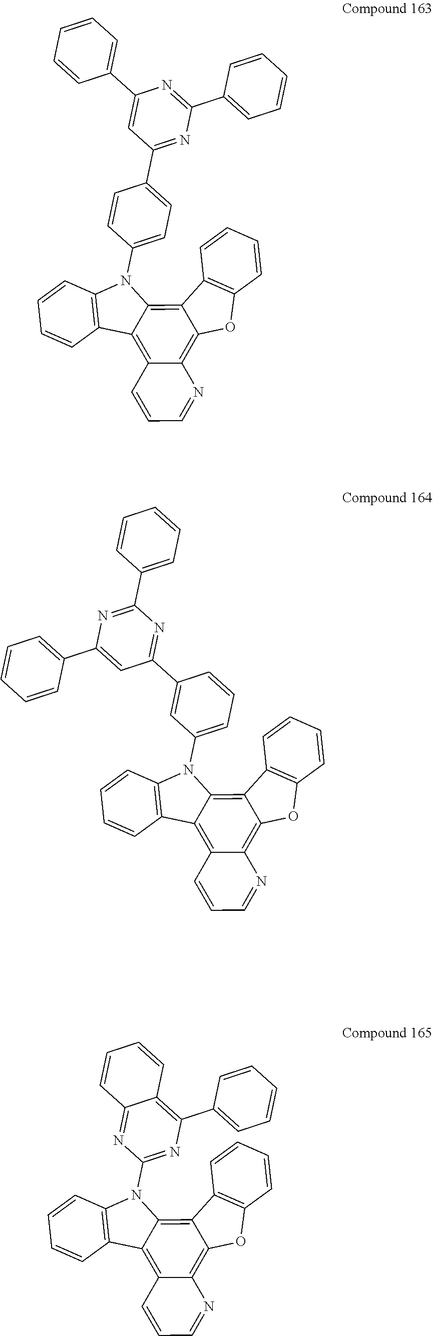 [SMILES: c1ccc(-c2cc(-c3ccc(-n4c5ccccc5c5c6cccnc6c6oc7ccccc7c6c54)cc3)nc(-c3ccccc3)n2)cc1 ; c1ccc(-c2cc(-c3cccc(-n4c5ccccc5c5c6cccnc6c6oc7ccccc7c6c54)c3)nc(-c3ccccc3)n2)cc1 ; c1ccc(-c2nc(-n3c4ccccc4c4c5cccnc5c5oc6ccccc6c5c43)nc3ccccc23)cc1]

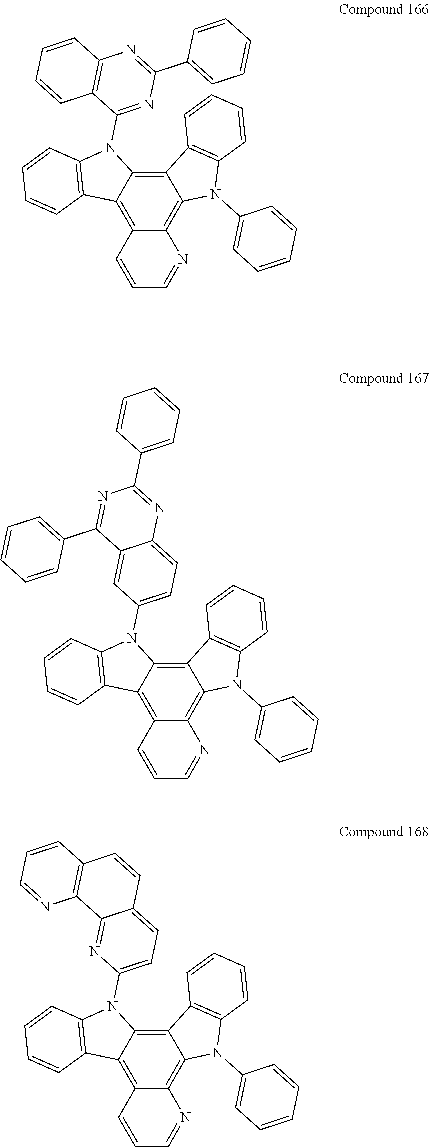 [SMILES: c1ccc(-c2nc(-n3c4ccccc4c4c5cccnc5c5c(c6ccccc6n5-c5ccccc5)c43)c3ccccc3n2)cc1 ; c1ccc(-c2nc(-c3ccccc3)c3cc(-n4c5ccccc5c5c6cccnc6c6c(c7ccccc7n6-c6ccccc6)c54)ccc3n2)cc1 ; c1ccc(-n2c3ccccc3c3c2c2ncccc2c2c4ccccc4n(-c4ccc5ccc6cccnc6c5n4)c23)cc1]

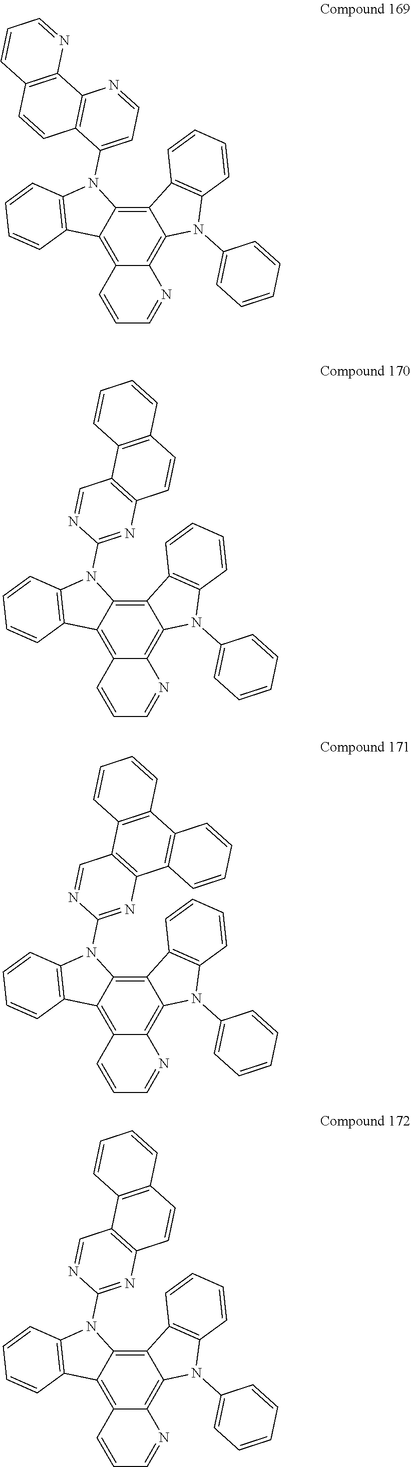

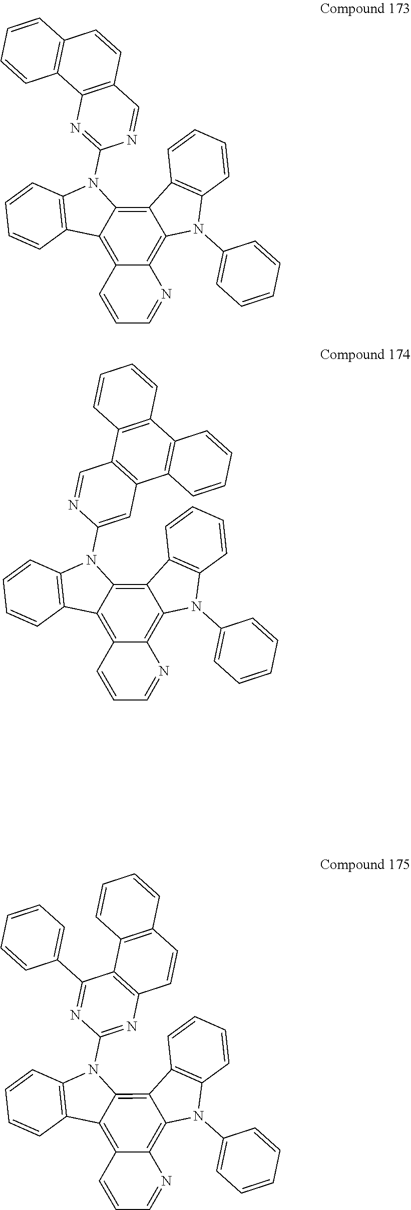

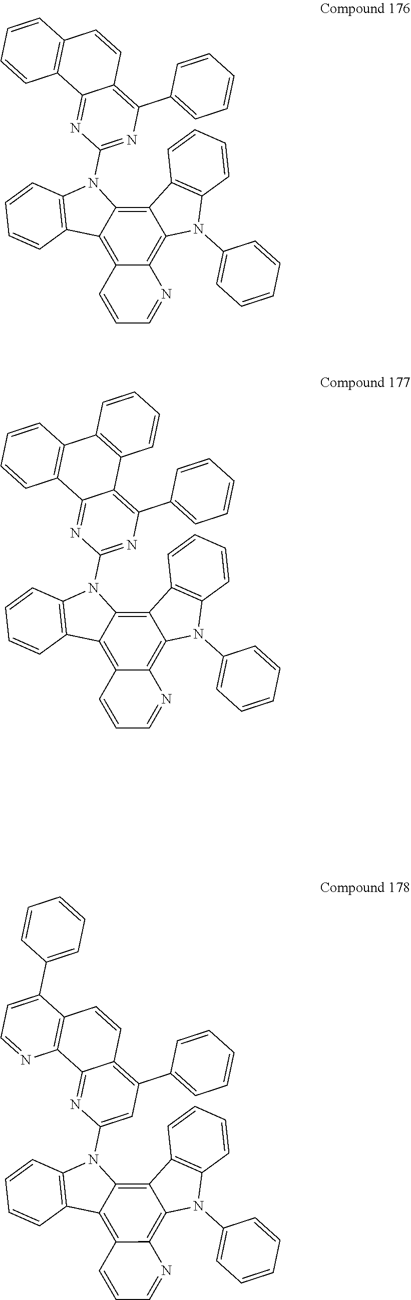 [SMILES: c1ccc(-c2nc(-n3c4ccccc4c4c5cccnc5c5c(c6ccccc6n5-c5ccccc5)c43)nc3c2ccc2ccccc23)cc1 ; c1ccc(-c2nc(-n3c4ccccc4c4c5cccnc5c5c(c6ccccc6n5-c5ccccc5)c43)nc3c4ccccc4c4ccccc4c23)cc1 ; c1ccc(-c2ccnc3c2ccc2c(-c4ccccc4)cc(-n4c5ccccc5c5c6cccnc6c6c(c7ccccc7n6-c6ccccc6)c54)nc23)cc1]

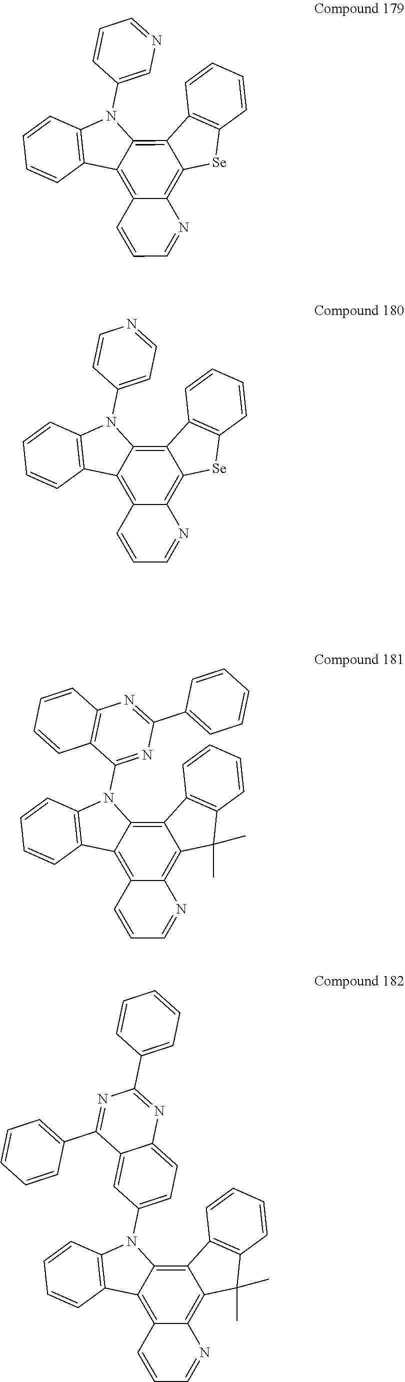 [SMILES: c1cncc(-n2c3ccccc3c3c4cccnc4c4[se]c5ccccc5c4c32)c1 ; c1ccc2c(c1)[se]c1c3ncccc3c3c4ccccc4n(-c4ccncc4)c3c21 ; CC1(C)c2ccccc2-c2c1c1ncccc1c1c3ccccc3n(-c3nc(-c4ccccc4)nc4ccccc34)c21 ; CC1(C)c2ccccc2-c2c1c1ncccc1c1c3ccccc3n(-c3ccc4nc(-c5ccccc5)nc(-c5ccccc5)c4c3)c21]

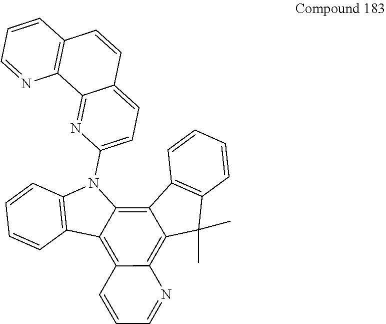 [SMILES: CC1(C)c2ccccc2-c2c1c1ncccc1c1c3ccccc3n(-c3ccc4ccc5cccnc5c4n3)c21]

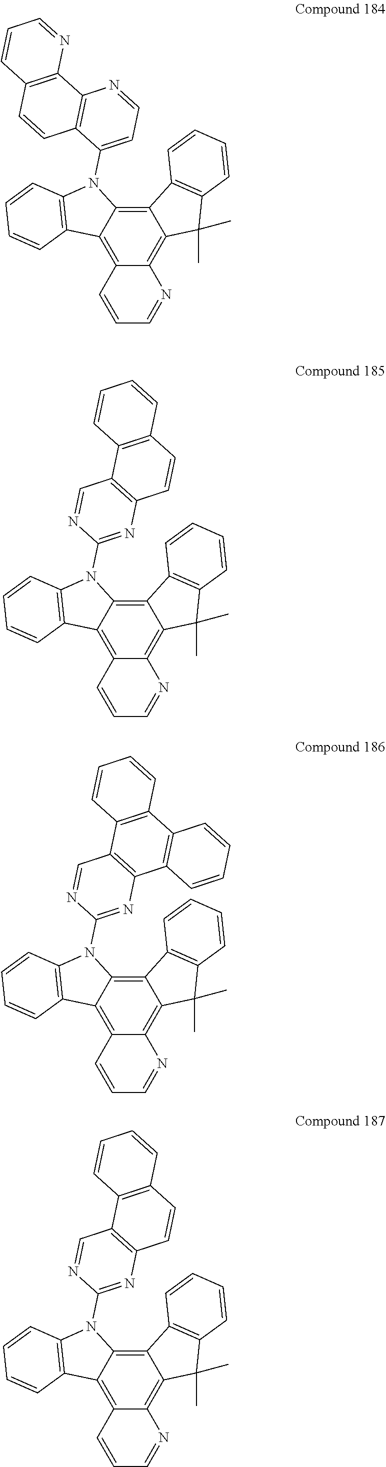

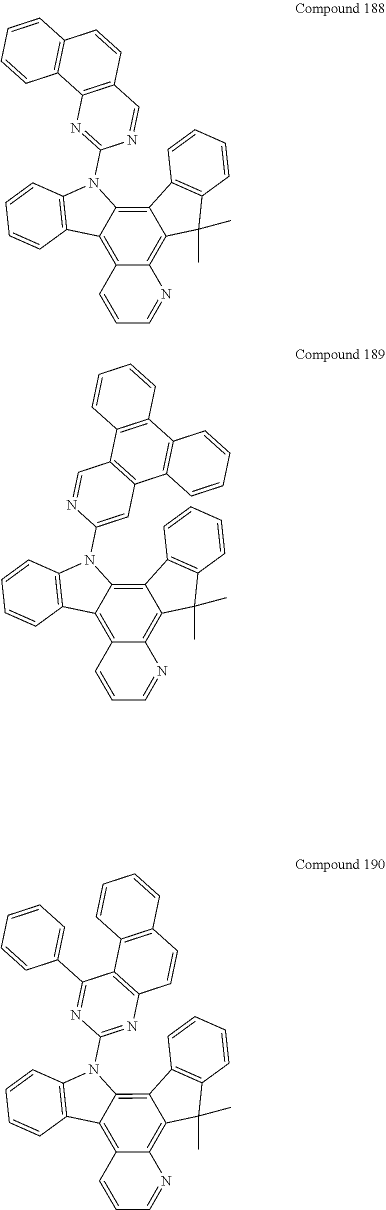

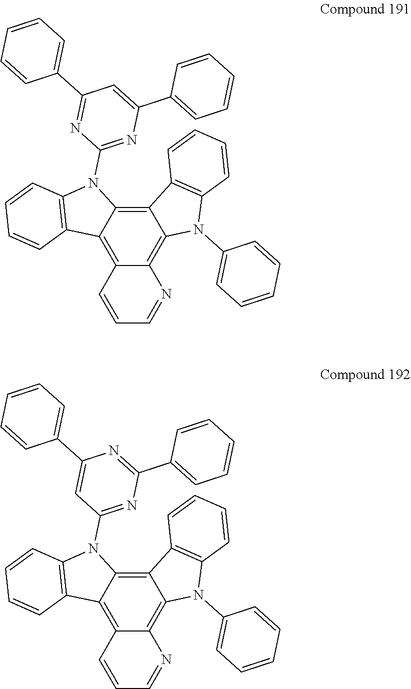 [SMILES: c1ccc(-c2cc(-c3ccccc3)nc(-n3c4ccccc4c4c5cccnc5c5c(c6ccccc6n5-c5ccccc5)c43)n2)cc1 ; c1ccc(-c2cc(-n3c4ccccc4c4c5cccnc5c5c(c6ccccc6n5-c5ccccc5)c43)nc(-c3ccccc3)n2)cc1]

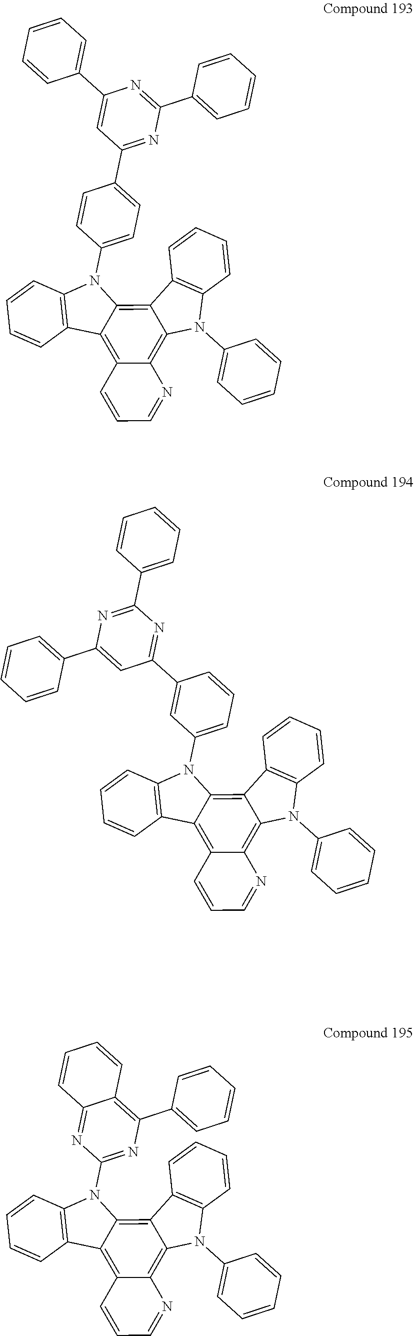 [SMILES: c1ccc(-c2cc(-c3ccc(-n4c5ccccc5c5c6cccnc6c6c(c7ccccc7n6-c6ccccc6)c54)cc3)nc(-c3ccccc3)n2)cc1 ; c1ccc(-c2cc(-c3cccc(-n4c5ccccc5c5c6cccnc6c6c(c7ccccc7n6-c6ccccc6)c54)c3)nc(-c3ccccc3)n2)cc1 ; c1ccc(-c2nc(-n3c4ccccc4c4c5cccnc5c5c(c6ccccc6n5-c5ccccc5)c43)nc3ccccc23)cc1]

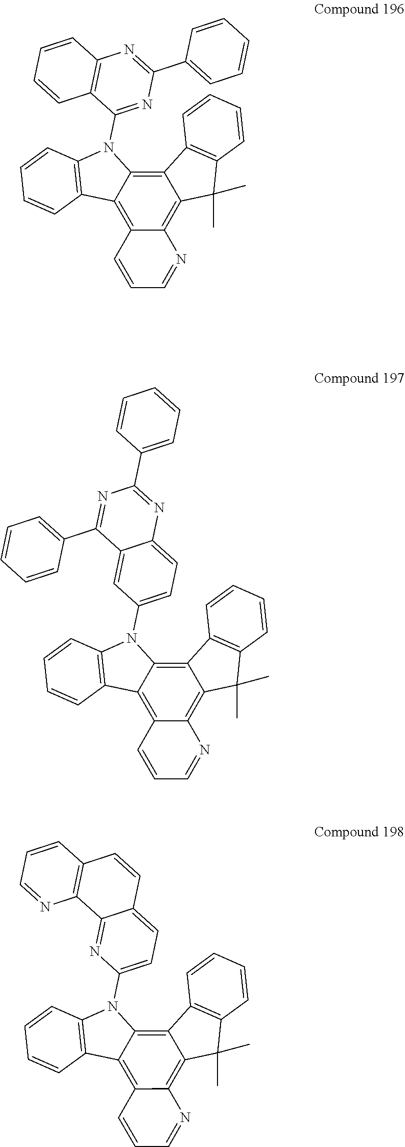 [SMILES: CC1(C)c2ccccc2-c2c1c1ncccc1c1c3ccccc3n(-c3nc(-c4ccccc4)nc4ccccc34)c21 ; CC1(C)c2ccccc2-c2c1c1ncccc1c1c3ccccc3n(-c3ccc4nc(-c5ccccc5)nc(-c5ccccc5)c4c3)c21 ; CC1(C)c2ccccc2-c2c1c1ncccc1c1c3ccccc3n(-c3ccc4ccc5cccnc5c4n3)c21]

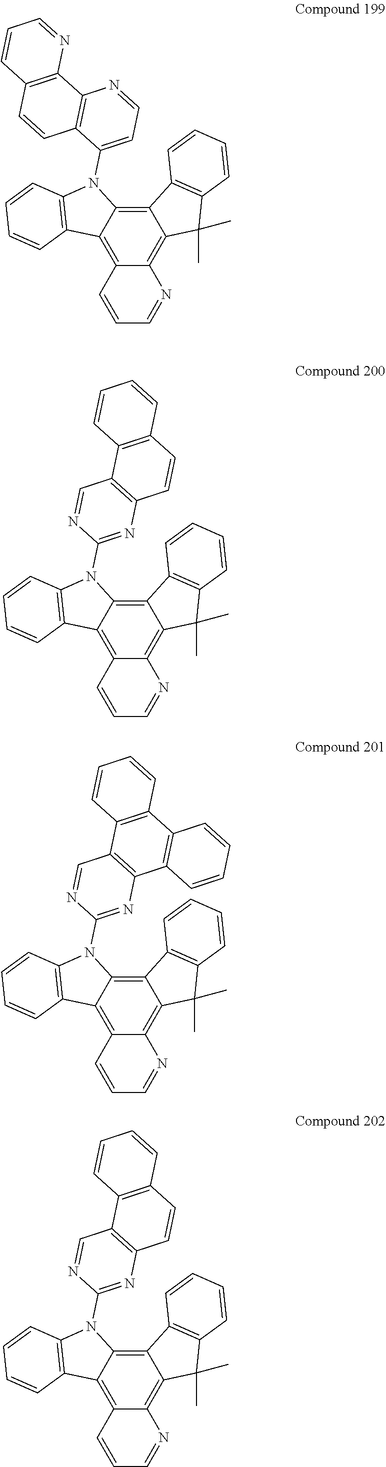

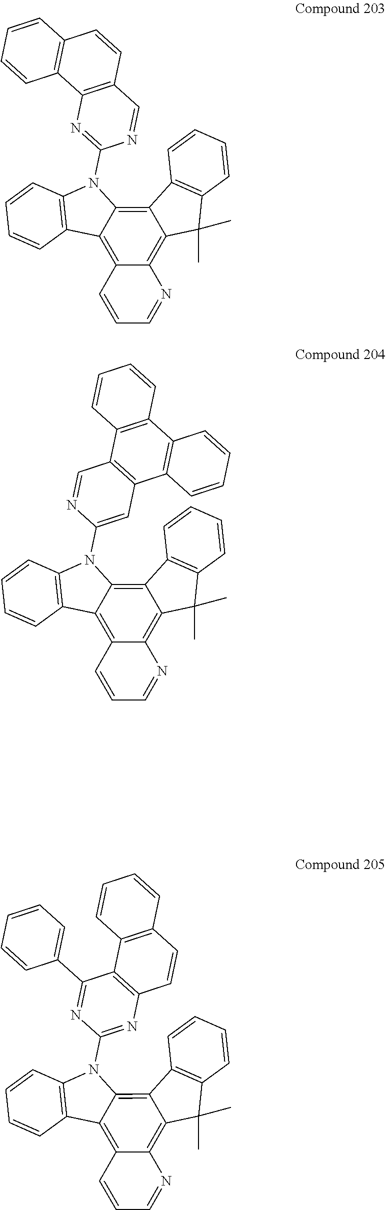

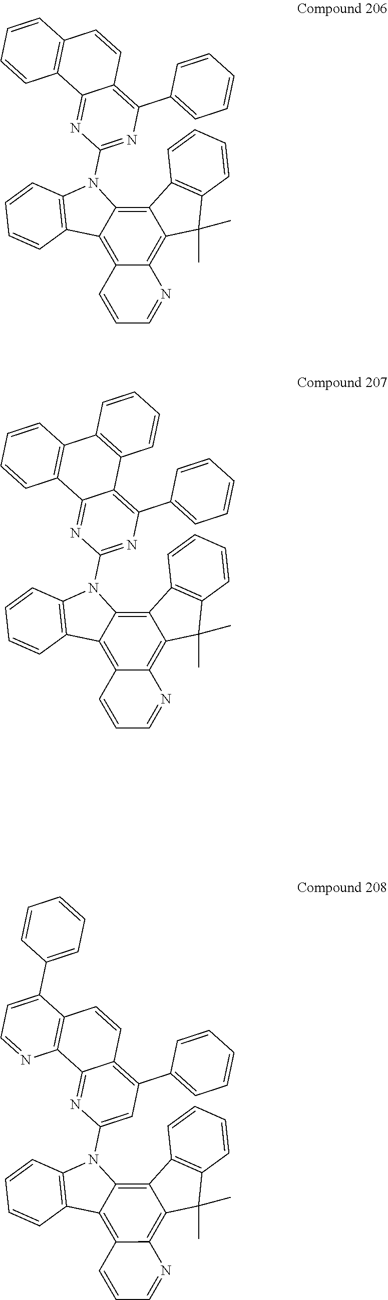 [SMILES: CC1(C)c2ccccc2-c2c1c1ncccc1c1c3ccccc3n(-c3nc(-c4ccccc4)c4ccc5ccccc5c4n3)c21 ; CC1(C)c2ccccc2-c2c1c1ncccc1c1c3ccccc3n(-c3nc(-c4ccccc4)c4c5ccccc5c5ccccc5c4n3)c21 ; CC1(C)c2ccccc2-c2c1c1ncccc1c1c3ccccc3n(-c3cc(-c4ccccc4)c4ccc5c(-c6ccccc6)ccnc5c4n3)c21]

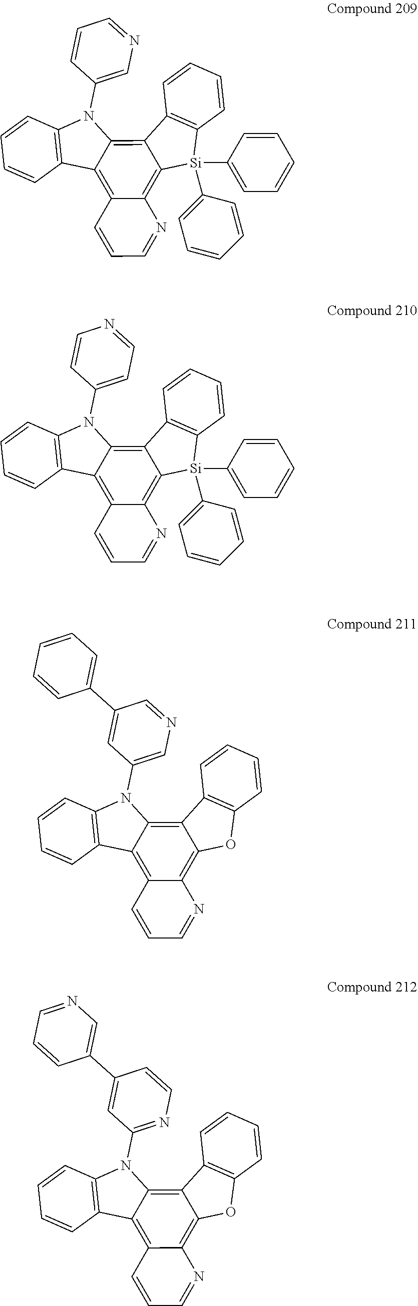 [SMILES: c1ccc([Si]2(c3ccccc3)c3ccccc3-c3c2c2ncccc2c2c4ccccc4n(-c4cccnc4)c32)cc1 ; c1ccc([Si]2(c3ccccc3)c3ccccc3-c3c2c2ncccc2c2c4ccccc4n(-c4ccncc4)c32)cc1 ; c1ccc(-c2cncc(-n3c4ccccc4c4c5cccnc5c5oc6ccccc6c5c43)c2)cc1 ; c1cncc(-c2ccnc(-n3c4ccccc4c4c5cccnc5c5oc6ccccc6c5c43)c2)c1]

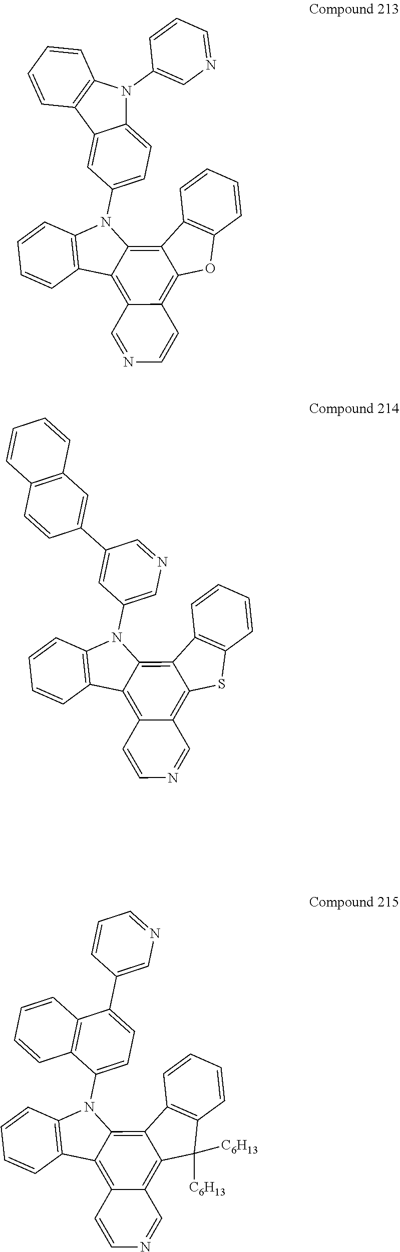

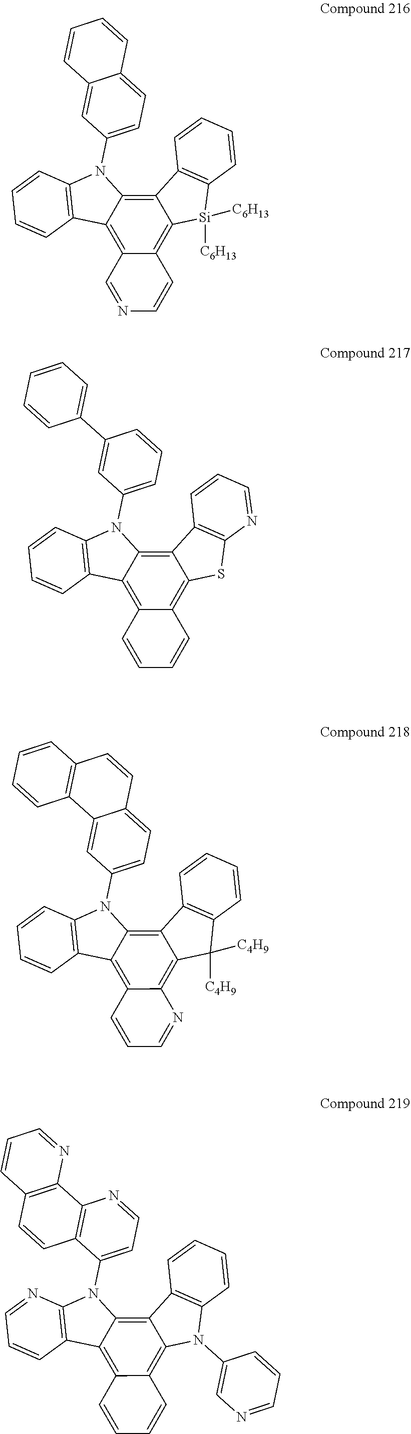

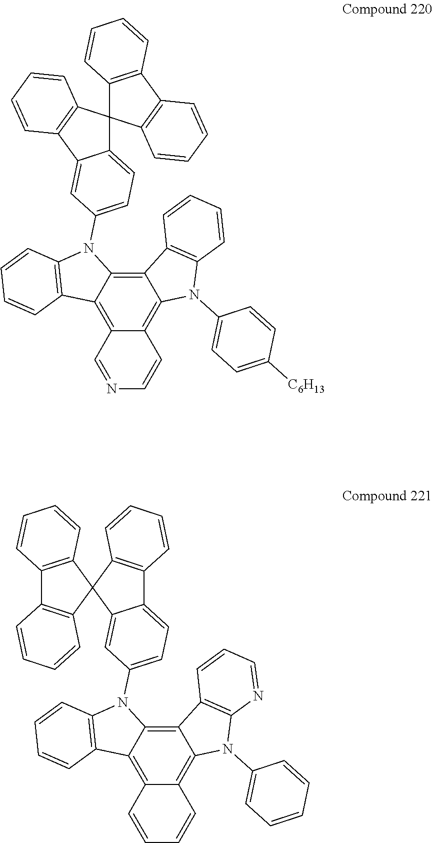

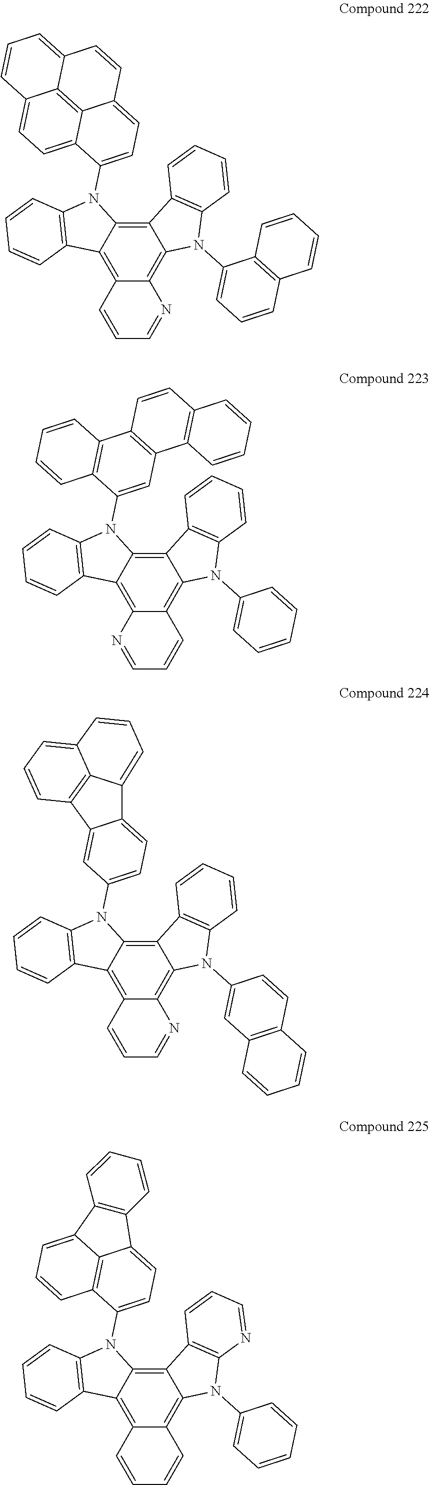 [SMILES: c1ccc2c(-n3c4ccccc4c4c3c3ncccc3c3c5ccccc5n(-c5ccc6ccc7cccc8ccc5c6c78)c34)cccc2c1 ; c1ccc(-n2c3ccccc3c3c2c2cccnc2c2c4ccccc4n(-c4cc5c6ccccc6ccc5c5ccccc45)c23)cc1 ; c1ccc2cc(-n3c4ccccc4c4c3c3ncccc3c3c5ccccc5n(-c5ccc6c(c5)-c5cccc7cccc-6c57)c34)ccc2c1 ; c1ccc(-n2c3ncccc3c3c4c(c5ccccc5c32)c2ccccc2n4-c2ccc3c4c(cccc24)-c2ccccc2-3)cc1]

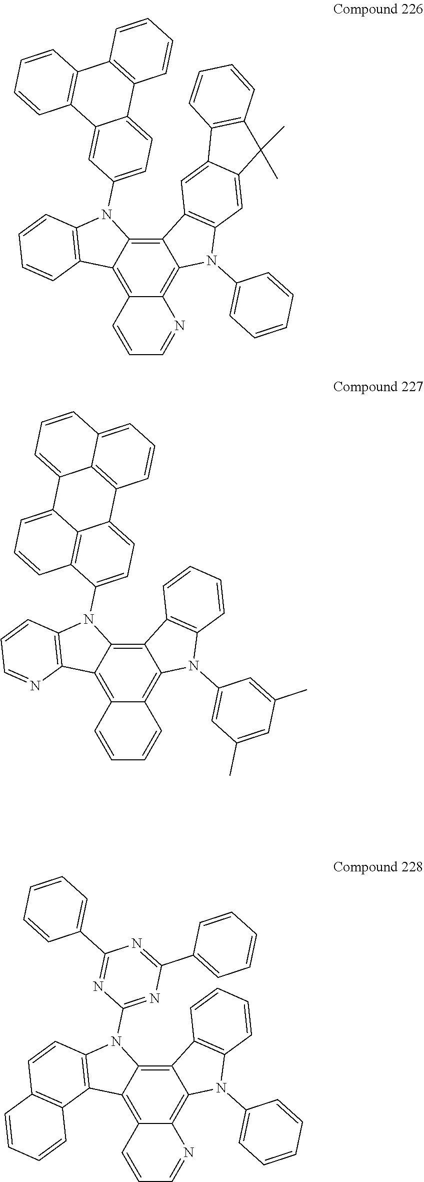 [SMILES: CC1(C)c2ccccc2-c2cc3c4c(c5ncccc5c5c6ccccc6n(-c6ccc7c8ccccc8c8ccccc8c7c6)c54)n(-c4ccccc4)c3cc21 ; Cc1cc(C)cc(-n2c3ccccc3c3c2c2ccccc2c2c4ncccc4n(-c4ccc5c6cccc7cccc(c8cccc4c85)c76)c23)c1 ; c1ccc(-c2nc(-c3ccccc3)nc(-n3c4ccc5ccccc5c4c4c5cccnc5c5c(c6ccccc6n5-c5ccccc5)c43)n2)cc1]

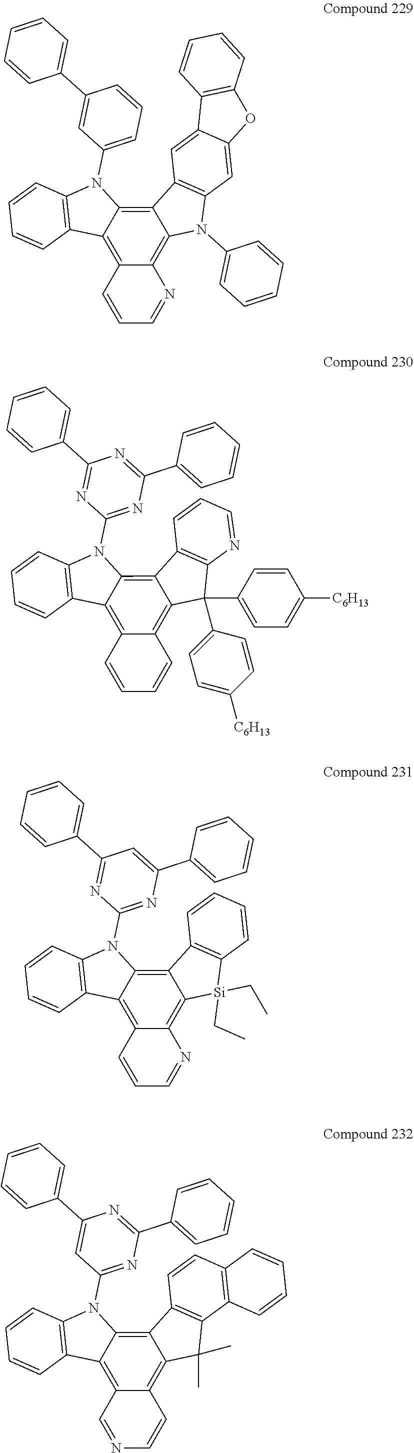

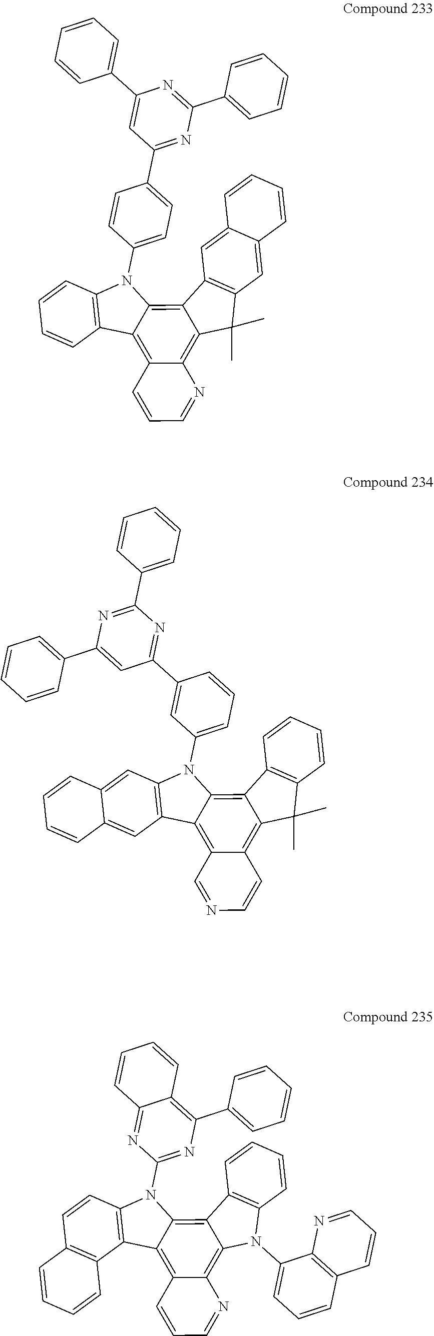 [SMILES: CC1(C)c2cc3ccccc3cc2-c2c1c1ncccc1c1c3ccccc3n(-c3ccc(-c4cc(-c5ccccc5)nc(-c5ccccc5)n4)cc3)c21 ; CC1(C)c2ccccc2-c2c1c1ccncc1c1c3cc4ccccc4cc3n(-c3cccc(-c4cc(-c5ccccc5)nc(-c5ccccc5)n4)c3)c21 ; c1ccc(-c2nc(-n3c4ccc5ccccc5c4c4c5cccnc5c5c(c6ccccc6n5-c5cccc6cccnc56)c43)nc3ccccc23)cc1]

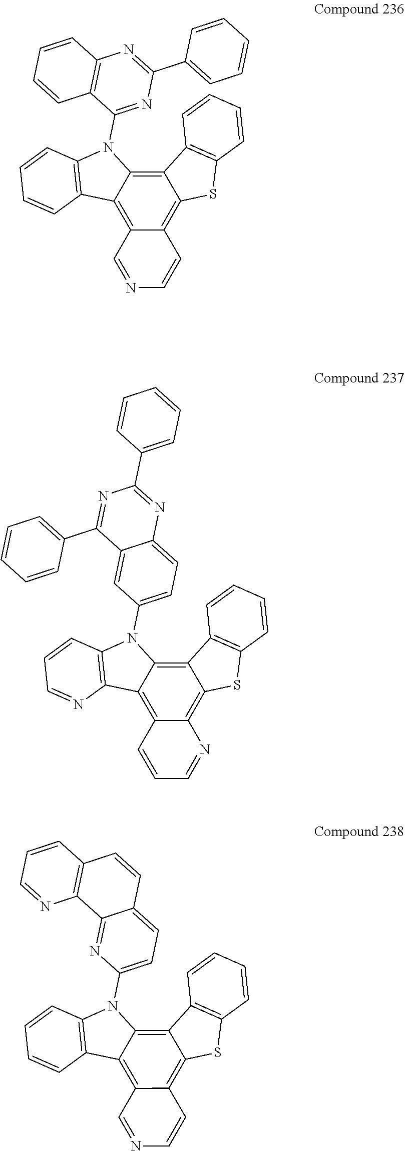 [SMILES: c1ccc(-c2nc(-n3c4ccccc4c4c5cnccc5c5sc6ccccc6c5c43)c3ccccc3n2)cc1 ; c1ccc(-c2nc(-c3ccccc3)c3cc(-n4c5cccnc5c5c6cccnc6c6sc7ccccc7c6c54)ccc3n2)cc1 ; c1cnc2c(c1)ccc1ccc(-n3c4ccccc4c4c5cnccc5c5sc6ccccc6c5c43)nc12]

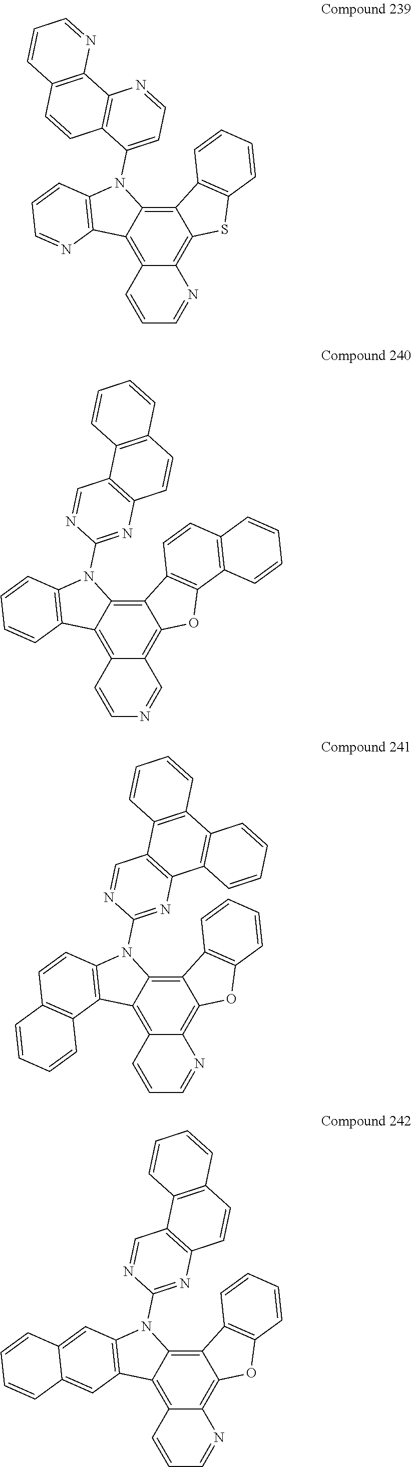

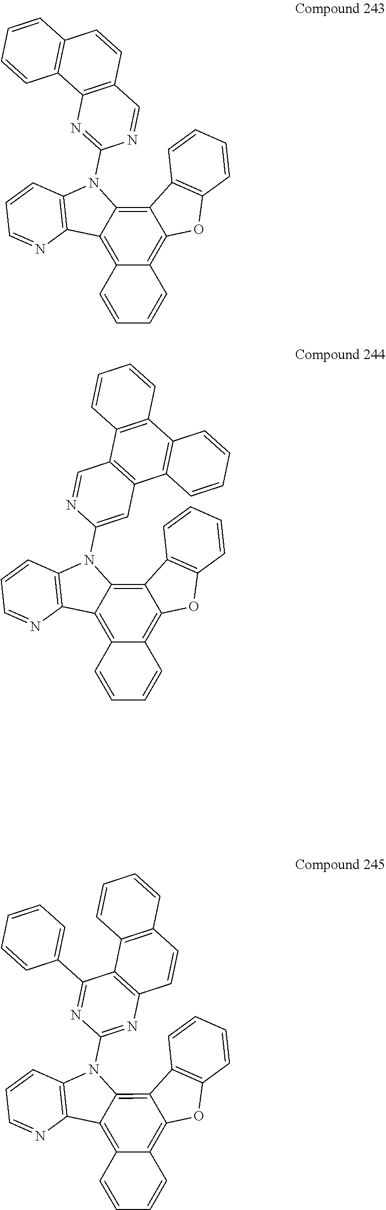

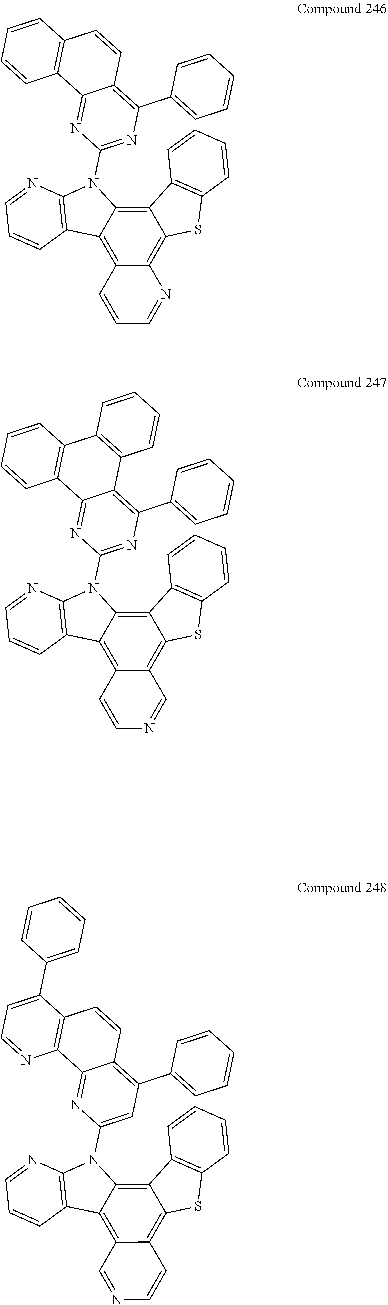 [SMILES: c1ccc(-c2nc(-n3c4ncccc4c4c5cccnc5c5sc6ccccc6c5c43)nc3c2ccc2ccccc23)cc1 ; c1ccc(-c2nc(-n3c4ncccc4c4c5ccncc5c5sc6ccccc6c5c43)nc3c4ccccc4c4ccccc4c23)cc1 ; c1ccc(-c2ccnc3c2ccc2c(-c4ccccc4)cc(-n4c5ncccc5c5c6cnccc6c6sc7ccccc7c6c54)nc23)cc1]

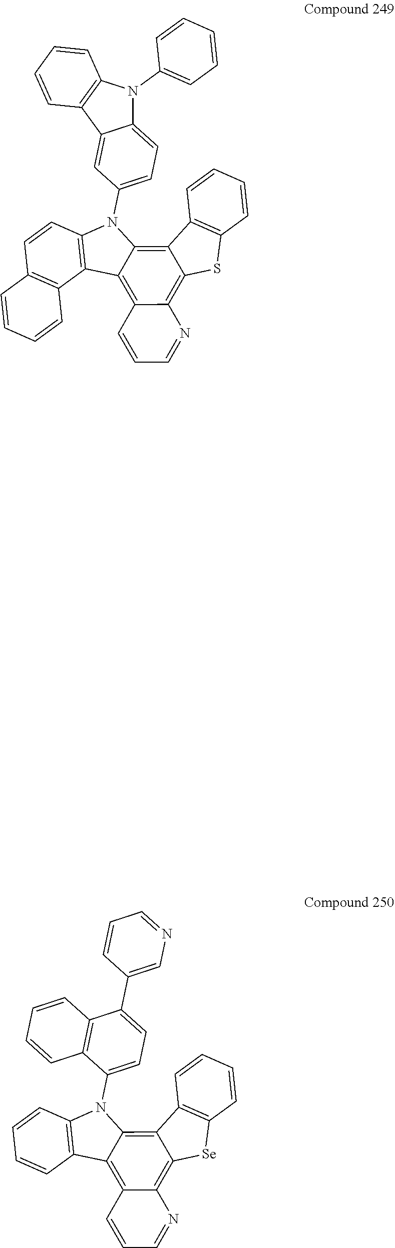 [SMILES: c1ccc(-n2c3ccccc3c3cc(-n4c5ccc6ccccc6c5c5c6cccnc6c6sc7ccccc7c6c54)ccc32)cc1 ; c1cncc(-c2ccc(-n3c4ccccc4c4c5cccnc5c5[se]c6ccccc6c5c43)c3ccccc23)c1]

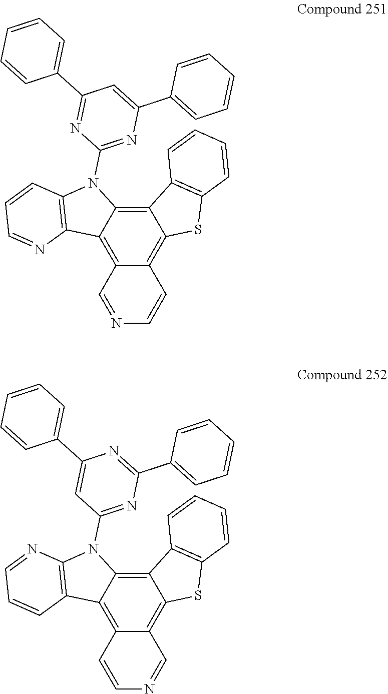 [SMILES: c1ccc(-c2cc(-c3ccccc3)nc(-n3c4cccnc4c4c5cnccc5c5sc6ccccc6c5c43)n2)cc1 ; c1ccc(-c2cc(-n3c4ncccc4c4c5ccncc5c5sc6ccccc6c5c43)nc(-c3ccccc3)n2)cc1]

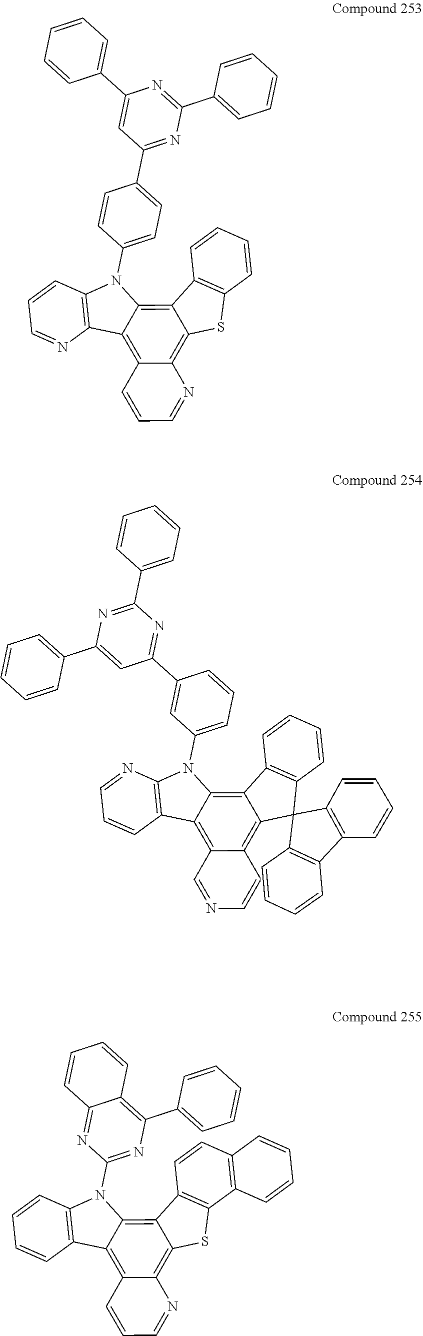 [SMILES: c1ccc(-c2cc(-c3ccc(-n4c5cccnc5c5c6cccnc6c6sc7ccccc7c6c54)cc3)nc(-c3ccccc3)n2)cc1 ; c1ccc(-c2cc(-c3cccc(-n4c5ncccc5c5c6cnccc6c6c(c54)-c4ccccc4C64c5ccccc5-c5ccccc54)c3)nc(-c3ccccc3)n2)cc1 ; c1ccc(-c2nc(-n3c4ccccc4c4c5cccnc5c5sc6c7ccccc7ccc6c5c43)nc3ccccc23)cc1]

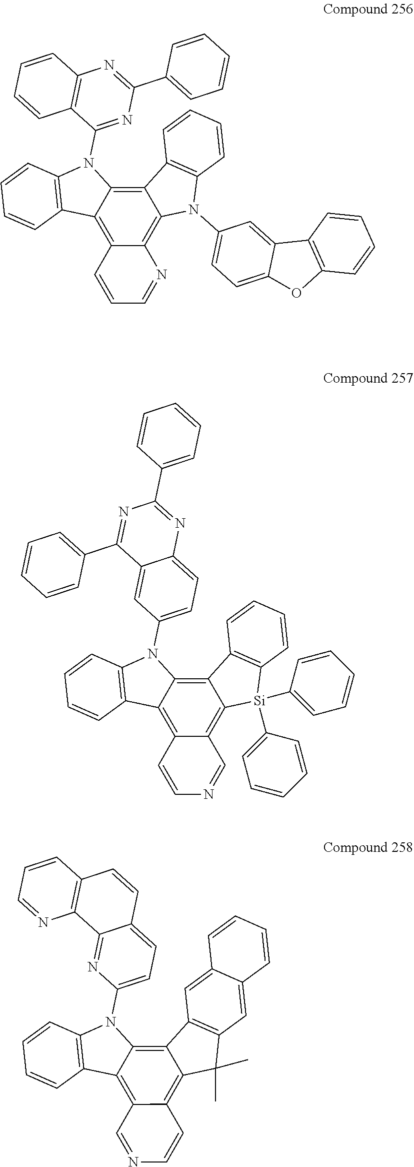 [SMILES: c1ccc(-c2nc(-n3c4ccccc4c4c5cccnc5c5c(c6ccccc6n5-c5ccc6oc7ccccc7c6c5)c43)c3ccccc3n2)cc1 ; c1ccc(-c2nc(-c3ccccc3)c3cc(-n4c5ccccc5c5c6ccncc6c6c(c54)-c4ccccc4[Si]6(c4ccccc4)c4ccccc4)ccc3n2)cc1 ; CC1(C)c2cc3ccccc3cc2-c2c1c1ccncc1c1c3ccccc3n(-c3ccc4ccc5cccnc5c4n3)c21]

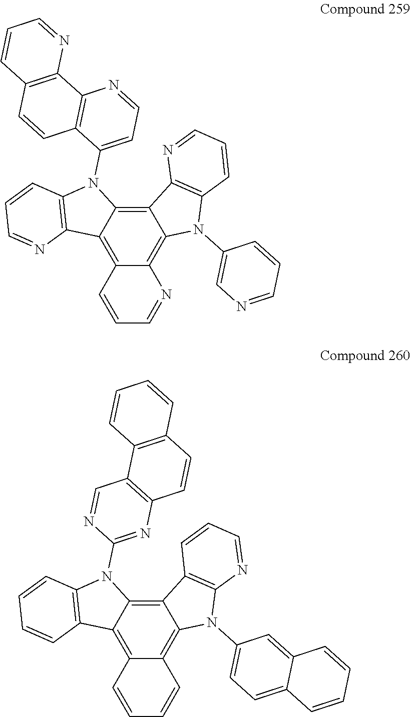 [SMILES: c1cncc(-n2c3cccnc3c3c2c2ncccc2c2c4ncccc4n(-c4ccnc5c4ccc4cccnc45)c23)c1 ; c1ccc2cc(-n3c4ncccc4c4c5c(c6ccccc6c43)c3ccccc3n5-c3ncc4c(ccc5ccccc54)n3)ccc2c1]

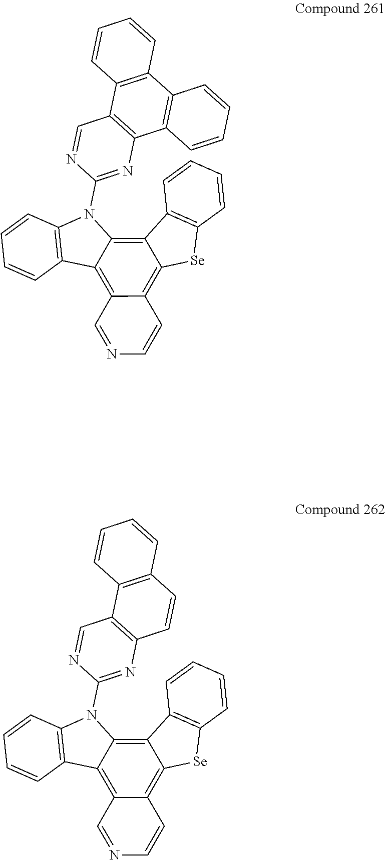 [SMILES: c1ccc2c(c1)[se]c1c3ccncc3c3c4ccccc4n(-c4ncc5c6ccccc6c6ccccc6c5n4)c3c21 ; c1ccc2c(c1)ccc1nc(-n3c4ccccc4c4c5cnccc5c5[se]c6ccccc6c5c43)ncc12]

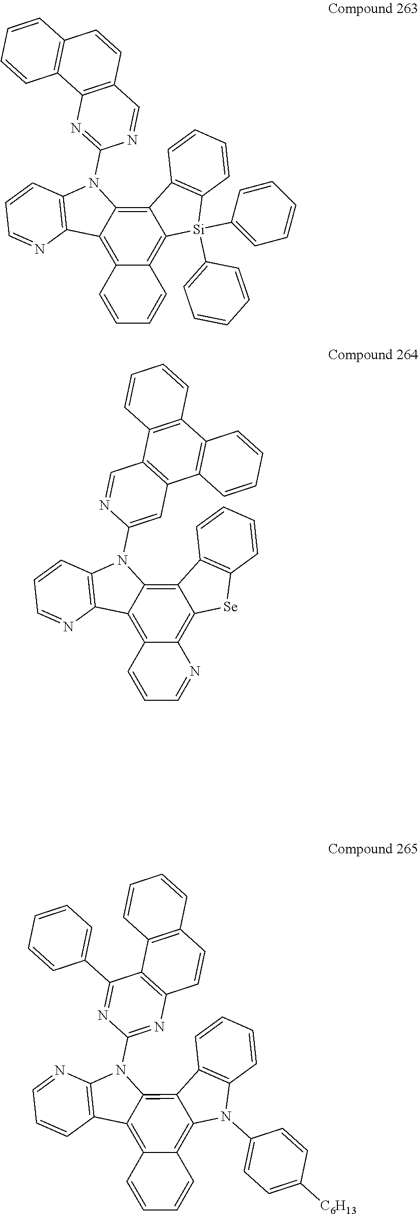

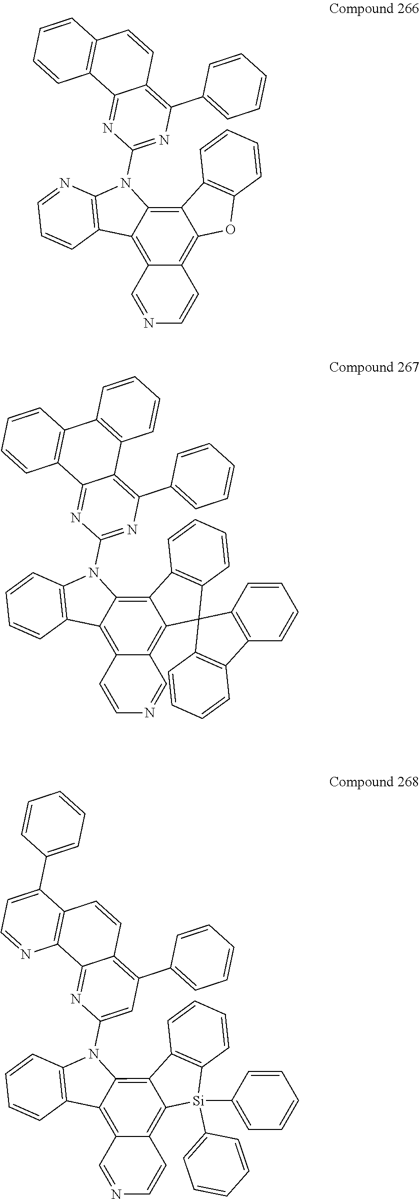 [SMILES: c1ccc(-c2nc(-n3c4ncccc4c4c5cnccc5c5oc6ccccc6c5c43)nc3c2ccc2ccccc23)cc1 ; c1ccc(-c2nc(-n3c4ccccc4c4c5ccncc5c5c(c43)-c3ccccc3C53c4ccccc4-c4ccccc43)nc3c4ccccc4c4ccccc4c23)cc1 ; c1ccc(-c2ccnc3c2ccc2c(-c4ccccc4)cc(-n4c5ccccc5c5c6cnccc6c6c(c54)-c4ccccc4[Si]6(c4ccccc4)c4ccccc4)nc23)cc1]

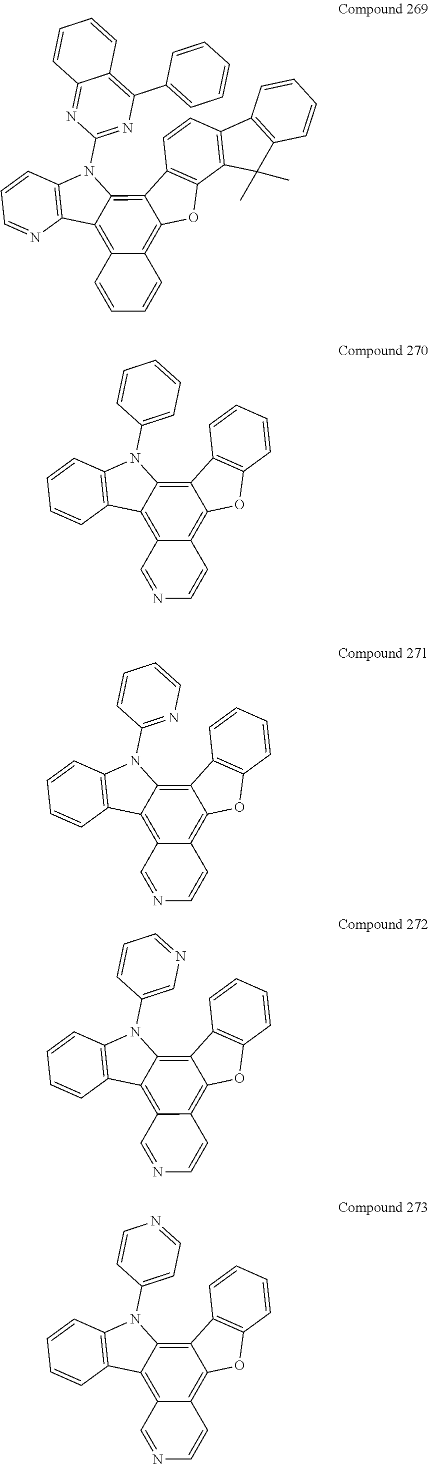

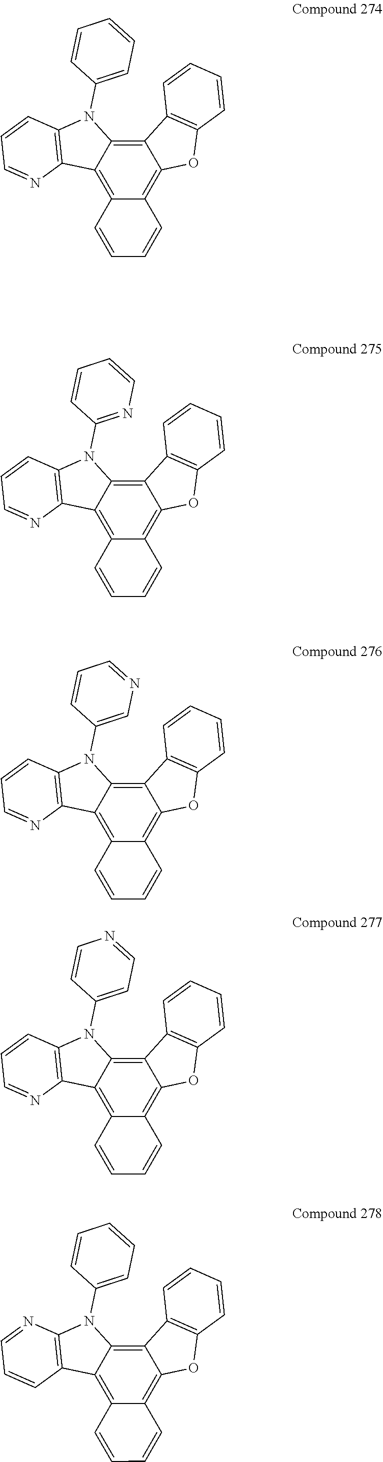

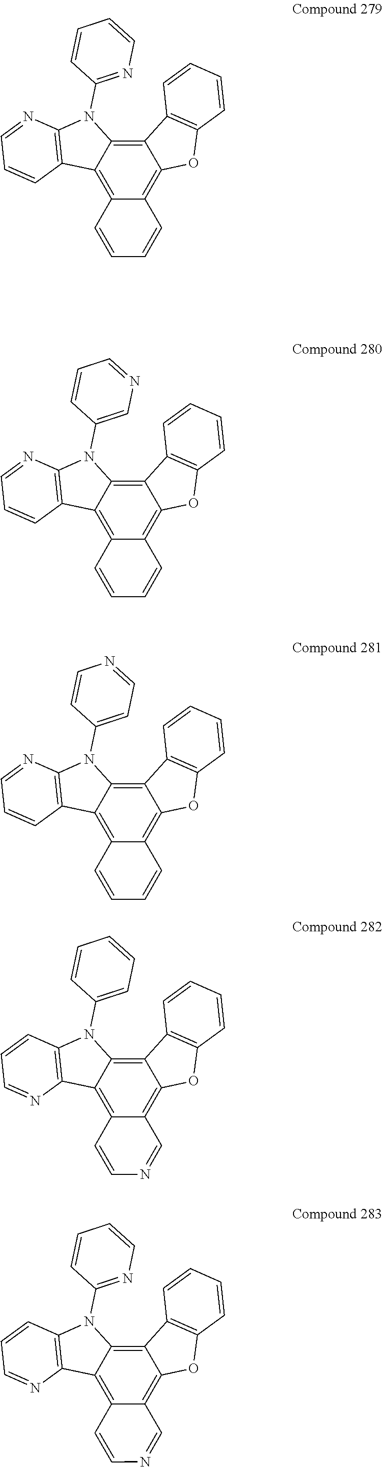

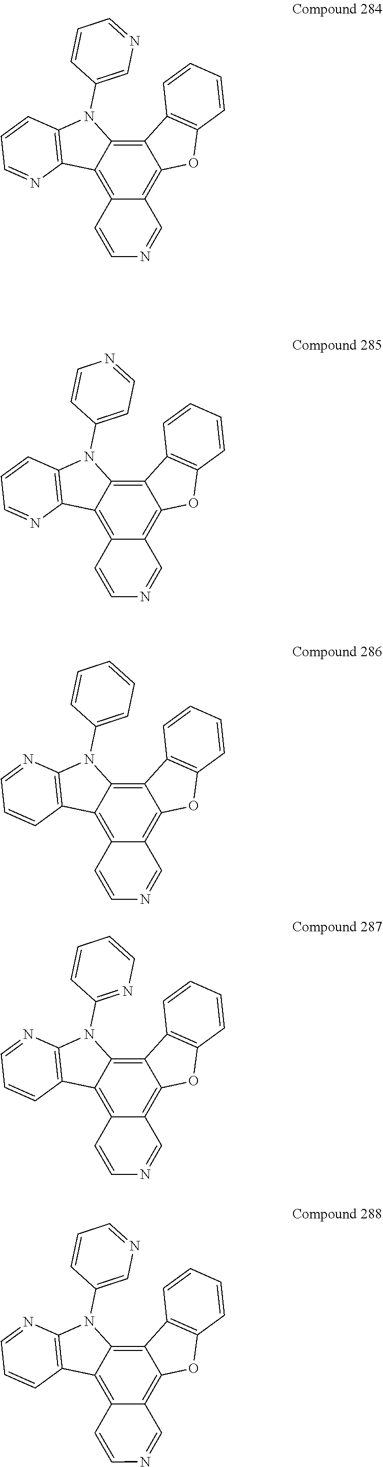

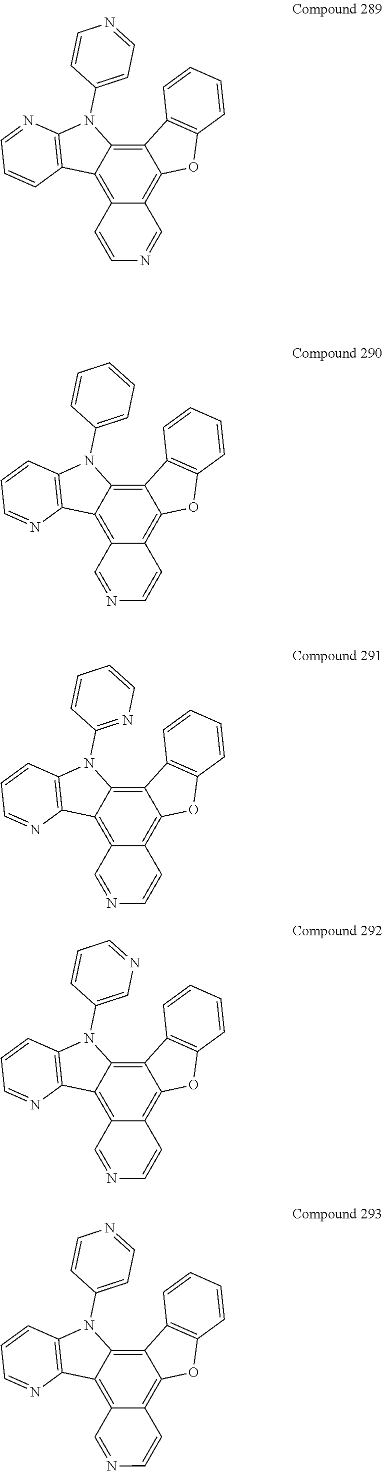

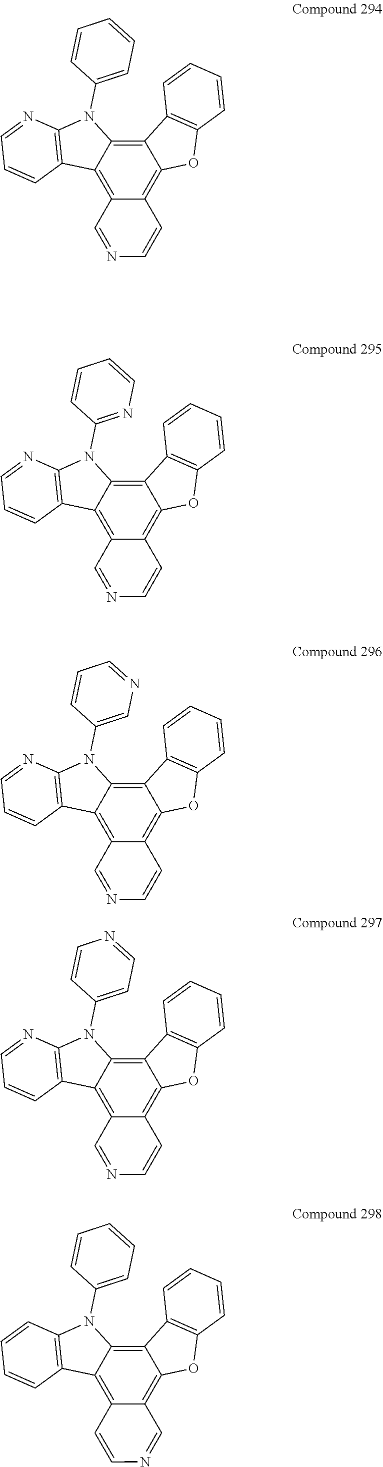

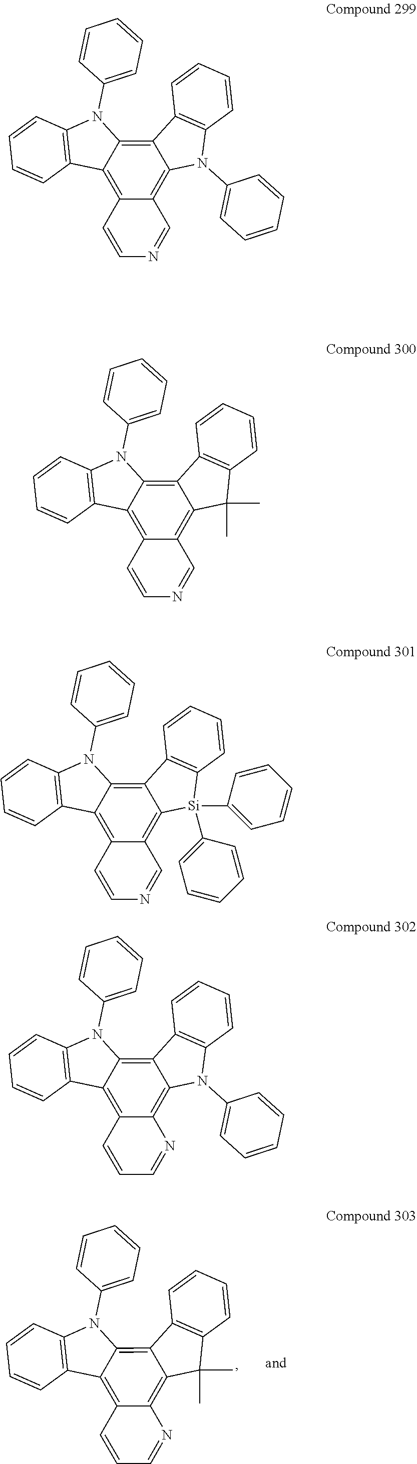

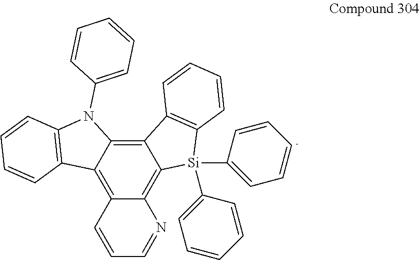 [SMILES: c1ccc(-n2c3ccccc3c3c4cccnc4c4c(c32)-c2ccccc2[Si]4(c2ccccc2)c2ccccc2)cc1]

6. An organic electroluminescence device comprising an anode, a cathode and one or more organic layers formed between the anode and the cathode, wherein at least one of the organic layers comprises the organic compound according to claim 1.

7. The organic electroluminescence device according to claim 5, wherein the organic layers comprise an emissive layer having a host, and wherein the organic compound is comprised as the host.

8. The organic electroluminescence device according to claim 5, wherein the organic layers comprise an electron transfer layer, and wherein the organic compound of claim 1 is comprised as the electron transfer layer.

9. The organic electroluminescence device according to claim 5, wherein the organic compound is a hole blocking material.

10. The organic electroluminescence device according to claim 5, wherein the organic electroluminescence device is a lighting panel.

11. The organic electroluminescence device according to claim 5, wherein the organic electroluminescence device is a backlight panel.

12. The organic compound according to claim 1, wherein the organic compound is represented by the following formula (2): 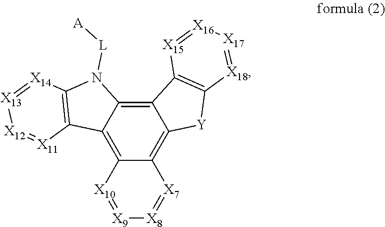
wherein X₇ is N or CR₇;
wherein X₈ is N or CR₈;
wherein X₉ is N or CR₉;
wherein X₁₀ is N or CR₁₀;
wherein X₁₁ is N or CR₁₁;
wherein X₁₂ is N or CR₁₂;
wherein X₁₃ is N or CR₁₃;
wherein X₁₄ is N or CR₁₄;
wherein X₁₅ is N or CR₁₅;
wherein X₁₆ is N or CR₁₆;
wherein X₁₇ is N or CR₁₇;
wherein X₁₈ is N or CR₁₈;
wherein at least one of X₇ to X₁₈ is N;
wherein Y is selected from the group consisting of O, S, Se, NR₁, CR₂R₃ and SiR₄R₅;
wherein R₁ to R₅ are independently selected from the group consisting of methyl, ethyl, phenyl, naphthyl, hexylbenzenyl, pyrimidinyl, quinolinyl, and combinations thereof;
wherein R₇ to R₁₈ are independently selected from the group consisting of H, an aryl group having 6 carbon atoms, an alkyl group having 1, 2 or 3 carbon atoms, and a heteroaryl group having 3, 4 or 5 carbon atoms; and
wherein adjacent two of X₇ to X₁₈ can form a five-membered ring, a six-membered ring or a combination thereof.

13. The organic compound according to claim 12, wherein at least one of X₇ to X₉ is N.

14. The organic compound according to claim 12, wherein at least one of X₇ and X₈ is N.

15. The organic compound according to claim 12, wherein one of X₇ to X₉ is N.

16. The organic compound according to claim 12, wherein one of X₇ and X₈ is N.

17. The organic compound according to claim 12, wherein only one of X₇ to X₉ is N.

18. The organic compound according to claim 12, wherein only one of X₇ and X₈ is N.

19. The organic compound according to claim 12, wherein X₈ is N.

20. The organic compound according to claim 12, wherein A represents a heteroaryl group including two heteroatoms of N.
